(19) United States
(12) Patent Application Publication
(10) Pub. No.: US 2023/0165863 A1
(43) Pub. Date: Jun. 1, 2023

(54) METHODS AND FORMULATIONS FOR ADMINISTRATION OF THIOCARBAMATE DERIVIATIVES A2A INHIBITORS

(71) Applicant: iTeos Belgium SA, Gosselies (BE)

(72) Inventors: Olivier De Henau, Watermael-Boitsfort (BE); Veronique Bodo, Gosselies (BE); Joanne Lager, Hollis, NH (US); Chiara Martinoli, Gosselies (BE); Manfred Schneider, Gosselies (BE)

(21) Appl. No.: 17/919,115

(22) PCT Filed: Apr. 16, 2021
(86) PCT No.: PCT/EP2021/059996

§ 371 Date: Oct. 14, 2022

Related U.S. Application Data
(60) Provisional application No. 63/011,155, filed on Apr. 16, 2020.

(51) Int. Cl. A61K 31/519

(52) U.S. Cl. CPC A61K 31/519

(57) ABSTRACT

The present invention relates to thiocarbamate derivatives of Formula (I) which are useful as A2A adenosine receptor (A2AR) inhibitors. Especially, the present invention relates to methods and formulations for treating and/or preventing cancers in a subject with a pharmaceutical composition comprising a thiocarbamate derivative as adenosine A2A receptor inhibitor. The pharmaceutical composition of the invention is particularly useful for oral dosing in the treatment of cancer.

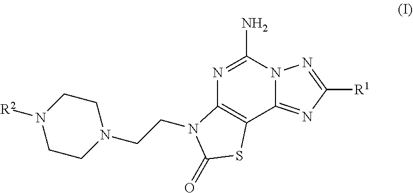

### FIELD OF INVENTION

The present invention relates to thiocarbamate derivatives which are useful as A2A adenosine receptor (A2AR) inhibitors.

Especially, the present invention relates to methods and formulations for treating cancers in a subject with a pharmaceutical composition comprising a thiocarbamate derivative as adenosine A2A receptor inhibitor. The pharmaceutical composition of the invention is particularly useful for oral dosing in the treatment of solid cancers.

### BACKGROUND OF INVENTION

Many of the immunosuppressive mechanisms in tumors are common to physiological immunoregulation in normal tissues. Such immunoregulation is very important in keeping the immune system under control in order to block a self-reactive immune response and to prevent an ongoing immune response from causing critical tissue damage. The lack of physiological immunoregulation often results in overwhelming immune activation that accompanies autoimmunity. For example, CTLA-4 is a physiological mechanism that negatively regulates T cell activity by blocking a costimulatory signal through CD28-B7 interaction. The lack of CTLA4 causes non-specific T cell activation, and CTLA-4-deficient mice die in several weeks with massive lymphocytic tissue infiltration. PD-1 also provides a T cell inhibitory signal upon interaction with its ligands, PD-L1 and PD-L2. Deficiency of PD-1 in mice is known to cause various types of autoimmune disorders depending on the genetic strains.

Besides cell surface transducers of immunosuppressive signal, e.g., CTLA-4 and PD-1, immunosuppression in the tumor microenvironment involves anti-inflammatory cytokines (IL-10, TGF-β), enzymes (indoleamine-2,3-dioxygenase), and professional immunoregulatory cells (regulatory T cells, myeloid-derived suppressor cells MDSCs). These immunosuppressive mechanisms play an important role in controlling immune response in normal tissues. Since tumors take advantage of such physiological immunoregulatory mechanisms to protect their tissue from immune attack, these mechanisms intended to prevent inflammatory complication, now turn out to be major obstacles hampering spontaneous cancer regression and immunological cancer treatment. The identification of immunosuppressive mechanisms in tumors pointed out molecular targets to restore the antitumor immune response. Thus, these negative immunoregulatory mechanisms, so-called immune checkpoints, became a focus in drug discovery. Antibodies against PD1, PDLL or CTLA4 have been approved as anticancer therapies on a large number of indications, such as Metastatic Melanoma, Non-Small Cell Lung Cancer, Renal Cell Carcinoma, Hodgkin's Lymphoma, Head and Neck Cancer, Urothelial Carcinoma, Hepatocellular Carcinoma, as well as treatment of for patients with solid tumors that have one of two specific genetic features known as mismatch repair deficiency and high microsatellite instability (irrespective of cancer type).

Extracellular adenosine has been known as an inhibitor of immune functions. While intracellular adenosine is involved in energy metabolism, nucleic acid metabolism, and the methionine cycle, extracellular adenosine plays an important role in intercellular signaling. Its signal is transmitted by G protein-coupled adenosine receptors on the cell surface, and it affects diverse physiological functions including neurological, cardiovascular, and immunological systems.

Tumors contain high levels of extracellular adenosine, suggesting that tumor cells may benefit from its immunosuppressive effect and catabolic energy production (Allard et al., Curr. Opin. Pharmacol., 2016, 29, 7-16; Otta A., Frontiers in Immunology, 2016, 7: 109). This high level of extracellular adenosine is probably due to overexpression of the enzyme CD73, which is responsible for production of extracellular adenosine. CD73 is overexpressed by a large number of tumors, with all the following tumors expressing medium or high levels of CD73 in >50% of tumor surface by immunohistochemistry (www.proteinatlas.org): Breast, Carcinoid, Cervical, Colorectal, Endometrial, Glioma, Head and Neck, Liver, Lung, Melanoma, Ovarian, Pancreatic, Prostate, Renal, Gastric, Thyroid, Urothelial.

Of the four known types of adenosine receptors, A2A adenosine receptor (A2AR) is the predominantly expressed subtype in most immune cells. Stimulation of A2AR generally provides an immunosuppressive signal that inhibits activities of T cells (proliferation, cytokine production, cytotoxicity), NK cells (cytotoxicity), NKT cells (cytokine production, CD40L upregulation), macrophages/dendritic cells (antigen presentation, cytokine production), and neutrophils (oxidative burst). The presence of high levels of extracellular adenosine in tumors was found to play a significant role in the evasion of antitumor immune response. Especially, it was shown that A2AR-deficient mice could spontaneously regress the inoculated tumor, whereas no wild-type mice showed similar tumor regression. A2AR antagonists were also beneficial in tumor-bearing wild-type animals Importantly, depletion of T cells and NK cells impaired the retardation of tumor growth by A2AR antagonists, suggesting improvement of antitumor cellular immune response. Effector functions of T cells and NK cells are susceptible to A2AR stimulation. In addition, when activated in the presence of A2AR agonist, the effector function of T cells is persistently impaired even after removal of A2AR agonist. This result suggests that the adenosine-rich environment in tumors may induce T cells that are anergic to the tumor cells.

Therefore, given that A2A receptor is expressed in most immune cells and particularly effector immune cells such as T cells and NK cells and given that A2A receptor is engaged in tissues where adenosine is produced, it is thought that A2A inhibitors can be helpful in all the cancer indications.

Consequently, there is a need for A2A inhibitors able to restore immune functions in tumors environment.

Adenosine is known to be an endogenous modulator of a number of other physiological functions. For example, at the central nervous system (CNS) level, adenosine in known to induce sedative, anxiolytic and antiepileptic effects level.

Thus, A2A inhibitors were previously developed for the treatment of depression and neurodegenerative diseases such as Parkinson's disease or Alzheimer's disease (Pinna A., CNS Drugs, 2014, 28, 455). One of the most advanced A2A inhibitors developed for the treatment of CNS diseases is Preladenant (Hodgson R A et al., J. Pharmacol. Exp. Ther., 2009, 330(1), 294-303; Hauser R A et al., JAMA Neurol., 2015, 72(12), 1491-500).

However, such previously developed A2A inhibitors were designed to cross the blood brain barrier, in order to target A2A receptor in the CNS.

Given the higher level of adenosine in tumors when compared to the brain, much higher amounts of compounds will be needed to achieve the desired effect on immune functions restoration for treating cancers. Thus, in order to avoid deleterious side effects, one should provide A2A inhibitors which have a limited, if any, CNS penetrance, contrary to all previously developed A2A inhibitors.

The Applicant provided a series of non-brain penetrant A2A inhibitors in international patent application PCT/EP2018/058301, being thiocarbamate derivatives, which are useful to restore immune functions in tumor environment.

Nevertheless, these compounds present a very low solubility in aqueous buffers, a low intestinal solubility and thus a low oral bioavailability. Consequently, there is a need for a pharmaceutical formulation of these compounds that would be suitable for oral administration. As evidenced in the experimental part below, the Applicant hereby provides a pharmaceutical composition that enables suitable oral bioavailability of the thiocarbamates A2A inhibitors.

In addition, the Applicant thereby provides a method for administration of a pharmaceutical composition comprising the thiocarbamates A2A inhibitors. Finding a proper method for the administration of the said compositions is an essential step in the development of anticancer drugs.

### SUMMARY

Described herein a method of treating a subject having cancer comprising administering to the subject a pharmaceutical composition comprising (a) therapeutically effective amount of compound of Formula (I) or a pharmaceutically acceptable salt or solvate thereof,

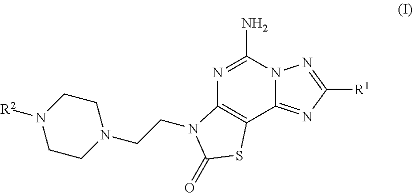

wherein:

the therapeutically effective amount is from about 20 mg per day to about 320 mg per day;

R¹ represents 5- or 6-membered heteroaryl or 5- or 6-membered aryl, wherein heteroaryl or aryl groups are optionally substituted by one or more substituent selected from C1-C6 alkyl (preferably methyl) and halo (preferably fluoro or chloro); preferably R¹ represents 5-membered heteroaryl; more preferably R¹ represents furyl;

R² represents 6-membered aryl or 6-membered heteroaryl,

wherein heteroaryl or aryl groups are optionally substituted by one or more substituent selected from halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino and alkylsulfonealkyl;

said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;

or the heteroaryl or aryl groups are optionally substituted with two substituents that form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;

(b) one or more pharmaceutically acceptable carrier, diluent, excipient and/or adjuvant, and

(c) optionally a lipid carrier.

In some embodiments, the compound is a compound of Formula (Ia):

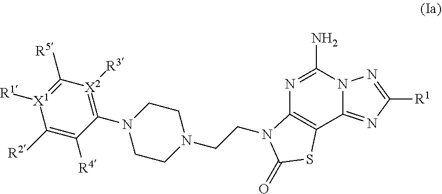

or a pharmaceutically acceptable salt or solvate thereof, wherein:

R¹ is as defined in claim 1;

X¹ and X² represent each independently C or N;

R^{1′} is absent when X¹ is N; or when X¹ is C, R^{1′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino or alkylsulfonealkyl;

said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;

R^{2′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino, or alkylsulfonealkyl;

said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;

or R^{1′} and R^{2′} form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;

R^{3′} is absent when X² is N; or when X² is C, R^{3′} represents H or halo, preferably H or F;

R^{4′} represents H or halo, preferably H or F; and

R^{5′} represents H or halo, preferably H or F. In some embodiments, the compound is a compound of Formula (Ia-1)

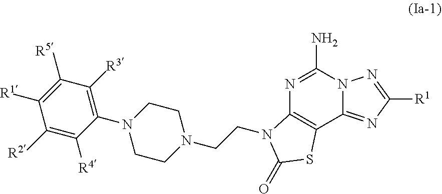

or a pharmaceutically acceptable salt or solvate thereof, wherein R¹, R^{1′}, R^{2′}, R^{3′}, R^{4′} and R^{5′} are as defined in claim 2.

In some embodiments, the compound is a compound of Formula (Ia-1a)

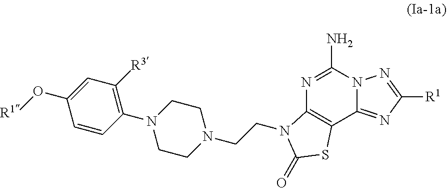

or a pharmaceutically acceptable salt or solvate thereof, wherein:

R¹ and R^{3′} are as defined in claim 2; and

R^{1″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In another embodiment, the compound is of a compound of Formula (Ia-1b)

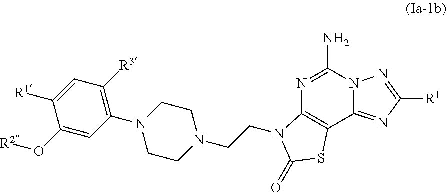

or a pharmaceutically acceptable salt or solvate thereof, wherein:

R¹ and R^{3′} are as defined in claim 2;

R^{1′} represents H or halo, preferably H or F; and

R^{2″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In other embodiments, the compound is of a compound of Formula (Ia-1c) or (Ia-1d)

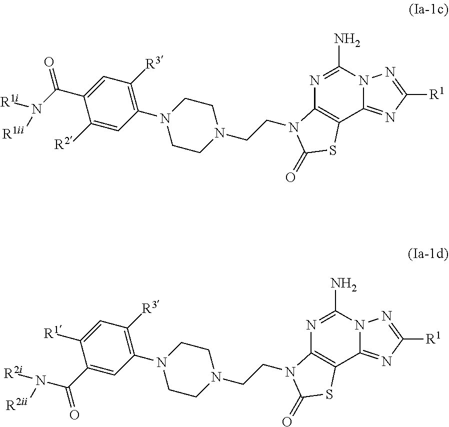

or a pharmaceutically acceptable salt or solvate thereof, wherein:

R¹ and R^{3′} are as defined in claim 2;

R^{1′} represents H or halo, preferably H or F;

R^{2′} represents H or halo, preferably H or F;

R¹ⁱ and R¹ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl; and

R²ⁱ and R²ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl.

In some embodiments, the compound of Formula (I) is selected from the group consisting of:

3-(2-(4-(4-((1H-1,2,3-triazolo-4yl)methoxy-2fluorophenyl)piperazine-1-yl)ethyl)-5-amino-(8-(furan-2-yl)thiazolo[5,4-e][1,2,4]triazolo[1,5-c]pyrimidine-2(3H)-one;

5-((4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)methyl)-1,3,4-oxadiazol-2(3H)-one;

5-amino-3-(2-(4-(3-fluoropyridin-4-yl)piperazin-1-yl)ethyl)-8-(furan-2-yl)thiazolo[5,4-e][1,2,4]triazolo[1,5-c]pyrimidin-2(3H)-one;

2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)acetamide;

(S)-5-amino-3-(2-(4-(2-fluoro-4-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;

(R)-5-amino-3-(2-(4-(2-fluoro-4-(2-(methylsulfinyl)ethoxy)phenyl)-piperazin-1-yl)ethyl)-8-(furan-2-yl)thiazolo[5,4-e][1,2,4]triazolo[1,5-c]pyrimidin-2(3H)-one;

(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;

(R)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;

(S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;

5-amino-8-(furan-2-yl)-3-(2-(4-(4-(2-hydroxyethoxy)phenyl)piperazin-1-yl)ethyl)thiazolo[5,4-e][1,2,4]triazolo[1,5-c]pyrimidin-2(3H)-one;

2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)phenoxy)acetic acid;

2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)phenoxy)acetamide;

5-amino-3-(2-(4-(4-(2,3-dihydroxypropoxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(4-(2-aminoethoxy)phenyl)piperazin-1-yl)ethyl)-8-(furan-2-yl)thiazolo[5,4-e][1,2,4]triazolo[1,5-c]pyrimidin-2(3H)-one;

4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)benzamide;

4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-methylbenzamide;

5-amino-8-(furan-2-yl)-3-(2-(4-(4-(2-morpholinoethoxy)phenyl)piperazin-1-yl)ethyl)thiazolo[5,4-e][1,2,4]triazolo[1,5-c]pyrimidin-2(3H)-one;

5-amino-3-(2-(4-(4-(2-(dimethylamino)ethoxy)phenyl)piperazin-1-yl)ethyl)-8-(furan-2-yl)thiazolo[5,4-e][1,2,4]triazolo[1,5-c]pyrimidin-2(3H)-one;

4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)benzenesulfonamide;

4-(4-(2-(5-amino-8-(furan-2-yl)-2-oxothiazolo[5,4-e][1,2,4]triazolo[1,5-c]pyrimidin-3(2H)-yl)ethyl) piperazin-1-yl)-N-methylbenzenesulfonamide;

5-amino-8-(furan-2-yl)-3-(2-(4-(4-(methylsulfonyl)phenyl)piperazin-1-yl)ethyl)thiazolo[5,4-e][1,2,4]triazolo[1,5-c]pyrimidin-2(3H)-one;

5-amino-8-(furan-2-yl)-3-(2-(4-(4-(methylsulfinyl)phenyl)piperazin-1-yl)ethyl)thiazolo[5,4-e][1,2,4]triazolo[1,5-c]pyrimidin-2(3H)-one;

3-(4-(2-(5-amino-8-(furan-2-yl)-2-oxothiazolo[5,4-e][1,2,4]triazolo[1,5-c]pyrimidin-3(2H)-yl)ethyl)piperazin-1-yl)benzamide;

5-amino-8-(furan-2-yl)-3-(2-(4-(3-(2-hydroxyethoxy) phenyl)piperazin-1-yl)ethyl)thiazolo[5,4-e][1,2,4]triazolo[1,5-c]pyrimidin-2(3H)-one;

5-amino-3-(2-(4-(2-fluoro-4-(2-oxo-2-(piperazin-1-yl)ethoxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2-fluoro-4-(piperidin-4-ylmethoxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2-fluoro-4-(piperazine-1-carbonyl)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2-fluoro-4-(2-(piperazin-1-yl)ethoxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2-fluoro-4-(piperazin-1-ylsulfonyl)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2-fluoro-4-(methylsulfonyl)phenyl) piperazin-1-yl)ethyl)-8-(furan-2-yl)thiazolo[5,4-e][1,2,4]triazolo[1,5-c]pyrimidin-2(3H)-one;

4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-aminoethyl)-3-fluorobenzamide;

4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylamino)ethyl)benzamide;

4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-3-fluorobenzamide;

4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-hydroxyethyl)benzamide;

4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-3-fluorobenzamide;

2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)acetic acid;

2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3,5-difluorophenoxy)acetic acid;

2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)propanoic acid;

(S)-2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)propanoic acid;

2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-2-methylpropanoic acid;

3-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenyl)propanoic acid;

4-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)butanoic acid;

2-(3-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,6-difluorophenoxy)acetic acid;

2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)acetic acid;

4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorobenzoic acid;

2-((2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)ethyl)amino)acetamide;

2-((2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)ethyl)(methyl)amino)acetamide;

5-amino-3-(2-(4-(2-fluoro-4-(piperidin-4-yloxy) phenyl)piperazin-1-yl)ethyl)-8-(furan-2-yl) thiazolo[5,4-e][1,2,4]triazolo[1,5-c]pyrimidin-2(3H)-one;

5-amino-3-(2-(4-(2-fluoro-4-(pyrrolidin-3-yloxy)phenyl)piperazin-1-yl)ethyl)-8-(furan-2-yl)thiazolo[5,4-e][1,2,4]triazolo[1,5-c]pyrimidin-2(3H)-one;

3-(2-(4-(4-((1H-1,2,4-triazol-3-yl)methoxy)-2-fluorophenyl)piperazin-1-yl)ethyl)-5-amino-8-(furan-2-yl)thiazolo[5,4-e][1,2,4]triazolo[1,5-c]pyrimidin-2(3H)-one;

2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-(2-(methylamino)ethyl)acetamide;

2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-(2-(dimethylamino)ethyl)acetamide;

2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-(2-aminoethyl)acetamide;

(R)-2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)propanoic acid;

2-(4-(4(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)acetamide;

4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-methyl-N-(2-(methylamino)ethyl)benzamide;

4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-3-fluoro-N-methylbenzamide;

(R)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(1-(dimethylamino)propan-2-yl)-3-fluorobenzamide;

2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-methyl-N-(2-(methylamino)ethyl)acetamide;

2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-2-methylpropanoic acid;

(S)-2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)propanoic acid;

(R)-2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)propanoic acid;

2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-N-(2-(methylamino)ethyl)acetamide;

2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-N-(2-(dimethylamino)ethyl)acetamide;

5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-2,4-difluoro-N-methylbenzamide;

4-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)butanoic acid;

3-(2-(4-(5-((1H-tetrazol-5-yl)methoxy)-2,4-difluorophenyl)piperazin-1-yl)ethyl)-5-amino-8-(furan-2-yl)thiazolo[5,4-e][1,2,4]triazolo[1,5-c]pyrimidin-2(3H)-one;

5-amino-3-(2-(4-(2-fluoro-4-((1-methyl-1H-1,2,4-triazol-3-yl)methoxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2,4-difluoro-5-((1-methyl-1H-1,2,4-triazol-3-yl)methoxy)phenyl)piperazin-1-yl)ethyl)-8-(furan-2-yl)thiazolo[5,4-e][1,2,4]triazolo[1,5-c]pyrimidin-2(3H)-one;

4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methyl(oxetan-3-yl)amino)ethyl)benzamide;

4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-((2-hydroxyethyl)amino)ethyl)benzamide;

2-amino-N-(2-(4-(4-(2-(5-amino-8-(furan-2-yl)-2-oxothiazolo[5,4-e][1,2,4]triazolo[1,5-c] pyrimidin-3(2H)-yl)ethyl)piperazin-1-yl)-3-fluorophenoxy)ethyl)acetamide;

(S)-2-amino-N-(2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)ethyl)-3-methylbutanamide;

ethyl 2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)acetate;

2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)acetonitrile;

5-amino-8-(furan-2-yl)-3-(2-(4-(pyridin-4-yl)piperazin-1-yl)ethyl)thiazolo[5,4-e][1,2,4]triazolo[1,5-c]pyrimidin-2(3H)-one;

5-amino-8-(furan-2-yl)-3-(2-(4-(pyrimidin-4-yl)piperazin-1-yl)ethyl)thiazolo[5,4-e][1,2,4]triazolo[1,5-c]pyrimidin-2(3H)-one;

5-amino-3-(2-(4-(2,4-difluoro-5-(2-(methylsulfonyl)ethoxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2-fluoro-4-(2-(methylsulfonyl)ethoxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(6-fluoro-2-oxoindolin-5-yl)piperazin-1-yl)ethyl)-8-(furan-2-yl)thiazolo[5,4-e][1,2,4]triazolo[1,5-c]pyrimidin-2(3H)-one;

5-amino-3-(2-(4-(2-fluoro-4-(S-methylsulfonimidoyl)phenyl)piperazin-1-yl)ethyl)-8-(furan-2-yl)thiazolo[5,4-e][1,2,4]triazolo[1,5-c]pyrimidin-2(3H)-one;

5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-2,4-difluorobenzamide;

5-amino-3-(2-(4-(5-fluoro-2-methylpyridin-4-yl)piperazin-1-yl)ethyl)-8-(furan-2-yl)thiazolo[5,4-e][1,2,4]triazolo[1,5-c]pyrimidin-2(3H)-one;

5-amino-3-(2-(4-(2-fluoro-4-(((3R,4R)-4-hydroxytetrahydrofuran-3-yl)oxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2-fluoro-4-(((3S,4S)-4-hydroxytetrahydrofuran-3-yl)oxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2-fluoro-4-(2-hydroxy-2-methylpropoxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2-fluoro-4-(2-hydroxypropan-2-yl)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2-fluoro-4-(3,3,3-trifluoro-2-hydroxypropoxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2-fluoro-5-(2-hydroxyethoxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2,4-difluoro-5-(morpholin-2-ylmethoxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2,4-difluoro-5-(morpholin-3-ylmethoxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2,4-difluoro-5-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2,4-difluoro-5-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2,4-difluoro-5-(((3R,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2,4-difluoro-5-(((3S,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;

(S)-5-amino-3-(2-(4-(2,4-difluoro-5-((2-oxopyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;

(R)-5-amino-3-(2-(4-(2,4-difluoro-5-((2-oxopyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;

2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-N-(2-morpholinoethyl)acetamide;

5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(morpholin-3-ylmethyl)benzamide;

5-amino-3-(2-(4-(2-fluoro-4-(morpholin-3-ylmethoxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2-fluoro-4-(morpholin-2-ylmethoxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2-fluoro-4-(((3R,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2-fluoro-4-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2-fluoro-4-(((3R,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2-fluoro-4-(((3S ,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;

2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-(2-morpholinoethyl)acetamide;

4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-morpholinoethyl)benzamide;

4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(morpholin-3-ylmethyl)benzamide;

5-amino-3-(2-(4-(4-(azetidin-3-yloxy)phenyl)piperazin-1-yl)ethyl)-8-(furan-2-yl)thiazolo[5,4-e][1,2,4]triazolo[1,5-c]pyrimidin-2(3H)-one;

(S)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one;

(R)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2,4-difluoro-5-(((1s,4s)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2,4-difluoro-5-(((1r,4r)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one;

(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(2-(methylsulfinyl)ethyl)benzamide;

(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(2-(methylsulfinyl)ethyl)benzamide;

(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-methyl-N-(2-(methylsulfinyl)ethyl)benzamide;

(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-methyl-N-(2-(methylsulfinyl)ethyl)benzamide;

5-amino-3-(2-(4-(2,4-difluoro-5-(1-oxidothiomorpholine-4-carbonyl)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2,4-difluoro-5-(1-oxidothiomorpholino)phenyl)piperazin-1-yl)ethyl)-8-(furan-2-yl)thiazolo[5,4-e][1,2,4]triazolo[1,5-c]pyrimidin-2(3H)-one;

(R)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one;

(S)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2-fluoro-4-(((1s,4s)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2-fluoro-4-(((1r,4r)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one;

(S)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide;

(R)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide;

5-amino-3-(2-(4-(2-fluoro-4-(1-oxidothiomorpholine-4-carbonyl)phenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(2-fluoro-4-(1-oxidothiomorpholino)phenyl)piperazin-1-yl)ethyl)-8-(furan-2-yl)thiazolo[5,4-e][1,2,4]triazolo[1,5-c]pyrimidin-2(3H)-one;

(S)-5-amino-3-(2-(4-(5-(2,3-dihydroxypropoxy)-2,4-difluorophenyl)piperazin-1-yl)ethyl)-8-(furan-2-yl)thiazolo[5,4-e][1,2,4]triazolo[1,5-c]pyrimidin-2(3H)-one;

(R)-5-amino-3-(2-(4-(5-(2,3-dihydroxypropoxy)-2,4-difluorophenyl)piperazin-1-yl)ethyl)-8-(furan-2-yl)thiazolo[5,4-e][1,2,4]triazolo[1,5-c]pyrimidin-2(3H)-one;

(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-2,4-difluorobenzamide;

(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-2,4-difluorobenzamide;

5-amino-3-(2-(4-(4-(azetidin-3-yloxy)-2-fluorophenyl)piperazin-1-yl)ethyl)-8-(furan-2-yl)thiazolo[5,4-e][1,2,4]triazolo[1,5-c]pyrimidin-2(3H)-one;

5-amino-3-(2-(4-(5-(azetidin-3-yloxy)-2,4-difluorophenyl)piperazin-1-yl)ethyl)-8-(furan-2-yl)thiazolo[5,4-e][1,2,4]triazolo[1,5-c]pyrimidin-2(3H)-one;

(S)-5-amino-3-(2-(4-(2,4-difluoro-5-(3-(methylsulfinyl)propoxy)phenyl)piperazin-1-yl)ethyl)-8-(furan-2-yl)thiazolo[5,4-e][1,2,4]triazolo[1,5-c]pyrimidin-2(3H)-one;

and pharmaceutically acceptable salts or solvates thereof.

In a particular embodiment, the compound of Formula (I) is selected from:

(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one; and

(S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one.

5-amino-3-(2-(4-(2,4-difluoro-5-(2-(methylsulfonyl)ethoxy)phenyl)piperazin-1-yl)ethyl)-8-(furan-2-yl)thiazolo[5,4-e][1,2,4]triazolo[1,5-c]pyrimidin-2(3H)-one

In some embodiments, the lipid carrier comprises lauroyl polyoxyl-32 glycerides. In some embodiments, lauroyl polyoxyl-32 glycerides is present in an amount ranging from 55% to 99% w/w, preferably from 60% to 95% w/w, more preferably from 70% to 85% w/w.

In some embodiments, the pharmaceutical composition is administered in the form of a tablet, a suspension, granules or a capsule.

In some embodiments, the pharmaceutical composition is administered orally.

In some embodiments, the therapeutically effective amount is administered in separate administrations of 2, 3, 4, or 6 equal doses. In another embodiment, the therapeutically effective amount is administered as a single dose. In another embodiment, the therapeutically effective amount is about 160 mg per day, wherein the therapeutically effective amount is administered in separate administrations of 2, 3, 4, or 6 equal doses.

In a particular embodiment, the therapeutically effective amount is about 160 mg per day, wherein the therapeutically effective amount is administered in separate administrations of 2 equal doses, whereby each dose is about 80 mg.

In a particular embodiment, the subject is a human In some embodiments, the human subject is older than 18 years of age.

In some embodiments, the human subject has a confirmed metastatic solid tumor. In some embodiments, the metastatic solid tumor is confirmed by international RECIST v1.1 criteria.

In some embodiments, the pharmaceutical composition is administered to the subject for at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the subject.

In some embodiments, the method according to the invention further comprises orally administering a beverage with a pH lower than or equal to about 3, about 5, or about 7. In some embodiments, the beverage and the pharmaceutical composition according to the invention are to be administered to a subject in need thereof sequentially and/or concurrently.

In some embodiments, the method of the invention further comprises administering a gastric acid secretion stimulating agent prior to administering the pharmaceutical composition. In some embodiments, the gastric acid secretion stimulating agent comprises pentagastrin.

In some embodiments, the level of pCREB in T-cells of the subject is decreased after administering to the subject the pharmaceutical composition according to the invention.

In another embodiment, the level of TNFα in the subject is increased after administering to the subject the pharmaceutical composition according to the invention.

In some embodiments, wherein the salt of the compound according to the invention is the hydrochloride or esylate salt.

In some embodiments, the pharmaceutical composition according to the invention further comprises PEG 400, PEG 2000 and/or caprylic acid.

In some embodiments the pharmaceutical composition according to the invention further comprises an antioxidant; preferably the antioxidant is selected from butylated hydroxytoluene (BHT), butylated hydroxyanisole (BHA), citric acid, sodium metabisulfite, ascorbic acid, methionine and vitamin E; more preferably the antioxidant is BHT.

In some embodiments, the pharmaceutical composition according to the invention further comprises a wetting agent; preferably the wetting agent is selected from sodium lauryl sulphate, vitamin E TPGS, sodium docusate, polysorbate 80 and poloxamer 407; more preferably the wetting agent is sodium lauryl sulphate.

In some embodiments, the pharmaceutical composition according to the invention further comprises a precipitation inhibitor, preferably the precipitation inhibitor is selected from hydroxypropylmethylcellulose, methylcellulose, polyvinylpyrrolidone, polyvinylpyrrolidone polyvinylacetate copolymer; more preferably hydroxypropylmethylcellulose.

In some embodiments, the compound of Formula (I) is present in an amount ranging from 1% to 20% w/w, preferably from 5% to 15% w/w, more preferably about 10% w/w.

In one embodiment, the pharmaceutical composition according to the invention is formulated as capsules, wherein the capsule shells are constructed from gelatin and wherein additional components are optionally included in the capsule shells, such as for example polyethylene glycol and sodium lauryl sulphate.

In some embodiments, the cancer is selected from breast, bladder, carcinoid, cervical, colorectal, endometrial, glioma, head and neck, liver, lung, melanoma, ovarian, pancreatic, prostate, metastatic castrate resistant prostate, renal, gastric, sarcoma, thyroid and urothelial cancers.

In some embodiments, the method according to the invention further comprises administering at least one other pharmaceutically active ingredient. In one embodiment, the other pharmaceutically active ingredient is selected from anticancer agents. In a particular embodiment, the anticancer agent comprises pembrolizumab.

In one aspect, the anticancer agent is selected from immunotherapeutic agents, chemotherapeutic agents, antiangiogenic agents, multidrug resistance-associated proteins inhibitors, radiotherapeutic agents, and any combination thereof.

In one aspect, the immunotherapeutic agent is selected from checkpoint inhibitors, checkpoint agonists, IDO inhibitors, PI3K inhibitors, adenosine receptor inhibitors, adenosine-producing enzymes inhibitors, immune cells, therapeutic vaccines, or any combination thereof.

In one aspect, the checkpoint inhibitor is an inhibitor of a checkpoint protein selected from PD-1, PD-L1, CTLA-4 and TIGIT. In another aspect, the inhibitor of PD-1 is an anti-PD-1 antibody; the inhibitor of PD-L1 is an anti-PD-L1 antibody; the inhibitor of CTLA-4 is an anti-CTLA-4 antibody and the inhibitor of TIGIT is an anti-TIGIT antibody.

In some embodiments, the chemotherapeutic agent is selected from anticancer alkylating agents, anticancer antimetabolites, anticancer antibiotics, plant-derived anticancer agents, anticancer platinum coordination compounds and any combination thereof.

In a particular embodiment, the anticancer antibiotic is doxorubicin; and the anticancer platinum coordination compound is oxaliplatin.

In one aspect, the anticancer agent and the pharmaceutical composition according to the invention are to be administered to a patient in need thereof sequentially and/or concurrently.

In one aspect, the anticancer agent and the pharmaceutical composition according to the invention are to be administered to a subject in need thereof via different administration routes.

Definitions

In the present invention, the following terms have the following meanings:

The term “aldehyde” refers to a group —CHO.

The term “alkenyl” refers to unsaturated hydrocarbyl group, which may be linear or branched, comprising one or more carbon-carbon double bonds. Suitable alkenyl groups comprise between 2 and 6 carbon atoms, preferably between 2 and 4 carbon atoms, still more preferably between 2 and 3 carbon atoms. Examples of alkenyl groups are ethenyl, 2-propenyl, 2-butenyl, 3-butenyl, 2-pentenyl and its isomers, 2-hexenyl and its isomers, 2,4-pentadienyl and the like.

The term “alkenylcarbonyl” refers to a group —(C═O)-alkenyl wherein alkenyl is as herein defined.

The term “alkenylcarbonylamino” refers to a group —NH—(C═O)-alkenyl wherein alkenyl is as herein defined.

The term “alkoxy” refers to a group —O-alkyl wherein alkyl is as herein defined.

The term “alkyl” refers to a hydrocarbyl radical of formula CₙH₂ₙ₊₁ wherein n is a number greater than or equal to 1. Generally, alkyl groups of this invention comprise from 1 to 8 carbon atoms, more preferably, alkyl groups of this invention comprise from 1 to 6 carbon atoms. Alkyl groups may be linear or branched. Suitable alkyl groups include methyl, ethyl, propyl, butyl, pentyl, hexyl, heptyl and octyl.

The term “alkylaminoalkyl” refers to a group -alkyl-NH-alkyl wherein alkyl is as herein defined.

The term “alkylaminoalkylaminocarbonyl” refers to a group —(C═O)—NH-alkyl-NH-alkyl wherein alkyl is as herein defined.

The term “(alkylaminoalkyl)(alkyl)aminocarbonyl” refers to a group —(C═O)—NR¹R² wherein R¹ is an alkyl group and R² is a -alkyl-NH-alkyl group, wherein alkyl is as herein defined.

The term “alkylaminoalkylcarbonyl” refers to a group —(C═O)-alkyl-NH-alkyl wherein alkyl is as herein defined.

The term “alkylcarbonyl” refers to a group —(C═O)-alkyl wherein alkyl is as herein defined.

The term “alkylheteroaryl” refers to any heteroaryl substituted by an alkyl group wherein alkyl is as herein defined.

The term “alkyloxycarbonyl” refers to a group —(C═O)—O-alkyl wherein alkyl is as herein defined.

The term “alkylsulfonyl” refers to a group —SO₂-alkyl wherein alkyl is as herein defined.

The term “alkylsulfonealkyl” refers to a group -alkyl-SO₂-alkyl wherein alkyl is as herein defined.

The term “alkylsulfonimidoyl” refers to a group —S(═O)(═NH)-alkyl wherein alkyl is as herein defined.

The term “alkylsulfoxide” refers to a group —(S═O)-alkyl wherein alkyl is as herein defined.

The term “alkylsulfoxidealkyl” refers to a group -alkyl-SO-alkyl wherein alkyl is as herein defined.

The term “alkyne” refers to a class of monovalent unsaturated hydrocarbyl groups, wherein the unsaturation arises from the presence of one or more carbon-carbon triple bonds. Alkynyl groups typically, and preferably, have the same number of carbon atoms as described above in relation to alkyl groups. Non-limiting examples of alkynyl groups are ethynyl, 2-propynyl, 2-butynyl, 3-butynyl, 2-pentynyl and its isomers, 2-hexynyl and its isomers-and the like.

The term “alkynealkyl” refers to a group -alkyl-alkyne wherein alkyl and alkyne are as herein defined.

The term “amino” refers to a group —NH₂.

The term “aminoalkyl” refers to a group -alkyl-NH₂ wherein alkyl is as herein defined.

The term “aminoalkylaminocarbonyl” refers to a group —(C═O)—NH-alkyl-NH₂ wherein alkyl is as herein defined.

The term “aminoalkylcarbonylamino” refers to a group —NH—(C═O)-alkyl-NH₂ wherein alkyl is as herein defined.

The term “aminocarbonyl” refers to a group —(C═O)—NH₂.

The term “(aminocarbonylalkyl)(alkyl)amino” refers to a group —NR¹R² wherein R¹ is an alkyl group and R² is a -alkyl-(C═O)—NH₂ group, wherein alkyl is as herein defined.

The term “aminocarbonylalkylamino” refers to a group —NH-alkyl-(C═O)—NH₂ wherein alkyl is as herein defined.

The term “aminosulfonyl” refers to a group —SO₂—NH₂.

The term “antioxidant” refers to an agent that diminishes or avoids the oxidation of other substances.

The term “aryl” refers to a polyunsaturated, aromatic hydrocarbyl group having a single ring (i.e. phenyl) or multiple aromatic rings fused together (e.g. naphtyl), typically containing 5 to 12 atoms; preferably 5 to 10; more preferably the aryl is a 5- or 6-membered aryl. Non-limiting examples of aryl comprise phenyl, naphthalenyl.

The term “carbonyl” refers to a group —(C═O)—.

The term “carbonylamino” refers to a group —NH—(C═O)—.

The term “cycloalkyl” refers to a cyclic alkyl group, that is to say, a monovalent, saturated, or unsaturated hydrocarbyl group having 1 or 2 cyclic structures. Cycloalkyl includes monocyclic or bicyclic hydrocarbyl groups. Cycloalkyl groups may comprise 3 or more carbon atoms in the ring and generally, according to this invention comprise from 3 to 10, more preferably from 3 to 8 carbon atoms; still more preferably more preferably the cycloalkyl is a 5- or 6-membered cycloalkyl. Examples of cycloalkyl groups include but are not limited to cyclopropyl, cyclobutyl, cyclopentyl, cyclohexyl.

The term “cycloalkyloxy” refers to a group —O-cycloalkyl wherein cycloalkyl is as herein defined.

The term “dialkylamino” refers to a group —NR¹R² wherein R¹ and R² are both independently alkyl group as herein defined.

The term “dialkylaminoalkyl” refers to a group -alkyl-NR¹R² wherein R¹ and R² are both independently alkyl group, as herein defined.

The term “dialkylaminoalkylaminocarbonyl” refers to a group —(C═O)—NH-alkyl-NR¹R² wherein R¹ and R² are both alkyl group, as herein defined.

The term “dialkylaminoalkylcarbonyl” refers to a group —(C═O)-alkyl-NR¹R² wherein R¹ and R² are both alkyl group, as herein defined.

The term “dihydroxyalkyl” refers to a group alkyl is as herein defined substituted by two hydroxyl (—OH) groups.

The term “halo” or “halogen” refers to fluoro, chloro, bromo, or iodo.

The term “heteroaryl” refers to an aryl group as herein defined wherein at least one carbon atom is replaced with a heteroatom. In other words, it refers to 5 to 12 carbon-atom aromatic single rings or ring systems containing 2 rings which are fused together, typically containing 5 to 6 atoms; in which one or more carbon atoms is replaced by oxygen, nitrogen and/or sulfur atoms where the nitrogen and sulfur heteroatoms may optionally be oxidized and the nitrogen heteroatoms may optionally be quaternized. Non-limiting examples of such heteroaryl, include: oxazolyl, thiazolyl, imidazolyl, furanyl and pyrrolyl. Preferably the heteroaryl is a 5- or 6-membered heteroaryl, more preferably the 5- or 6-membered heteroaryl is a furyl.

The term “heterocyclyl” refers to non-aromatic, fully saturated or partially unsaturated cyclic groups (for example, 3 to 7 member monocyclic, 7 to 11 member bicyclic, or containing a total of 3 to 10 ring atoms) which have at least one heteroatom in at least one carbon atom-containing ring. Preferably the heterocyclyl is a 5- or 6-membered heterocyclyl. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valence allows. The rings of multi-ring heterocycles may be fused, bridged and/or joined through one or more spiro atoms. Non limiting exemplary heterocyclic groups include aziridinyl, oxiranyl, thiiranyl, piperidinyl, azetidinyl, 2-imidazolinyl, pyrazolidinyl imidazolidinyl, isoxazolinyl, oxazolidinyl, isoxazolidinyl, thiazolidinyl, isothiazolidinyl, piperidinyl, succinimidyl, 3H-indolyl, indolinyl, isoindolinyl, 2H-pyrrolyl, 1-pyrrolinyl, 2-pyrrolinyl, 3-pyrrolinyl, pyrrolidinyl, 4H-quinolizinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, oxetanyl, thietanyl, 3-dioxolanyl, 1,4-dioxanyl, 2,5-dioximidazolidinyl, 2-oxopiperidinyl, 2-oxopyrrolodinyl, indolinyl, tetrahydropyranyl, tetrahydrofuranyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, 1-oxido-1-thiomorpholin-4-yl, 1-dioxido-1-thiomorpholin-4-yl, 1,3-dioxolanyl, 1,4-oxathianyl, 1,4-dithianyl, 1,3,5-trioxanyl, 1H-pyrrolizinyl, tetrahydro-1,1-dioxothiophenyl, N-formylpiperazinyl, and morpholin-4-yl.

The term “heterocyclylalkylaminocarbonyl” refers to a group —(C═O)—NH-alkyl-heterocyclyl, wherein alkyl and heterocyclyl are as herein defined.

The term “(heterocyclyl)(alkyl)aminoalkyl” refers to a group -alkyl-NR¹R² wherein R¹ is an alkyl group and R² is a heterocyclyl group, wherein alkyl and heterocyclyl are as herein defined.

The term “heterocyclylcarbonyl” refers to a group —(C═O)-heterocyclyl wherein heterocyclyl is as herein defined.

The term “heterocyclylalkyl” refers to a group -alkyl-heterocyclyl wherein alkyl and heterocyclyl are as herein defined.

The term “heterocyclyloxy” to a group —O-heterocyclyl wherein heterocyclyl is as herein defined.

The term “heterocyclylsulfonyl” refers to a group —SO₂-heterocyclyl wherein heterocyclyl is as herein defined.

The term “hydroxyalkyl” refers to a group -alkyl-OH wherein alkyl is as herein defined.

The term “hydroxyalkylaminoalkyl” refers to a group -alkyl-NH-alkyl-OH wherein alkyl is as herein defined.

The term “hydroxycarbonyl” refers to a group —C(═O)—OH wherein carbonyl is as herein defined.

In other words, “hydroxycarbonyl” corresponds to a carboxylic acid group.

The term “oxo” refers to a ═O substituent.

The term “sulfonylamino” refers to a group —NH—SO₂.

The term “about”, preceding a figure encompasses plus or minus 10%, or less (such as plus or less 1%), of the value of said figure. It is to be understood that the value to which the term “about” refers is itself also specifically, and preferably, disclosed.

The term “administration”, or a variant thereof (e.g. “administering”), means providing the active agent or active ingredient (e.g. an A2AR inhibitor or an anticancer agent), alone or as part of a pharmaceutically acceptable composition, to the patient in whom/which the condition, symptom, or disease is to be treated or prevented.

The term “autologous” refers to any material derived from the same individual to whom it is later to be re-introduced.

The term “allogenic” refers to any material derived from a different individual of the same specie as the individual to whom the material is introduced. Two or more individuals are said to be allogenic to one another when the genes at one or more loci are not identical. In some aspects, allogenic material from individuals of the same species may be sufficiently unlike genetically to interact antigenically.

The terms “IC₅₀” or “half maximal inhibitory concentration” represent the concentration of an inhibitor that is required for 50% inhibition in vitro.

The term “inhibitor” refers to a natural or synthetic compound that has a biological effect to inhibit or significantly reduce or down-regulate the expression of a gene and/or a protein or that has a biological effect to inhibit or significantly reduce the biological activity of a protein. Consequently, an “A2AR inhibitor” refers to a compound that has a biological effect to inhibit or significantly reduce or down-regulate the biological activity of A2A receptor.

The term “human” refers to a subject of both genders and at any stage of development (i.e. neonate, infant, juvenile, adolescent, adult).

The term “patient” refers to a warm-blooded animal or a mammal, more preferably a human, who/which is awaiting the receipt of, or is receiving medical care or is/will be the object of a medical procedure.

The expression “pharmaceutically acceptable” refers to the ingredients of a pharmaceutical composition are compatible with each other and not deleterious to the subject to which it is administered.

The expression “pharmaceutically acceptable carrier, diluent, excipient and/or adjuvant” refers to a substance that does not produce an adverse, allergic or other untoward reaction when administered to an animal, preferably a human. It includes any and all inactive substances such as for example solvents, cosolvents, antioxidants, surfactants, stabilizing agents, emulsifying agents, buffering agents, pH modifying agents, preserving agents (or preservating agents), antibacterial and antifungal agents, isotonifiers, granulating agents or binders, lubricants, disintegrants, glidants, diluents or fillers, adsorbents, dispersing agents, suspending agents, coating agents, bulking agents, release agents, absorption delaying agents, sweetening agents, flavoring agents and the like. For human administration, preparations should meet sterility, pyrogenicity, general safety and purity standards as required by regulatory offices, such as, e.g., FDA Office or EMA.

The term “predrug”, as used herein, means any compound that will be modified to form a drug species, wherein the modification may take place either inside or outside of the body, and either before or after the predrug reaches the area of the body where administration of the drug is indicated.

The terms “prevent”, “preventing” and “prevention”, as used herein, refer to a method of delaying or precluding the onset of a condition or disease and/or its attendant symptoms, barring a patient from acquiring a condition or disease, or reducing a patient's risk of acquiring a condition or disease.

The term “prodrug” as used herein means the pharmacologically acceptable derivatives of compounds of Formula (I), such as for example esters or amides, whose in vivo biotransformation product generates the biologically active drug. Prodrugs are generally characterized by increased bio-availability and are readily metabolized into biologically active compounds in vivo.

The terms “therapeutically effective amount” or “therapeutically effective dose” refer to the amount or dose of active ingredient that is aimed at, without causing significant negative or adverse side effects to the subject, (1) delaying or preventing the onset of a cancer in the subject; (2) reducing the severity or incidence of a cancer; (3) slowing down or stopping the progression, aggravation, or deterioration of one or more symptoms of a cancer affecting the subject; (4) bringing about ameliorations of the symptoms of a cancer affecting the subject; or (5) curing a cancer affecting the subject. A therapeutically effective amount may be administered prior to the onset of a cancer for a prophylactic or preventive action. Alternatively, or additionally, a therapeutically effective amount may be administered after initiation of a cancer for a therapeutic action.

The terms “treating” or “treatment” refer to therapeutic treatment; wherein the object is to prevent or slow down the targeted pathologic condition or disease. A subject or mammal is successfully “treated” for a disease or affection or condition if, after receiving the treatment according to the present invention, the subject or mammal shows observable and/or measurable reduction in or absence of one or more of the following: reduction of the number of cancer cells; and/or relief to some extent, for one or more of the symptoms associated with the specific disease or condition; reduced morbidity and mortality, and improvement in quality of life issues. The above parameters for assessing successful treatment and improvement in the disease are readily measurable by routine procedures familiar to a physician.

The term “subject” refers to a mammal, preferably a human In one embodiment, the subject is diagnosed with a cancer. In one embodiment, the subject is a patient, preferably a human patient, who/which is awaiting the receipt of, or is receiving, medical care or was/is/will be the subject of a medical procedure or is monitored for the development or progression of a disease, such as a cancer. In one embodiment, the subject is a human patient who is treated and/or monitored for the development or progression of a cancer. In one embodiment, the subject is a male. In another embodiment, the subject is a female. In one embodiment, the subject is an adult. In another embodiment, the subject is a child.

The terms “tumor-specific antigen” or “tumor-associated antigen” refer to an antigen specifically and/or abundantly expressed by cancer cells or tumor cells. T cells expressing T cell receptors recognizing and binding said antigens may be referred to as T cells recognizing a tumor-specific or tumor-associated antigen, T cells specific for a tumor-specific or tumor-associated antigen, T cells specific of a tumor-specific or tumor-associated antigen, or T cells directed to a tumor-specific or tumor-associated antigen.

The term “vaccine” refers to a preparation comprising a substance or a group of substances (i.e., a vaccine) meant to induce and/or enhance in a subject a targeted immune response towards an infectious agent (such as viruses, bacteria, fungi or parasites) or towards cancer cells. Prophylactic vaccination is used to prevent a subject from ever having a particular disease or to only have a mild case of the disease. Therapeutic vaccination is intended to treat a particular disease in a subject. For example, therapeutic anti-cancer vaccines may comprise a tumor-associated antigen or tumor-associated antigens, aiming at inducing and/or enhancing a cell-mediated immune response, in particular a T cell immune response, directed towards the cancer cells expressing said tumor-associated antigen(s).

The term “RECIST v1.1” refers to response evaluation criteria in solid tumors. RECIST is a set of international published rules ((Eisenhauer et al. 2009) that define when tumors in cancer patients improve (“respond”), stay the same (“stabilize”), or worsen (“progress”) during treatment.

The term “pCREB” refers to cyclic AMP response element binding protein phosphorylation.

The term “TNF-α” refers to tumor necrosis factor-α.

### DETAILED DESCRIPTION

A2AR Inhibitor

The present invention relates to pharmaceutical compositions and combinations of anticancer agents comprising at least one A2A adenosine receptor (A2AR) inhibitor. The A2AR inhibitor is a thiocarbamate derivative, especially a thiocarbamate derivative as those disclosed in PCT/EP2018/058301. More preferably the A2AR inhibitor is a thiocarbamate derivative of formula (I) as described below.

In one embodiment, the thiocarbamate derivative A2AR inhibitor is of Formula (I):

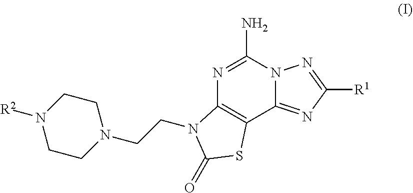

or a pharmaceutically acceptable salt or solvate thereof, wherein:

R¹ represents 5- or 6-membered heteroaryl or 5- or 6-membered aryl, wherein heteroaryl or aryl groups are optionally substituted by one or more substituent selected from C1-C6 alkyl (preferably methyl) and halo (preferably fluoro or chloro); preferably R¹ represents 5-membered heteroaryl; more preferably R¹ represents furyl;

R² represents 6-membered aryl or 6-membered heteroaryl,
wherein heteroaryl or aryl groups are optionally substituted by one or more substituent selected from halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino and alkylsulfonealkyl;
   said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxy alkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
or the heteroaryl or aryl groups are optionally substituted with two substituents that form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, amino alkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one embodiment, preferred compounds of Formula (I) are of Formula (Ia):

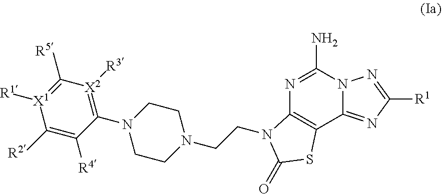

or a pharmaceutically acceptable salt or solvate thereof, wherein:
R¹ represents 5- or 6-membered heteroaryl or 5- or 6-membered aryl, wherein heteroaryl or aryl groups are optionally substituted by one or more substituent selected from C1-C6 alkyl (preferably methyl) and halo (preferably fluoro or chloro); preferably R¹ represents 5-membered heteroaryl; more preferably R¹ represents furyl;
X¹ and X² represent each independently C or N;
R^{1′} is absent when X¹ is N; or when X¹ is C, R^{1′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino or alkylsulfonealkyl;
   said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
R^{2′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino, or alkylsulfonealkyl;
   said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
or R^{1′} and R^{2′} form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, amino alkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
R^{3′} is absent when X² is N; or when X² is C, R^{3′} represents H or halo, preferably H or F;
R^{4′} represents H or halo, preferably H or F; and
R^{5′} represents H or halo, preferably H or F.

In one specific embodiment of the invention, R¹ represents 5- or 6-membered heteroaryl or 5- or 6-membered aryl, wherein heteroaryl or aryl groups are optionally substituted by one or more substituent selected from C1-C6 alkyl (preferably methyl) and halo (preferably fluoro or chloro). In a preferred embodiment, R¹ represents 5-membered heteroaryl; more preferably, R¹ represents furyl.

In one specific embodiment of the invention, X¹ and X² represent each independently C or N. In another specific embodiment, X¹ and X² both represent C.

In one specific embodiment of the invention, R^{1′} is absent when X¹ is N.

In another specific embodiment, when X¹ is C, R^{1′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino or alkylsulfonealkyl; said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In a preferred embodiment, R^{1′} substituents are optionally substituted by one or more substituent selected from halo, hydroxy, alkyl, heterocyclylalkyl, hydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkoxy, amino, dialkylamino, aminoalkylcarbonylamino, aminocarbonylalkylamino, heterocyclylalkylaminocarbonyl, (aminocarbonylalkyl)(alkyl)amino, hydroxycarbonyl, aminocarbonyl, aminoalkylaminocarbonyl, alkylaminoalkylaminocarbonyl, dialkylaminoalkylaminocarbonyl, (alkylaminoalkyl)(alkyl)aminocarbonyl, heterocyclylcarbonyl, alkylsulfoxide and alkylsulfonealkyl.

In one specific embodiment of the invention, R^{2′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino, or alkylsulfonealkyl; said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In a preferred embodiment, R^{2′} substituents are optionally substituted by one or more substituent selected from oxo, halo, hydroxy, cyano, alkyl, heterocyclylalkyl, dihydroxyalkyl, dialkylaminoalkyl, heteroaryl, alkylheteroaryl, hydroxycarbonyl, alkyloxycarbonyl, aminocarbonyl, heterocyclylalkylaminocarbonyl, alkylaminoalkylcarbonyl, dialkylaminoalkylcarbonyl, alkylsulfoxide, alkylsulfonealkyl.

In another specific embodiment of the invention, R^{1′} and R^{2′} form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one specific embodiment of the invention, R^{3′} is absent when X² is N. In another specific embodiment of the invention, when X² is C, R^{3′} represents H or halo. In a preferred embodiment, when X² is C, R^{3′} represents H or F.

In one specific embodiment of the invention, R^{4′} represents H or halo. In a preferred embodiment, R^{4′} represents H or F.

In one specific embodiment of the invention, R^{5′} represents H or halo. In a preferred embodiment, R^{5′} represents H or F.

In one embodiment, preferred compounds of Formula (Ia) are those of Formula (Ia-1):

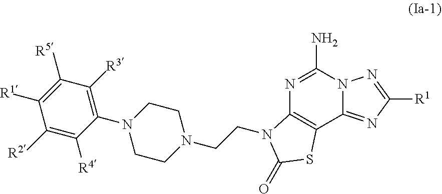

or a pharmaceutically acceptable salt or solvate thereof, wherein R¹, R^{1′}, R^{2′}, R^{3′}, R^{4′} and R^{5′} are as defined in Formula (Ia).

In one embodiment, preferred compounds of Formula (Ia-1) are those of Formula (Ia-1a):

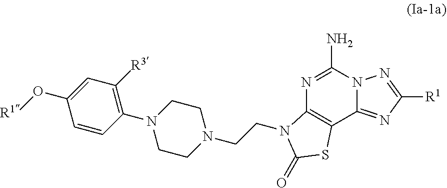

or a pharmaceutically acceptable salt or solvate thereof, wherein:
R¹ and R^{3′} are as defined in Formula (Ia); and
R^{1″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one specific embodiment of the invention, R^{1″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In a preferred embodiment, R^{1″} represents an alkyl or heterocyclyl group substituted by one or more group selected from hydroxy, heterocyclyl, heteroaryl, alkylheteroaryl, alkyne, alkoxy, amino, dialkylamino, amino alkylcarbonylamino, aminocarbonylalkylamino, (aminocarbonylalkyl)(alkyl)amino, hydroxycarbonyl, aminocarbonyl, aminoalkylaminocarbonyl, alkylaminoalkylaminocarbonyl, dialkylaminoalkylaminocarbonyl, (alkylaminoalkyl)(alkyl)aminocarbonyl, heterocyclylcarbonyl, alkylsulfoxide, alkylsulfonealkyl.

In one embodiment, preferred compounds of Formula (Ia-1) are those of Formula (Ia- 1b):

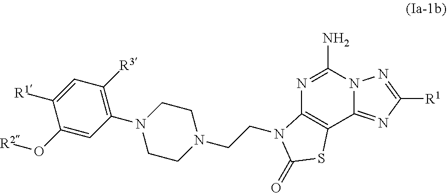

or a pharmaceutically acceptable salt or solvate thereof, wherein:
R¹ and R^{3′} are as defined in Formula (Ia);
R^{1′} represents H or halo, preferably H or F; and
R^{2″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, amino alkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one specific embodiment of the invention, R^{1′} represents H or halo. In a preferred embodiment, R^{1′} represents H or F.

In one specific embodiment of the invention, R^{2″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In a preferred embodiment, R^{2″} represents an alkyl or heterocyclyl group substituted by one or more group selected from hydroxy, cyano, heteroaryl, alkylheteroaryl, alkyne, hydroxycarbonyl, alkyloxycarbonyl, aminocarbonyl, alkylaminoalkylcarbonyl, dialkylaminoalkylcarbonyl, alkylsulfoxide, alkylsulfonealkyl.

In one embodiment, preferred compounds of Formula (Ia-1) are those of Formula (Ia-1c) or (Ia-1d):

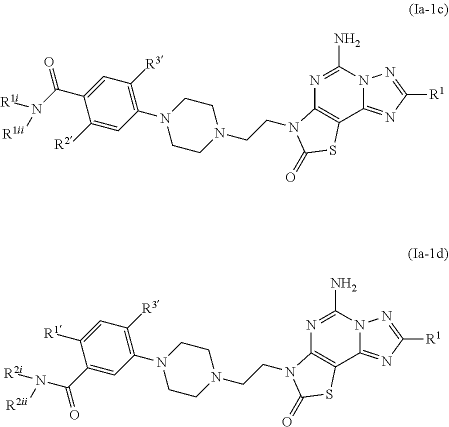

or a pharmaceutically acceptable salt or solvate thereof, wherein:
R¹ and R^{3′} are as defined in Formula (Ia);
R^{1′} represents H or halo, preferably H or F;
R^{2′} represents H or halo, preferably H or F;
R¹ⁱ and R¹ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl; and
R²ⁱ and R²ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl.

In one specific embodiment of the invention, R^{1′} represents H or halo. In a preferred embodiment, R^{1′} represents H or F.

In one specific embodiment of the invention, R^{2′} represents H or halo. In a preferred embodiment, R^{2′} represents H or F.

In one specific embodiment of the invention, R¹ⁱ and R¹ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl.

In a preferred embodiment, R¹ⁱ and R¹ⁱⁱ represent each independently hydrogen, alkyl, heterocyclylalkyl, hydroxyalkyl, hydroxyalkylaminoalkyl, aminoalkyl, alkylaminoalkyl, dialkylaminoalkyl, (heterocyclyl)(alkyl)aminoalkyl or heterocyclylalkylaminocarbonyl.

In one specific embodiment of the invention, R²ⁱ and R²ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl.

In a preferred embodiment, R²ⁱ and R²ⁱⁱ represent each independently hydrogen, alkyl, heterocyclylalkyl, dihydroxyalkyl, dialkylaminoalkyl or heterocyclylalkylaminocarbonyl. In a preferred embodiment, R²ⁱ and R²ⁱⁱ represent each independently hydrogen, alkyl or dialkylaminoalkyl.

In one embodiment, preferred compounds of Formula (Ia) are those of Formulae (Ia-2) or (Ia-3):

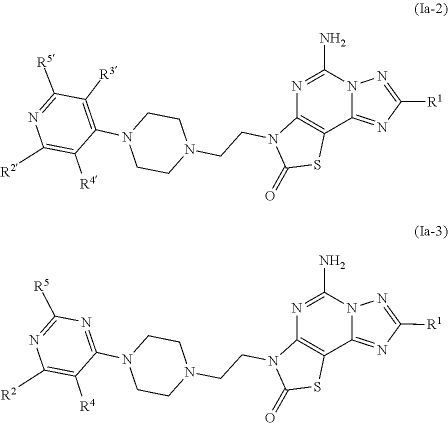

or a pharmaceutically acceptable salt or solvate thereof, wherein R¹, R^{2′}, R^{3′}, R^{4′} and R^{5′} are as defined in Formula (Ia).

Particularly preferred compounds of Formula (I) of the invention are those listed in Table 1 hereafter.

TABLE 1

| Cpd | | | |
|---|---|---|---|
| no | Structure | Chemical name | MW |
| 1 | 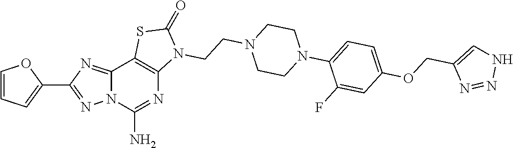 [SMILES: Nc1nc2c(sc(=O)n2CCN2CCN(c3ccc(OCc4c[nH]nn4)cc3F)CC2)c2nc(-c3ccco3)nn12] | 3-(2-(4-(4-((1H-1,2,3- triazolo-4yl)methoxy- 2fluorophenyl)piperazine-1- yl)ethyl)-5-amino-(8-(furan- 2-yl)thiazolo[5,4- e][1,2,4]triazolo[1,5- c]pyrimidine-2(3H)-one | 577.60 |
| 2 | 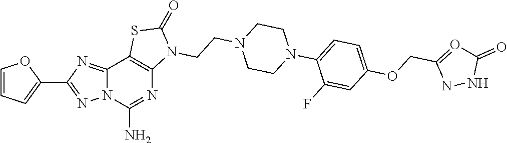 | 5-((4-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-3- fluorophenoxy)methyl)- 1,3,4-oxadiazol-2(3H)-one | 594.58 |
| 3 | 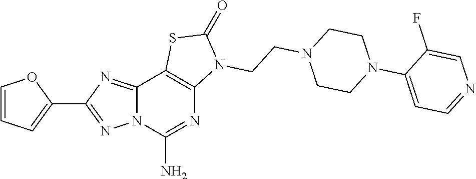 [SMILES: Nc1nc2c(sc(=O)n2CCN2CCN(c3ccncc3F)CC2)c2nc(-c3ccco3)nn12] | 5-amino-3-(2-(4-(3- fluoropyridin-4- yl)piperazin-1-yl)ethyl)-8- (furan-2-yl)thiazolo[5,4- e][1,2,4]triazolo[1,5- c]pyrimidin-2(3H)-one | 481.51 |
| 4 | 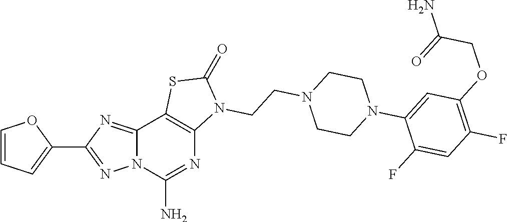 [SMILES: NC(=O)COc1cc(N2CCN(CCn3c(=O)sc4c3nc(N)n3nc(-c5ccco5)nc43)CC2)c(F)cc1F] | 2-(5-(4-(2-(5-amino-8- (furan-2-yl)-2- oxothiazolo[5,4-e] [1,2,4]triazolo [1,5-c] pyrimidin-3(2H)- yl)ethyl)piperazin-1-yl)-2,4- difluorophenoxy)acetamide | 571.56 |
| 5 | 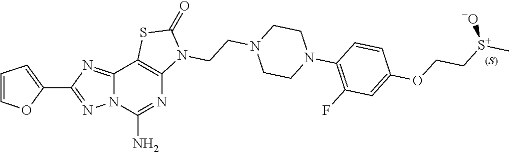 | (S)-5-amino-3-(2-(4-(2- fluoro-4-(2-(methyl- sulfinyl)ethoxy)phenyl) piperazin-1-yl)ethyl)-8- (furan-2-yl)thiazolo[5,4- e][1,2,4]triazolo[1,5- c]pyrimidin-2(3H)-one | 586.66 |
| 6 | 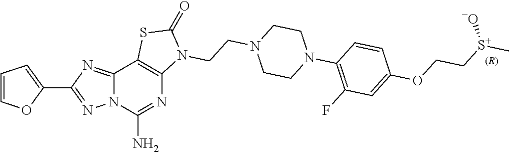 | (R)-5-amino-3-(2-(4-(2- fluoro-4-(2-(methyl- sulfinyl)ethoxy)phenyl)- piperazin-1-yl)ethyl)-8- (furan-2-yl)thiazolo[5,4- e][1,2,4]triazolo[1,5- c]pyrimidin-2(3H)-one | 586.66 |
| 7 | 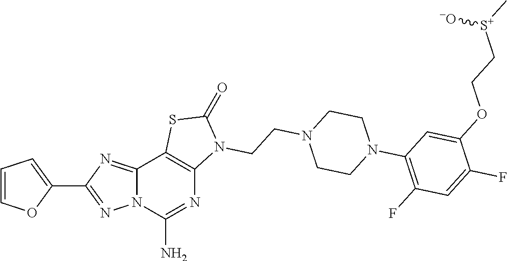 | (R,S)-5-amino-3-(2-(4-(2,4- difluoro-5-(2-(methyl- sulfinyl)ethoxy)phenyl) piperazin-1-yl)ethyl)-8- (furan-2-yl)thiazolo[5,4- e][1,2,4]triazolo[1,5- c]pyrimidin-2(3H)-one | 604.65 |
| 8a | 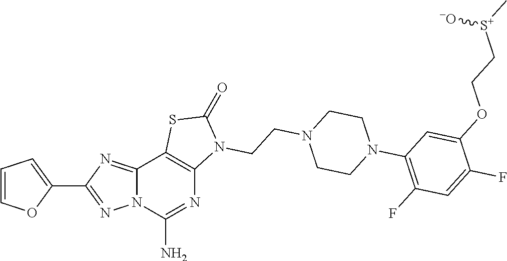 | (S)-5-amino-3-(2-(4-(2,4- difluoro-5-(2-(methyl- sulfinyl)ethoxy)phenyl) piperazin-1-yl)ethyl)-8- (furan-2-yl)thiazolo[5,4- e][1,2,4]triazolo[1,5- c]pyrimidin-2(3H)-one | 604.65 |
| 8b | 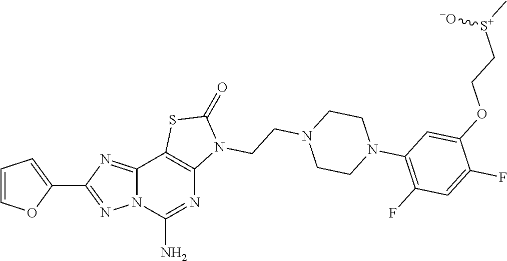 | (R)-5-amino-3-(2-(4-(2,4- difluoro-5-(2-(methyl- sulfinyl)ethoxy)phenyl) piperazin-1-yl)ethyl)-8- (furan-2-yl)thiazolo[5,4- e][1,2,4]triazolo[1,5- c]pyrimidin-2(3H)-one | 604.65 |
| 9 | 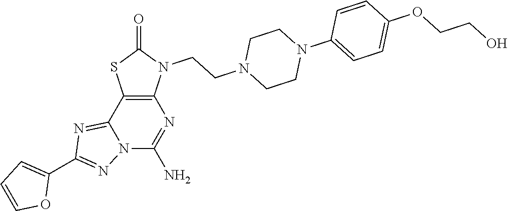 [SMILES: Nc1nc2c(sc(=O)n2CCN2CCN(c3ccc(OCCO)cc3)CC2)c2nc(-c3ccco3)nn12] | 5-amino-8-(furan-2-yl)-3- (2-(4-(4-(2-hydroxyethoxy) phenyl)piperazin-1- yl)ethyl)thiazolo[5,4- e][1,2,4]triazolo[1,5- c]pyrimidin-2(3H)-one | 522.58 |
| 10 | 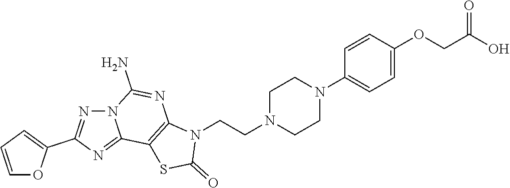 [SMILES: Nc1nc2c(sc(=O)n2CCN2CCN(c3ccc(OCC(=O)O)cc3)CC2)c2nc(-c3ccco3)nn12] | 2-(4-(4-(2-(5-amino-8- (furan-2-yl)-2-oxothiazolo [5,4-e][1,2,4]triazolo[1,5- c]pyrimidin-3(2H)- yl)ethyl)piperazin-1- yl)phenoxy)acetic acid | 536.56 |
| 11 | 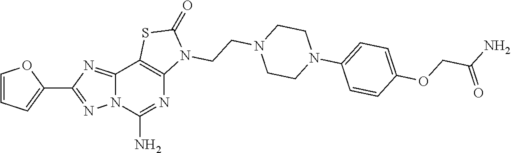 [SMILES: NC(=O)COc1ccc(N2CCN(CCn3c(=O)sc4c3nc(N)n3nc(-c5ccco5)nc43)CC2)cc1] | 2-(4-(4-(2-(5-amino-8- (furan-2-yl)-2-oxothiazolo [5,4-e][1,2,4]triazolo[1,5- c]pyrimidin-3(2H)- yl)ethyl)piperazin-1- yl)phenoxy)acetamide | 535.58 |
| 12 | 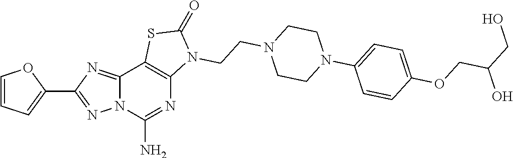 [SMILES: Nc1nc2c(sc(=O)n2CCN2CCN(c3ccc(OCC(O)CO)cc3)CC2)c2nc(-c3ccco3)nn12] | 5-amino-3-(2-(4-(4-(2,3- dihydroxypropoxy)phenyl) piperazin-1-yl)ethyl)-8- (furan-2-yl)thiazolo[5,4- e][1,2,4]triazolo[1,5- c]pyrimidin-2(3H)-one | 552.61 |
| 13 | 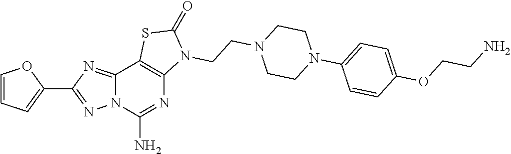 [SMILES: NCCOc1ccc(N2CCN(CCn3c(=O)sc4c3nc(N)n3nc(-c5ccco5)nc43)CC2)cc1] | 5-amino-3-(2-(4-(4-(2- aminoethoxy)phenyl) piperazin-1-yl)ethyl)-8-(furan- 2-yl)thiazolo[5,4-e] [1,2,4]triazolo[1,5- c]pyrimidin-2(3H)-one | 521.60 |
| 14 | 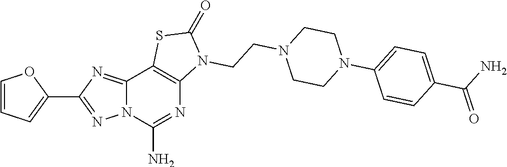 [SMILES: NC(=O)c1ccc(N2CCN(CCn3c(=O)sc4c3nc(N)n3nc(-c5ccco5)nc43)CC2)cc1] | 4-(4-(2-(5-amino-8-(furan- 2-yl)-2-oxothiazolo[5,4-e] [1,2,4]triazolo[1,5- c]pyrimidin-3(2H)-yl) ethyl)piperazin-1- yl)benzamide | 505.55 |
| 15 | 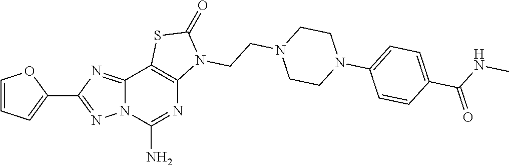 [SMILES: CNC(=O)c1ccc(N2CCN(CCn3c(=O)sc4c3nc(N)n3nc(-c5ccco5)nc43)CC2)cc1] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-N- methylbenzamide | 519.58 |
| 16 | 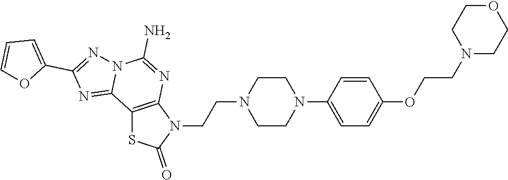 [SMILES: Nc1nc2c(sc(=O)n2CCN2CCN(c3ccc(OCCN4CCOCC4)cc3)CC2)c2nc(-c3ccco3)nn12] | 5-amino-8-(furan-2-yl)-3- (2-(4-(4-(2- morpholinoethoxy)phenyl) piperazin-1- yl)ethyl)thiazolo[5,4- e][1,2,4]triazolo[1,5- c]pyrimidin-2(3H)-one | 591.68 |
| 17 | 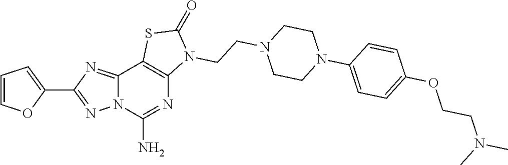 [SMILES: CN(C)CCOc1ccc(N2CCN(CCn3c(=O)sc4c3nc(N)n3nc(-c5ccco5)nc43)CC2)cc1] | 5-amino-3-(2-(4-(4-(2- (dimethylamino)ethoxy) phenyl)piperazin-1-yl)ethyl)- 8-(furan-2-yl)thiazolo[5,4- e][1,2,4]triazolo[1,5- c]pyrimidin-2(3H)-one | 549.65 |
| 18 | 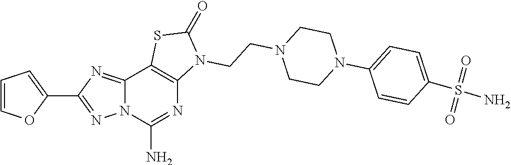 [SMILES: Nc1nc2c(sc(=O)n2CCN2CCN(c3ccc(S(N)(=O)=O)cc3)CC2)c2nc(-c3ccco3)nn12] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1- yl)benzenesulfonamide | 541.61 |
| 19 | 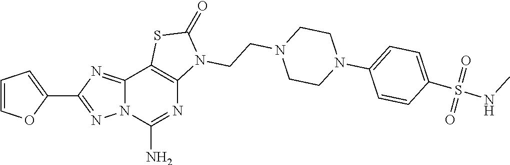 [SMILES: CNS(=O)(=O)c1ccc(N2CCN(CCn3c(=O)sc4c3nc(N)n3nc(-c5ccco5)nc43)CC2)cc1] | 4-(4-(2-(5-amino-8-(furan- 2-yl)-2-oxothiazolo[5,4- e][1,2,4]triazolo[1,5-c] pyrimidin-3(2H)-yl)ethyl) piperazin-1-yl)-N- methylbenzenesulfonamide | 555.63 |
| 20 | 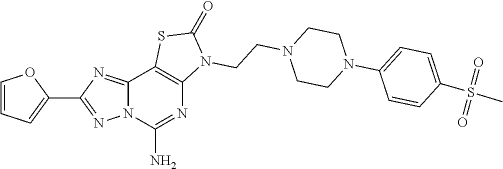 [SMILES: CS(=O)(=O)c1ccc(N2CCN(CCn3c(=O)sc4c3nc(N)n3nc(-c5ccco5)nc43)CC2)cc1] | 5-amino-8-(furan-2-yl)-3- (2-(4-(4-(methylsulfonyl) phenyl)piperazin-1- yl)ethyl)thiazolo[5,4- e][1,2,4]triazolo[1,5- c]pyrimidin-2(3H)-one | 540.62 |
| 21 | 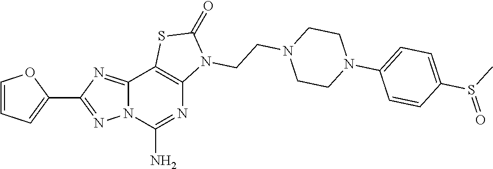 [SMILES: CS(=O)c1ccc(N2CCN(CCn3c(=O)sc4c3nc(N)n3nc(-c5ccco5)nc43)CC2)cc1] | 5-amino-8-(furan-2-yl)-3- (2-(4-(4-(methylsulfinyl) phenyl)piperazin-1- yl)ethyl)thiazolo[5,4- e][1,2,4]triazolo[1,5- c]pyrimidin-2(3H)-one | 524.62 |
| 22 | 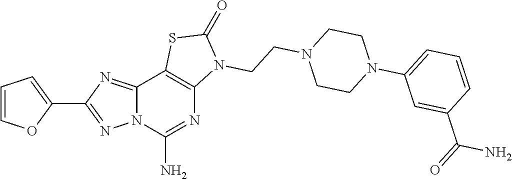 [SMILES: NC(=O)c1cccc(N2CCN(CCn3c(=O)sc4c3nc(N)n3nc(-c5ccco5)nc43)CC2)c1] | 3-(4-(2-(5-amino-8-(furan- 2-yl)-2-oxothiazolo[5,4- e][1,2,4]triazolo[1,5- c]pyrimidin-3(2H)- yl)ethyl)piperazin-1- yl)benzamide | 505.55 |
| 23 | 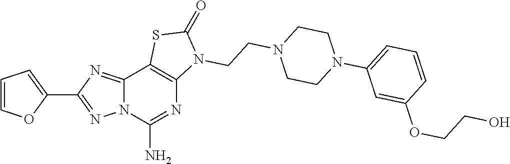 [SMILES: Nc1nc2c(sc(=O)n2CCN2CCN(c3cccc(OCCO)c3)CC2)c2nc(-c3ccco3)nn12] | 5-amino-8-(furan-2-yl)-3- (2-(4-(3-(2-hydroxyethoxy) phenyl)piperazin-1- yl)ethyl)thiazolo[5,4- e][1,2,4]triazolo[1,5- c]pyrimidin-2(3H)-one | 522.58 |
| 24 | 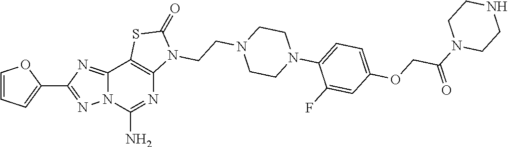 [SMILES: Nc1nc2c(sc(=O)n2CCN2CCN(c3ccc(OCC(=O)N4CCNCC4)cc3F)CC2)c2nc(-c3ccco3)nn12] | 5-amino-3-(2-(4-(2-fluoro- 4-(2-oxo-2-(piperazin-1- yl)ethoxy)phenyl)piperazin- 1-yl)ethyl)-8-(furan-2- yl)thiazolo[5,4- e][1,2,4]triazolo[1,5- c]pyrimidin-2(3H)-one | 622.67 |
| 25 | 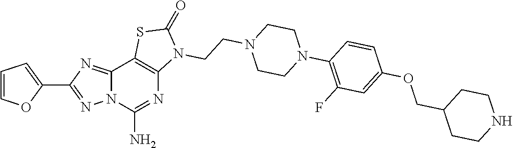 [SMILES: Nc1nc2c(sc(=O)n2CCN2CCN(c3ccc(OCC4CCNCC4)cc3F)CC2)c2nc(-c3ccco3)nn12] | 5-amino-3-(2-(4-(2-fluoro- 4-(piperidin-4-ylmethoxy) phenyl)piperazin-1- yl)ethyl)-8-(furan-2- yl)thiazolo[5,4- e][1,2,4]triazolo[1,5- c]pyrimidin-2(3H)-one | 593.68 |
| 26 | 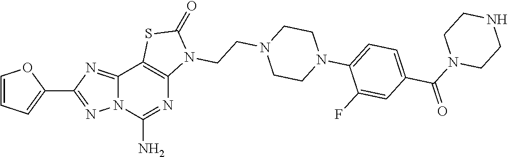 [SMILES: Nc1nc2c(sc(=O)n2CCN2CCN(c3ccc(C(=O)N4CCNCC4)cc3F)CC2)c2nc(-c3ccco3)nn12] | 5-amino-3-(2-(4-(2-fluoro- 4-(piperazine-1-carbonyl) phenyl)piperazin-1- yl)ethyl)-8-(furan-2- yl)thiazolo[5,4- e][1,2,4]triazolo[1,5- c]pyrimidin-2(3H)-one | 592.65 |
| 27 | 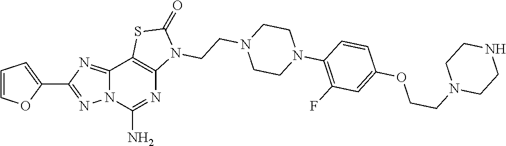 [SMILES: Nc1nc2c(sc(=O)n2CCN2CCN(c3ccc(OCCN4CCNCC4)cc3F)CC2)c2nc(-c3ccco3)nn12] | 5-amino-3-(2-(4-(2-fluoro- 4-(2-(piperazin-1- yl)ethoxy)phenyl)piperazin- 1-yl)ethyl)-8-(furan-2- yl)thiazolo[5,4- e][1,2,4]triazolo[1,5- c]pyrimidin-2(3H)-one | 608.69 |
| 28 | 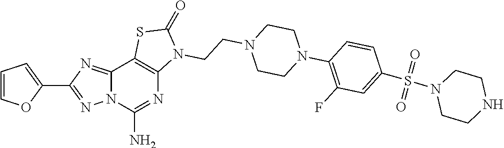 [SMILES: Nc1nc2c(sc(=O)n2CCN2CCN(c3ccc(S(=O)(=O)N4CCNCC4)cc3F)CC2)c2nc(-c3ccco3)nn12] | 5-amino-3-(2-(4-(2-fluoro- 4-(piperazin-1- ylsulfonyl)phenyl)piperazin- 1-yl)ethyl)-8-(furan-2- yl)thiazolo[5,4- e][1,2,4]triazolo[1,5- c]pyrimidin-2(3H)-one | 628.70 |
| 29 | 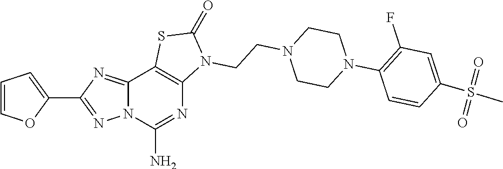 [SMILES: CS(=O)(=O)c1ccc(N2CCN(CCn3c(=O)sc4c3nc(N)n3nc(-c5ccco5)nc43)CC2)c(F)c1] | 5-amino-3-(2-(4-(2-fluoro- 4-(methylsulfonyl)phenyl) piperazin-1-yl)ethyl)-8- (furan-2-yl)thiazolo[5,4- e][1,2,4]triazolo[1,5- c]pyrimidin-2(3H)-one | 558.61 |
| 30 | 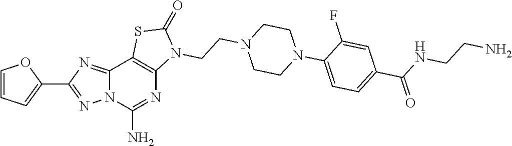 [SMILES: NCCNC(=O)c1ccc(N2CCN(CCn3c(=O)sc4c3nc(N)n3nc(-c5ccco5)nc43)CC2)c(F)c1] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-N- (2-aminoethyl)-3- fluorobenzamide | 566.61 |
| 31 | 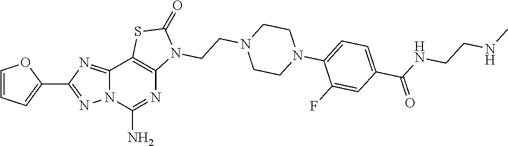 [SMILES: CNCCNC(=O)c1ccc(N2CCN(CCn3c(=O)sc4c3nc(N)n3nc(-c5ccco5)nc43)CC2)c(F)c1] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-3- fluoro-N-(2- (methylamino)ethyl) benzamide | 580.64 |
| 32 | 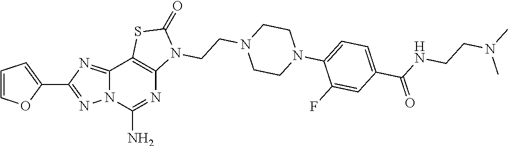 [SMILES: CN(C)CCNC(=O)c1ccc(N2CCN(CCn3c(=O)sc4c3nc(N)n3nc(-c5ccco5)nc43)CC2)c(F)c1] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-N- (2-(dimethylamino)ethyl)-3- fluorobenzamide | 594.66 |
| 33 | 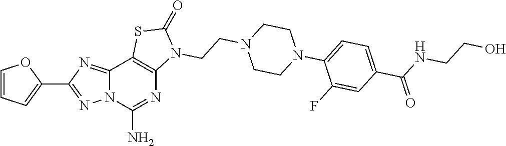 [SMILES: Nc1nc2c(sc(=O)n2CCN2CCN(c3ccc(C(=O)NCCO)cc3F)CC2)c2nc(-c3ccco3)nn12] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-3- fluoro-N-(2- hydroxyethyl)benzamide | 567.60 |
| 34 | 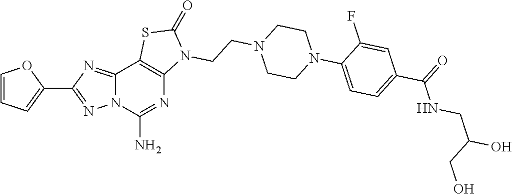 [SMILES: Nc1nc2c(sc(=O)n2CCN2CCN(c3ccc(C(=O)NCC(O)CO)cc3F)CC2)c2nc(-c3ccco3)nn12] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-N- (2,3-dihydroxypropyl)-3- fluorobenzamide | 597.62 |
| 35 | 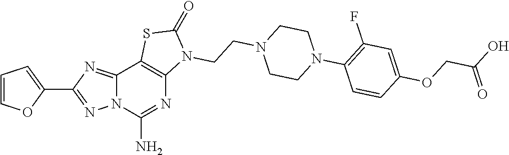 [SMILES: Nc1nc2c(sc(=O)n2CCN2CCN(c3ccc(OCC(=O)O)cc3F)CC2)c2nc(-c3ccco3)nn12] | 2-(4-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-3- fluorophenoxy)acetic acid | 554.55 |
| 36 | 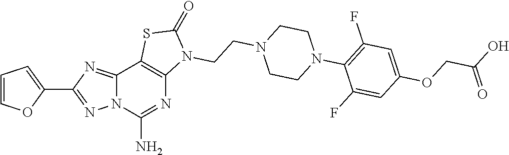 [SMILES: Nc1nc2c(sc(=O)n2CCN2CCN(c3c(F)cc(OCC(=O)O)cc3F)CC2)c2nc(-c3ccco3)nn12] | 2-(4-(4-(2-(5-amino-8- (furan-2-yl)-2-oxothiazolo [5,4-e][1,2,4]triazolo[1,5- c]pyrimidin-3(2H)-yl)ethyl) piperazin-1-yl)-3,5- difluorophenoxy) acetic acid | 572.54 |
| 37 | 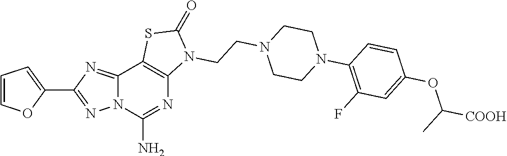 [SMILES: CC(Oc1ccc(N2CCN(CCn3c(=O)sc4c3nc(N)n3nc(-c5ccco5)nc43)CC2)c(F)c1)C(=O)O] | 2-(4-(4-(2-(5-amino-8- (furan-2-yl)-2- oxothiazolo[5,4-e] [1,2,4]triazolo[1,5- c]pyrimidin-3(2H)- yl)ethyl)piperazin-1-yl)-3- fluorophenoxy)propanoic acid | 568.58 |
| 38 | 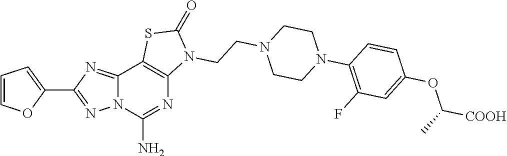 [SMILES: C[C@H](Oc1ccc(N2CCN(CCn3c(=O)sc4c3nc(N)n3nc(-c5ccco5)nc43)CC2)c(F)c1)C(=O)O] | (S)-2-(4-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-3- fluorophenoxy)propanoic acid | 568.58 |
| 39 | 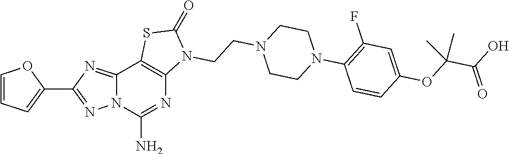 [SMILES: CC(C)(Oc1ccc(N2CCN(CCn3c(=O)sc4c3nc(N)n3nc(-c5ccco5)nc43)CC2)c(F)c1)C(=O)O] | 2-(4-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-3- fluorophenoxy)-2- methylpropanoic acid | 582.61 |
| 40 | 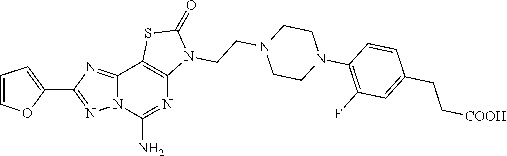 [SMILES: Nc1nc2c(sc(=O)n2CCN2CCN(c3ccc(CCC(=O)O)cc3F)CC2)c2nc(-c3ccco3)nn12] | 3-(4-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-3- fluorophenyl)propanoic acid | 552.58 |
| 41 | 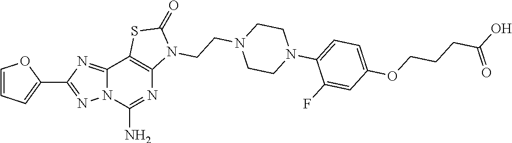 [SMILES: Nc1nc2c(sc(=O)n2CCN2CCN(c3ccc(OCCCC(=O)O)cc3F)CC2)c2nc(-c3ccco3)nn12] | 4-(4-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-3- fluorophenoxy)butanoic acid | 582.61 |
| 42 | 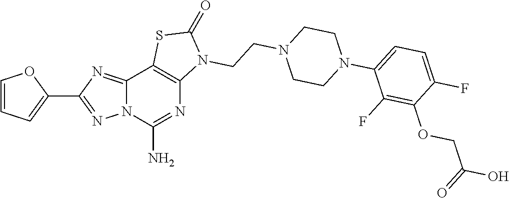 [SMILES: Nc1nc2c(sc(=O)n2CCN2CCN(c3ccc(F)c(OCC(=O)O)c3F)CC2)c2nc(-c3ccco3)nn12] | 2-(3-(4-(2-(5-amino-8- (furan-2-yl)-2- oxothiazolo[5,4-e] [1,2,4]triazolo[1,5- c]pyrimidin-3(2H)- yl)ethyl)piperazin-1-yl)-2,6- difluorophenoxy) acetic acid | 572.54 |
| 43 | 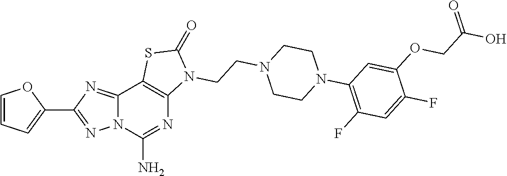 [SMILES: Nc1nc2c(sc(=O)n2CCN2CCN(c3cc(OCC(=O)O)c(F)cc3F)CC2)c2nc(-c3ccco3)nn12] | 2-(5-(4-(2-(5-amino-8- (furan-2-yl)-2- oxothiazolo[5,4-e] [1,2,4]triazolo[1,5- c]pyrimidin-3(2H)- yl)ethyl)piperazin-1-yl)-2,4- difluorophenoxy) acetic acid | 572.54 |
| 44 | 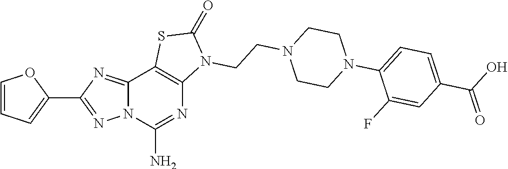 [SMILES: Nc1nc2c(sc(=O)n2CCN2CCN(c3ccc(C(=O)O)cc3F)CC2)c2nc(-c3ccco3)nn12] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-3- fluorobenzoic acid | 524.53 |
| 45 | 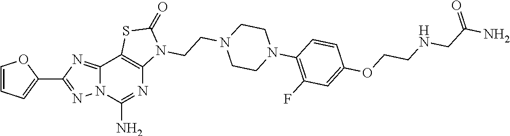 [SMILES: NC(=O)CNCCOc1ccc(N2CCN(CCn3c(=O)sc4c3nc(N)n3nc(-c5ccco5)nc43)CC2)c(F)c1] | 2-((2-(4-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-3- fluorophenoxy)ethyl) amino)acetamide | 596.64 |
| 46 | 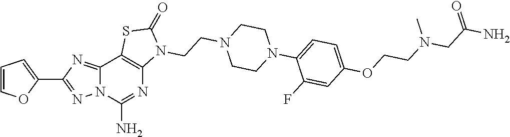 [SMILES: CN(CCOc1ccc(N2CCN(CCn3c(=O)sc4c3nc(N)n3nc(-c5ccco5)nc43)CC2)c(F)c1)CC(N)=O] | 2-((2-(4-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-3- fluorophenoxy)ethyl)(methyl) amino)acetamide | 610.66 |
| 47 | 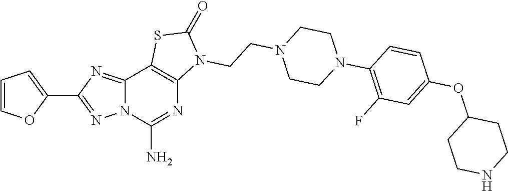 [SMILES: Nc1nc2c(sc(=O)n2CCN2CCN(c3ccc(OC4CCNCC4)cc3F)CC2)c2nc(-c3ccco3)nn12] | 5-amino-3-(2-(4-(2-fluoro- 4-(piperidin-4-yloxy) phenyl)piperazin-1-yl) ethyl)-8-(furan-2-yl) thiazolo[5,4-e][1,2,4] triazolo[1,5-c]pyrimidin- 2(3H)-one | 579.65 |
| 48 | 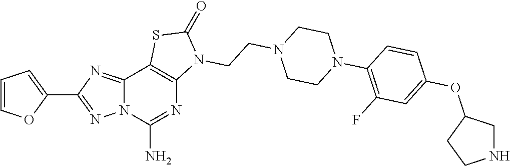 [SMILES: Nc1nc2c(sc(=O)n2CCN2CCN(c3ccc(OC4CCNC4)cc3F)CC2)c2nc(-c3ccco3)nn12] | 5-amino-3-(2-(4-(2-fluoro- 4-(pyrrolidin-3- yloxy)phenyl)piperazin-1- yl)ethyl)-8-(furan-2- yl)thiazolo[5,4- e][1,2,4]triazolo[1,5- c]pyrimidin-2(3H)-one | 565.62 |
| 49 | 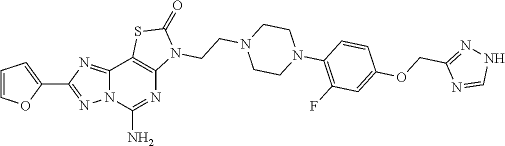 [SMILES: Nc1nc2c(sc(=O)n2CCN2CCN(c3ccc(OCc4nc[nH]n4)cc3F)CC2)c2nc(-c3ccco3)nn12] | 3-(2-(4-(4-((1H-1,2,4- triazol-3-yl)methoxy)-2- fluorophenyl)piperazin-1- yl)ethyl)-5-amino-8-(furan- 2-yl)thiazolo[5,4- e][1,2,4]triazolo[1,5- c]pyrimidin-2(3H)-one | 577.59 |
| 50 | 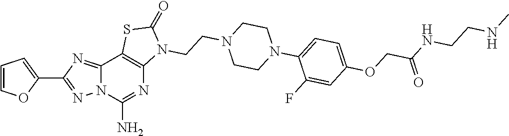 [SMILES: CNCCNC(=O)COc1ccc(N2CCN(CCn3c(=O)sc4c3nc(N)n3nc(-c5ccco5)nc43)CC2)c(F)c1] | 2-(4-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-3- fluorophenoxy)-N-(2- (methylamino)ethyl) acetamide | 610.66 |
| 51 | 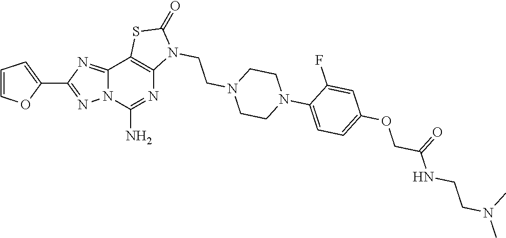 [SMILES: CN(C)CCNC(=O)COc1ccc(N2CCN(CCn3c(=O)sc4c3nc(N)n3nc(-c5ccco5)nc43)CC2)c(F)c1] | 2-(4-(4-(2-(5-amino-8- (furan-2-yl)-2-oxothiazolo [5,4-e][1,2,4]triazolo[1,5- c]pyrimidin-3(2H)-yl) ethyl)piperazin-1-yl)-3- fluorophenoxy)-N-(2- (dimethylamino)ethyl) acetamide | 624.69 |
| 52 | 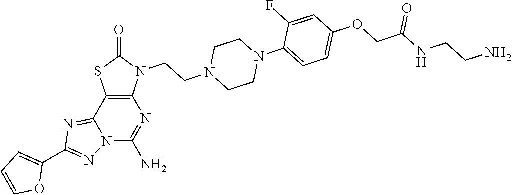 [SMILES: NCCNC(=O)COc1ccc(N2CCN(CCn3c(=O)sc4c3nc(N)n3nc(-c5ccco5)nc43)CC2)c(F)c1] | 2-(4-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-3- fluorophenoxy)-N-(2- aminoethyl)acetamide | 596.64 |
| 53 | 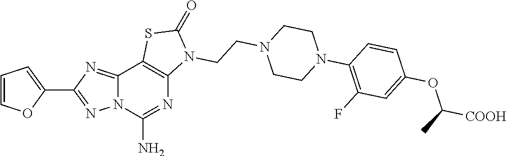 [SMILES: C[C@@H](Oc1ccc(N2CCN(CCn3c(=O)sc4c3nc(N)n3nc(-c5ccco5)nc43)CC2)c(F)c1)C(=O)O] | (R)-2-(4-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-3- fluorophenoxy)propanoic acid | 568.58 |
| 54 | 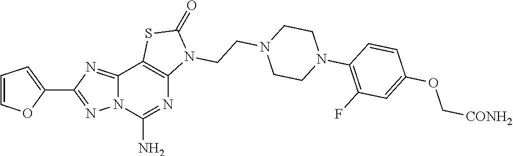 [SMILES: NC(=O)COc1ccc(N2CCN(CCn3c(=O)sc4c3nc(N)n3nc(-c5ccco5)nc43)CC2)c(F)c1] | 2-(4-(4-(2-(5-amino-8- (furan-2-yl)-2-oxothiazolo [5,4-e][1,2,4]triazolo[1,5- c]pyrimidin-3(2H)-yl) ethyl)piperazin-1-yl)-3- fluorophenoxy)acetamide | 553.57 |
| 55 | 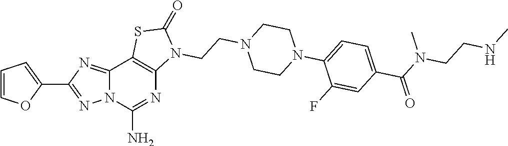 [SMILES: CNCCN(C)C(=O)c1ccc(N2CCN(CCn3c(=O)sc4c3nc(N)n3nc(-c5ccco5)nc43)CC2)c(F)c1] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-3- fluoro-N-methyl-N-(2- (methylamino)ethyl) benzamide | 594.66 |
| 56 | 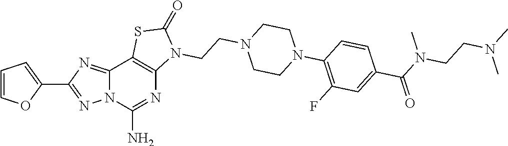 [SMILES: CN(C)CCN(C)C(=O)c1ccc(N2CCN(CCn3c(=O)sc4c3nc(N)n3nc(-c5ccco5)nc43)CC2)c(F)c1] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-N- (2-(dimethylamino)ethyl)-3- fluoro-N-methylbenzamide | 608.69 |
| 57 | 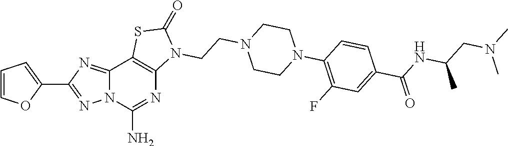 [SMILES: C[C@H](CN(C)C)NC(=O)c1ccc(N2CCN(CCn3c(=O)sc4c3nc(N)n3nc(-c5ccco5)nc43)CC2)c(F)c1] | (R)-4-(4-(2-(5-amino-8- (furan-2-yl)-2-oxothiazolo [5,4-e][1,2,4]triazolo[1,5- c]pyrimidin-3(2H)-yl) ethyl)piperazin-1-yl)-N-(1- (dimethylamino) propan-2- yl)-3-fluorobenzamide | 608.69 |
| 58 | 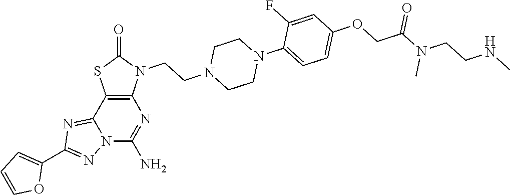 [SMILES: CNCCN(C)C(=O)COc1ccc(N2CCN(CCn3c(=O)sc4c3nc(N)n3nc(-c5ccco5)nc43)CC2)c(F)c1] | 2-(4-(4-(2-(5-amino-8- (furan-2-yl)-2-oxothiazolo [5,4-e][1,2,4]triazolo[1,5- c]pyrimidin-3(2H)-yl) ethyl)piperazin-1-yl)-3- fluorophenoxy)-N-methyl- N-(2-(methylamino)ethyl) acetamide | 624.69 |
| 59 | 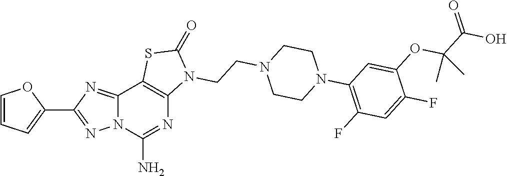 [SMILES: CC(C)(Oc1cc(N2CCN(CCn3c(=O)sc4c3nc(N)n3nc(-c5ccco5)nc43)CC2)c(F)cc1F)C(=O)O] | 2-(5-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-2,4- difluorophenoxy)-2- methylpropanoic acid | 600.60 |
| 60 | 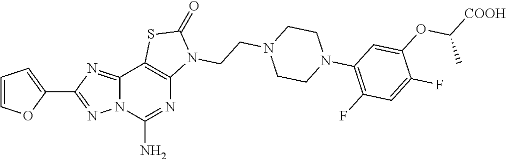 [SMILES: C[C@H](Oc1cc(N2CCN(CCn3c(=O)sc4c3nc(N)n3nc(-c5ccco5)nc43)CC2)c(F)cc1F)C(=O)O] | (S)-2-(5-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-2,4- difluorophenoxy) propanoic acid | 586.57 |
| 61 | 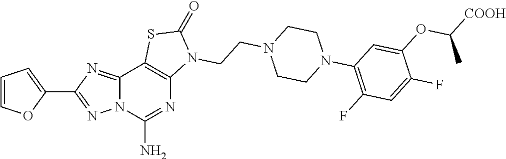 [SMILES: C[C@@H](Oc1cc(N2CCN(CCn3c(=O)sc4c3nc(N)n3nc(-c5ccco5)nc43)CC2)c(F)cc1F)C(=O)O] | (R)-2-(5-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-2,4- difluorophenoxy) propanoic acid | 586.57 |
| 62 | 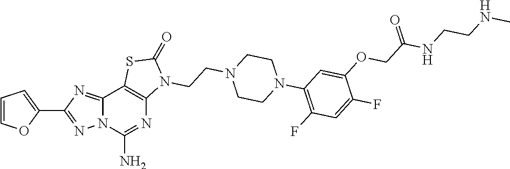 [SMILES: CNCCNC(=O)COc1cc(N2CCN(CCn3c(=O)sc4c3nc(N)n3nc(-c5ccco5)nc43)CC2)c(F)cc1F] | 2-(5-(4-(2-(5-amino-8- (furan-2-yl)-2- oxothiazolo[5,4-e] [1,2,4]triazolo[1,5- c]pyrimidin-3(2H)- yl)ethyl)piperazin-1-yl)-2,4- difluorophenoxy)-N-(2- (methylamino)ethyl) acetamide | 628.65 |
| 63 | 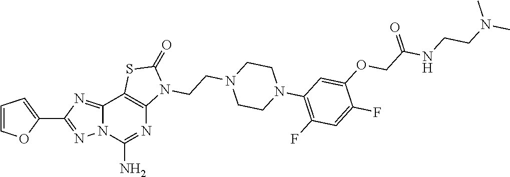 [SMILES: CN(C)CCNC(=O)COc1cc(N2CCN(CCn3c(=O)sc4c3nc(N)n3nc(-c5ccco5)nc43)CC2)c(F)cc1F] | 2-(5-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-2,4- difluorophenoxy)-N-(2- (dimethylamino)ethyl) acetamide | 642.68 |
| 64 | 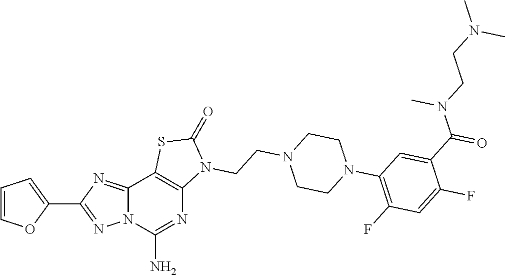 [SMILES: CN(C)CCN(C)C(=O)c1cc(N2CCN(CCn3c(=O)sc4c3nc(N)n3nc(-c5ccco5)nc43)CC2)c(F)cc1F] | 5-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-N- (2-(dimethylamino)ethyl)- 2,4-difluoro-N- methylbenzamide | 626.69 |
| 65 | 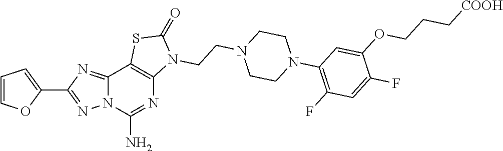 [SMILES: Nc1nc2c(sc(=O)n2CCN2CCN(c3cc(OCCCC(=O)O)c(F)cc3F)CC2)c2nc(-c3ccco3)nn12] | 4-(5-(4-(2-(5-amino-8- (furan-2-yl)-2-oxothiazolo [5,4-e][1,2,4]triazolo[1,5- c]pyrimidin-3(2H)- yl)ethyl)piperazin-1-yl)-2,4- difluorophenoxy) butanoic acid | 600.60 |
| 66 | 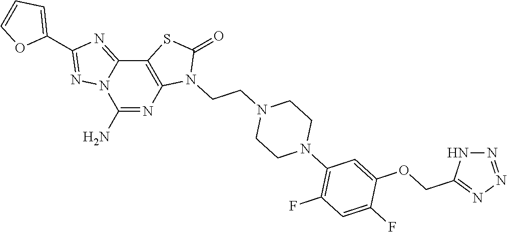 [SMILES: Nc1nc2c(sc(=O)n2CCN2CCN(c3cc(OCc4nnn[nH]4)c(F)cc3F)CC2)c2nc(-c3ccco3)nn12] | 3-(2-(4-(5-((1H-tetrazol-5- yl)methoxy)-2,4- difluorophenyl)piperazin-1- yl)ethyl)-5-amino-8-(furan- 2-yl)thiazolo[5,4- e][1,2,4]triazolo[1,5- c]pyrimidin-2(3H)-one | 596.58 |
| 67 | 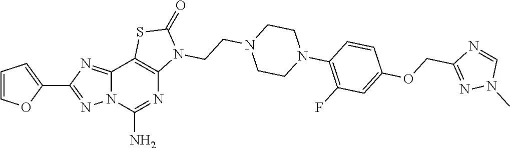 [SMILES: Cn1cnc(COc2ccc(N3CCN(CCn4c(=O)sc5c4nc(N)n4nc(-c6ccco6)nc54)CC3)c(F)c2)n1] | 5-amino-3-(2-(4-(2-fluoro- 4-((1-methyl-1H-1,2,4- triazol-3-yl)methoxy) phenyl)piperazin-1- yl)ethyl)-8-(furan-2- yl)thiazolo[5,4- e][1,2,4]triazolo[1,5- c]pyrimidin-2(3H)-one | 591.62 |
| 68 | 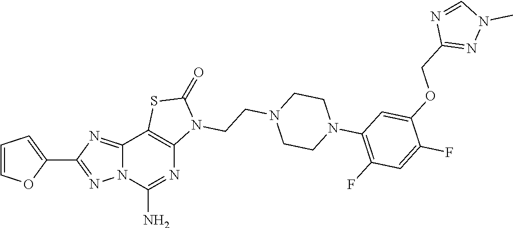 [SMILES: Cn1cnc(COc2cc(N3CCN(CCn4c(=O)sc5c4nc(N)n4nc(-c6ccco6)nc54)CC3)c(F)cc2F)n1] | 5-amino-3-(2-(4-(2,4- difluoro-5-((1-methyl-1H- 1,2,4-triazol-3-yl) methoxy)phenyl)piperazin- 1-yl)ethyl)-8-(furan-2- yl)thiazolo[5,4- e][1,2,4]triazolo[1,5- c]pyrimidin-2(3H)-one | 609.62 |
| 69 | 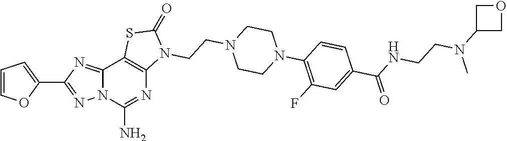 [SMILES: CN(CCNC(=O)c1ccc(N2CCN(CCn3c(=O)sc4c3nc(N)n3nc(-c5ccco5)nc43)CC2)c(F)c1)C1COC1] | 4-(4-(2-(5-amino-8-(furan- 2-yl)-2-oxothiazolo[5,4- e][1,2,4]triazolo[1,5- c]pyrimidin-3(2H)-yl) ethyl)piperazin-1-yl)-3- fluoro-N-(2-(methyl (oxetan-3-yl)amino)ethyl) benzamide | 636.70 |
| 70 | 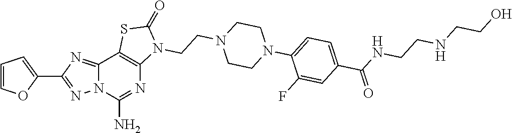 [SMILES: Nc1nc2c(sc(=O)n2CCN2CCN(c3ccc(C(=O)NCCNCCO)cc3F)CC2)c2nc(-c3ccco3)nn12] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-3- fluoro-N-(2-((2- hydroxyethyl)amino)ethyl) benzamide | 610.67 |
| 71 | 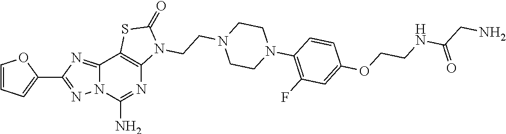 [SMILES: NCC(=O)NCCOc1ccc(N2CCN(CCn3c(=O)sc4c3nc(N)n3nc(-c5ccco5)nc43)CC2)c(F)c1] | 2-amino-N-(2-(4-(4-(2-(5- amino-8-(furan-2-yl)-2- oxothiazolo[5,4-e] [1,2,4]triazolo[1,5-c] pyrimidin-3(2H)-yl) ethyl)piperazin-1-yl)-3- fluorophenoxy)ethyl) acetamide | 596.64 |
| 72 | 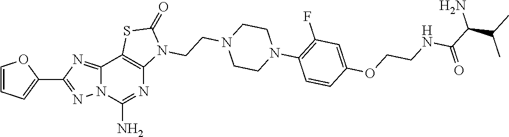 [SMILES: CC(C)[C@H](N)C(=O)NCCOc1ccc(N2CCN(CCn3c(=O)sc4c3nc(N)n3nc(-c5ccco5)nc43)CC2)c(F)c1] | (S)-2-amino-N-(2-(4-(4-(2- (5-amino-8-(furan-2-yl)-2- oxothiazolo[5,4- e][1,2,4]triazolo[1,5- c]pyrimidin-3(2H)- yl)ethyl)piperazin-1-yl)-3- fluorophenoxy)ethyl)-3- methylbutanamide | 638.72 |
| 73 | 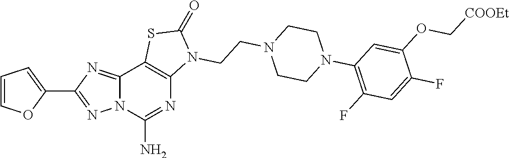 [SMILES: CCOC(=O)COc1cc(N2CCN(CCn3c(=O)sc4c3nc(N)n3nc(-c5ccco5)nc43)CC2)c(F)cc1F] | ethyl 2-(5-(4-(2-(5-amino-8- (furan-2-yl)-2- oxothiazolo[5,4-e] [1,2,4]triazolo[1,5-c] pyrimidin-3(2H)-yl) ethyl)piperazin-1-yl)-2,4- difluorophenoxy) acetate | 600.60 |
| 74 | 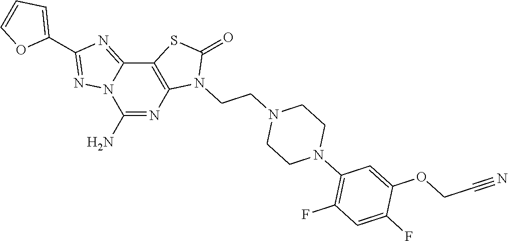 [SMILES: N#CCOc1cc(N2CCN(CCn3c(=O)sc4c3nc(N)n3nc(-c5ccco5)nc43)CC2)c(F)cc1F] | 2-(5-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-2,4- difluorophenoxy) acetonitrile | 553.54 |
| 75 | 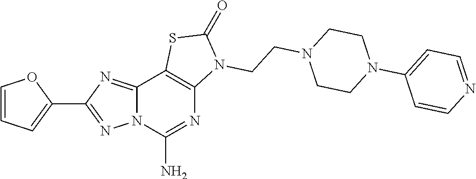 [SMILES: Nc1nc2c(sc(=O)n2CCN2CCN(c3ccncc3)CC2)c2nc(-c3ccco3)nn12] | 5-amino-8-(furan-2-yl)-3- (2-(4-(pyridin-4-yl) piperazin-1- yl)ethyl)thiazolo[5,4- e][1,2,4]triazolo[1,5- c]pyrimidin-2(3H)-one | 463.52 |
| 76 | 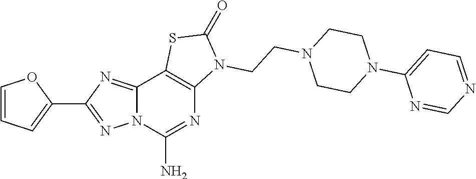 [SMILES: Nc1nc2c(sc(=O)n2CCN2CCN(c3ccncn3)CC2)c2nc(-c3ccco3)nn12] | 5-amino-8-(furan-2-yl)-3- (2-(4-(pyrimidin-4- yl)piperazin-1- yl)ethyl)thiazolo[5,4- e][1,2,4]triazolo[1,5- c]pyrimidin-2(3H)-one | 464.50 |
| 77 | 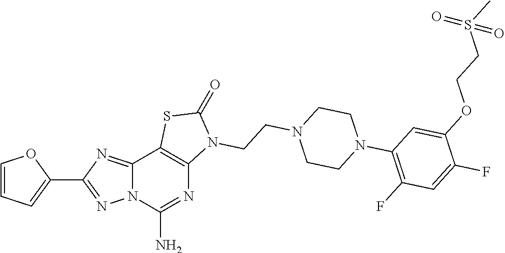 [SMILES: CS(=O)(=O)CCOc1cc(N2CCN(CCn3c(=O)sc4c3nc(N)n3nc(-c5ccco5)nc43)CC2)c(F)cc1F] | 5-amino-3-(2-(4-(2,4- difluoro-5-(2-(methyl- sulfonyl)ethoxy)phenyl) piperazin-1-yl)ethyl)-8- (furan-2-yl)thiazolo[5,4- e][1,2,4]triazolo[1,5- c]pyrimidin-2(3H)-one | 620.65 |
| 78 | 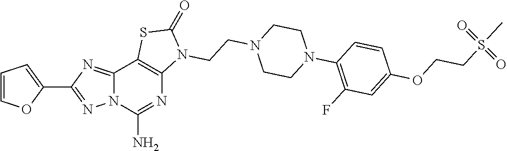 [SMILES: CS(=O)(=O)CCOc1ccc(N2CCN(CCn3c(=O)sc4c3nc(N)n3nc(-c5ccco5)nc43)CC2)c(F)c1] | 5-amino-3-(2-(4-(2-fluoro- 4-(2-(methyl- sulfonyl)ethoxy)phenyl) piperazin-1-yl)ethyl)-8- (furan-2-yl)thiazolo[5,4- e][1,2,4]triazolo[1,5- c]pyrimidin-2(3H)-one | 602.66 |
| 79 | 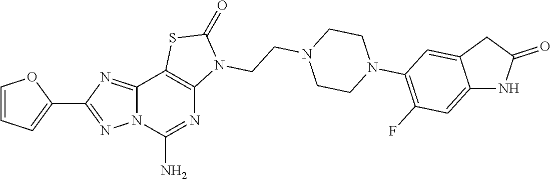 [SMILES: Nc1nc2c(sc(=O)n2CCN2CCN(c3cc4c(cc3F)NC(=O)C4)CC2)c2nc(-c3ccco3)nn12] | 5-amino-3-(2-(4-(6-fluoro- 2-oxoindolin-5- yl)piperazin-1-yl)ethyl)-8- (furan-2-yl)thiazolo[5,4- e][1,2,4]triazolo[1,5- c]pyrimidin-2(3H)-one | 535.55 |
| 80 | 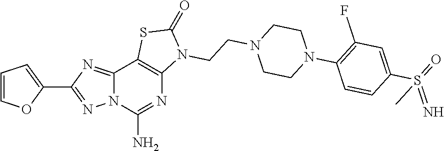 [SMILES: CS(=N)(=O)c1ccc(N2CCN(CCn3c(=O)sc4c3nc(N)n3nc(-c5ccco5)nc43)CC2)c(F)c1] | 5-amino-3-(2-(4-(2-fluoro- 4-(S- methylsulfonimidoyl)phenyl) piperazin-1-yl)ethyl)-8- (furan-2-yl)thiazolo[5,4- e][1,2,4]triazolo[1,5- c]pyrimidin-2(3H)-one | 557.62 |
| 81 | 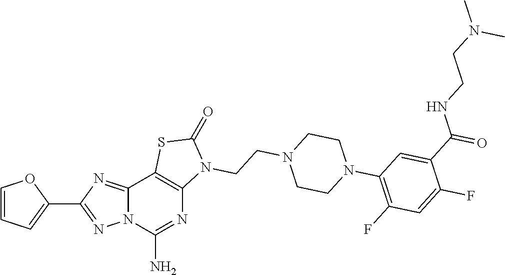 [SMILES: CN(C)CCNC(=O)c1cc(N2CCN(CCn3c(=O)sc4c3nc(N)n3nc(-c5ccco5)nc43)CC2)c(F)cc1F] | 5-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-N- (2-(dimethylamino)ethyl)- 2,4-difluorobenzamide | 612.65 |
| 82 | 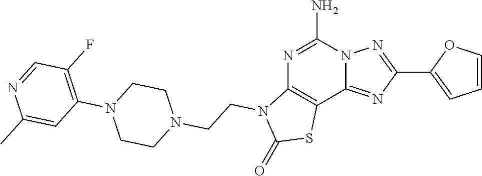 [SMILES: Cc1cc(N2CCN(CCn3c(=O)sc4c3nc(N)n3nc(-c5ccco5)nc43)CC2)c(F)cn1] | 5-amino-3-(2-(4-(5-fluoro- 2-methylpyridin-4- yl)piperazin-1-yl)ethyl)-8- (furan-2-yl)thiazolo[5,4- e][1,2,4]triazolo[1,5- c]pyrimidin-2(3H)-one | 495.53 |
| 83 | 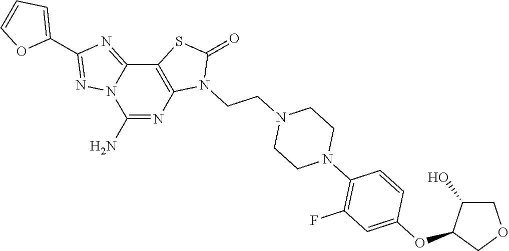 [SMILES: Nc1nc2c(sc(=O)n2CCN2CCN(c3ccc(O[C@@H]4COC[C@H]4O)cc3F)CC2)c2nc(-c3ccco3)nn12] | 5-amino-3-(2-(4-(2-fluoro- 4-(((3R,4R)-4- hydroxytetrahydrofuran-3- yl)oxy)phenyl)piperazin-1- yl)ethyl)-8-(furan-2- yl)thiazolo[5,4- e][1,2,4]triazolo[1,5- c]pyrimidin-2(3H)-one | 582.61 |
| 84 | 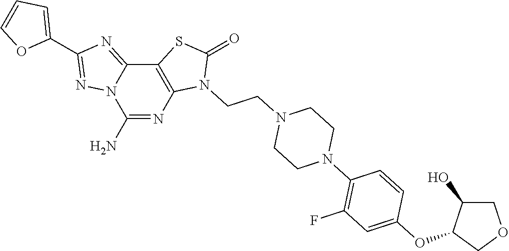 [SMILES: Nc1nc2c(sc(=O)n2CCN2CCN(c3ccc(O[C@H]4COC[C@@H]4O)cc3F)CC2)c2nc(-c3ccco3)nn12] | 5-amino-3-(2-(4-(2-fluoro- 4-(((3S,4S)-4- hydroxytetrahydrofuran-3- yl)oxy)phenyl)piperazin-1- yl)ethyl)-8-(furan-2- yl)thiazolo[5,4- e][1,2,4]triazolo[1,5- c]pyrimidin-2(3H)-one | 582.61 |
| 85 | 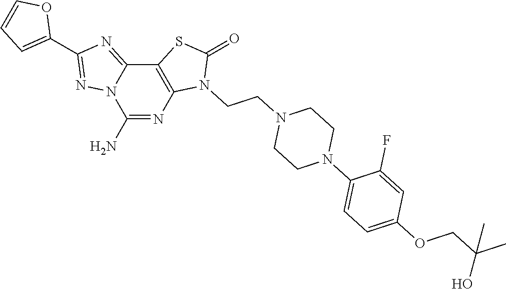 [SMILES: CC(C)(O)COc1ccc(N2CCN(CCn3c(=O)sc4c3nc(N)n3nc(-c5ccco5)nc43)CC2)c(F)c1] | 5-amino-3-(2-(4-(2-fluoro- 4-(2-hydroxy-2-methyl- propoxy)phenyl)piperazin- 1-yl)ethyl)-8-(furan-2- yl)thiazolo[5,4- e][1,2,4]triazolo[1,5- c]pyrimidin-2(3H)-one | 568.62 |
| 86 | 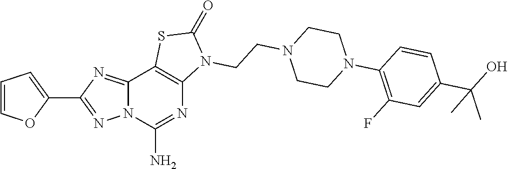 [SMILES: CC(C)(O)c1ccc(N2CCN(CCn3c(=O)sc4c3nc(N)n3nc(-c5ccco5)nc43)CC2)c(F)c1] | 5-amino-3-(2-(4-(2-fluoro- 4-(2-hydroxypropan-2- yl)phenyl)piperazin-1- yl)ethyl)-8-(furan-2- yl)thiazolo[5,4- e][1,2,4]triazolo[1,5- c]pyrimidin-2(3H)-one | 538.60 |
| 87 | 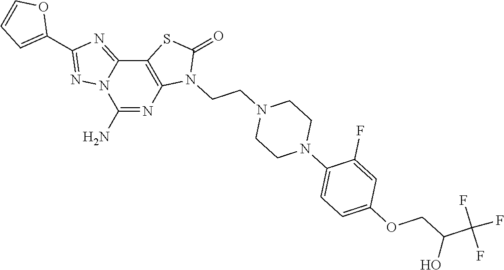 [SMILES: Nc1nc2c(sc(=O)n2CCN2CCN(c3ccc(OCC(O)C(F)(F)F)cc3F)CC2)c2nc(-c3ccco3)nn12] | 5-amino-3-(2-(4-(2-fluoro- 4-(3,3,3-trifluoro-2-hydroxy- propoxy)phenyl)piperazin- 1-yl)ethyl)-8-(furan- 2-yl)thiazolo[5,4- e][1,2,4]triazolo[1,5- c]pyrimidin-2(3H)-one | 608.57 |
| 88 | 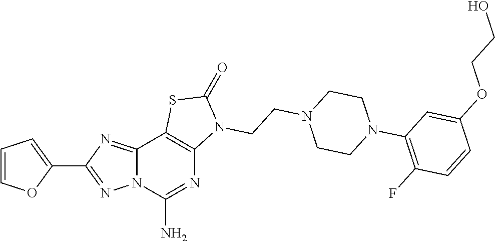 [SMILES: Nc1nc2c(sc(=O)n2CCN2CCN(c3cc(OCCO)ccc3F)CC2)c2nc(-c3ccco3)nn12] | 5-amino-3-(2-(4-(2-fluoro- 5-(2-hydroxy- ethoxy)phenyl)piperazin- 1-yl)ethyl)-8-(furan-2- yl)thiazolo[5,4- e][1,2,4]triazolo[1,5- c]pyrimidin-2(3H)-one | 540.57 |
| 89 | 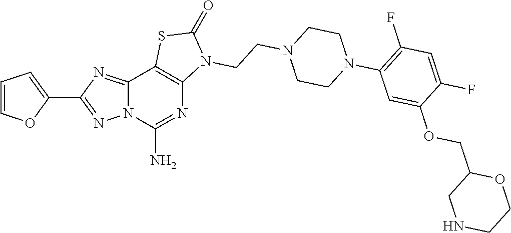 [SMILES: Nc1nc2c(sc(=O)n2CCN2CCN(c3cc(OCC4CNCCO4)c(F)cc3F)CC2)c2nc(-c3ccco3)nn12] | 5-amino-3-(2-(4-(2,4- difluoro-5-(morpholin-2- ylmethoxy)phenyl)piperazin- 1-yl)ethyl)-8-(furan-2- yl)thiazolo[5,4- e][1,2,4]triazolo[1,5- c]pyrimidin-2(3H)-one | 613.64 |
| 90 | 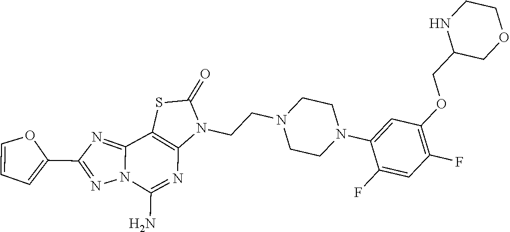 [SMILES: Nc1nc2c(sc(=O)n2CCN2CCN(c3cc(OCC4COCCN4)c(F)cc3F)CC2)c2nc(-c3ccco3)nn12] | 5-amino-3-(2-(4-(2,4- difluoro-5-(morpholin-3- ylmethoxy)phenyl)piperazin- 1-yl)ethyl)-8-(furan-2- yl)thiazolo[5,4- e][1,2,4]triazolo[1,5- c]pyrimidin-2(3H)-one | 613.64 |
| 91 | 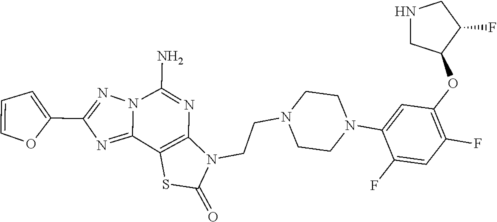 [SMILES: Nc1nc2c(sc(=O)n2CCN2CCN(c3cc(O[C@H]4CNC[C@@H]4F)c(F)cc3F)CC2)c2nc(-c3ccco3)nn12] | 5-amino-3-(2-(4-(2,4- difluoro-5-(((3S,4S)-4- fluoropyrrolidin-3- yl)oxy)phenyl)piperazin-1- yl)ethyl)-8-(furan-2- yl)thiazolo[5,4- e][1,2,4]triazolo[1,5- c]pyrimidin-2(3H)-one | 601.60 |
| 92 | 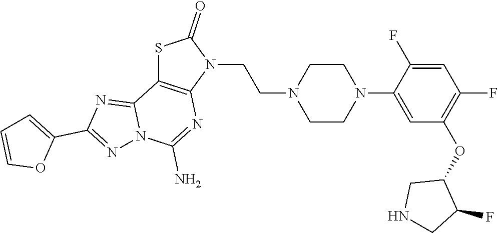 [SMILES: Nc1nc2c(sc(=O)n2CCN2CCN(c3cc(O[C@H]4CNC[C@@H]4F)c(F)cc3F)CC2)c2nc(-c3ccco3)nn12] | 5-amino-3-(2-(4-(2,4- difluoro-5-(((3S,4S)-4- fluoropyrrolidin-3- yl)oxy)phenyl)piperazin-1- yl)ethyl)-8-(furan-2- yl)thiazolo[5,4- e][1,2,4]triazolo[1,5- c]pyrimidin-2(3H)-one | 601.60 |
| 93 | 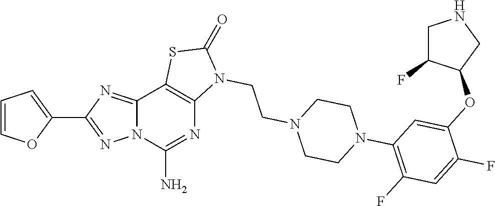 [SMILES: Nc1nc2c(sc(=O)n2CCN2CCN(c3cc(O[C@@H]4CNC[C@@H]4F)c(F)cc3F)CC2)c2nc(-c3ccco3)nn12] | 5-amino-3-(2-(4-(2,4- difluoro-5-(((3R,4S)-4- fluoropyrrolidin-3- yl)oxy)phenyl)piperazin-1- yl)ethyl)-8-(furan-2- yl)thiazolo[5,4- e][1,2,4]triazolo[1,5- c]pyrimidin-2(3H)-one | 601.60 |
| 94 | 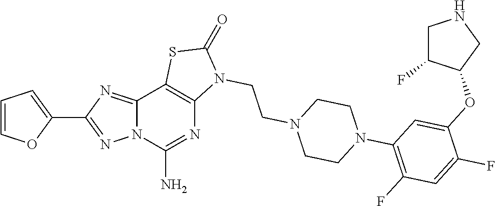 [SMILES: Nc1nc2c(sc(=O)n2CCN2CCN(c3cc(O[C@H]4CNC[C@H]4F)c(F)cc3F)CC2)c2nc(-c3ccco3)nn12] | 5-amino-3-(2-(4-(2,4- difluoro-5-(((3S,4R)-4- fluoropyrrolidin-3- yl)oxy)phenyl)piperazin-1- yl)ethyl)-8-(furan-2- yl)thiazolo[5,4- e][1,2,4]triazolo[1,5- c]pyrimidin-2(3H)-one | 601.60 |
| 95 | 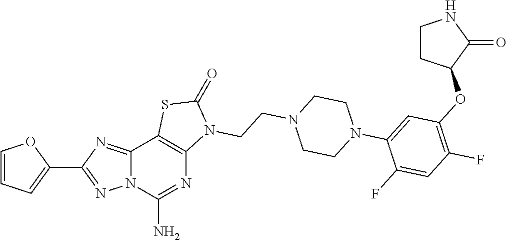 [SMILES: Nc1nc2c(sc(=O)n2CCN2CCN(c3cc(O[C@H]4CCNC4=O)c(F)cc3F)CC2)c2nc(-c3ccco3)nn12] | (S)-5-amino-3-(2-(4-(2,4- difluoro-5-((2- oxopyrrolidin-3- yl)oxy)phenyl)piperazin-1- yl)ethyl)-8-(furan-2- yl)thiazolo[5,4- e][1,2,4]triazolo[1,5- c]pyrimidin-2(3H)-one | 597.60 |
| 96 | 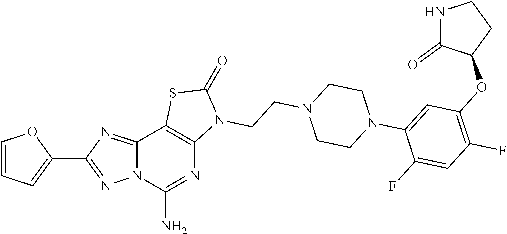 [SMILES: Nc1nc2c(sc(=O)n2CCN2CCN(c3cc(O[C@@H]4CCNC4=O)c(F)cc3F)CC2)c2nc(-c3ccco3)nn12] | (R)-5-amino-3-(2-(4-(2,4- difluoro-5-((2- oxopyrrolidin-3- yl)oxy)phenyl)piperazin-1- yl)ethyl)-8-(furan-2- yl)thiazolo[5,4- e][1,2,4]triazolo[1,5- c]pyrimidin-2(3H)-one | 597.60 |
| 97 | 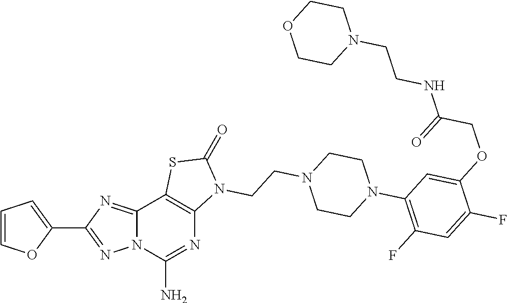 [SMILES: Nc1nc2c(sc(=O)n2CCN2CCN(c3cc(OCC(=O)NCCN4CCOCC4)c(F)cc3F)CC2)c2nc(-c3ccco3)nn12] | 2-(5-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-2,4- difluorophenoxy)-N-(2- morpholinoethyl)acetamide | 684.72 |
| 98 | 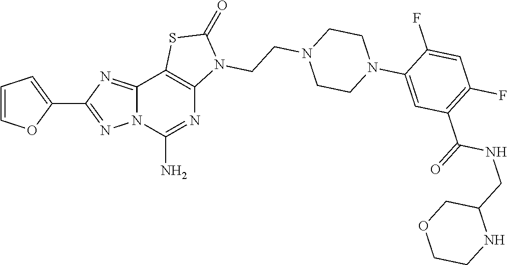 [SMILES: Nc1nc2c(sc(=O)n2CCN2CCN(c3cc(C(=O)NCC4COCCN4)c(F)cc3F)CC2)c2nc(-c3ccco3)nn12] | 5-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-2,4- difluoro-N-(morpholin-3- ylmethyl)benzamide | 640.66 |
| 99 | 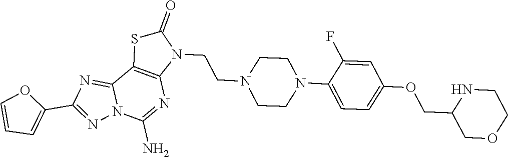 [SMILES: Nc1nc2c(sc(=O)n2CCN2CCN(c3ccc(OCC4COCCN4)cc3F)CC2)c2nc(-c3ccco3)nn12] | 5-amino-3-(2-(4-(2-fluoro- 4-(morpholin-3- ylmethoxy)phenyl)piperazin- 1-yl)ethyl)-8-(furan-2- yl)thiazolo[5,4- e][1,2,4]triazolo[1,5- c]pyrimidin-2(3H)-one | 595.65 |
| 100 | 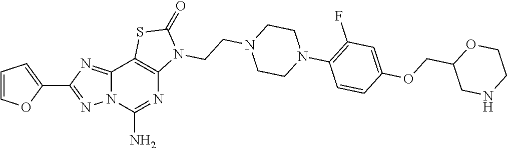 [SMILES: Nc1nc2c(sc(=O)n2CCN2CCN(c3ccc(OCC4CNCCO4)cc3F)CC2)c2nc(-c3ccco3)nn12] | 5-amino-3-(2-(4-(2-fluoro- 4-(morpholin-2- ylmethoxy)phenyl)piperazin- 1-yl)ethyl)-8-(furan-2- yl)thiazolo[5,4- e][1,2,4]triazolo[1,5- c]pyrimidin-2(3H)-one | 595.65 |
| 101 | 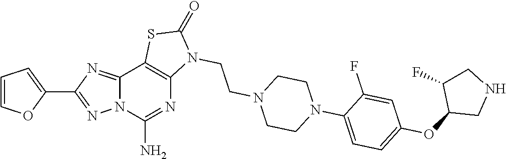 [SMILES: Nc1nc2c(sc(=O)n2CCN2CCN(c3ccc(O[C@@H]4CNC[C@H]4F)cc3F)CC2)c2nc(-c3ccco3)nn12] | 5-amino-3-(2-(4-(2-fluoro- 4-(((3R,4R)-4- fluoropyrrolidin-3- yl)oxy)phenyl)piperazin-1- yl)ethyl)-8-(furan-2- yl)thiazolo[5,4- e][1,2,4]triazolo[1,5- c]pyrimidin-2(3H)-one | 583.61 |
| 102 | 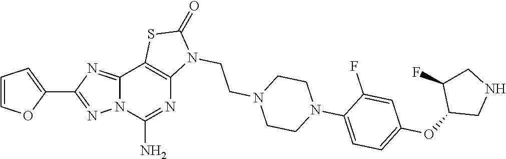 [SMILES: Nc1nc2c(sc(=O)n2CCN2CCN(c3ccc(O[C@H]4CNC[C@@H]4F)cc3F)CC2)c2nc(-c3ccco3)nn12] | 5-amino-3-(2-(4-(2-fluoro- 4-(((3S,4S)-4- fluoropyrrolidin-3- yl)oxy)phenyl)piperazin-1- yl)ethyl)-8-(furan-2- yl)thiazolo[5,4- e][1,2,4]triazolo[1,5- c]pyrimidin-2(3H)-one | 583.61 |
| 103 | 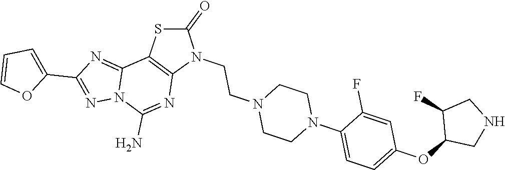 [SMILES: Nc1nc2c(sc(=O)n2CCN2CCN(c3ccc(O[C@@H]4CNC[C@@H]4F)cc3F)CC2)c2nc(-c3ccco3)nn12] | 5-amino-3-(2-(4-(2-fluoro- 4-(((3R,4S)-4- fluoropyrrolidin-3- yl)oxy)phenyl)piperazin-1- yl)ethyl)-8-(furan-2- yl)thiazolo[5,4- e][1,2,4]triazolo[1,5- c]pyrimidin-2(3H)-one | 583.61 |
| 104 | 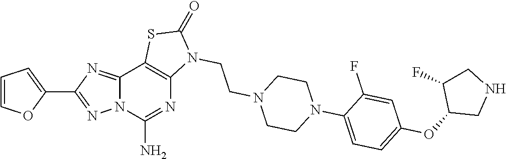 [SMILES: Nc1nc2c(sc(=O)n2CCN2CCN(c3ccc(O[C@H]4CNC[C@H]4F)cc3F)CC2)c2nc(-c3ccco3)nn12] | 5-amino-3-(2-(4-(2-fluoro- 4-(((3S,4R)-4- fluoropyrrolidin-3- yl)oxy)phenyl)piperazin-1- yl)ethyl)-8-(furan-2- yl)thiazolo[5,4- e][1,2,4]triazolo[1,5- c]pyrimidin-2(3H)-one | 583.61 |
| 105 | 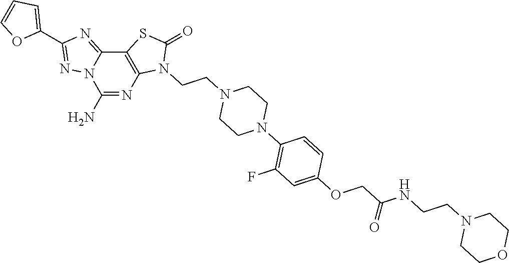 [SMILES: Nc1nc2c(sc(=O)n2CCN2CCN(c3ccc(OCC(=O)NCCN4CCOCC4)cc3F)CC2)c2nc(-c3ccco3)nn12] | 2-(4-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-3- fluorophenoxy)-N-(2- morpholinoethyl)acetamide | 666.73 |
| 106 | 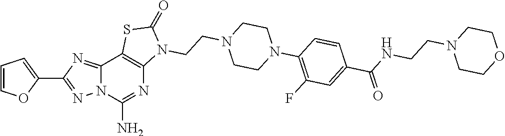 [SMILES: Nc1nc2c(sc(=O)n2CCN2CCN(c3ccc(C(=O)NCCN4CCOCC4)cc3F)CC2)c2nc(-c3ccco3)nn12] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-3- fluoro-N-(2- morpholinoethyl)benzamide | 636.70 |
| 107 | 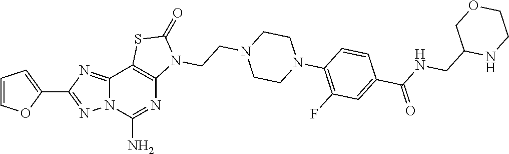 [SMILES: Nc1nc2c(sc(=O)n2CCN2CCN(c3ccc(C(=O)NCC4COCCN4)cc3F)CC2)c2nc(-c3ccco3)nn12] | 4-(4-(2-(5-amino-8-(furan- 2-yl)-2-oxothiazolo[5,4- e][1,2,4]triazolo[1,5- c]pyrimidin-3(2H)- yl)ethyl)piperazin-1-yl)-3- fluoro-N-(morpholin-3- ylmethyl)benzamide | 622.67 |
| 108 | 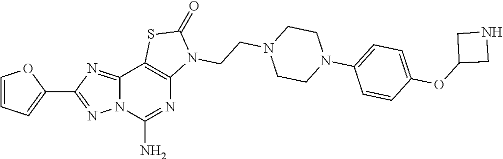 [SMILES: Nc1nc2c(sc(=O)n2CCN2CCN(c3ccc(OC4CNC4)cc3)CC2)c2nc(-c3ccco3)nn12] | 5-amino-3-(2-(4-(4- (azetidin-3- yloxy)phenyl)piperazin-1- yl)ethyl)-8-(furan-2- yl)thiazolo[5,4- e][1,2,4]triazolo[1,5- c]pyrimidin-2(3H)-one | 533.61 |
| 109 | 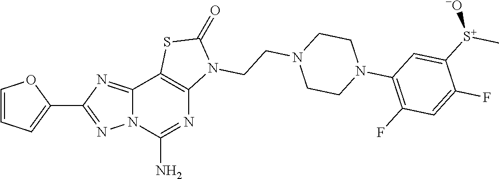 | (S)-5-amino-3-(2-(4-(2,4- difluoro-5-(methyl- sulfinyl)phenyl)piperazin- 1-yl)ethyl)-8-(furan-2- yl)thiazolo[5,4- e][1,2,4]triazolo[1,5- c]pyrimidin-2(3H)-one | 560.60 |
| 110 | 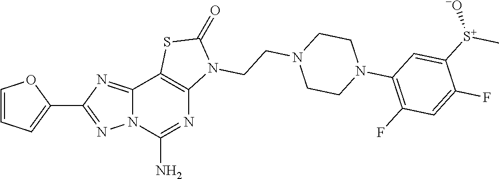 | (R)-5-amino-3-(2-(4-(2,4- difluoro-5-(methyl- sulfinyl)phenyl)piperazin- 1-yl)ethyl)-8-(furan-2- yl)thiazolo[5,4- e][1,2,4]triazolo[1,5- c]pyrimidin-2(3H)-one | 560.60 |
| 111 | 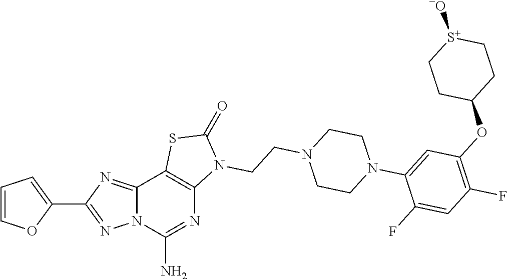 | 5-amino-3-(2-(4-(2,4- difluoro-5-(((1s,4s)-1- oxidotetrahydro-2H- thiopyran-4- yl)oxy)phenyl)piperazin-1- yl)ethyl)-8-(furan-2- yl)thiazolo[5,4- e][1,2,4]triazolo[1,5- c]pyrimidin-2(3H)-one | 630.69 |
| 112 | 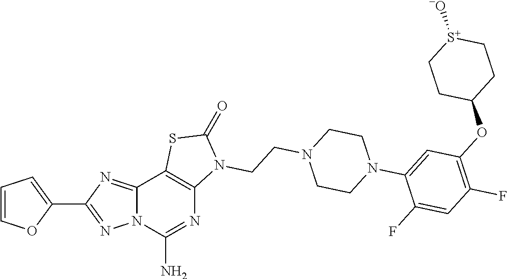 | 5-amino-3-(2-(4-(2,4- difluoro-5-(((1r,4r)-1- oxidotetrahydro-2H- thiopyran-4- yl)oxy)phenyl)piperazin-1- yl)ethyl)-8-(furan-2- yl)thiazolo[5,4- e][1,2,4]triazolo[1,5- c]pyrimidin-2(3H)-one | 630.69 |
| 113 | 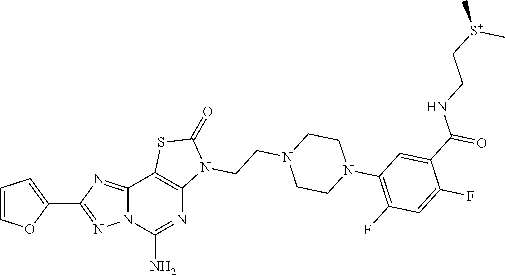 | (S)-5-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-2,4- difluoro-N-(2- (methylsulfinyl)ethyl) benzamide | 631.68 |
| 114 | 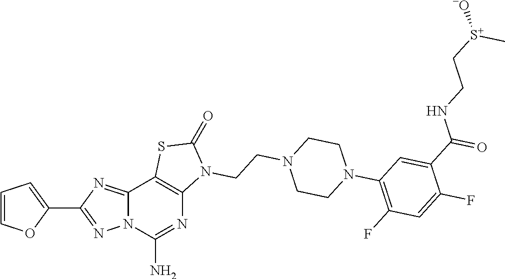 | (R)-5-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-2,4- difluoro-N-(2- (methylsulfinyl)ethyl) benzamide | 631.68 |
| 115 | 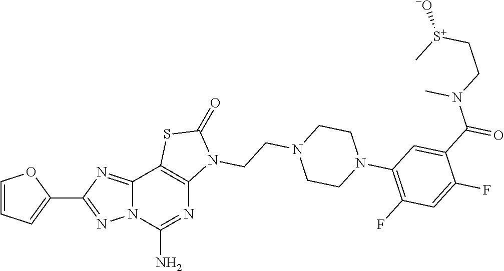 | (S)-5-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-2,4- difluoro-N-methyl-N-(2- (methylsulfinyl)ethyl) benzamide | 645.70 |
| 116 | 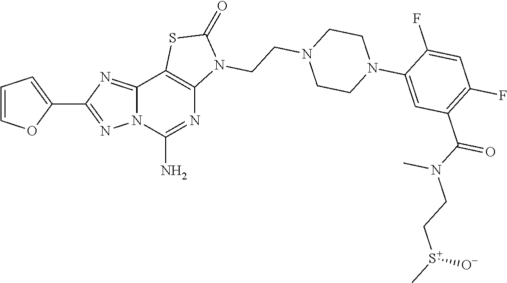 [SMILES: CN(CC[S@@+](C)[O-])C(=O)c1cc(N2CCN(CCn3c(=O)sc4c3nc(N)n3nc(-c5ccco5)nc43)CC2)c(F)cc1F] | (R)-5-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-2,4- difluoro-N-methyl-N-(2- (methylsulfinyl)ethyl) benzamide | 645.70 |
| 117 | 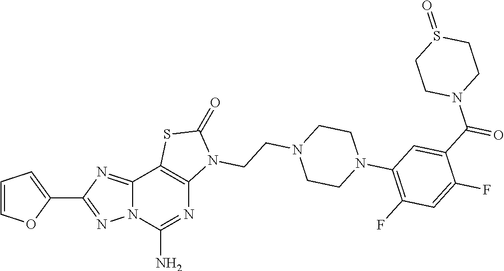 [SMILES: Nc1nc2c(sc(=O)n2CCN2CCN(c3cc(C(=O)N4CCS(=O)CC4)c(F)cc3F)CC2)c2nc(-c3ccco3)nn12] | 5-amino-3-(2-(4-(2,4- difluoro-5-(1- oxidothiomorpholine-4- carbonyl)phenyl)piperazin- 1-yl)ethyl)-8-(furan-2- yl)thiazolo[5,4- e][1,2,4]triazolo[1,5- c]pyrimidin-2(3H)-one | 643.69 |
| 118 | 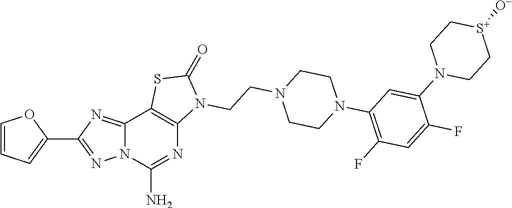 | 5-amino-3-(2-(4-(2,4- difluoro-5-(1- oxidothiomorpholino)phenyl) piperazin-1-yl)ethyl)-8- (furan-2-yl)thiazolo[5,4- e][1,2,4]triazolo[1,5- c]pyrimidin-2(3H)-one | 615.68 |
| 119 | 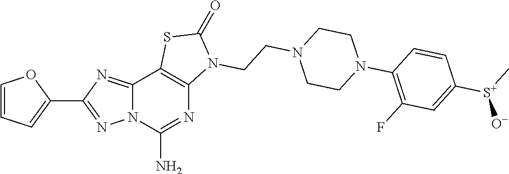 [SMILES: C[S@@+]([O-])c1ccc(N2CCN(CCn3c(=O)sc4c3nc(N)n3nc(-c5ccco5)nc43)CC2)c(F)c1] | (R)-5-amino-3-(2-(4-(2- fluoro-4-(methyl- sulfinyl)phenyl)piperazin- 1-yl)ethyl)-8-(furan-2- yl)thiazolo[5,4- e][1,2,4]triazolo[1,5- c]pyrimidin-2(3H)-one | 542.61 |
| 120 | 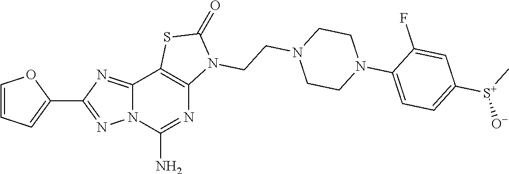 [SMILES: C[S@+]([O-])c1ccc(N2CCN(CCn3c(=O)sc4c3nc(N)n3nc(-c5ccco5)nc43)CC2)c(F)c1] | (S)-5-amino-3-(2-(4-(2- fluoro-4-(methyl- sulfinyl)phenyl)piperazin- 1-yl)ethyl)-8-(furan-2- yl)thiazolo[5,4- e][1,2,4]triazolo[1,5- c]pyrimidin-2(3H)-one | 542.61 |
| 121 | 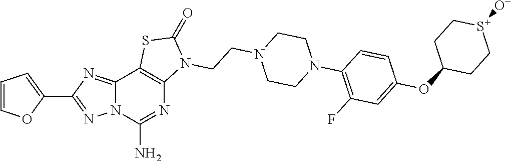 [SMILES: Nc1nc2c(sc(=O)n2CCN2CCN(c3ccc(O[C@H]4CC[S@@+]([O-])CC4)cc3F)CC2)c2nc(-c3ccco3)nn12] | 5-amino-3-(2-(4-(2-fluoro- 4-(((1s,4s)-1- oxidotetrahydro-2H- thiopyran-4- yl)oxy)phenyl)piperazin-1- yl)ethyl)-8-(furan-2- yl)thiazolo[5,4- e][1,2,4]triazolo[1,5- c]pyrimidin-2(3H)-one | 612.70 |
| 122 | 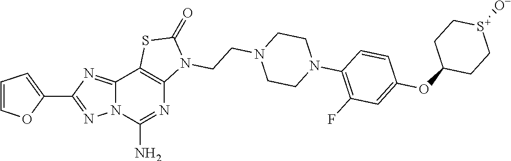 [SMILES: Nc1nc2c(sc(=O)n2CCN2CCN(c3ccc(O[C@H]4CC[S@+]([O-])CC4)cc3F)CC2)c2nc(-c3ccco3)nn12] | 5-amino-3-(2-(4-(2-fluoro- 4-(((1r,4r)-1- oxidotetrahydro-2H- thiopyran-4- yl)oxy)phenyl)piperazin-1- yl)ethyl)-8-(furan-2- yl)thiazolo[5,4- e][1,2,4]triazolo[1,5- c]pyrimidin-2(3H)-one | 612.70 |
| 123 | 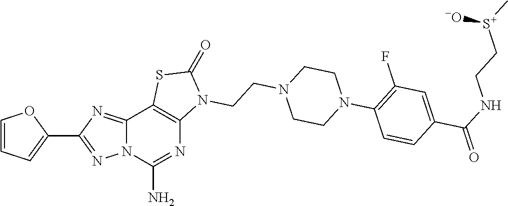 | (S)-4-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-3- fluoro-N-(2- (methylsulfinyl)ethyl) benzamide | 613.69 |
| 124 | 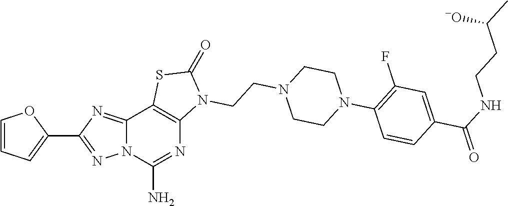 | (R)-4-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-3- fluoro-N-(2- (methylsulfinyl)ethyl) benzamide | 613.69 |
| 125 | 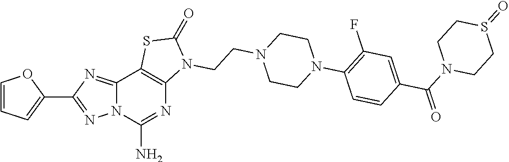 [SMILES: Nc1nc2c(sc(=O)n2CCN2CCN(c3ccc(C(=O)N4CCS(=O)CC4)cc3F)CC2)c2nc(-c3ccco3)nn12] | 5-amino-3-(2-(4-(2-fluoro- 4-(1-oxidothiomorpholine- 4- carbonyl)phenyl)piperazin- 1-yl)ethyl)-8-(furan-2- yl)thiazolo[5,4- e][1,2,4]triazolo[1,5- e]pyrimidin-2(3H)-one | 625.70 |
| 126 | 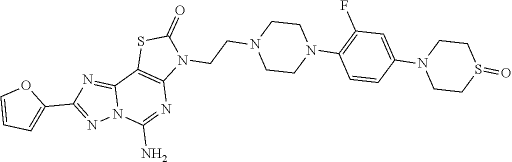 [SMILES: Nc1nc2c(sc(=O)n2CCN2CCN(c3ccc(N4CCS(=O)CC4)cc3F)CC2)c2nc(-c3ccco3)nn12] | 5-amino-3-(2-(4-(2-fluoro- 4-(1- oxidothiomorpholino)phenyl) piperazin-1-yl)ethyl)-8- (furan-2-yl)thiazolo[5,4- e][1,2,4]triazolo[1,5- c]pyrimidin-2(3H)-one | 597.69 |
| 127 | 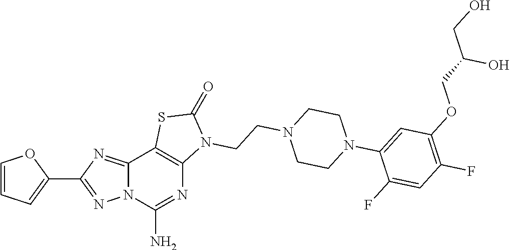 [SMILES: Nc1nc2c(sc(=O)n2CCN2CCN(c3cc(OC[C@@H](O)CO)c(F)cc3F)CC2)c2nc(-c3ccco3)nn12] | (S)-5-amino-3-(2-(4-(5-(2,3- dihydroxypropoxy)-2,4- difluorophenyl)piperazin-1- yl)ethyl)-8-(furan-2- yl)thiazolo[5,4- e][1,2,4]triazolo[1,5- c]pyrimidin-2(3H)-one | 588.59 |
| 128 | 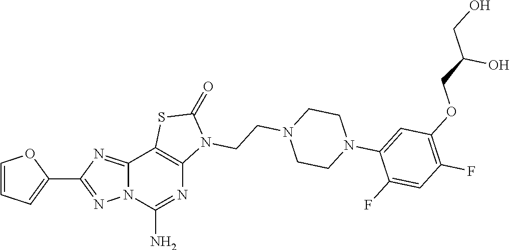 [SMILES: Nc1nc2c(sc(=O)n2CCN2CCN(c3cc(OC[C@H](O)CO)c(F)cc3F)CC2)c2nc(-c3ccco3)nn12] | (R)-5-amino-3-(2-(4-(5- (2,3-dihydroxypropoxy)- 2,4- difluorophenyl)piperazin-1- yl)ethyl)-8-(furan-2- yl)thiazolo[5,4- e][1,2,4]triazolo[1,5- c]pyrimidin-2(3H)-one | 588.59 |
| 129 | 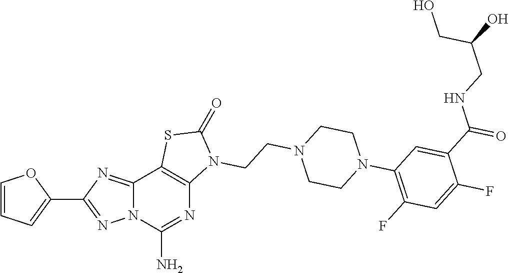 [SMILES: Nc1nc2c(sc(=O)n2CCN2CCN(c3cc(C(=O)NC[C@H](O)CO)c(F)cc3F)CC2)c2nc(-c3ccco3)nn12] | (S)-5-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-N- (2,3-dihydroxypropyl)-2,4- difluorobenzamide | 615.61 |
| 130 | 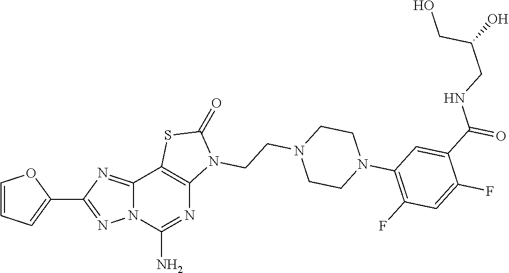 [SMILES: Nc1nc2c(sc(=O)n2CCN2CCN(c3cc(C(=O)NC[C@@H](O)CO)c(F)cc3F)CC2)c2nc(-c3ccco3)nn12] | (R)-5-(4-(2-(5-amino-8- (furan-2-yl)-2- oxothiazolo[5,4- e][1,2,4]triazolo[1,5- c]pyrimidin-3(2H)- yl)ethyl)piperazin-1-yl)-N- (2,3-dihydroxypropyl)-2,4- difluorobenzamide | 615.61 |
| 131 | 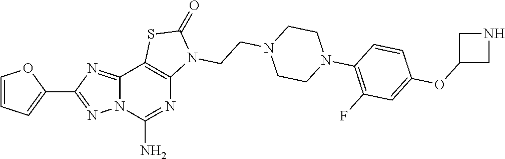 [SMILES: Nc1nc2c(sc(=O)n2CCN2CCN(c3ccc(OC4CNC4)cc3F)CC2)c2nc(-c3ccco3)nn12] | 5-amino-3-(2-(4-(4- (azetidin-3-yloxy)-2- fluorophenyl)piperazin-1- yl)ethyl)-8-(furan-2- yl)thiazolo[5,4- e][1,2,4]triazolo[1,5- c]pyrimidin-2(3H)-one | 551.60 |
| 132 | 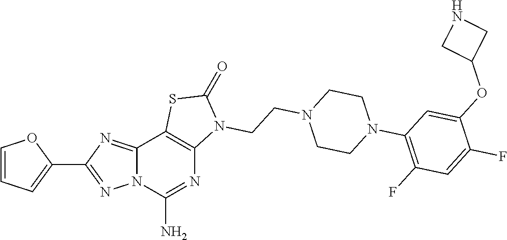 [SMILES: Nc1nc2c(sc(=O)n2CCN2CCN(c3cc(OC4CNC4)c(F)cc3F)CC2)c2nc(-c3ccco3)nn12] | 5-amino-3-(2-(4-(5- (azetidin-3-yloxy)-2,4- difluorophenyl)piperazin-1- yl)ethyl)-8-(furan-2- yl)thiazolo[5,4- e][1,2,4]triazolo[1,5- c]pyrimidin-2(3H)-one | 569.59 |
| 133 | 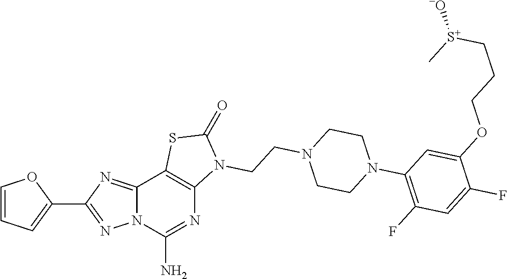 | (S)-5-amino-3-(2-(4-(2,4- difluoro-5-(3-(methyl- sulfinyl)propoxy)phenyl) piperazin-1-yl)ethyl)- 8-(furan-2-yl)thiazolo[5,4- e][1,2,4]triazolo[1,5- c]pyrimidin-2(3H)-one | 618.68 |

and pharmaceutically acceptable salts and solvates thereof.

In Table 1, the term “Cpd” means compound.

The compounds of Table 1 were named using ChemBioDraw® Ultra version 12.0 (PerkinElmer).

In one embodiment, the the compound of Formula (I) is selected from:
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 7);
(+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 8a) and
(−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 8b).

In a specific embodiment, the compound of Formula (I) is selected from:
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 7); and
(+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 8a).

In preferred embodiment, the compound of Formula (I) is (+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one (compound 8a).

In one embodiment, the present invention also relates to enantiomers, salts, solvates, polymorphs, multi-component complexes and liquid crystals of compounds of Formula (I) and subformulae thereof.

In one embodiment, the present invention also relates to polymorphs and crystal habits of compounds of Formula (I) and subformulae thereof, prodrugs and isomers thereof (including optical, geometric and tautomeric isomers) and isotopically-labeled compounds of Formula (I) and subformulae thereof.

The compounds of Formula (I) and subformulae thereof may contain an asymmetric center and thus may exist as different stereoisomeric forms. Accordingly, the present invention includes all possible stereoisomers and includes not only racemic compounds but the individual enantiomers and their non-racemic mixtures as well. When a compound is desired as a single enantiomer, such may be obtained by stereospecific synthesis, by resolution of the final product or any convenient intermediate, or by chiral chromatographic methods as each are known in the art. Resolution of the final product, an intermediate, or a starting material may be performed by any suitable method known in the art.

The compounds of the invention may be in the form of pharmaceutically acceptable salts. Pharmaceutically acceptable salts of the compounds of Formula (I) and subformulae thereof include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine, 2-(diethylamino)ethanol, ethanolamine, morpholine, 4-(2-hydroxyethyl)morpholine and zinc salts.

Hemisalts of acids and bases may also be formed, for example, hemisulphate and hemicalcium salts. Preferred, pharmaceutically acceptable salts include hydrochloride/chloride, hydrobromide/bromide, bisulphate/sulphate, nitrate, citrate, tosylate, esylate and acetate. In a particularly preferred embodiment, the compounds of Formula (I) is under the form of a HCl salt or esylate salt.

When the compounds of the invention contain an acidic group as well as a basic group the compounds of the invention may also form internal salts, and such compounds are within the scope of the invention. When the compounds of the invention contain a hydrogen-donating heteroatom (e.g. NH), the invention also covers salts and/or isomers formed by transfer of said hydrogen atom to a basic group or atom within the molecule.

Pharmaceutically acceptable salts of compounds of Formula (I) and subformulae thereof may be prepared by one or more of these methods:
(i) by reacting the compound of Formula (I) with the desired acid;
(ii) by reacting the compound of Formula (I) with the desired base;
(iii) by removing an acid- or base-labile protecting group from a suitable precursor of the compound of Formula (I) or by ring-opening a suitable cyclic precursor, for example, a lactone or lactam, using the desired acid; or
(iv) by converting one salt of the compound of Formula (I) to another by reaction with an appropriate acid or by means of a suitable ion exchange column.

All these reactions are typically carried out in solution. The salt, may precipitate from solution and be collected by filtration or may be recovered by evaporation of the solvent. The degree of ionization in the salt may vary from completely ionized to almost non-ionized.

The compounds of the present invention may be administered in the form of pharmaceutically acceptable salts. The term “pharmaceutically acceptable salt” is intended to include all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. Depending on the particular functionality of the compound of the present invention, pharmaceutically acceptable salts of the compounds of this invention include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylene-diamine, chloroprocaine, diethanolamine, procaine, N-benzylphenethyl-amine, diethylamine, piperazine, tris(hydroxymethyl)aminomethane, and tetramethylammonium hydroxide.

These salts may be prepared by standard procedures, e.g. by reacting a free acid with a suitable organic or inorganic base. Where a basic group is present, such as amino, an acidic salt, i.e. hydrochloride, hydrobromide, acetate, palmoate, esylate, tosylate and the like, can be used as the dosage form.

In addition, although generally, with respect to the salts of the compounds of the invention, pharmaceutically acceptable salts are preferred, it should be noted that the invention in its broadest sense also included non-pharmaceutically acceptable salts, which may for example be used in the isolation and/or purification of the compounds of the invention. For example, salts formed with optically active acids or bases may be used to form diastereoisomeric salts that can facilitate the separation of optically active isomers of the compounds of Formula (I) above.

The compounds of the invention may be in the form of pharmaceutically acceptable solvates. Pharmaceutically acceptable solvates of the compounds of Formula (I) and subformulae thereof contains stoichiometric or sub-stoichiometric amounts of one or more pharmaceutically acceptable solvent molecule such as ethanol or water. The term “hydrate” refers to when the said solvent is water.

The invention also generally covers all pharmaceutically acceptable predrugs and prodrugs of the compounds of Formula (I) and subformulae thereof.

Also, in the case of an alcohol group being present, pharmaceutically acceptable esters can be employed, e.g. acetate, maleate, pivaloyloxymethyl, and the like, and those esters known in the art for modifying solubility or hydrolysis characteristics for use as sustained release or prodrug formulations.

Pharmaceutical Composition

The invention thus relates to a pharmaceutical composition comprising as pharmaceutically active ingredient an A2A inhibitor, preferably being a thiocarbamate derivative, more preferably a thiocarbamate derivative of formula (I) as described above, and at least one pharmaceutically acceptable carrier, diluent, excipient and/or adjuvant.

In one embodiment, the pharmaceutical composition of the invention comprises an A2A inhibitor, such as a thiocarbamate derivative of formula (I) as described above, and at least one lipid carrier. In one embodiment, the lipid carrier is lauroyl polyoxyl-32 glycerides.

In one embodiment, the invention thus provides a pharmaceutical composition comprising:

(a) a compound of Formula (I);

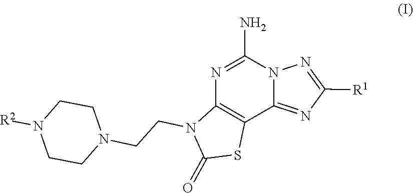

or a pharmaceutically acceptable salt or solvate thereof, wherein R¹ and R² are as defined above;

(b) a lipid carrier, preferably lauroyl polyoxyl-32 glycerides; and

(c) optionally one or more other pharmaceutically acceptable carrier, diluent, excipient and/or adjuvant.

Active Ingredient

The pharmaceutical composition of the invention thus comprises as pharmaceutically active ingredient a compound of Formula (I):

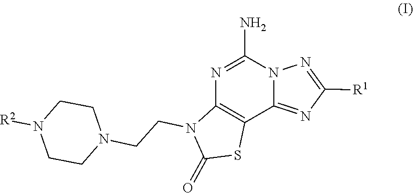

or a pharmaceutically acceptable salt or solvate thereof, wherein R¹ and R² are as defined above.

All the embodiment relative to the A2AR inhibitor detailed above apply to the pharmaceutical composition of the invention.

Lauroyl Polyoxyl-32 Glycerides

The pharmaceutical composition of the invention comprises a lipid carrier, preferably lauroyl polyoxyl-32 glycerides.

This excipient corresponds to Gelucire® 44/14 manufactured by Gattefossé (Saint-Priest—France). This excipient is also known under the following references:
lauroyl polyoxyl-32 glycerides NF/USP (NF: National Formulary; USP: US Pharmacopeia);
lauroyl macrogol-32 glycerides EP (European Pharmacopeia);
hydrogenated coconut PEG-32 esters (INCI);
CAS number 57107-95-6.

Gelucire® 44/14 corresponds to a well-defined multi-constituent substance constituted of mono-, di- and triglycerides and PEG-32 mono- and diesters of lauric acid (C₁₂). Gelucire® 44/14 has a melting point ranging from 42.5° C. to 47.5° C. (with a mean at 44° C.) and a hydrophilic/lipophilic balance (HLB) value of 14.

Gelucire® 44/14 is used in order to enhance wetting, dissolution, solubility and bioavailability of the active ingredient.

Daily Dosage

In one embodiment, the pharmaceutical composition of the invention comprising a thiocarbamate derivative of formula (I) as described above, is in a form suitable for oral administration. Such suitable administration form may be solid, semi-solid or liquid. Such suitable administration form will be clear to the skilled person; reference is made to the latest edition of Remington's Pharmaceutical Sciences.

Some preferred, but non-limiting examples of such forms include capsules (including soft and hard gelatin capsules), tablets, pills, powders, lozenges, sachets, cachets, elixirs, suspensions, emulsions, solutions and syrups.

In some embodiments, the pharmaceutical composition comprising compound of formula (I) is administered in the form of a tablet, a suspension, granules or a capsule.

In a particular embodiment, the pharmaceutical composition comprising (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one with a molecular weight of 604.65 is administered in the form of a tablet, a suspension, granules or a capsule.

The pharmaceutical composition of the invention is preferably in a daily dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Such a daily dosage can contain for example about 5 mg to about 400 mg of the pharmaceutically active ingredient, preferably about 20 mg to about 320 mg. In some embodiments, the pharmaceutically active compound is compound of Formula (I) described above. In a particular embodiment, the pharmaceutically active compound is (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one.

In one embodiment, the pharmaceutical composition of the invention is administered as a daily dose such that it corresponds administering about 5 mg to about 400 mg of compound of Formula (I) described above (free base equivalent) to the subject per day, preferably about 20 mg to about 320 mg.

In some embodiments, daily dose 130-190 mg per day, wherein the daily dose is administered in separate administrations of 2 unit doses, whereby each dose is about 65-95 mg. In some embodiments, daily dose 140-180 mg per day, wherein the daily dose is administered in separate administrations of 2 unit doses, whereby each dose is about 70-90 mg. In some embodiments, daily dose 150-170 mg per day, wherein the daily dose is administered in separate administrations of 2 unit doses, whereby each dose is about 75-85 mg. In some embodiments, daily dose is about 160 mg per day, wherein the daily dose is administered in separate administrations of 2 equal unit doses, whereby each dose is about 80 mg.

In some embodiments, about 80 mg of Compound 8a is administered to a patient in need thereof twice daily (BID). In some embodiments, a total daily administered dose of Compound 8a is about 160 mg. In some embodiments, 75-85 mg of Compound 8a is administered to a patient in need thereof twice daily (BID). In some embodiments, 70-90 mg of Compound 8a is administered to a patient in need thereof twice daily (BID).

In some embodiments, daily dosage is administered in separate administrations of 2, 3, 4, or 6 equal unit doses throughout the day. In some embodiments, the daily dose is about 160 mg per day, wherein the daily dose is administered in separate administrations of 2, 3, 4, or 6 equal unit doses.

In some embodiments, the daily dose is about 160 mg per day, wherein the daily dose is administered in separate administrations of 2, 3, 4, or 6 equal unit doses.

In another embodiment, daily dosage is administered as a single unit dose.

In some embodiments, each unit dose is administered in the form of one, two, three, or four tablet, suspension, granule or capsule.

The pharmaceutical composition of the invention may also be formulated so as to provide rapid, sustained or delayed release of the compound of Formula (I) described above contained therein.

Further Ingredients

The pharmaceutical composition of the invention may optionally comprise one or more other pharmaceutically acceptable carrier, diluent, excipient and/or adjuvant. Such suitable carrier, diluent, excipient and/or adjuvant for use in the preparation of the administration forms will be clear to the skilled person; reference is made to the latest edition of Remington's Pharmaceutical Sciences.

Especially, the pharmaceutical composition of the invention can optionally contain such inactive substances that are commonly used in pharmaceutical formulations, such as for example cosolvents, antioxidants, surfactants, wetting agents, emulsifying agents, buffering agents, pH modifying agents, preserving agents (or preservating agents), isotonifiers, stabilizing agents, granulating agents or binders, precipitation inhibitors, lubricants, disintegrants, glidants, diluents or fillers, adsorbents, dispersing agents, suspending agents, bulking agents, release agents, sweetening agents, flavoring agents, and the like.

According to one embodiment, the pharmaceutical composition of the invention comprises one or more pharmaceutically acceptable inactive ingredients selected from: caprylic acid, polyethylene glycol, propylene glycol, ethanol, glycerol, dimethylsulfoxide, dimethylacetamide, dimethylisosorbide, cellulose derivatives (including hydroxypropylmethylcellulose, methylcellulose, hydroxypropylcellulose, hydroxypropylmethylcellulose phthalate and hydroxypropylmethylcellulose acetate succinate), cremophor RH40 (polyoxyl 40 hydrogenated castor oil), cremophor EL (polyoxyl 35 hydrogenated castor oil), polysorbate 20 (polyoxyethylenesorbitan monolaurate), polysorbate 80 (polyoxyethylenesorbitan monooleate), poloxamer 188 (poly(ethylene glycol)-block-poly(propylene glycol)-block-poly(ethylene glycol)), poloxamer 407 (Poly(ethylene glycol)-block-poly(propylene glycol)-block-poly(ethylene glycol)), vitamin E TPGS (vitamin E polyethylene glycol succinate), solutol HS15 (polyoxyethylated 12-hydroxystearic acid), labrasol (caprylocaproyl polyoxyl-8 glycerides), labrafil M1944 (Oleoyl polyoxyl-6 glycerides), polyvinylpyrrolidone (also called povidone, preferably polyvinylpyrrolidone K17, K19, K29-K32, K90), polyvinylpyrrolidone polyvinylacetate copolymer, carboxymethylcellulose (Na/Ca), polyethylene glycol methyl ether-block-poly(D-L-lactide) copolymer, sodium lauryl sulfate, sodium docusate, propylene glycol monolaurate, propylene glycol dilaurate, propylene glycol monocaprylate, polyethylene glycol 660 12-monostearate, poly(butyl methacrylate-co-(2-dimethylaminoethyl) methacrylate-co-methyl methacrylate) 1:2:1, sodium lauryl sulphate.

In a preferred embodiment, the pharmaceutical composition of the invention comprises one or more pharmaceutically acceptable cosolvents. Preferably cosolvents are selected from caprylic acid, polyethylene glycol (PEG), propylene glycol, ethanol, dimethylsulfoxide, dimethylacetamide, dimethylisosorbide and mixtures thereof. In a specific embodiment, the pharmaceutical composition of the invention comprises caprylic acid and/or PEG. Advantageously, when the composition comprises PEG as cosolvent, PEG is of low molecular weight, preferably PEG is PEG 400. In an alternative embodiment, when the composition comprises PEG, it is of a moderate molecular weight, preferably PEG 2000.

In one embodiment, the pharmaceutical composition of the invention further comprises one or more antioxidant; preferably the antioxidant is selected from butylated hydroxytoluene (BHT), butylated hydroxyanisole (BHA), citric acid, sodium metabisulfite, ascorbic acid, methionine and vitamin E; more preferably the antioxidant is BHT.

In some embodiments, surfactants are added, such as for example polyethylene glycols, polyoxyethylene sorbitan fatty acid esters, sorbitan esters, sodium docusate, sodium lauryl sulfate, polysorbates (20, 80, etc.), poloxamers (188, 407 etc.), pluronic polyols, polyoxyethylene sorbitan monoethers (TWEEN®-20, TWEEN®-80, etc.), vitamin E TPGS (Vitamin E polyethylene glycol succinate), cremophor RH40 (polyoxyl 40 hydrogenated castor oil), cremophor EL (polyoxyl 35 hydrogenated castor oil), polyethylene glycol 660 12-monostearate, solutol HS15 (Polyoxyethylated 12-hydroxystearic acid), labrasol (caprylocaproyl polyoxyl-8 glycerides), labrafil M1944 (Oleoyl polyoxyl-6 glycerides).

In some embodiments, wetting agents are added, such as for example sodium lauryl sulphate, vitamin E TPGS, sodium docusate, polysorbate 80, poloxamer 407. A preferred wetting agent id sodium lauryl sulphate.

In some embodiments, emulsifying agents are added, such as for example carbomer, carrageenan, lanolin, lecithin, mineral oil, oleic acid, oleyl alcohol, pectin, poloxamer, polyoxyethylene sorbitan fatty acid esters, sorbitan esters, triethanolamine, propylene glycol monolaurate, propylene glycol dilaurate, propylene glycol monocaprylate. Preferred emulsifying agents are for example poloxamer, propylene glycol monolaurate, propylene glycol dilaurate, and propylene glycol monocaprylate.

In some embodiments, buffering agents are used to help to maintain the pH in the range that approximates physiological conditions Suitable buffering agents include both organic and inorganic acids and salts thereof, such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

In some embodiments, pH modifiers are added, such as for example sodium hydroxide, sodium bicarbonate, magnesium oxide, potassium hydroxide, meglumine, sodium carbonate, citric acid, tartaric acid, ascorbic acid, fumaric acid, succinic acid and malic acid.

In some embodiments, preservatives agents are added to retard microbial growth. Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol.

In some embodiments, isotonifiers sometimes known as “stabilizers” are added and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall or helps to inhibit the precipitation, particle growth or agglomeration of the active ingredient. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc.; organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone; cellulose derivatives such as hydroxypropylmethylcellulose, hydroxypropylmethylcellulose phthalate or hydroxypropylmethylcellulose acetate succinate; carboxymethylcellulose (Na/Ca); monosaccharides, such as xylose, mannose, fructose, glucose; disaccharides such as lactose, maltose, sucrose and trisaccacharides such as raffinose; polysaccharides such as dextran; polyethylene glycol methyl ether-block-poly(D-L-lactide) copolymer; poly(butyl methacrylate-co-(2-dimethylaminoethyl) methacrylate-co-methyl methacrylate) 1:2:1. Preferred stabilizers are for example glycerol; polyethylene glycol; polyvinylpyrrolidone; cellulose derivatives such as hydroxypropylmethylcellulose, hydroxypropylmethylcellulose phthalate or hydroxypropylmethylcellulose acetate succinate; carboxymethylcellulose (Na/Ca); polyethylene glycol methyl ether-block-poly(D-L-lactide) copolymer; and poly(butyl methacrylate-co-(2-dimethylaminoethyl) methacrylate-co-methyl methacrylate) 1:2:1.

In some embodiments granulating agent/binder(s) are added, such as for example starch, gums (inclusive of natural, semisynthetic and synthetic), microcrystalline cellulose, ethyl cellulose, methylcellulose, hydroxypropylcellulose, liquid glucose polymers such as povidone, polyvinylpyrrolidone polyvinylacetate copolymer and the like. Preferred granulating agents are for example methylcellulose, hydroxypropylcellulose, povidone and polyvinylpyrrolidone polyvinylacetate copolymer.

In some embodiments precipitation inhibitors are added, such as for example water soluble derivatives of cellulose including hydroxypropylmethylcellulose and methylcellulose, and water soluble polymers such as polyvinylpyrrolidone or polyvinylpyrrolidone polyvinylacetate copolymer. A preferred precipitation inhibitor is hydroxypropylmethylcellulose.

In some embodiments lubricants are added, such as for example magnesium stearate, glyceryl esters, behenoyl polyoxyl-8 glycerides Nf (Compritol HD5 ATO), sodium stearyl fumarate and the like.

In some embodiments disintegrants are added, such as for example synthetics like sodium starch glycolate, cross povidone, cross carmellose sodium, kollidon CL, and natural origin such as locust bean gum and the like.

In some embodiments glidants are added, such as for example talc, magnesium stearate, colloidal silicon dioxide, starch and the like.

In some embodiments diluents (or fillers) are added, such as for example dextrose, lactose, mannitol, microcrystalline cellulose, sorbitol, sucrose, dibasic calcium phosphate, calcium sulphate dehydrate, starch and the like.

In some embodiments adsorbents are added, such as for example silicon dioxide, purified aluminium silicate and the like.

In some embodiments, the pharmaceutical composition of the invention is in the form of tablets and tableting excipients are added, such as for example granulating agents, binders, lubricants, disintegrants, glidants, diluents, adsorbents and the like.

In some embodiments the pharmaceutical composition of the invention is in the form of capsules, in which the capsule shells are constructed from gelatin or from non-animal derived products such as cellulose and its derivatives such as hydroxypropylmethylcellulose. Other ingredients may be included in the capsule shells such as polyethyleneglycol to act as plasticizer; pigments such as titanium dioxide or iron oxide to provide opacity and colour differentiation; lubricants such as carnauba wax; gelling agents such as carrageenan and wetting agents such as sodium lauryl sulphate. In one embodiment, the pharmaceutical composition of the invention is formulated as capsules, wherein the capsule shells are constructed from gelatin and wherein additional components are optionally included in the capsule shells, such as for example polyethylene glycol and sodium lauryl sulphate.

Amounts

In one embodiment, the pharmaceutical composition of the invention comprises an amount of compound of Formula (I) ranging from 1% to 20% in weight to the total weight of the composition (w/w). Preferably the pharmaceutical composition of the invention comprises an amount of compound of Formula (I) ranging from 5% to 15% w/w, preferably from 8% to 12% w/w, more preferably from 9% to 11% w/w, more preferably about 10% w/w.

In one embodiment, the pharmaceutical composition of the invention comprises an amount of lauroyl polyoxyl-32 glycerides ranging from 55% to 99% in weight to the total weight of the composition (w/w). Preferably the pharmaceutical composition of the invention comprises an amount of lauroyl polyoxyl-32 glycerides ranging from 60% to 95% w/w, preferably from 65% to 90% w/w, more preferably from 70% to 85% w/w, more preferably about 70% w/w, about 71% w/w, about 72% w/w, about 73% w/w, about 74% w/w, about 75% w/w, about 76% w/w, about 77% w/w, about 78% w/w, about 79% w/w, about 80% w/w, about 81% w/w, about 82% w/w, about 83% w/w, about 84% w/w, about 85% w/w.

In one embodiment, the pharmaceutical composition of the invention may comprise PEG 400, in an amount ranging from 0% to 30% in weight to the total weight of the composition (w/w). Preferably the pharmaceutical composition of the invention comprises an amount of PEG 400 ranging from 5% to 30% w/w, preferably from 10% to 25% w/w, more preferably from 15% to 20% w/w, more preferably about 15% w/w, about 16% w/w, about 17% w/w, about 18% w/w, about 19% w/w, about 20% w/w, more preferably about 18% w/w.

In one embodiment, the pharmaceutical composition of the invention may comprise PEG 2000, in an amount ranging from 0% to 30% in weight to the total weight of the composition (w/w). Preferably the pharmaceutical composition of the invention comprises an amount of PEG 2000 ranging from 5% to 30% w/w, preferably from 10% to 25% w/w, more preferably from 15% to 20% w/w, more preferably about 15% w/w, about 16% w/w, about 17% w/w, about 18% w/w, about 19% w/w, about 20% w/w, more preferably about 18% w/w.

In one embodiment, the pharmaceutical composition of the invention may comprise caprylic acid, in an amount ranging from 0% to 20% in weight to the total weight of the composition (w/w). Preferably the pharmaceutical composition of the invention comprises an amount of caprylic acid ranging from 1% to 20% w/w, preferably from 3% to 15% w/w, more preferably from 5% to 10% w/w, more preferably about 5% w/w, about 6% w/w, about 7% w/w, about 8% w/w, about 9% w/w, more preferably about 9% w/w.

In one embodiment, the pharmaceutical composition of the invention may comprise an antioxidant agent, preferably BHT, in an amount ranging from 0% to 5% in weight to the total weight of the composition (w/w). Preferably the pharmaceutical composition of the invention comprises an amount of BHT ranging from 0.001% to 5% w/w, preferably from 0.005% to 1% w/w, more preferably from 0.01% to 0.5% w/w, more preferably about 0.01% w/w, about 0.05% w/w, about 0.10% w/w, about 0.15% w/w, about 0.20% w/w, about 0.25% w/w, about 0.30% w/w, about 0.40% w/w, about 0.50% w/w, more preferably about 0.10% w/w.

In one embodiment, the pharmaceutical composition of the invention may comprise a wetting agent, preferably sodium lauryl sulphate (SLS), in an amount ranging from 0% to 10% in weight to the total weight of the composition (w/w). Preferably the pharmaceutical composition of the invention comprises an amount of SLS ranging from 0.5% to 10% w/w, preferably from 2% to 8% w/w, more preferably from 3% to 7% w/w, more preferably from 4% to 6% w/w, more preferably about 4.0% w/w, about 4.25% w/w, about 4.5% w/w, about 4.75% w/w, about 5.0% w/w, about 5.25% w/w, about 5.5% w/w, about 5.75% w/w, about 6.0% w/w, more preferably about 5.0% w/w.

In one embodiment, the pharmaceutical composition of the invention may comprise a precipitation inhibitor, preferably hydroxypropylmethylcellulose, in an amount ranging from 0% to 10% in weight to the total weight of the composition (w/w). Preferably the pharmaceutical composition of the invention comprises an amount of hydroxypropylmethylcellulose ranging from 0.5% to 10% w/w, preferably from 2% to 8% w/w, more preferably from 3% to 7% w/w, more preferably from 4% to 6% w/w, more preferably about 4.0% w/w, about 4.25% w/w, about 4.5% w/w, about 4.75% w/w, about 5.0% w/w, about 5.25% w/w, about 5.5% w/w, about 5.75% w/w, about 6.0% w/w, more preferably about 5.0% w/w.

In one embodiment, the pharmaceutical composition of the invention comprises:

a) from 1% to 20% in weight to the total weight of the composition (w/w) of compound of Formula (I); preferably from 5% to 15% w/w, more preferably from 8% to 12% w/w, more preferably from 9% to 11% w/w, more preferably about 10% w/w; and

b) from 55% to 99% w/w of lauroyl polyoxyl-32 glycerides, preferably from 60% to 95% w/w, preferably from 65% to 90% w/w, more preferably from 70% to 85% w/w, more preferably about 70% w/w, about 71% w/w, about 72% w/w, about 73% w/w, about 74% w/w, about 75% w/w, about 76% w/w, about 77% w/w, about 78% w/w, about 79% w/w, about 80% w/w, about 81% w/w, about 82% w/w, about 83% w/w, about 84% w/w, about 85% w/w.

In one embodiment, the pharmaceutical composition of the invention comprises:

a) from 1% to 20% in weight to the total weight of the composition (w/w) of compound of Formula (I); preferably from 5% to 15% w/w, more preferably from 8% to 12% w/w, more preferably from 9% to 11% w/w, more preferably about 10% w/w;

b) from 55% to 99% w/w of lauroyl polyoxyl-32 glycerides, preferably from 60% to 95% w/w, preferably from 65% to 90% w/w, more preferably from 70% to 85% w/w, more preferably about 70% w/w, about 71% w/w, about 72% w/w, about 73% w/w, about 74% w/w, about 75% w/w, about 76% w/w, about 77% w/w, about 78% w/w, about 79% w/w, about 80% w/w, about 81% w/w, about 82% w/w, about 83% w/w, about 84% w/w, about 85% w/w;

c) from 0% to 30% w/w of PEG 400, preferably from 5% to 30% w/w, preferably from 10% to 25% w/w, more preferably from 15% to 20% w/w, more preferably about 15% w/w, about 16% w/w, about 17% w/w, about 18% w/w, about 19% w/w, about 20% w/w, more preferably about 18% w/w; and

d) from 0% to 5% w/w of BHT, preferably from 0.001% to 5% w/w, preferably from 0.005% to 1% w/w, more preferably from 0.01% to 0.5% w/w, more preferably about 0.01% w/w, about 0.05% w/w, about 0.10% w/w, about 0.15% w/w, about 0.20% w/w, about 0.25% w/w, about 0.30% w/w, about 0.40% w/w, about 0.50% w/w, more preferably about 0.10% w/win weight to the total weight of the composition (w/w).

In one embodiment, the pharmaceutical composition of the invention comprises:

a) from 1% to 20% in weight to the total weight of the composition (w/w) of compound of Formula (I); preferably from 5% to 15% w/w, more preferably from 8% to 12% w/w, more preferably from 9% to 11% w/w, more preferably about 10% w/w;

b) from 55% to 99% w/w of lauroyl polyoxyl-32 glycerides, preferably from 60% to 95% w/w, preferably from 65% to 90% w/w, more preferably from 70% to 85% w/w, more preferably about 70% w/w, about 71% w/w, about 72% w/w, about 73% w/w, about 74% w/w, about 75% w/w, about 76% w/w, about 77% w/w, about 78% w/w, about 79% w/w, about 80% w/w, about 81% w/w, about 82% w/w, about 83% w/w, about 84% w/w, about 85% w/w;

c) from 0% to 30% w/w of PEG 2000, preferably from 5% to 30% w/w, preferably from 10% to 25% w/w, more preferably from 15% to 20% w/w, more preferably about 15% w/w, about 16% w/w, about 17% w/w, about 18% w/w, about 19% w/w, about 20% w/w, more preferably about 18% w/w; and

d) from 0% to 5% w/w of BHT, preferably from 0.001% to 5% w/w, preferably from 0.005% to 1% w/w, more preferably from 0.01% to 0.5% w/w, more preferably about 0.01% w/w, about 0.05% w/w, about 0.10% w/w, about 0.15% w/w, about 0.20% w/w, about 0.25% w/w, about 0.30% w/w, about 0.40% w/w, about 0.50% w/w, more preferably about 0.10% w/win weight to the total weight of the composition (w/w).

In one embodiment, the pharmaceutical composition of the invention comprises:

a) from 1% to 20% in weight to the total weight of the composition (w/w) of compound of Formula (I); preferably from 5% to 15% w/w, more preferably from 8% to 12% w/w, more preferably from 9% to 11% w/w, more preferably about 10% w/w;

b) from 55% to 99% w/w of lauroyl polyoxyl-32 glycerides, preferably from 60% to 95% w/w, preferably from 65% to 90% w/w, more preferably from 70% to 85% w/w, more preferably about 70% w/w, about 71% w/w, about 72% w/w, about 73% w/w, about 74% w/w, about 75% w/w, about 76% w/w, about 77% w/w, about 78% w/w, about 79% w/w, about 80% w/w, about 81% w/w, about 82% w/w, about 83% w/w, about 84% w/w, about 85% w/w;

c) from 0% to 20% w/w of caprylic acid, preferably from 1% to 20% w/w, preferably from 3% to 15% w/w, more preferably from 5% to 10% w/w, more preferably about 5% w/w, about 6% w/w, about 7% w/w, about 8% w/w, about 9% w/w, more preferably about 9% w/w; and

d) from 0% to 5% w/w of BHT, preferably from 0.001% to 5% w/w, preferably from 0.005% to 1% w/w, more preferably from 0.01% to 0.5% w/w, more preferably about 0.01% w/w, about 0.05% w/w, about 0.10% w/w, about 0.15% w/w, about 0.20% w/w, about 0.25% w/w, about 0.30% w/w, about 0.40% w/w, about 0.50% w/w, more preferably about 0.10% w/win weight to the total weight of the composition (w/w).

In one embodiment, the pharmaceutical composition of the invention comprises:

a) from 1% to 20% in weight to the total weight of the composition (w/w) of compound of Formula (I) in the form of a salt, preferably as either the HCl salt or the esylate salt; preferably from 5% to 15% w/w, more preferably from 8% to 12% w/w, more preferably from 9% to 11% w/w, more preferably about 10% w/w;

b) from 55% to 99% w/w of lauroyl polyoxyl-32 glycerides, preferably from 60% to 95% w/w, preferably from 65% to 90% w/w, more preferably from 70% to 85% w/w, more preferably about 70% w/w, about 71% w/w, about 72% w/w, about 73% w/w, about 74% w/w, about 75% w/w, about 76% w/w, about 77% w/w, about 78% w/w, about 79% w/w, about 80% w/w, about 81% w/w, about 82% w/w, about 83% w/w, about 84% w/w, about 85% w/w;

c) from 0% to 30% w/w of PEG400, preferably from 5% to 30% w/w, preferably from 10% to 25% w/w, more preferably from 15% to 20% w/w, more preferably about 15% w/w, about 16% w/w, about 17% w/w, about 18% w/w, about 19% w/w, about 20% w/w, more preferably about 18% w/w; and

d) from 0% to 5% w/w of BHT, preferably from 0.001% to 5% w/w, preferably from 0.005% to 1% w/w, more preferably from 0.01% to 0.5% w/w, more preferably about 0.01% w/w, about 0.05% w/w, about 0.10% w/w, about 0.15% w/w, about 0.20% w/w, about 0.25% w/w, about 0.30% w/w, about 0.40% w/w, about 0.50% w/w, more preferably about 0.10% w/win weight to the total weight of the composition (w/w).

In one embodiment, the pharmaceutical composition of the invention comprises:

a) from 1% to 20% in weight to the total weight of the composition (w/w) of compound of Formula (I) in the form of a salt, preferably as either the HCl salt or the esylate salt; preferably from 5% to 15% w/w, more preferably from 8% to 12% w/w, more preferably from 9% to 11% w/w, more preferably about 10% w/w;

b) from 55% to 99% w/w of lauroyl polyoxyl-32 glycerides, preferably from 60% to 95% w/w, preferably from 65% to 90% w/w, more preferably from 70% to 85% w/w, more preferably about 70% w/w, about 71% w/w, about 72% w/w, about 73% w/w, about 74% w/w, about 75% w/w, about 76% w/w, about 77% w/w, about 78% w/w, about 79% w/w, about 80% w/w, about 81% w/w, about 82% w/w, about 83% w/w, about 84% w/w, about 85% w/w;

c) from 0% to 30% w/w of PEG2000, preferably from 5% to 30% w/w, preferably from 10% to 25% w/w, more preferably from 15% to 20% w/w, more preferably about 15% w/w, about 16% w/w, about 17% w/w, about 18% w/w, about 19% w/w, about 20% w/w, more preferably about 18% w/w; and

d) from 0% to 5% w/w of BHT, preferably from 0.001% to 5% w/w, preferably from 0.005% to 1% w/w, more preferably from 0.01% to 0.5% w/w, more preferably about 0.01% w/w, about 0.05% w/w, about 0.10% w/w, about 0.15% w/w, about 0.20% w/w, about 0.25% w/w, about 0.30% w/w, about 0.40% w/w, about 0.50% w/w, more preferably about 0.10% w/win weight to the total weight of the composition (w/w).

In one embodiment, the pharmaceutical composition of the invention comprises:

a) from 5% to 15% w/w of compound of Formula (I);

b) from 70% to 85% w/w of lauroyl polyoxyl-32 glycerides;

c) from 10% to 25% w/w of PEG 400; and

d) optionally from 0.01% to 0.5% w/w of BHT.

In one embodiment, the pharmaceutical composition of the invention comprises:

a) from 5% to 15% w/w of compound of Formula (I);

b) from 70% to 85% w/w of lauroyl polyoxyl-32 glycerides;

c) from 10% to 25% w/w of PEG 400;

d) optionally from 0.01% to 0.5% w/w of BHT;

e) optionally from 0.5% to 10% w/w of SLS; and

f) optionally from 0.5% to 10% w/w of hydroxypropylmethylcellulose.

In one embodiment, the pharmaceutical composition of the invention comprises:

a) from 5% to 15% w/w of compound of Formula (I);

b) from 70% to 85% w/w of lauroyl polyoxyl-32 glycerides;

c) from 10% to 25% w/w of PEG 2000; and

d) optionally from 0.01% to 0.5% w/w of BHT.

In one embodiment, the pharmaceutical composition of the invention comprises:

a) from 5% to 15% w/w of compound of Formula (I);

b) from 70% to 85% w/w of lauroyl polyoxyl-32 glycerides;

c) from 10% to 25% w/w of PEG 2000;

d) optionally from 0.01% to 0.5% w/w of BHT;

e) optionally from 0.5% to 10% w/w of SLS; and

f) optionally from 0.5% to 10% w/w of hydroxypropylmethylcellulose.

In one embodiment, the pharmaceutical composition of the invention comprises:

a) from 5% to 15% w/w of compound of Formula (I);

b) from 70% to 85% w/w of lauroyl polyoxyl-32 glycerides;

c) from 5% to 10% w/w of caprylic acid; and

d) optionally from 0.01% to 0.5% w/w of BHT.

In one embodiment, the pharmaceutical composition of the invention comprises:

a) from 5% to 15% w/w of compound of Formula (I);

b) from 70% to 85% w/w of lauroyl polyoxyl-32 glycerides;

c) from 5% to 10% w/w of caprylic acid;

d) optionally from 0.01% to 0.5% w/w of BHT;

e) optionally from 0.5% to 10% w/w of SLS; and

f) optionally from 0.5% to 10% w/w of hydroxypropylmethylcellulose.

In one embodiment, the pharmaceutical composition of the invention comprises:

a) from 5% to 15% w/w of compound of Formula (I) in the form of a salt, preferably as the HCl salt or the esylate salt;

b) from 70% to 85% w/w of lauroyl polyoxyl-32 glycerides;

c) from 10% to 25% w/w of PEG 400;

d) optionally from 0.01% to 0.5% w/w of BHT;

e) optionally from 0.5% to 10% w/w of SLS; and

f) optionally from 0.5% to 10% w/w of hydroxypropylmethylcellulose.

In one embodiment, the pharmaceutical composition of the invention comprises:

a) from 5% to 15% w/w of compound of Formula (I) in the form of a salt, preferably as the HCl salt or the esylate salt;

b) from 70% to 85% w/w of lauroyl polyoxyl-32 glycerides;

c) from 10% to 25% w/w of PEG 2000;

d) optionally from 0.01% to 0.5% w/w of BHT;

e) optionally from 0.5% to 10% w/w of SLS; and

f) optionally from 0.5% to 10% w/w of hydroxypropylmethylcellulose.

In one embodiment, the pharmaceutical composition of the invention comprises:

a) from 5% to 15% w/w of compound of Formula (I) in the form of a salt, preferably as the HCl salt or the esylate salt;

b) from 70% to 85% w/w of lauroyl polyoxyl-32 glycerides;

c) from 5% to 10% w/w of caprylic acid;

d) optionally from 0.01% to 0.5% w/w of BHT;

e) optionally from 0.5% to 10% w/w of SLS; and

f) optionally from 0.5% to 10% w/w of hydroxypropylmethylcellulose.

In some embodiment, the pharmaceutical composition of the present disclosure is selected from Formulations used in the subsequent examples are summarized below

| | | | | Composition | |
|---|---|---|---|---|---|
| | | | | for 20, 40, | |
| | Composition | Composition | Composition | and 80 mg | |
| | for 20 mg | for 40 mg | for 80 mg | Compound 8a | |
| | Compound 8a | Compound 8a | Compound 8a | capsules | |
| Component | capsules (mg) | capsules (mg) | capsules (mg) | (% w/w) | Function |
| Compound 8a | 20.00 | 40.00 | 80.00 | 10.00 | Active |
| Gelucire 44/14 | 143.8 | 287.6 | 575.20 | 71.90 | Wetting/ |
| lauroyl macrogol- | | | | | Dissolution |
| 32 glycerides EP | | | | | aid |
| Polyethylene | 36.00 | 72.00 | 144.00 | 18.00 | Wetting/ |
| glycol 400 | | | | | Dissolution |
| | | | | | aid |
| Butylated | 0.20 | 0.40 | 0.80 | 0.10 | Antioxidant |
| hydroxytoluene | | | | | |
| White body, dark | 1 capsule | — | — | — | Encapsulation |
| green cap opaque | | | | | |
| gelatin capsule, | | | | | |
| size 00 | | | | | |
| White body, dark | — | 1 capsule | — | — | Encapsulation |
| green cap opaque | | | | | |
| gelatin capsule, | | | | | |
| size 00 | | | | | |
| White opaque | — | — | 1 capsule | — | Encapsulation |
| gelatin capsule, | | | | | |
| size 00 | | | | | |
| Total per capsule | 200.00 mg | 400.00 mg | 800.00 mg | 100.00 | |

Manufacturing of the Pharmaceutical Composition

The pharmaceutical composition of the invention may be manufactured by methods well known by one skilled in the art.

In one embodiment, the pharmaceutical composition of the invention is under solid or semi-solid form. Solid dispersion may be prepared conventionally using methods such as for example fusion (melt), melt granulation, solvent evaporation, spray drying, lyophilization (freeze drying), hotmeltextrusion, electrostatic spinning method, coating on sugar beads using fluidized bed coating system or supercritical fluid technology.

In one embodiment, the pharmaceutical composition of the invention is under the form of capsules, preferably hard gelatin capsules. In such case, the capsules may be manufactured from a common blend using conventional mixing and capsule filling processes according to Good Manufacturing Practice.

In one embodiment, the manufacturing process of the capsules comprises the following steps:
i) lauroyl polyoxyl-32 glycerides is melted at a temperature not less than 50° C. but not exceeding 80° C.;
ii) optionally, further excipients, such as for example caprylic acid, sodium lauryl sulphate, hydroxypropylmethylcellulose, PEG and/or BHT, are then added to the lauroyl polyoxyl-32 glycerides and mixed together using a suitable mixer;
iii) the compound of Formula (I) as either the free base or suitable salt form, preferably the HCl salt or esylate salt, is then added gradually under continuous mixing using a suitable mixer to produce a visually uniform distribution of the drug substance with no observable lumps or agglomerates;
iv) mixing is then continued for at least 30 minutes to ensure that the drug substance is homogeneously distributed as determined visually;
v) the blend is then maintained in the molten state with continued mixing and is filled into appropriately sized gelatin capsule shells to the target capsule fill weight.

As already mention above, the gelatin capsule shells may optionally comprise additional components such as for example polyethylene glycol and sodium lauryl sulphate.

Capsule filling is undertaken using conventional capsule filling methods and equipment suitable for use with molten semi-solid formulations.

Uses of the Pharmaceutical Composition

Another object of this invention is a medicament comprising the pharmaceutical composition of the invention.

The invention is further directed to the use of the pharmaceutical composition of the invention to inhibit A2A receptor.

According to a further feature of the present invention there is provided a method for modulating A2A activity, in a patient, preferably a warm-blooded animal, and even more preferably a human, in need of such treatment, which comprises administering to said patient an effective amount of the pharmaceutical composition of the invention.

According to a further feature of the present invention there is provided the use of the pharmaceutical composition of the invention for the manufacture of a medicament for modulating A2A activity in a patient, in need of such treatment, which comprises administering to said patient an effective amount of the pharmaceutical composition of the invention.

In one embodiment, the invention relates to the use of the pharmaceutical composition of the invention, for increasing immune recognition and destruction of the cancer cells.

The pharmaceutical composition of the invention is therefore useful for the prevention and/or treatment of cancer, especially useful for the treatment of cancer.

The invention further relates to a method for treatment of cancer, which comprises administering to a mammalian species in need thereof a therapeutically effective amount of the pharmaceutical composition of the invention.

The invention further provides the use of the pharmaceutical composition of the invention for the manufacture of a medicament for treating and/or preventing cancer.

The invention also provides for a method for delaying in patient the onset of cancer comprising the administration of a pharmaceutically effective amount of the pharmaceutical composition of the invention to a patient in need thereof.

Preferably, the patient is a warm-blooded animal, more preferably a human.

Various cancers are known in the art. The cancer may be metastatic or non-metastatic. The cancer may be familial or sporadic. In some embodiments, the cancer is selected from the group consisting of: leukemia and multiple myeloma. Additional cancers that can be treated using the methods of the invention include, for example, benign and malignant solid tumors and benign and malignant non-solid tumors. In a specific embodiment, the cancer is selected from breast, bladder, carcinoid, cervical, colorectal, endometrial, glioma, head and neck, liver, lung, melanoma, ovarian, parotid, pancreatic, prostate, metastatic castrate resistant prostate cancer, renal, gastric, sinus, nasal cavity, thyroid, renal transitional cell carcinoma (TCC), renal urothelial carcinoma (UC), non-small cell lung (NSCLC), and urothelial cancers. In a specific embodiment, the cancer is breast cancer. In a specific embodiment, the cancer is carcinoid cancer. In a specific embodiment, the cancer is cervical cancer. In a specific embodiment, the cancer is colorectal cancer. In a specific embodiment, the cancer is endometrial cancer. In a specific embodiment, the cancer is glioma. In a specific embodiment, the cancer is head and neck cancer. In a specific embodiment, the cancer is liver cancer. In a specific embodiment, the cancer is lung cancer. In a specific embodiment, the cancer is melanoma. In a specific embodiment, the cancer is ovarian cancer. In a specific embodiment, the cancer is pancreatic cancer. In a specific embodiment, the cancer is prostate cancer. In a specific embodiment, the cancer is renal cancer. In a specific embodiment, the cancer is gastric cancer. In a specific embodiment, the cancer is thyroid cancer. In a specific embodiment, the cancer is urothelial cancer.

Examples of solid tumors include, but are not limited to: biliary tract cancer, brain cancer (including glioblastomas and medulloblastomas), breast cancer, carcinoid, cervical cancer, choriocarcinoma, colon cancer, colorectal cancer, endometrial cancer, esophageal cancer, gastric cancer, glioma, head and neck cancer, intraepithelial neoplasms (including Bowen's disease and Paget's disease), liver cancer, lung cancer, neuroblastomas, oral cancer (including squamous cell carcinoma), ovarian cancer (including those arising from epithelial cells, stromal cells, germ cells and mesenchymal cells), pancreatic cancer, prostate cancer, rectal cancer, renal cancer (including adenocarcinoma and Wilms tumor), sarcomas (including leiomyosarcoma, rhabdomyosarcoma, liposarcoma, fibrosarcoma and osteosarcoma), skin cancer (including melanoma, Kaposi's sarcoma, basocellular cancer and squamous cell cancer), testicular cancer including germinal tumors (seminomas, and non-seminomas such as teratomas and choriocarcinomas), stromal tumors, germ cell tumors, thyroid cancer (including thyroid adenocarcinoma and medullary carcinoma) and urothelial cancer.

Examples of solid tumors include, but are not limited to: biliary tract cancer, brain cancer (including glioblastomas and medulloblastomas), breast cancer, cervical cancer, choriocarcinoma, colon cancer, endometrial cancer, esophageal cancer, gastric cancer, intraepithelial neoplasms (including Bowen's disease and Paget's disease), liver cancer, lung cancer, neuroblastomas, oral cancer (including squamous cell carcinoma), ovarian cancer (including those arising from epithelial cells, stromal cells, germ cells and mesenchymal cells), pancreatic cancer, prostate cancer, rectal cancer, renal cancer (including adenocarcinoma and Wilms tumor), sarcomas (including leiomyosarcoma, rhabdomyosarcoma, liposarcoma, fibrosarcoma and osteosarcoma), skin cancer (including melanoma, Kaposi's sarcoma, basocellular cancer and squamous cell cancer), testicular cancer including germinal tumors (seminomas, and non-seminomas such as teratomas and choriocarcinomas), stromal tumors, germ cell tumors, and thyroid cancer (including thyroid adenocarcinoma and medullary carcinoma).

Examples of non-solid tumors include but are not limited to hematological neoplasms. As used herein, a hematologic neoplasm is a term of art which includes lymphoid disorders, myeloid disorders, and AIDS associated leukemias.

Lymphoid disorders include but are not limited to acute lymphocytic leukemia and chronic lymphoproliferative disorders (e.g., lymphomas, myelomas, and chronic lymphoid leukemias). Lymphomas include, for example, Hodgkin's disease, non-Hodgkin's lymphoma lymphomas, and lymphocytic lymphomas). Chronic lymphoid leukemias include, for example, T cell chronic lymphoid leukemias and B cell chronic lymphoid leukemias.

The invention further relates to the use of the pharmaceutical composition of the invention for the prevention and/or treatment of radiation-induced fibrosis, connective tissue diseases (such as for example Sjogrën syndrome, i.e. scleroderma), chronic bacterial infection (such as for example Helicobacter Pylori), abnormal scarring (keloids) and polymicrobial sepsis.

The invention further relates to a method for treatment or prevention of radiation-induced fibrosis, connective tissue diseases (such as for example Sjogrën syndrome, i.e. scleroderma), chronic bacterial infection (such as for example Helicobacter Pylori), abnormal scarring (keloids) and polymicrobial sepsis, which comprises administering to a mammalian species in need thereof a therapeutically effective amount of the pharmaceutical composition of the invention.

The invention further provides the use of the pharmaceutical composition of the invention for the manufacture of a medicament for treating and/or preventing radiation-induced fibrosis, connective tissue diseases (such as for example Sjogrën syndrome, i.e. scleroderma), chronic bacterial infection (such as for example Helicobacter Pylori), abnormal scarring (keloids) and polymicrobial sepsis.

The invention also provides for a method for delaying in patient the onset of radiation-induced fibrosis, connective tissue diseases (such as for example Sjogrën syndrome, i.e. scleroderma), chronic bacterial infection (such as for example Helicobacter Pylori), abnormal scarring (keloids) and polymicrobial sepsis, comprising the administration of a pharmaceutically effective amount of the pharmaceutical composition of the invention to a patient in need thereof.

Further Pharmaceutically Active Compounds

The invention also relates to a combination comprising:
(a) at least one A2A adenosine receptor (A2AR) inhibitor, and
(b) at least one anticancer agent.

In a preferred embodiment, the invention provides a combination comprising:
(a) at least one A2AR inhibitor being a thiocarbamate derivative, more preferably a thiocarbamate derivative of Formula (I)

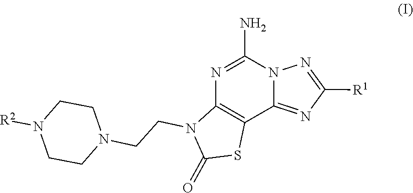

or a pharmaceutically acceptable salt or solvate thereof, wherein R¹ and R² are as defined above; and
(b) at least one anticancer agent.

As detailed below, the anticancer agent may be selected from immunotherapeutic agents, chemotherapeutic agents, antiangiogenic agents, multidrug resistance-associated proteins inhibitors, radiotherapeutic agents, and any combination thereof.

In the context of the present invention the term “combination” preferably means a combined occurrence of an A2AR inhibitor and of an anticancer agent. Therefore, the combination of the invention may occur either as one composition, comprising all the components in one and the same mixture (e.g. a pharmaceutical composition), or may occur as a kit of parts, wherein the different components form different parts of such a kit of parts. The administration of the A2AR inhibitor and of the anticancer agent may occur either simultaneously or timely staggered, with similar or different timing of administration (i.e. similar or different numbers of administration of each component), either at the same site of administration or at different sites of administration, under similar of different dosage forms.

The invention is based on the surprising finding that the combination of an A2AR inhibitor and an anticancer agent (such as for example an immunotherapeutic agent, especially a checkpoint inhibitor), shows an extremely advantageous inhibition of tumor growth and/or reduction in the number of cancer cells, resulting in enhanced survival which could not be expected from the prior art. Thus, the combined treatment with an A2AR inhibitor and with an anticancer agent, could strongly decrease the harmful impact of a disease to be treated, e.g. the growth rate of a tumor. These effects are illustrated in the Examples hereinafter.

A2AR Inhibitor

As a first component, the combination of the invention includes an A2AR inhibitor. Preferably, the A2AR inhibitor is a thiocarbamate derivative, especially a thiocarbamate derivative as those disclosed in PCT/EP2018/058301. More preferably the A2AR inhibitor is a thiocarbamate derivative of formula (I) as described above.

In a preferred embodiment, the combination of the invention thus comprises as A2AR inhibitor a compound of Formula (I):

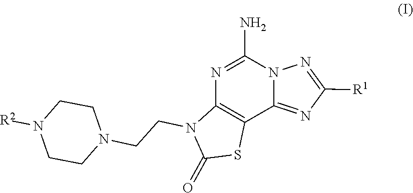

or a pharmaceutically acceptable salt or solvate thereof, wherein R¹ and R² are as defined above.

All the embodiment relative to the A2AR inhibitor detailed above apply to the combination of the invention.

Anticancer Agent

As a second component, the combination of the invention includes at least one anticancer agent.

In one embodiment, the anticancer agent is selected from immunotherapeutic agents, chemotherapeutic agents, antiangiogenic agents, multidrug resistance-associated proteins inhibitors, radiotherapeutic agents, and any combination thereof.

In one embodiment, the combination of the invention comprises a single anticancer agent. In another embodiment, the combination of the invention comprises a plurality of anticancer agents;

preferably two, three or four anticancer agents as defined below. In case of use of a combination of anticancer agents in the combination of the invention, the anticancer agents may be of the same class of agents or of different classes of agents. For example, a combination of a immunotherapeutic agent and of a chemotherapeutic agent may be used with the A2AR inhibitor.

Immunotherapeutic Agent

In one embodiment, the combination of the invention includes an immunotherapeutic agent as anticancer agent.

In such case the invention relates to a combination comprising:
(a) at least one A2A adenosine receptor (A2AR) inhibitor, and
(b) at least one immunotherapeutic agent.

In a preferred embodiment, the invention provides a combination comprising:
(a) at least one A2AR inhibitor being a thiocarbamate derivative, more preferably a thiocarbamate derivative of Formula (I)

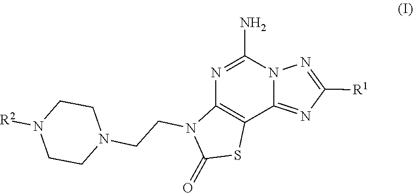

or a pharmaceutically acceptable salt or solvate thereof, wherein R¹ and R² are as defined above; and
(b) at least one immunotherapeutic agent.

In the present invention, “immunotherapy” refers to a therapy aiming at inducing and/or enhancing an immune response towards a specific target, for example towards cancer cells. In such last case, it is referred to as cancer immunotherapy.

The immunotherapeutic agent is for example selected from checkpoint inhibitors, checkpoint agonists (also called T-cell agonists), IDO inhibitors, PI3K inhibitors, adenosine receptor inhibitors, adenosine-producing enzymes inhibitors, immune cells (for conducting adoptive transfer), therapeutic vaccines, and combinations thereof. In a specific embodiment, the immunotherapeutic agent is a checkpoint inhibitor.

In one embodiment, the immunotherapeutic agent to be combined with the A2AR inhibitor of Formula (I) as described hereinabove comprises or consists of checkpoint inhibitors, checkpoint agonists, IDO inhibitors, PI3K inhibitors, adenosine receptor inhibitors, adenosine-producing enzymes inhibitors, immune cells (for conducting adoptive transfer), therapeutic vaccines, or any mixes thereof.

In the context of the present invention the term “combination” preferably means a combined occurrence of an A2AR inhibitor and of an immunotherapeutic agent. Therefore, the combination of the invention may occur either as one composition, comprising all the components in one and the same mixture (e.g. a pharmaceutical composition), or may occur as a kit of parts, wherein the different components form different parts of such a kit of parts. The administration of the A2AR inhibitor and of the immunotherapeutic agent may occur either simultaneously or timely staggered, with similar or different timing of administration (i.e. similar or different numbers of administration of each component), either at the same site of administration or at different sites of administration, under similar of different dosage forms. Such combination may induce an active immune response and thereby prevents e.g. tumor growth or induces tumor regression.

Checkpoint Inhibitors

In one embodiment, the combination of the invention includes at least one checkpoint inhibitor as immunotherapeutic agent.

Checkpoint inhibitors (CPI), that may also be referred to as immune checkpoint inhibitors (ICI), block the interactions between inhibitory receptors expressed on T cells and their ligands. As a cancer treatment, the use of checkpoint inhibitor aims at preventing the activation of inhibitory receptors expressed on T cells by ligands expressed by the tumor cells. The use of checkpoint inhibitors thus aims at preventing the inhibition of T cells present in the tumor, i.e., tumor infiltrating T cells, and thus at enhancing the subject immune response towards the tumor cells.

Thus, the combination of the invention can restore immune functions in tumor environments by using as a first component an A2AR inhibitor, and to antagonize checkpoint pathway signaling by preferably inhibiting or suppressing signal transduction by using as second component a checkpoint inhibitor as immunotherapeutic agent.

Examples of checkpoint inhibitors include, without being limited to:
inhibitors of the cell surface receptor PD-1 (programmed cell death protein 1), also known as CD279 (cluster differentiation 279);
inhibitors of the ligand PD-L1 (programmed death-ligand 1), also known as CD274 (cluster of differentiation 274) or B7-H1 (B7 homolog 1);
inhibitors of the cell surface receptor CTLA4 or CTLA-4 (cytotoxic T-lymphocyte-associated protein 4), also known as CD152 (cluster of differentiation 152);
inhibitors of LAG-3 (lymphocyte-activation gene 3), also known as CD223 (cluster differentiation 223);
inhibitors of TIM-3 (T-cell immunoglobulin and mucin-domain containing-3), also known as HAVCR2 (hepatitis A virus cellular receptor 2) or CD366 (cluster differentiation 366);
inhibitors of TIGIT (T cell immunoreceptor with Ig and ITIM domains), also known as VSIG9 (V-Set And Immunoglobulin Domain-Containing Protein 9) or VSTM3 (V-Set And Transmembrane Domain-Containing Protein 3);
inhibitors of BTLA (B and T lymphocyte attenuator), also known as CD272 (cluster differentiation 272);
inhibitors of CEACAM-1 (carcinoembryonic antigen-related cell adhesion molecule 1) also known as CD66a (cluster differentiation 66a); and
inhibitors of GITR (glucocorticoid-induced TNFR-related protein) also known as TNFRSF18 (tumor necrosis factor receptor superfamily member 18) or AITR (activation-inducible TNFR family receptor).

In one embodiment, the checkpoint inhibitor is selected from the group comprising or consisting of inhibitors of PD-1, inhibitors of PD-L1, inhibitors of CTLA4, inhibitors of LAG-3, inhibitors of TIM-3, inhibitors of TIGIT, inhibitors of BTLA, inhibitors of CEACAM-1, inhibitors of GITR and any mixtures thereof.

In one embodiment, the checkpoint inhibitor is selected from the group comprising or consisting of inhibitors of PD-1, inhibitors of PD-L1, inhibitors of CTLA-4, inhibitors of TIGIT and any mixtures thereof.

In one embodiment, the checkpoint inhibitor is selected from the group comprising or consisting of inhibitors of PD-1, inhibitors of PD-L1, inhibitors of CTLA-4 and any mixtures thereof.

In one embodiment, the checkpoint inhibitor is an inhibitor of PD-1, also referred to as an anti-PD-1. Inhibitors of PD-1 may include antibodies targeting PD-1, in particular monoclonal antibodies, and non-antibody inhibitors such as small molecule inhibitors.

Examples of inhibitors of PD-1 include, without being limited to, pembrolizumab, nivolumab, cemiplimab, tislelizumab, spartalizumab, ABBV-181, JNJ-63723283, BI 754091, MAG012, TSR-042, AGEN2034. Pembrolizumab is also known as MK-3475, MK03475, lambrolizumab, or SCH-900475. The trade name of pembrolizumab is Keytruda®. Nivolumab is also known as ONO-4538, BMS-936558, MDX1106, or GTPL7335. The trade name of nivolumab is Opdivo®. Cemiplimab is also known as REGN2810 or REGN-2810. Tislelizumab is also known as BGB-A317. Spartalizumab is also known as PDR001 or PDR-001.

In one embodiment, the checkpoint inhibitor is selected from the group comprising or consisting of pembrolizumab, nivolumab, cemiplimab, tislelizumab, spartalizumab, ABBV-181, JNJ-63723283, BI 754091, MAG012, TSR-042, AGEN2034, and any mixtures thereof.

In one embodiment, the checkpoint inhibitor is an inhibitor of PD-L1, also referred to as an anti-PD-L1. Inhibitors of PD-L1 may include antibodies targeting PD-L1, in particular monoclonal antibodies, and non-antibody inhibitors such as small molecule inhibitors.

Examples of inhibitors of PD-L1 include, without being limited to, avelumab, atezolizumab, durvalumab and LY3300054. Avelumab is also known as MSB0010718C, MSB-0010718C, MSB0010682, or MSB-0010682. The trade name of avelumab is Bavencio®. Atezolizumab is also known as MPDL3280A (clone YW243.55.S70), MPDL-3280A, RG-7446 or RG7446. The trade name of atezolizumab is Tecentriq®. Durvalumab is also known as MEDI4736 or MEDI-4736. The trade name of durvalumab is Imfinzi®.

In one embodiment, the checkpoint inhibitor is selected from the group comprising or consisting of avelumab, atezolizumab, durvalumab, LY3300054, and any mixtures thereof.

In one embodiment, the checkpoint inhibitor is an inhibitor of CTLA-4, also referred to as an anti-CTLA-4.

Inhibitors of CTLA-4 may include antibodies targeting CTLA-4, in particular monoclonal antibodies, and non-antibody inhibitors such as small molecule inhibitors.

Examples of inhibitors of CTLA-4 include, without being limited to, ipilimumab and tremelimumab. Ipilimumab is also known as BMS-734016, MDX-010, or MDX-101. The trade name of ipilimumab is Yervoy®. Tremelimumab is also known as ticilimumab, CP-675, or CP-675,206.

In one embodiment, the at least one checkpoint inhibitor is selected from the group comprising or consisting of ipilimumab, tremelimumab, and any mixtures thereof.

In one embodiment, the checkpoint inhibitor is an inhibitor of TIGIT, also referred to as an anti-TIGIT.

Checkpoint Agonists (T-Cell Agonists)

In one embodiment, the combination of the invention includes at least one checkpoint agonist (also referred to as T-cell agonist) as immunotherapeutic agent.

T-cell agonists act by activating stimulatory receptors expressed on immune cells, such as T cells. As used herein, the term “stimulatory receptors” refer to receptors that induce a stimulatory signal upon activation, and thus lead to an enhancement of the immune response. As a cancer treatment, T-cell agonist therapy aims at activating stimulatory receptors expressed on immune cells present in a tumor. In particular, T-cell agonist therapy aims at enhancing the activation of T cells present in a tumor, i.e., tumor infiltrating T cells, and thus at enhancing the subject immune response towards the tumor cells.

Examples of T-cell agonists include, without being limited to:
agonists of CD137 (cluster differentiation 137) also known as 4-1BB or TNFRS9 (tumor necrosis factor receptor superfamily, member 9);
agonists of OX40 receptor also known as CD134 (cluster differentiation 134) or TNFRSF4 (tumor necrosis factor receptor superfamily, member 4).

In one embodiment, the checkpoint agonist is selected from the group comprising or consisting of agonists of CD137, agonists of OX40 and any mixtures thereof.

Examples of agonists of CD137 include, without being limited, utomilumab and urelumab.

IDO Inhibitors

In one embodiment, the combination of the invention includes at least one inhibitor of indoleamine-2,3-dioxygenase (IDO) as immunotherapeutic agent.

Indoleamine 2,3-dioxygenase enzyme catalyzes the first and rate-limiting step of L-tryptophan (Trp) catabolism. IDO is implicated in immune modulation through its ability to limit T cell function and engage mechanisms of immune tolerance. IDO activity in tumor cells serves to impair anti-tumor responses. Inhibiting IDO thus enables to restore tumor immune surveillance.

Examples of IDO inhibitors include beta-carboline (also known as norharmane), rosmarinic acid, 1-methyl-L-tryptophan (also known as L-1-MT), epacadostat, navoximod or those disclosed in WO2015/173764, and more preferably those of formula II, II′ or II″.

In a preferred embodiment, the IDO inhibitor is selected among those disclosed in WO2015/173764, and more preferably those of formula II, II′ or II″.

PI3Kgamma Inhibitors

In one embodiment, the combination of the invention includes at least one PI3K inhibitor as immunotherapeutic agent.

A phosphoinositide 3-kinase inhibitor (PI3K inhibitor) is a class of medical drug that functions by inhibiting one or more of the phosphoinositide 3-kinase enzymes, which are part of the PI3K/AKT/mTOR pathway, an important signaling pathway for many cellular functions such as growth control, metabolism and translation initiation. Many types of cancers have activated PI3K pathway, which prohibit tumor cells from cell death.

There are a number of different classes and isoforms of PI3Ks. Class 1 PI3Ks have a catalytic subunit known as p110, with four types (isoforms)—p110 alpha, p110 beta, p110 gamma and p110 delta.

In a preferred embodiment, the PI3K inhibitor is a PI3K-gamma inhibitor.

Examples of PI3K inhibitors include wortmannin, LY294002, demethoxyviridon, hibiscone C, Idelalisib, Copanlisib, Duvelisib, Taselisib, Buparlisib, Alpelisib, Umbralisib, Dactolisib, Voxtalisib, IPI-549, RP6530, IC87114 and TG100-115.

Examples of PI3K-gamma inhibitors include Copanlisib, Duvelisib, IPI-549, RP6530, IC87114 and TG100-115.

Adenosine Receptor Inhibitor

In one embodiment, the combination of the invention includes at least one further inhibitor of adenosine receptors as immunotherapeutic agent.

As mentioned in the introduction, the adenosine receptors are a class of purinergic G protein-coupled receptors with adenosine as endogenous ligand. There are four known types of adenosine receptors in humans: A1, A2A, A2B and A3.

The combination of the invention comprises as first component an inhibitor of A2A receptor, of formula (I) as defined above. The second component of the combination may be a further inhibitor of an adenosine receptor, especially an inhibitor of A1, A2A, A2B or A3 receptors. Preferably the second component of the combination of the invention is an inhibitor of A2B receptor or an inhibitor of A3 receptor.

Examples of inhibitors of A2B receptor include ATL-801, CVT-6883, MRS-1706, MRS-1754, OSIP-339,391, PSB-603, PSB-0788 and PSB-1115.

Examples of inhibitors of A3 receptor include KF-26777, MRS-545, MRS-1191, MRS-1220, MRS-1334, MRS-1523, MRS-3777, MRE-3005-F20, MRE-3008-F20, PSB-11, OT-7999, VUF-5574 and SSR161421.

Adenosine-Producing Enzymes Inhibitors

In one embodiment, the combination of the invention includes at least one adenosine-producing enzymes inhibitor as immunotherapeutic agent.

Ectonucleotidases are families of nucleotide metabolizing enzymes that metabolize nucleotides to nucleosides. Subfamilies of ectonucleotidases include: CD39/NTPDases (ecto-nucleotide triphosphate diphosphohydrolases), nucleotide pyrophosphatase/phosphodiesterase (NPP)-type ecto-phosphodiesterases, alkaline phosphatases and ecto-5′-nucleotidases/CD73.

Among other functions, ectonucleotidases generate extracellular adenosine, the first step involving the conversion of ATP/ADP to AMP, carried out by ENTPD1, also known as CD39. The second step involves the conversion of AMP to adenosine. It is carried out by NT5E, also known as CD73. Thus ectonucleotidases are adenosine-producing enzymes.

As mentioned in the introduction, high levels of extracellular adenosine play a significant role in the evasion of antitumor immune response. Thus using inhibitors of adenosine-producing enzymes, by enabling to reduce extracellular adenosine levels is beneficial in cancer therapy.

Examples of adenosine-producing enzymes inhibitors include:
inhibitors of CD39, also known as ENTPD1 or Ecto-nucleoside triphosphate diphosphohydrolases (EC 3.6.1.5, apyrase),
inhibitors of CD73, also known as 5′-nucleotidase (5′-NT) or ecto-5′-nucleotidase or NT5E,
inhibitors of Ecto-nucleotide pyrophosphatase/PDEs (EC 3.6.1.9 and EC 3.1.4.1) and
inhibitors of alkaline phosphatases (APs; EC 3.1.3.1),
inhibitors of CD38, also known as cyclic ADP ribose hydrolase or ADP-ribosyl cyclase/cyclic ADP-ribose (cADPR) hydrolase).

Examples of adenosine-producing enzymes inhibitors include IPH5201, A001485, SRF617, ARL67156, POM-1, IPH5301, A000830, A001190, A001421, SRF373/NZV930, Darutumumab. More precisely, examples of CD39 inhibitors include IPH5201, A001485, SRF617, ARL67156 and POM-1; examples of CD73 inhibitors include IPH5301, A000830, A001190, A001421 and SRF373/NZV930; and examples of CD38 inhibitors include Darutumumab.

Immune Cells—Adoptive Cell Transfer

According to one embodiment, the immunotherapeutic agent is immune cells to be used in an adoptive transfer of cells, also referred to as adoptive cell therapy (both also referred to as ACT), particularly an adoptive transfer of T cells, also referred to as adoptive T cell therapy.

As used herein, an adoptive transfer of cells or adoptive cell therapy is defined as the transfer, for example as an infusion, of immune cells to a subject. As a cancer treatment, the adoptive transfer of immune cells to a subject aims at enhancing the subject immune response towards the cancer cells.

In one embodiment, the immune cells are T cells, in particular effector T cells. Examples of effector T cells include CD4⁺ T cells and CD8⁺ T cells.

In one embodiment, the transferred T cells are cytotoxic cells. Examples of cytotoxic T cells include CD8⁺ T cells and natural killer (NK) cells, in particular natural killer (NK) T cells.

In one embodiment, the transferred immune cells as described hereinabove are antigen-specific cells. In one embodiment, the transferred immune cells as described hereinabove are antigen-specific immune cells, wherein said antigen is specifically and/or abundantly expressed by cancer cells. In one embodiment, the transferred immune cells as described hereinabove are cancer-specific immune cells, in other words the transferred immune cells as described hereinabove specifically recognize cancer cells through an antigen specifically and/or abundantly expressed by said cancer cells. In one embodiment, the transferred immune cells as described hereinabove are cancer-specific effector T cells. In one embodiment, the transferred immune cells as described hereinabove are cancer-specific CD8⁺ effector T cells, in particular cancer-specific cytotoxic CD8⁺ T cells. In one embodiment, the transferred immune cells as described hereinabove are cancer-specific cytotoxic cells. In one embodiment, the transferred immune cells as described hereinabove are cancer-specific NK cells. In one embodiment, the transferred immune cells as described hereinabove are tumor-specific immune cells, in other words the transferred immune cells as described hereinabove specifically recognize tumor cells through an antigen specifically and/or abundantly expressed by said tumor cells. In one embodiment, the transferred immune cells as described hereinabove are tumor-specific effector T cells. In one embodiment, the transferred immune cells as described hereinabove are tumor-specific CD8⁺ effector T cells, in particular tumor-specific cytotoxic CD8⁺ T cells. In one embodiment, the transferred immune cells as described hereinabove are tumor-specific cytotoxic cells. In one embodiment, the transferred immune cells as described hereinabove are tumor-specific NK cells.

In one embodiment, the transferred immune cells as described hereinabove are autologous immune cells, in particular autologous T cells. In another embodiment, the transferred immune cells as described hereinabove are allogenic (or allogenous) immune cells, in particular allogenic NK cells.

Methods to isolate T cells from a subject, in particular antigen-specific T cells, e.g., tumor-specific T cells, are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71). Methods to expand T cells ex vivo are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71). Protocols for infusion of T cells in a subject, including pre-infusion conditioning regimens, are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71).

In one embodiment, the immune cells are CAR immune cells, in particular a CAR T cells, in the context respectively of CAR immune cell therapy and CAR T cell therapy.

As used herein, CAR immune cell therapy is an adoptive cell therapy wherein the transferred cells are immune cells as described hereinabove, such as T cells or NK cells, genetically engineered to express a chimeric antigen receptor (CAR). As a cancer treatment, the adoptive transfer of CAR immune cells to a subject aims at enhancing the subject immune response towards the cancer cells.

CARs are synthetic receptors consisting of a targeting moiety that is associated with one or more signaling domains in a single fusion molecule or in several molecules. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are usually derived from the cytoplasmic region of the CD3zeta or the Fc receptor gamma chains. First generation CARs have been shown to successfully redirect T cell cytotoxicity, however, they failed to provide prolonged expansion and anti-tumor activity in vivo. Thus, signaling domains from co-stimulatory molecules including CD28, OX-40 (CD134), and 4-1BB (CD137) have been added alone (second generation) or in combination (third generation) to enhance survival and increase proliferation of CAR modified T cells.

Thus, in one embodiment, the transferred T cells as described hereinabove are CAR T cells. The expression of a CAR allows the T cells to be redirected against a selected antigen, such as an antigen expressed at the surface of cancer cells. In one embodiment, the transferred CAR T cells recognize a tumor-specific antigen.

In another embodiment, the transferred NK cells as described hereinabove are CAR NK cells. The expression of a CAR allows the NK cells to be redirected against a selected antigen, such as an antigen expressed at the surface of cancer cells. In one embodiment, the transferred CAR NK cells recognize a tumor-specific antigen.

In one embodiment, the CAR immune cells as described hereinabove are autologous CAR immune cells, in particular autologous CAR T cells. In another embodiment, the CAR immune cells as described hereinabove are allogenic (or allogenous) CAR immune cells, in particular allogenic CAR NK cells.

Therapeutic Vaccines

According to one embodiment, the immunotherapeutic agent is a therapeutic vaccine (sometimes also referred to as a treatment vaccine).

As used herein, a therapeutic vaccine is defined as the administration of at least one tumor-specific antigen (e.g., synthetic long peptides or SLP), or of the nucleic acid encoding said tumor-specific antigen; the administration of recombinant viral vectors selectively entering and/or replicating in tumor cells; the administration of tumor cells; and/or the administration of immune cells (e.g., dendritic cells) engineered to present tumor-specific antigens and trigger an immune response against these antigens.

As a cancer treatment, therapeutic vaccines aim at enhancing the subject immune response towards the tumor cells.

Examples of therapeutic vaccines aiming at enhancing the subject immune response towards the tumor cells include, without being limited to, viral-vector based therapeutic vaccines such as adenoviruses (e.g., oncolytic adenoviruses), vaccinia viruses (e.g., modified vaccinia Ankara (MVA)), alpha viruses (e.g., Semliki Forrest Virus (SFV)), measles virus, Herpes simplex virus (HSV), and coxsackievirus; synthetic long peptide (SLP) vaccines; and dendritic cell vaccines.

Chemotherapeutic Agent

In one embodiment, the combination of the invention includes at least one chemotherapeutic agent as anticancer agent.

The chemotherapeutic agent is for example selected from anticancer alkylating agents, anticancer antimetabolites, anticancer antibiotics, plant-derived anticancer agents, anticancer platinum coordination compounds and any combination thereof.

In one embodiment, the chemotherapeutic agent to be combined with the A2AR inhibitor of Formula (I) as described hereinabove comprises or consists of anticancer alkylating agents, anticancer antimetabolites, anticancer antibiotics, plant-derived anticancer agents, anticancer platinum coordination compounds and any combination thereof.

Anticancer Alkylating Agent

In one embodiment, the combination of the invention includes at least one anticancer alkylating agent as chemotherapeutic agent.

An anticancer alkylating agent refers to an alkylating agent having anticancer activity, and the term “alkylating agent” herein generally refers to an agent giving an alkyl group in the alkylation reaction in which a hydrogen atom of an organic compound is substituted with an alkyl group.

Examples of anticancer alkylating agents include nitrogen mustard N-oxide, cyclophosphamide, ifosfamide, melphalan, busulfan, mitobronitol, carboquone, thiotepa, rammustine, nimustine, temozolomide and carmustine.

Anticancer Antimetabolite

In one embodiment, the combination of the invention includes at least one anticancer antimetabolite as chemotherapeutic agent.

An anticancer antimetabolite refers to an antimetabolite having anticancer activity, and the term “antimetabolite” herein includes, in a broad sense, substances which disturb normal metabolism and substances which inhibit the electron transfer system to prevent the production of energy-rich intermediates, due to their structural or functional similarities to metabolites that are important for living organisms (such as vitamins, coenzymes, amino acids and saccharides).

Examples of anticancer antimetabolites include methotrexate, 6-mercaptopurine riboside, rnercaptopurine, 5-fluorouracil (also called “5-FU”), tegafur, doxifluridine, carrnofur, cytarabine, cytarabine ocfosfate, enocitabine, S-1, gemcitabine, fludarabine and pemetrexed disodium. Preferably the anticancer antimetabolite is selected from 5-FU, gemcitabine and pemetrexed.

Anticancer Antibiotic

In one embodiment, the combination of the invention includes at least one anticancer antibiotic as chemotherapeutic agent.

An “anticancer antibiotic” refers to an antibiotic having anticancer activity, and the “antibiotic” herein includes substances that are produced by microorganisms or by partial or total synthesis, and derivatives thereof; and inhibit cell growth and other functions of microorganisms and of other living organisms.

Examples of anticancer antibiotic include actinomycin D, doxorubicin, daunorubicin, neocarzinostatin, bleomycin, peplomycin, mitomycin C, aclarubicin, pirarubicin, epirubicin, zinostatin stimalamer, idarubicin, sirolimus and valrabicin. Preferably, then anticancer antibiotic is doxorubicin.

Plant-Derived Anticancer Agent

In one embodiment, the combination of the invention includes at least one plant-derived anticancer agent as chemotherapeutic agent.

A “plant-derived anticancer agent” as used in the specification includes compounds having anticancer activities which originate from plants, or compounds prepared by applying chemical modification to the foregoing compounds.

Examples of plant-derived anticancer agent include vincristine, vinblastine, vindesine, etoposide, sobuzoxane, docetaxel, paclitaxel and vinorelbine. Preferably, the plant-derived anticancer agent is docetaxel.

Anticancer Platinum Coordination Compound

In one embodiment, the combination of the invention includes at least one anticancer platinum coordination compound as chemotherapeutic agent.

An “anticancer platinum coordination compound” refers to a platinum coordination compound having anticancer activity, and the term “platinum coordination compound” herein refers to a platinum coordination compound which provides platinum in ion form.

Preferred platinum compounds include cisplatin; cis-diamminediaquoplatinum (O)-ion; chloro(diethylenetriamine)-platinum (II) chloride; dichloro(ethylenediamine)-platinum (II); diamine(1,1-cyclobutanedicarboxylato) platinum (II) (carboplatin); spiroplatin; iproplatin; diamine(2-ethylmalonato)platinum (II); ethylenediaminemalonatoplatinum (H); aqua(1,2-diaminodicyclohexane)sulfatoplatinum (II); aqua(1,2-diaminodicyclohexane)malonatoplatinum (II); (1,2-diaminocyclohexane)malonatoplatinum (II); (4-carboxyphthalato)(1,2-diaminocyclohexane) platinum (II); (1,2-diaminocyclohexane)-(isocitrato)platinum (II); (1,2-diaminocyclohexane)oxalatoplatinum (II); ormaplatin; tetraplatin; carboplatin, nedaplatin and oxaliplatin. Preferably the anticancer platinum coordination compound is selected from carboplatin and oxaliplatin.

Combinations of Chemotherapeutic Agents

Combinations of chemotherapeutic agents may be used as the second component of the combination of the invention.

For example the combination known as folfox may be used. Folfox comprises the combined use of fluorouracil (antimetabolite), oxaliplatin (platinum compound) and folinic acid (chemoprotectant).

Antiangiogenic Agent

In one embodiment, the combination of the invention includes at least one antiangiogenic agent as anticancer agent.

Angiogenesis, i.e. growth of new blood vessels, plays an important role in the development of tumors and the progression of malignancies. Inhibiting angiogenesis has been shown to suppress tumor growth and metastasis. The most prominent target of antiangiogenic agents is vascular endothelial growth factor (VEGF) and its receptors. Several other factors are of interest as well, including integrins, matrix metalloproteinases, and endogenous antiangiogenic factors.

Antiangiogenic agents thus include VEGF inhibitors, integrins inhibitors and matrix metalloproteinases inhibitors.

Examples of antiangiogenic agents include Ramucirumab, IMC-18F1, Bevacizumab, Zivaflibercept, Sorafenib, Sunitinib, Axitinib, Nintedanib, Regorafenib, Pazobanib, Cabozantinib, Vandetanib and Thalidomide. In a specific embodment, the antiangiogenic agent is a VEGF inhibitor, for example Ramucirumab.

Multidrug Resistance-Associated Proteins Inhibitors

In one embodiment, the combination of the invention includes at least one multidrug resistance-associated protein inhibitor as anticancer agent.

Multidrug resistance-associated proteins (MRP/ABCC) are a subfamily of ATP-binding cassette transporters, which are capable of actively pumping a wide variety of organic anionic compounds across the plasma membrane against their concentration gradient. These proteins are involved in multi-drug resistance by transporting a wide variety of drugs outside cells, among which anticancer drugs. Inhibiting multidrug resistance-associated proteins can thus improve efficacy of anticancer drugs.

Examples of multidrug resistance-associated protein inhibitor include inhibitors of MRP4/ABCC4, inhibitors of MRP5/ABCC5 and inhibitors of MRP8/ABCC11.

Radiotherapeutic Agents—Radiation Therapy

In one embodiment, the combination of the invention includes at least one radiotherapeutic agent as anticancer agent.

“Radiation therapy” refers to a method of treatment of cancer employing various radiations such as X-ray, γ-ray, neutron ray, electron beam, proton beam and radiation sources. It is used as part of cancer treatment to control or kill malignant cells. Radiation therapy may be curative in a number of types of cancer if they are localized to one area of the body. It may also be used as part of adjuvant therapy, to prevent tumor recurrence after surgery to remove a primary malignant tumor.

The three main divisions of radiation therapy are: external beam radiation therapy (EBRT or XRT); brachytherapy or sealed source radiation therapy; and systemic radioisotope therapy (RIT) or unsealed source radiotherapy. The differences relate to the position of the radiation source; external is outside the body, brachytherapy uses sealed radioactive sources placed precisely in the area under treatment, and systemic radioisotopes are given by infusion or oral ingestion. Particle therapy is a special case of external beam radiation therapy where the particles are protons or heavier ions. Radiations may be delivered by a linear accelerator.

Systemic radioisotope therapy (RIT) is a form of targeted therapy. Targeting can be due to the chemical properties of the isotope such as radioiodine which is specifically absorbed by the thyroid gland a thousand-fold better than other bodily organs. Targeting can also be achieved by attaching the radioisotope to another molecule or antibody to guide it to the target tissue, forming a radiopharmaceutical agent.

In order to enhance the radiosensitivity of the cancer, radiosensitizing agents may be administered during a radiation therapy. Examples of radiosensitizing agents include: Cisplatin, Nimorazole, and Cetuximab.

Thus, in one embodiment, radiotherapeutic agent is selected from sealed radiation sources, radioisotopes, radiopharmaceutical agents, radiosensitizing agents and the like useful in the course of radiation therapy.

In another embodiment, the invention also provides the use of the A2AR inhibitor as described above, in combination with radiation therapy, including radiation therapy performed by external beam radiations or X-ray radiations; brachytherapy; and systemic radioisotope therapy.

Methods Comprising Administrating Further Pharmaceutically Active Ingredients

In one embodiment, the method of the invention comprises administrating a pharmaceutical composition comprising at least one A2AR inhibitor as defined above and administrating at least one anticancer agent as defined above.

In one embodiment, the method of the invention comprises administrating a pharmaceutical composition comprising at least one A2AR inhibitor as defined above and administrating at least one anticancer agent as defined above, preferably pembrolizumab.

In one embodiment, the method of the invention comprises administrating a pharmaceutical composition comprising at least one A2AR inhibitor as defined above and administrating at least one immunotherapeutic agent as defined above.

In one embodiment, the method of the invention comprises administrating a pharmaceutical composition comprising at least one A2AR inhibitor as defined above and administrating at least one checkpoint inhibitor as defined above, preferably an inhibitor of PD-1, PD-L1, CTLA-4 or of TIGIT, or any mixture thereof.

In one embodiment, the method of the invention comprises administrating a pharmaceutical composition comprising at least one A2AR inhibitor as defined above and administrating at least one adenosine-producing enzymes inhibitor as defined above, preferably at least one inhibitor of CD39, such as for example ARL67156 and POM-1.

In one embodiment, the method of the invention comprises administrating a pharmaceutical composition comprising at least one A2AR inhibitor as defined above and administrating at least one chemotherapeutic agent as defined above.

In one embodiment, the method of the invention comprises administrating a pharmaceutical composition comprising at least one A2AR inhibitor as defined above and administrating at least one anticancer antibiotic as defined above, such as for example doxorubicin.

In one embodiment, the method of the invention comprises administrating a pharmaceutical composition comprising at least one A2AR inhibitor as defined above and administrating at least one anticancer platinum coordination compound as defined above, such as for example oxaliplatin.

In one embodiment, the method of the invention comprises administrating a pharmaceutical composition comprising at least one A2AR inhibitor as defined above and administrating at least one immunotherapeutic agent as defined above and at least one chemotherapeutic agent as defined above.

In a specific embodiment, the method of the invention comprises administrating a pharmaceutical composition comprising least one A2AR inhibitor as defined above, at least one checkpoint inhibitor as defined above and at least one chemotherapeutic agent as defined above.

In a specific embodiment, the method of the invention comprises administrating pharmaceutical composition comprising at least one A2AR inhibitor as defined above, at least one inhibitor of PD-L1, CTLA-4 or TIGIT and at least one chemotherapeutic agent as defined above. In a specific embodiment, the method of the invention comprises administrating a pharmaceutical composition comprising at least one A2AR inhibitor as defined above, at least one checkpoint inhibitor as defined above and at least one. In a specific embodiment, the method of the invention comprises administrating a pharmaceutical composition comprising at least one A2AR inhibitor as defined above, at least one inhibitor of PD-L1, CTLA-4 or TIGIT as defined above and at least one anticancer antibiotic as defined above, such as for example doxorubicin.

In a specific embodiment, the method of the invention comprises administrating a pharmaceutical composition comprising at least one A2AR inhibitor as defined above and at least two checkpoint inhibitors as defined above. In a specific embodiment, the combination of the invention comprises a pharmaceutical composition comprising at least one A2AR inhibitor as defined above, at least one inhibitor of PD-L1 as defined above and at least one inhibitor of TIGIT as defined above.

Pharmaceutical Composition Further Comprising Pharmaceutically Active Compounds

The invention further relates to a method administrating a pharmaceutical composition comprising the combination of at least one A2AR inhibitor as defined above and further pharmaceutically active compounds.

The invention further relates to a pharmaceutical composition comprising the combination of at least one A2AR inhibitor as defined above and further pharmaceutically active compounds.

In one embodiment, the pharmaceutical composition comprises:
(a) at least one A2AR inhibitor,
(b) at least one anticancer agent, and
(c) at least one pharmaceutically acceptable carrier, diluent, excipient and/or adjuvant.

In a preferred embodiment, the invention provides a pharmaceutical composition comprising:
(a) at least one A2AR inhibitor being a thiocarbamate derivative, more preferably a thiocarbamate derivative of Formula (I)

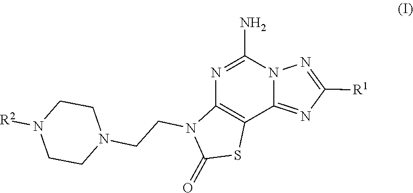

or a pharmaceutically acceptable salt or solvate thereof, as defined above;
(b) at least one anticancer agent as defined above, such as for example immunotherapeutic agents, chemotherapeutic agents, antiangiogenic agents, multidrug resistance-associated proteins inhibitors, radiotherapeutic agents, or any combination thereof, and
(c) at least one pharmaceutically acceptable carrier, diluent, excipient and/or adjuvant.

The at least one pharmaceutically acceptable carrier, diluent, excipient and/or adjuvant for use in the preparation of the administration forms will be clear to the skilled person; reference is made to the latest edition of Remington's Pharmaceutical Sciences.

Especially, the pharmaceutical composition comprising the combination of the invention can optionally contain such inactive substances that are commonly used in pharmaceutical formulations, such as for example cosolvents, lipid carrier, antioxidants, surfactants, wetting agents, emulsifying agents, buffering agents, pH modifying agents, preserving agents (or preservating agents), isotonifiers, stabilizing agents, granulating agents or binders, precipitation inhibitors, lubricants, disintegrants, glidants, diluents or fillers, adsorbents, dispersing agents, suspending agents, bulking agents, release agents, sweetening agents, flavoring agents, and the like.

In a preferred embodiment, the pharmaceutical composition comprising the combination of the invention comprises one or more pharmaceutically acceptable cosolvent. Preferably cosolvents are selected from caprylic acid, polyethylene glycol (PEG), propylene glycol, ethanol, dimethylsulfoxide, dimethylacetamide, dimethylisosorbide and mixtures thereof. In a specific embodiment, the pharmaceutical composition of the invention comprises caprylic acid and/or PEG. Advantageously, when the composition comprises PEG as cosolvent, PEG is of low molecular weight, preferably PEG is PEG400. In an alternative embodiment, when the composition comprises PEG, it is of a moderate molecular weight, preferably PEG 2000.

In a specific embodiment, the pharmaceutical composition comprising the combination of the invention comprises an one or more pharmaceutically acceptable lipid carrier. In a preferred embodiment, the lipid carrier is lauroyl polyoxyl-32 glycerides. This excipient corresponds to Gelucire® 44/14 manufactured by Gattefossé (Saint-Priest—France). This excipient is also known under the following references: lauroyl polyoxyl-32 glycerides NF/USP (NF: National Formulary; USP: US Pharmacopeia); lauroyl macrogol-32 glycerides EP (European Pharmacopeia); hydrogenated coconut PEG-32 esters (INCI); CAS number 57107-95-6. Gelucire® 44/14 corresponds to a well-defined multi-constituent substance constituted of mono-, di- and triglycerides and PEG-32 mono- and diesters of lauric acid (C₁₂). Gelucire® 44/14 has a melting point ranging from 42.5° C. to 47.5° C. (with a mean at 44° C.) and an hydrophilic/lipophilic balance (HLB) value of 14.

In one embodiment, the pharmaceutical composition comprising the combination of the invention further comprises one or more antioxidant; preferably the antioxidant is selected from butylated hydroxytoluene (BHT), butylated hydroxyanisole (BHA), citric acid, sodium metabisulfite, ascorbic acid, methionine and vitamin E; more preferably the antioxidant is BHT.

In some embodiments, surfactants are added, such as for example polyethylene glycols, polyoxyethylene sorbitan fatty acid esters, sorbitan esters, sodium docusate, sodium lauryl sulfate, polysorbates (20, 80, etc.), poloxamers (188, 407 etc.), pluronic polyols, polyoxyethylene sorbitan monoethers (TWEEN®-20, TWEEN®-80, etc.), vitamin E TPGS (Vitamin E polyethylene glycol succinate), cremophor RH40 (polyoxyl 40 hydrogenated castor oil), cremophor EL (polyoxyl 35 hydrogenated castor oil), polyethylene glycol 660 12-monostearate, solutol HS15 (Polyoxyethylated 12-hydroxystearic acid), labrasol (caprylocaproyl polyoxyl-8 glycerides), labrafil M1944 (Oleoyl polyoxyl-6 glycerides).

In some embodiments, wetting agents are added, such as for example sodium lauryl sulphate, vitamin E TPGS, sodium docusate, polysorbate 80, poloxamer 407. A preferred wetting agent id sodium lauryl sulphate.

In some embodiments, emulsifying agents are added, such as for example carbomer, carrageenan, lanolin, lecithin, mineral oil, oleic acid, oleyl alcohol, pectin, poloxamer, polyoxyethylene sorbitan fatty acid esters, sorbitan esters, triethanolamine, propylene glycol monolaurate, propylene glycol dilaurate, propylene glycol monocaprylate. Preferred emulsifying agents are for example poloxamer, propylene glycol monolaurate, propylene glycol dilaurate, and propylene glycol monocaprylate.

In some embodiments, buffering agents are used to help to maintain the pH in the range that approximates physiological conditions Suitable buffering agents include both organic and inorganic acids and salts thereof, such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

In some embodiments, pH modifiers are added, such as for example sodium hydroxide, sodium bicarbonate, magnesium oxide, potassium hydroxide, meglumine, sodium carbonate, citric acid, tartaric acid, ascorbic acid, fumaric acid, succinic acid and malic acid;

In some embodiments, preservatives agents are added to retard microbial growth. Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol.

In some embodiments, isotonifiers sometimes known as “stabilizers” are added and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall or helps to inhibit the precipitation, particle growth or agglomeration of the active ingredient. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc.; organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone; cellulose derivatives such as hydroxypropylmethylcellulose, hydroxypropylmethylcellulose phthalate or hydroxypropylmethylcellulose acetate succinate; carboxymethylcellulose (Na/Ca); monosaccharides, such as xylose, mannose, fructose, glucose; disaccharides such as lactose, maltose, sucrose and trisaccacharides such as raffinose; polysaccharides such as dextran; polyethylene glycol methyl ether-block-poly(D-L-lactide) copolymer; poly(butyl methacrylate-co-(2-dimethylaminoethyl) methacrylate-co-methyl methacrylate) 1:2:1. Preferred stabilizers are for example glycerol; polyethylene glycol; polyvinylpyrrolidone; cellulose derivatives such as hydroxypropylmethylcellulose, hydroxypropylmethylcellulose phthalate or hydroxypropylmethylcellulose acetate succinate; carboxymethylcellulose (Na/Ca); polyethylene glycol methyl ether-block-poly(D-L-lactide) copolymer; and poly(butyl methacrylate-co-(2-dimethylaminoethyl) methacrylate-co-methyl methacrylate) 1:2:1.

In some embodiments granulating agent/binder(s) are added, such as for example starch, gums (inclusive of natural, semisynthetic and synthetic), microcrystalline cellulose, ethyl cellulose, methylcellulose, hydroxypropylcellulose, liquid glucose polymers such as povidone, polyvinylpyrrolidone polyvinylacetate copolymer and the like. Preferred granulating agents are for example methylcellulose, hydroxypropylcellulose, povidone and polyvinylpyrrolidone polyvinylacetate copolymer.

In some embodiments precipitation inhibitors are added, such as for example water soluble derivatives of cellulose including hydroxypropylmethylcellulose and methylcellulose, and water soluble polymers such as polyvinylpyrrolidone or polyvinylpyrrolidone polyvinylacetate copolymer. A preferred precipitation inhibitor is hydroxypropylmethylcellulose.

In some embodiments lubricants are added, such as for example magnesium stearate, glyceryl esters, behenoyl polyoxyl-8 glycerides Nf (Compritol HD5 ATO), sodium stearyl fumarate and the like.

In some embodiments disintegrants are added, such as for example synthetics like sodium starch glycolate, cross povidone, cross carmellose sodium, kollidon CL, and natural origin such as locust bean gum and the like.

In some embodiments glidants are added, such as for example talc, magnesium stearate, colloidal silicon dioxide, starch and the like.

In some embodiments diluents (or fillers) are added, such as for example dextrose, lactose, mannitol, microcrystalline cellulose, sorbitol, sucrose, dibasic calcium phosphate, calcium sulphate dehydrate, starch and the like.

In some embodiments adsorbents are added, such as for example silicon dioxide, purified aluminium silicate and the like.

In some embodiments, the pharmaceutical composition comprising the combination of the invention is in the form of tablets and tableting excipients are added, such as for example granulating agents, binders, lubricants, disintegrants, glidants, diluents, adsorbents and the like.

In some embodiments the pharmaceutical composition comprising the combination of the invention is in the form of capsules, in which the capsule shells are constructed from gelatin or from non-animal derived products such as cellulose and its derivatives such as hydroxypropylmethylcellulose. Other ingredients may be included in the capsule shells such as polyethyleneglycol to act as plasticizer; pigments such as titanium dioxide or iron oxide to provide opacity and colour differentiation; lubricants such as carnauba wax; gelling agents such as carrageenan and wetting agents such as sodium lauryl sulphate. In one embodiment, the pharmaceutical composition comprising the combination of the invention is formulated as capsules, wherein the capsule shells are constructed from gelatin and wherein additional components are optionally included in the capsule shells, such as for example polyethylene glycol and sodium lauryl sulphate.

By means of non-limiting examples, the pharmaceutical composition comprising the combination may be in a form suitable for oral administration, for parenteral administration (such as by intravenous, intramuscular or subcutaneous injection or intravenous infusion), for topical administration (including ocular), for rectal administration, for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is made to the latest edition of Remington's Pharmaceutical Sciences.

The compositions may be formulated so as to provide rapid, sustained or delayed release of the active compound(s) contained therein.

According to one embodiment, the pharmaceutical composition comprising the combination is in an adapted form for an oral administration. Forms adapted to oral administration may be solid, semi-solid or liquid. Some preferred, but non-limiting examples of such forms include liquid, paste or solid compositions, and more particularly tablets, tablets formulated for extended or sustained release, capsules (including soft and hard gelatin capsules), pills, dragees, lozenges, sachets, cachets, powder, liquids, gels, syrups, slurries, elixirs, emulsions, solutions, and suspensions.

According to another embodiment, the pharmaceutical composition comprising the combination is in an adapted form for an injection, especially to be injected to the subject by intravenous, intramuscular, intraperitoneal, intrapleural, subcutaneous, transdermal injection or infusion.

Sterile injectable forms of the pharmaceutical composition of the invention include sterile injectable solutions and sterile packaged powders (which are usually reconstituted prior to use) for administration as a bolus and/or for continuous administration.

Sterile injectable forms of the pharmaceutical composition of the invention may be a solution or an aqueous or oleaginous suspension. These suspensions may be formulated according to techniques known in the art using suitable dispersing or wetting agents and suspending agents. The sterile injectable preparation may also be a sterile injectable solution or suspension in a non-toxic pharmaceutically acceptable diluent or solvent. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

According to another embodiment, the pharmaceutical composition comprising the combination of the invention is in an adapted form for a topical administration. Examples of forms adapted for topical administration include, without being limited to, liquid, paste or solid compositions, and more particularly aqueous solutions, drops, dispersions, sprays, ointments, cremes, lotions, microcapsules, micro- or nanoparticles, polymeric patch, or controlled-release patch, and the like.

According to another embodiment, the pharmaceutical composition comprising the combination of the invention is in an adapted form for a rectal administration. Examples of forms adapted for rectal administration include, without being limited to, suppository, micro enemas, enemas, gel, rectal foam, cream, ointment, and the like.

According to another embodiment, the pharmaceutical composition comprising the combination of the invention is in an adapted form for an administration by inhalation. Examples of forms adapted for administration by inhalation include, without being limited to aerosols.

The pharmaceutical preparations of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use.

Regimen and Doses of Pharmaceutical Composition Further Comprising Pharmaceutically Active Compounds

In the context of the present invention, the administration of the A2AR inhibitor and the anticancer agent may occur either simultaneously or timely staggered, with similar or different timing of administration (i.e. similar or different numbers of administration of each component), either at the same site of administration or at different sites of administration, under similar of different dosage forms, as further outlined below.

To ensure that the separate mechanisms elicited by the A2AR inhibitor and the anticancer agent are not negatively influenced by each other, the anticancer agent and the A2AR inhibitor are preferably administered separated in time (in a time-staggered manner), i.e. sequentially, and/or are administered at different administration sites. This means that the A2AR inhibitor may be administrated e.g. prior, concurrent or subsequent to the anticancer agent, or vice versa. Alternatively or additionally, the A2AR inhibitor and the anticancer agent may be administered at different administration sites, or at the same administration site, preferably, when administered in a time staggered manner.

In one embodiment, the A2AR inhibitor is to be administered prior to and/or concomitantly with an immunotherapeutic agent as described hereinabove. In one embodiment, the immunotherapeutic agent is a checkpoint inhibitor and the A2AR inhibitor is to be administered prior to the day or on the same day that the checkpoint inhibitor as described hereinabove is administered.

In one embodiment, the A2AR inhibitor is to be administered prior to and/or concomitantly with an immunotherapeutic agent as described hereinabove and continuously thereafter.

In one embodiment, the A2AR inhibitor is to be administered prior to or concomitantly with an immunotherapeutic agent as described hereinabove and subsequently for at least 1, 2, 3, 4, 5, 6, 7, 8, 9, or 10 days thereafter. In another embodiment, the A2AR inhibitor is to be administered prior to or concomitantly with an immunotherapeutic agent as described hereinabove and subsequently for at least 1, 2, 3, 4, 5, 6, 7, 8, 9, or 10 weeks thereafter. In another embodiment, the A2AR inhibitor is to be administered prior to or concomitantly with an immunotherapeutic agent as described hereinabove and subsequently for at least 1, 2, 3, 4, 5, 6, 7, 8, 9, or 10 months thereafter.

In one embodiment, the immunotherapeutic agent is a checkpoint inhibitor and the A2AR inhibitor is to be administered prior to or concomitantly with said checkpoint inhibitor. In one embodiment, the immunotherapeutic agent is a checkpoint inhibitor and the A2AR inhibitor is to be administered prior to or concomitantly with said checkpoint inhibitor and continuously thereafter. In one embodiment, the immunotherapeutic agent is a checkpoint inhibitor and the A2AR inhibitor is to be administered prior to or concomitantly with said checkpoint inhibitor and subsequently for at least 1, 2, 3, 4, 5, 6, 7, 8, 9, or 10 weeks thereafter.

Depending on the condition to be prevented or treated and the form of administration, the combination of the invention may be administered as a single daily dose, divided over one or more daily doses.

According to one embodiment, a therapeutically effective dose of A2AR inhibitor as described hereinabove is to be administered for use in the treatment of a cancer in a subject in need thereof, wherein said A2AR inhibitor is used in combination with an anticancer agent, such as for example an immunotherapeutic agent. Thus, in one embodiment, the pharmaceutical combination or kit of parts of the invention comprises a therapeutically effective dose of A2AR inhibitor as described hereinabove and a therapeutically effective dose of anticancer agent as described hereinabove.

It will be understood that the total daily usage of A2AR inhibitor and anticancer agent will be decided by the attending physician within the scope of sound medical judgment. The specific dose for any particular subject will depend upon a variety of factors such as the cancer to be treated; the age, body weight, general health, sex and diet of the patient; and like factors well-known in the medical arts.

In one embodiment, the subject is a mammal, preferably a human, and the dose of A2AR inhibitor, preferably a therapeutically effective dose, is a dose ranging from about 0.01 mg per kilo body weight (mg/kg) to about 5 mg/kg, preferably about 0.08 mg/kg to about 3.3 mg/kg, more preferably about 0.15 mg/kg to about 1.7 mg/kg.

In one embodiment, the subject is a mammal, preferably a human, and the dose of A2AR inhibitor, preferably a therapeutically effective dose, is a dose ranging from about 0.01 mg per kilo body weight per day (mg/kg/day) to about 5 mg/kg/day, preferably about 0.08 mg/kg/day to about 3.3 mg/kg/day, more preferably about 0.15 mg/kg/day to about 1.7 mg/kg/day.

In one embodiment, the subject is a mammal, preferably a human, and the dose of A2AR inhibitor, preferably a therapeutically effective dose, is a dose ranging from about 1 mg to about 500 mg, preferably about 5 mg to about 200 mg, more preferably from about 20 mg to about 320 mg.

In one embodiment, the subject is a mammal, preferably a human, and the dose of A2AR inhibitor, preferably a therapeutically effective dose, is a dose ranging from about 1 mg to about 500 mg per administration, preferably about 5 mg to about 200 mg per administration, more preferably from about 10 mg to about 100 mg per administration.

In one embodiment, the subject is a mammal, preferably a human, and the dose of A2AR inhibitor, preferably a therapeutically effective dose, is a daily dose ranging from about 1 mg to about 500 mg, preferably about 5 mg to about 200 mg, more preferably from about 10 mg to about 100 mg.

In one embodiment, the subject is a mammal, preferably a human, and the dose of A2AR inhibitor, preferably a therapeutically effective dose, is a daily dose to be administered in one, two, three or more takes. In one embodiment, the subject is a mammal, preferably a human, and the dose of A2AR inhibitor, preferably a therapeutically effective dose, is a daily dose to be administered in one or two takes.

Uses of Pharmaceutical Composition Further Comprising Pharmaceutically Active Compounds

Another object of this invention is the use of the combination of the invention as a medicament. Thus, in one embodiment, the invention provides the use of the combination of the invention for the manufacturing of a medicament. Especially, the invention provides the use of the pharmaceutical composition of the invention or the kit of the invention for the manufacturing of a medicament.

Especially, the invention provides the combination, the pharmaceutical composition or the kit of parts of the invention, for use in the treatment and/or prevention of cancer.

In one embodiment, the invention relates to a treatment and/or prevention of cancer, which comprises administering to a mammal species in need thereof a therapeutically effective amount of the combination, pharmaceutical composition or kit of parts of the invention.

The invention further provides the use of the combination, pharmaceutical composition or kit of parts of the invention for the manufacture of a medicament for treating and/or preventing cancer.

The invention also provides for a method for delaying in patient the onset of cancer comprising the administration of a pharmaceutically effective amount of the combination, pharmaceutical composition or kit of parts of the invention to a patient in need thereof.

Various cancers are known in the art. Cancers that can be treated using the methods of the invention include solid cancers and non-solid cancers, especially benign and malignant solid tumors and benign and malignant non-solid tumors. The cancer may be metastatic or non-metastatic. The cancer may be may be familial or sporadic.

In one embodiment, the cancer to be treated according to the present invention is a solid cancer. As used herein, the term “solid cancer” encompasses any cancer (also referred to as malignancy) that forms a discrete tumor mass, as opposed to cancers (or malignancies) that diffusely infiltrate a tissue without forming a mass.

Examples of solid tumors include, but are not limited to: biliary tract cancer, brain cancer (including glioblastomas and medulloblastomas), breast cancer, carcinoid, cervical cancer, choriocarcinoma, colon cancer, colorectal cancer, endometrial cancer, esophageal cancer, gastric cancer, glioma, head and neck cancer, intraepithelial neoplasms (including Bowen's disease and Paget's disease), liver cancer, lung cancer, neuroblastomas, oral cancer (including squamous cell carcinoma), ovarian cancer (including those arising from epithelial cells, stromal cells, germ cells and mesenchymal cells), pancreatic cancer, prostate cancer, rectal cancer, renal cancer (including adenocarcinoma and Wilms tumor), sarcomas (including leiomyosarcoma, rhabdomyosarcoma, liposarcoma, fibrosarcoma and osteosarcoma), skin cancer (including melanoma, Kaposi's sarcoma, basocellular cancer and squamous cell cancer), testicular cancer including germinal tumors (seminomas, and non-seminomas such as teratomas and choriocarcinomas), stromal tumors, germ cell tumors, thyroid cancer (including thyroid adenocarcinoma and medullary carcinoma) and urothelial cancer.

In another embodiment, the cancer to be treated according to the present invention is a non-solid cancer. Examples of non-solid tumors include but are not limited to hematological neoplasms. As used herein, a hematologic neoplasm is a term of art which includes lymphoid disorders, myeloid disorders, and AIDS associated leukemias.

Lymphoid disorders include but are not limited to acute lymphocytic leukemia and chronic lymphoproliferative disorders (e.g., lymphomas, myelomas, and chronic lymphoid leukemias). Lymphomas include, for example, Hodgkin's disease, non-Hodgkin's lymphoma lymphomas, and lymphocytic lymphomas). Chronic lymphoid leukemias include, for example, T cell chronic lymphoid leukemias and B cell chronic lymphoid leukemias.

In a specific embodiment, the cancer is selected from breast, carcinoid, cervical, colorectal, endometrial, glioma, head and neck, liver, lung, melanoma, ovarian, pancreatic, prostate, renal, gastric, thyroid and urothelial cancers.

In a specific embodiment, the cancer is breast cancer. In a specific embodiment, the cancer is carcinoid cancer. In a specific embodiment, the cancer is cervical cancer. In a specific embodiment, the cancer is colorectal cancer. In a specific embodiment, the cancer is endometrial cancer. In a specific embodiment, the cancer is glioma. In a specific embodiment, the cancer is head and neck cancer. In a specific embodiment, the cancer is liver cancer. In a specific embodiment, the cancer is lung cancer. In a specific embodiment, the cancer is melanoma. In a specific embodiment, the cancer is ovarian cancer. In a specific embodiment, the cancer is pancreatic cancer. In a specific embodiment, the cancer is prostate cancer. In a specific embodiment, the cancer is renal cancer. In a specific embodiment, the cancer is gastric cancer. In a specific embodiment, the cancer is thyroid cancer. In a specific embodiment, the cancer is urothelial cancer.

In another specific embodiment, the cancer is selected from the group consisting of: leukemia and multiple myeloma.

In one embodiment, the invention relates to the combination, pharmaceutical composition or kit of parts as herein above defined for use in immunotherapy, preferable as cancer immunotherapy.

In one embodiment, the invention relates to a method of immunotherapy, preferably of cancer immunotherapy, which comprises administering to a mammal species in need thereof a therapeutically effective amount of the combination, pharmaceutical composition or kit of parts of the invention.

The invention further provides the use of the combination, pharmaceutical composition or kit of parts of the invention for the manufacture of a medicament for conducting immunotherapy, preferably of cancer immunotherapy.

In one embodiment, the invention relates to the use of the combination, pharmaceutical composition or kit of parts of the invention, for increasing immune recognition and destruction of the cancer cells.

In one embodiment, the invention relates to a method for increasing immune recognition and destruction of the cancer cells, which comprises administering to a mammal species in need thereof a therapeutically effective amount of the combination, pharmaceutical composition or kit of parts of the invention.

The invention further provides the use of the combination, pharmaceutical composition or kit of parts of the invention for the manufacture of a medicament for increasing immune recognition and destruction of the cancer cells.

Preferably, the patient is a warm-blooded animal, more preferably a human

In one embodiment, the patient receiving the A2AR inhibitor as herein described is also receiving an immunotherapy, a chemotherapy, radiotherapy or a combination thereof.

In one embodiment, the subject is resistant to an immunotherapy. In one embodiment, the subject is resistant to a cancer immunotherapy.

In one embodiment, the subject is resistant to a chemotherapy. In one embodiment, the subject is resistant to a cancer chemotherapy.

In one embodiment, the subject is resistant to a radiotherapy. In one embodiment, the subject is resistant to a cancer radiotherapy.

The invention also relates to a compound of Formula (I) as defined above for use in therapy in combination with an anticancer agent as defined above, especially immunotherapeutic agents, chemotherapeutic agents, antiangiogenic agents, multidrug resistance-associated proteins inhibitors, radiotherapeutic agents, or any combination thereof.

The invention also relates to a compound of Formula (I) as defined above for use in a patient treated by immunotherapy, a chemotherapy, radiotherapy or a combination thereof.

The invention thus also relates to a method for treating cancer in a subject resistant to an anticancer agent, comprising administering to the patient a compound of Formula (I) as defined above and said anticancer agent. In one embodiment, said anticancer agent is an immunotherapy, a chemotherapy, radiotherapy or a combination thereof.

The invention further relates to a method for increasing the therapeutic response of a subject to an anticancer agent, comprising further administering to the patient a compound of Formula (I) as defined above. In one embodiment, said anticancer agent is an immunotherapy, a chemotherapy, radiotherapy or a combination thereof.

The invention also relates to an anticancer agent as defined above for use in therapy in combination with a compound of Formula (I) as defined above.

### BRIEF DESCRIPTION OF THE DRAWINGS

FIG. 1 shows study schematic. Abbreviations: BID=twice daily; MTD=maximum tolerated dose; QD=once daily; RP2D=recommended Phase 2 dose

FIG. 2A-D shows schedule of assessments for dose-escalation phase. Abbreviations: AEs=adverse events, C1D1=Cycle 1 Day 1, C1D8=Cycle 1 Day 8, etc., CT=computerized tomography, ECG=electrocardiogram, ECOG=Eastern Cooperative Oncology Group, EOT=end of treatment, FU=follow-up, IP=investigational product, MRI=magnetic resonance imaging, pCREB=cyclic adenosine monophosphate response element binding protein phosphorylation

a EOT assessments were performed within 7 days following the last dose of Compound 8a

b Phone calls every 8 weeks (±7 days) for 6 months after last dose and every 12 weeks (±14 days) thereafter until withdrawal of consent or start of a new anti-cancer therapy

c Vital signs were obtained within 30 minutes before dosing and within 90 minutes after dosing with Compound 8a on C1D1, C1D8, C1D15, and C1D21.

d The physical examination and ECOG evaluation for C1D1 were performed up to 7 days before the C1D1 visit.

e At screening, only 1 ECG were obtained for local reading. At C1D1, C1D8, C1D15, and C1D21, ECG were obtained in triplicate within 30 minutes before dosing and within 90 minutes after dosing with Compound 8a. At C2D1, C3D1 triplicate ECGs were obtained before dosing. At EOT, triplicate ECGs were obtained at any time at the EOT visit. For C4D1 and subsequent cycles, only 1 ECG were obtained at the time of visit, regardless of dosing.

f Echocardiogram or any validated left ventricular ejection fraction assessment method used by site (ie, multigated acquisition), were performed at screening, C2D1 (±7 days), every subsequent 8 weeks (±7 days), and between EOT and the 30-day follow-up visit

g The clinical laboratory test and urinalysis on Day 1 in each cycle were performed up to 72 hours before the visit.

h Overnight stay was optional

i Blood samples for PK analysis were obtained at the following time points:

C1D1: pre-dose, and 0.25, 0.5, 1, 2, 4, 6, 8, 12, and 24 hours after dosing

C1D8: pre-dose

C1D15: pre-dose, and 0.25, 0.5, 1, 2, 4, 6, 8, and 12 hours (before the second daily administration, if applicable) after dosing C2D1: pre-dose, and 2, 6, and 12 hours (before the second daily administration, if applicable) after dosing

C3D1: pre-dose

j There are 2 types of sampling for PD (pCREB=PD1 and cytokines=PD2)

Blood samples for pharmacodynamic analysis were obtained at the following time points:

Screening (between Day −28 and Day −1)

C1D1: pre-dose, and 2, 6, 12, and 24 hours after dosing

C1D15: pre-dose, and 2, 6, and 12 hours (before the second daily administration, if applicable) after dosing C2D1: pre-dose, and 2, 6, and 12 hours (before the second daily administration, if applicable) after dosing C3D1: pre-dose

k The pre-treatment biopsy was performed at any time during the screening period (Day −28 to Day −1). An existing biopsy taken within 1 month before enrollment into the study was acceptable if no intervening anti-cancer therapy had been administered. One treatment biopsy occurred between C1D21 and C1D27 before C2D1.

l CT and/or MRI scans to assess tumor status were obtained at baseline (during screening) and every 8 weeks starting from C1D1 until EOT.

m In women of child bearing potential: a serum pregnancy test at screening and serum or urine dipstick thereafter, according to site regular practice. The urine pregnancy test were performed before dosing, if applicable

n Serum samples for exploratory analysis were taken at screening and pre-dose on C1D1, C1D8, C1D15, C2D1, and C3D1

FIG. 3 shows partial response of two patients. FIG. 3A shows partial response in a 67-year-old Male with mCRPC (metastatic castrate-resistant prostate cancer). Patient had documented progression after hormonal therapy and two lines of chemotherapy. He received Compound 8a monotherapy at 80 mg BID. His second disease assessment at 16 weeks of therapy showed a partial response by RECIST with a 41% reduction. The patient also reported a reduction in bone pain. FIG. 3B shows partial response in a 67-year-old Male with BRAF Wild-Type Cutaneous Melanoma. The patient had received two lines of immunotherapy, pembrolizumab followed by ipilimumab, with documented progressive disease. He received Compound 8a monotherapy at 160 mg BID. After 16 weeks of therapy, a partial response was observed with a 44% reduction in the size of the target lesions and reduced pain and lymphedema.

FIG. 4 shows principle of the assay and assay readouts. In peripheral blood lymphocytes, the A2AR agonist CGS-21680 (CGS) induces phosphorylation of CREB (pCREB) at serine 133, while Compound 8a dose-dependently inhibits pCREB induced by CGS. The extent of pCREB is estimated through flow cytometry (MeFI pCREB) for each stimulation condition, visit and time point. The ratio of pCREB measured with CGS stimulation to the null (DMSO) condition (pCREBCGS nMeFI) is presented for each visit and time point.

FIG. 5 shows PD activity of Compound 8a administered at 20 mg QD. CGS-21680 induced pCREB in CD4+ (red circles), CD8+ (blue squares) and CD19+ (violet triangles) gated cells in ex-vivo stimulated whole blood at each nominal time point. Data are presented as geomean±SD.

FIG. 6 shows inhibition of pCREB at C1D1 of Compound 8a administered at 20 mg QD. Percent inhibition of CGS-21680 induced pCREB in CD4+ (red circles), CD8+ (blue squares) and CD19+ (violet triangles) gated cells at each nominal time point. Data are presented as arithmetic mean±SEM.

FIG. 7 shows PD activity of Compound 8a administered at 40 mg QD. CGS-21680 induced pCREB in CD4+ (red circles), CD8+ (blue squares) and CD19+ (violet triangles) gated cells in ex-vivo stimulated whole blood at each nominal time point. Data are presented as geomean±SD.

FIG. 8 shows Inhibition of pCREB at C1D1 of Compound 8a administered at 40 mg QD. Percent inhibition of CGS-21680 induced pCREB in CD4+ CD4+ (red circles), CD8+ (blue squares) and CD19+ (violet triangles) gated cells at each nominal time point. Data are presented as arithmetic mean±SEM.

FIG. 9 shows PD activity of Compound 8a administered at 40 mg BID. CGS-21680 induced pCREB in CD4+ (red circles), CD8+ (blue squares) and CD19+ (violet triangles) gated cells in ex-vivo stimulated whole blood at each nominal time point. Data are presented as geomean±SD.

FIG. 10 shows inhibition of pCREB at C1D1 of Compound 8a administered at 40 mg BID. Percent inhibition of CGS-21680 induced pCREB in CD4+ (red circles), CD8+ (blue squares) and CD19+ (violet triangles) gated cells at each nominal time point. Data are presented as arithmetic mean±SEM.

FIG. 11 shows PD activity of Compound 8a administered at 80 mg BID. CGS-21680 induced pCREB in CD4+ (red circles), CD8+ (blue squares) and CD19+ (violet triangles) gated cells in ex-vivo stimulated whole blood at each nominal time point. Data are presented as geomean±SD.

FIG. 12 shows inhibition of pCREB at C1D1 of Compound 8a administered at 80 mg BID. Percent inhibition of CGS-21680 induced pCREB in CD4+ (red circles), CD8+ (blue squares) and CD19+ (violet triangles) gated cells at each nominal time point. Data are presented as arithmetic mean±SEM.

FIG. 13 shows PD activity of Compound 8a administered at 160 mg BID. CGS-21680 induced pCREB in CD4+ (red circles), CD8+ (blue squares) and CD19+ (violet triangles) gated cells in ex-vivo stimulated whole blood at each nominal time point. Data are presented as geomean±SD.

FIG. 14 shows inhibition of pCREB at C1D1 of Compound 8a administered at 160 mg BID. Percent inhibition of CGS-21680 induced pCREB in CD4+ (red circles), CD8+ (blue squares) and CD19+ (violet triangles) gated cells at each nominal time point. Data are presented as arithmetic mean±SEM.

FIG. 15 shows PD activity of Compound 8a administered at 20 mg QD. Log-transformed changes in CGS-21680 induced alterations of ENA-78 (brown, circles), IL-8 (blue, squares) and TNF-alpha (black, triangles) secretion in ex-vivo stimulated whole blood at each nominal time point. Data are presented as mean±SEM.

FIG. 16 shows inhibition of CGS effect at C1D1 of Compound 8a administered at 20 mg QD. Percent inhibition of CGS-21680 effect on ENA-78 (brown, circles), IL-8 (blue, squares) and TNF-alpha (black, triangles) secretion at each nominal time point at first drug exposure (C1D1). Data are presented as mean±SEM.

FIG. 17 shows PD activity of Compound 8a administered at 40 mg QD. Log-transformed changes in CGS-21680 induced alterations of ENA-78 (brown, circles), IL-8 (blue, squares) and TNF-alpha (black, triangles) secretion in ex-vivo stimulated whole blood at each nominal time point. Data are presented as mean±SEM.

FIG. 18 shows Inhibition of CGS effect at C1D1 of Compound 8a administered at 40 mg QD. Percent inhibition of CGS-21680 effect on ENA-78 (brown, circles), IL-8 (blue, squares) and TNF-alpha (black, triangles) secretion at each nominal time point at first drug exposure (C1D1). Data are presented as mean±SEM.

FIG. 19 shows PD activity of Compound 8a administered at 40 mg BID. Log-transformed changes in CGS-21680 induced alterations of ENA-78 (brown, circles), IL-8 (blue, squares) and TNF-alpha (black, triangles) secretion in ex-vivo stimulated whole blood at each nominal time point. Data are presented as mean±SEM.

FIG. 20 shows Inhibition of CGS effect at C1D1 of Compound 8a administered at 40 mg BID. Percent inhibition of CGS-21680 effect on ENA-78 (brown, circles), IL-8 (blue, squares) and TNF-alpha (black, triangles) secretion at each nominal time point at first drug exposure (C1D1). Data are presented as mean±SEM.

FIG. 21 shows PD activity of Compound 8a administered at 80 mg BID. Log-transformed changes in CGS-21680 induced alterations of ENA-78 (brown, circles), IL-8 (blue, squares) and TNF-alpha (black, triangles) secretion in ex-vivo stimulated whole blood at each nominal time point. Data are presented as mean±SEM. For subject 01-006, T6 and T12 results for ENA-78 were excluded from the analysis.

FIG. 22 shows Inhibition of CGS effect at C1D1 of Compound 8a administered at 80 mg BID. Percent inhibition of CGS-21680 effect on ENA-78 (brown, circles), IL-8 (blue, squares) and TNF-alpha (black, triangles) secretion at each nominal time point at first drug exposure (C1D1). Data are presented as mean±SEM.

FIG. 23 shows PD activity of Compound 8a administered at 160 mg BID. Log-transformed changes in CGS-21680 induced alterations of ENA-78 (brown, circles), IL-8 (blue, squares) and TNF-alpha (black, triangles) secretion in ex-vivo stimulated whole blood at each nominal time point. Data are presented as mean±SEM.

FIG. 24 shows Inhibition of CGS effect at C1D1 of Compound 8a administered at 160 mg BID. Percent inhibition of CGS-21680 effect on ENA-78 (brown, circles), IL-8 (blue, squares) and TNF-alpha (black, triangles) secretion at each nominal time point at first drug exposure (C1D1). Data are presented as mean±SEM.

FIG. 25 shows Overview of CGS-targeted analytes in IO-001 patients. Heatmap of percent change of analyte secretion following stimulation. Dark red: Analytes with >30% increased levels in LPS+CGS compared to LPS. Dark blue: Analytes with >30% decreased levels in LPS+CGS compared to LPS.

FIG. 26 shows Representative Mean Parent (Compound 8a; Top) and Metabolite (Compound 77; Bottom) Plasma Concentration Profiles for QD (left) and BID (right) Cohorts on Day 1 showing quasi-parallel behavior

FIG. 27 shows Compound 8a Plasma Concentration versus Time Profiles for QD Cohorts on Day 1 (Top) and Day 15 (Bottom)

FIG. 28 shows Compound 8a Plasma Concentration versus Time Profiles for BID Cohorts on Day 1 (Top) and Day 15 (Bottom)

### EXAMPLES

The present invention will be better understood with reference to the following examples. These examples are intended to representative of specific embodiments of the invention, and are not intended as limiting the scope of the invention.

The following abbreviations are used:

BHT: butylated hydroxytoluene

BID: bis in die (i.e. twice a day)

ca.: circa

CR: complete responder

DMSO: dimethylsulfoxide

EDTA: ethylenediaminetetraacetic acid

FeSSIF: fed-state simulated intestinal fluid

HPLC: high-performance liquid chromatography

LC-MS: liquid chromatography-mass spectrometry

mAb: monoclonal antibody

mg: milligram

MS: mass spectrometry

PBS: phosphate buffered saline

PEG: polyethylene glycol

QD: quaque die (i.e. once a day)

Q3D: quaque 3 die (i.e. every 3 days)

rpm: revolutions per minutes

TGI: tumor growth inhibition

TILs: tumor infiltrating lymphocytes

UV: ultraviolet

μL: microliter

% v/v: percentage in volume to the total volume of the composition

% w/w: percentage in weight to the total weight of the composition

AE Adverse event

ALK Anaplastic lymphoma kinase

ALT Alanine transaminase

AMP Adenosine monophosphate

AST Aspartate transaminase

AUC Area under the concentration-time curve

BID Twice daily

BNP Brain natriuretic peptide or B-type natriuretic peptide

CA Competent authority

CNS Central nervous system

CREB Cyclic AMP response element binding protein

CT Computerized tomography

CTCAE Common Terminology Criteria for Adverse Events

CTLA-4 Cytotoxic T lymphocyte-associated antigen-4

DLT Dose-limiting toxicity

ECG Electrocardiogram

ECOG Eastern Cooperative Oncology Group

eCRF Electronic case report form

EGFR Epidermal growth factor receptor

EOT End of treatment

FDA United States Food and Drug Administration

HIV Human immunodeficiency virus

ICF Informed consent form

ICH International Council for Harmonization

IEC Independent ethics committee

ILM Investigator laboratory manual

IP Investigational product

IRB Institutional review board

LD Longest diameter

mAb Monoclonal antibody

MRI Magnetic resonance imaging

MTD Maximum tolerated dose

ORR Objective response rate

OS Overall survival

pCREB cyclic AMP response element binding protein phosphorylation

PCWG3 Prostate Cancer Working Group 3

PD Pharmacodynamic(s)

PD-1 Programmed Cell Death-1

PK Pharmacokinetic(s)

PR PR interval; ie, the measure of the time between the start of the p wave and the end of the r wave in the heart's electrical cycle

QD Once daily

QRS QRS interval; ie, the measure of the time between the start of the q wave and the end of the s wave in the heart's electrical cycle

QT QT interval; ie, the measure of the time between the start of the q wave and the end of the t wave in the heart's electrical cycle

QTcB Corrected QT interval using Bazett's formula

QTcF Corrected QT interval using Fridericia's formula RECIST Response Evaluation Criteria In Solid Tumors

RP2D Recommended Phase 2 dose

SA Safety analysis set

SAE Serious adverse event

TME Tumor microenvironment

ULN Upper limit of normal

UK United Kingdom

US United States

1. Compounds

The compounds of Formula (I) were prepared as described in PCT/EP2018/058301. Pharmaceutical compositions were prepared as described in PCT/EP2019/074208. Pharmacological properties of pharmaceutical compositions were tested in the pre-clinical studies as described in PCT/EP2019/074208. Pharmacological properties of pharmaceutical compositions comprising further pharmaceutically active compounds were described in PCT/EP2019/074208.

Compound 8a and its metabolite Compound 77 were prepared as described in PCT/EP2018/058301.

The compound 8A is presented in Table 1 above, and the compound 77 is presented in Table 1 above.

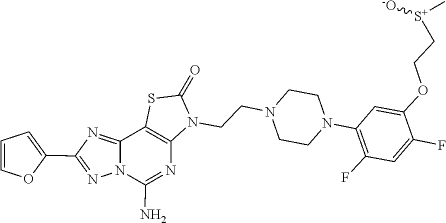

Compound 8a (MW: 604.65)

### (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one

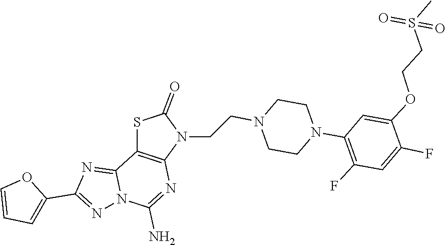 [SMILES: CS(=O)(=O)CCOc1cc(N2CCN(CCn3c(=O)sc4c3nc(N)n3nc(-c5ccco5)nc43)CC2)c(F)cc1F]

Compound 77 (MW: 620.65)

### 5-amino-3-(2-(4-(2,4-difluoro-5-(2-(methylsulfonyl)ethoxy)phenyl)piperazin-1-yl)ethyl)-8-(furan-2-yl)thiazolo[5,4-e][1,2,4]triazolo[1,5-c]pyrimidin-2(3H)-one

Formulations used in the subsequent examples are summarized below

| | | | | Composition | |
|---|---|---|---|---|---|
| | | | | for 20, 40, | |
| | Composition | Composition | Composition | and 80 mg | |
| | for 20 mg | for 40 mg | for 80 mg | Compound 8a | |
| | Compound 8a | Compound 8a | Compound 8a | capsules | |
| Component | capsules (mg) | capsules (mg) | capsules (mg) | (% w/w) | Function |
| Compound 8a | 20.00 | 40.00 | 80.00 | 10.00 | Active |
| Gelucire 44/14 | 143.8 | 287.6 | 575.20 | 71.90 | Wetting/ |
| lauroyl macrogol- | | | | | Dissolution |
| 32 glycerides EP | | | | | aid |
| Polyethylene | 36.00 | 72.00 | 144.00 | 18.00 | Wetting/ |
| glycol 400 | | | | | Dissolution |
| | | | | | aid |
| Butylated | 0.20 | 0.40 | 0.80 | 0.10 | Antioxidant |
| hydroxytoluene | | | | | |
| White body, dark | 1 capsule | — | — | — | Encapsulation |
| green cap opaque | | | | | |
| gelatin capsule, | | | | | |
| size 00 | | | | | |
| White body, dark | — | 1 capsule | — | — | Encapsulation |
| green cap opaque | | | | | |
| gelatin capsule, | | | | | |
| size 00 | | | | | |
| White opaque | — | — | 1 capsule | — | Encapsulation |
| gelatin capsule, | | | | | |
| size 00 | | | | | |
| Total per capsule | 200.00 mg | 400.00 mg | 800.00 mg | 100.00 | |

2. First-in-Human Study of Compound 8a in Patients with Advanced Solid Tumors

2.1. Study Objectives and Endpoints

The objectives and endpoints of the study are displayed in Table 2.

TABLE 2

| Objectives | Endpoints |
|---|---|
| Primary | |
| To define the maximum tolerated | Incidence of AEs, SAEs, DLTs, |
| dose (MTD) and the recommended | AEs leading to discontinuation, |
| Phase 2 dose (RP2D) of Compound | deaths, electrocardiogram |
| 8a in patients with advanced solid | (ECG) abnormalities, and |
| tumors | clinically significant laboratory |
| To characterize the safety and | abnormalities |
| tolerability of Compound 8a as | |
| measured by AEs and DLTs | |
| Secondary | |
| To assess the pharmacokinetics (PK) | Plasma concentrations of |
| of Compound 8a in blood | Compound 8a and its |
| | metabolite Compound 77 at |
| | each dose level |
| To assess the antitumor activity | Objective response rate (ORR), |
| of Compound 8a | duration of overall response, |
| | progression-free survival at 24, |
| | 36 and 48 weeks per Response |
| | Evaluation Criteria In Solid |
| | Tumors (RECIST) v1.1 |
| Exploratory | |
| To assess the pharmacodynamic (PD) | Blood levels of pCREB, |
| activity of Compound 8a by analyzing | cytokines, and chemokines at |
| cyclic adenosine monophosphate | each dose level |
| (AMP) response element binding | Correlation/measure of |
| protein (CREB) phosphorylation | association of plasma PK and |
| (pCREB) and cytokine and | relevant PD biomarkers in the |
| chemokine secretion in blood samples | peripheral blood |
| after ex vivo stimulation with | Change from baseline in |
| lipopolysaccharide and an A_{2A}R | exploratory biomarkers in the |
| agonist | tumor and peripheral blood |
| To explore the association of | Summary measures of antitumor |
| plasma PK, safety, efficacy, and | activity by pre-treatment level of |
| various PD biomarkers measures in | biomarkers of interest; |
| the tumor and peripheral blood | correlation/measure of |
| before treatment and following | association of antitumor activity |
| administration of IP | and change (or percent change) |
| To assess the antitumor activity of | from baseline in biomarkers of |
| Compound 8a | interest |
| | Overall Survival |

1.1. Overall Study Design and Plan

This multicenter, open-label, first-in-Human, dose-escalation Phase I/Ib clinical study was designed to evaluate the safety and tolerability, the MTD, RP2D, PK, pharmacodynamics (PD), and antitumor activity of Compound 8a in patients with advanced solid tumors.

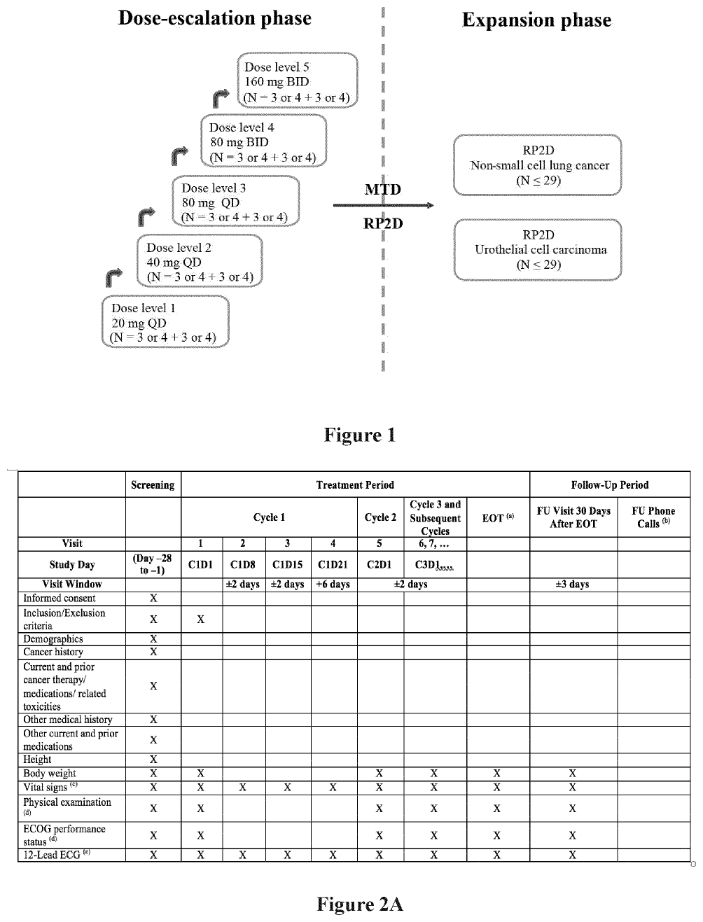

The study was planned to be conducted in 2 parts (FIG. 1). The first part of the study was a dose-escalation phase to determine the MTD and the RP2D, as well as the safety of Compound 8a in patients with advanced malignancies. The subsequent expansion phase of the study will further explore safety, PK and PD parameters, and antitumor activity of Compound 8a in patients with selected cancer types treated at the RP2D.

Tumor response to Compound 8a was assessed every 8 weeks throughout the treatment period according to the RECIST v1.1.

Pharmacokinetic and PD tests was performed at specified times that is indicated later in this document during the treatment period.

End of treatment (EOT) is defined as the day the decision was made and documented to permanently discontinue the patient from treatment for whatever reason.

1.2. Rationale for Starting Dose and Dosing Schedule

The starting dose of Compound 8a for this first-in-human study was determined based on the non-clinical safety and pharmacology data which was previously described in PCT/EP2019/074208.

Toxicologic results from rodent and non-rodent species were used to guide the selection of a safe human starting dose. From the highest non-severely toxic dose in the 28-day toxicity study in dogs (100 mg/kg/day) and using a safety factor of 6 (⅙th highest non-severely toxic dose), a calculated safe human starting dose resulted in 540 mg/day. The severely toxic dose in 10% of rodents was not established in the 28-day toxicity study in mice, but substituting the lowest dose tested (40 mg/kg/day) for the severely toxic dose in 10% of rodents resulted in a starting dose in humans of 20 mg/day. Based on non-clinical pharmacology studies, the area under the concentration-time curve (AUC) linked to efficacy in mouse models was determined as 100 ng·h/mL.

To ensure adequate patient safety while limiting the likelihood of administering a subtherapeutic dose to patients, a starting dose of Compound 8a at 20 mg/day was considered appropriate. This dose is close to the estimated efficacious AUC and approximately 5% of the group mean AUC at the end of dosing at 100 mg/kg/day. This proposed first-in-human starting dose of Compound 8a at 20 mg/day was considered appropriate to ensure adequate patient safety while limiting the likelihood of administering a subtherapeutic dose to patients with advanced cancer.

Because the estimated half-life in humans is about 3 hours but the residence time of Compound 8a on A2AR could be longer, patients in the first 3 cohorts was treated with a once daily (QD), 20, 40, or 80 mg, repeated dose to safely obtain an early evaluation of human pharmacokinetics (PK) and estimation of the residence time. Thereafter, considering the estimated short half-life in humans, the dosing was escalated in the next cohorts to a twice daily (BID) schedule, to sustain the plasma concentrations above the potential therapeutic level of Compound 8a constantly.

1.3. Dose-Escalation Phase

Total 21 patients were enrolled to the study and completed the DLT evaluation. Patients were treated in cohorts of 3 or 4 patients. The first 28 days of treatment (ie, Cycle 1) in the dose-escalation part was the evaluation period for DLTs (DLT period).

Based on the tolerability and safety of Compound 8a in patients, enrollment at the next dose level and/or additional patients into the ongoing cohort had been planned to occur according to the following:
If 0 of the 3 or 4 initially treated patients experience a DLT during the DLT period of 4 weeks (28 days), then dose escalation occurs.
If 1 of the 3 or 4 initially treated patients experiences a DLT during the DLT period, then an additional 3 or 4 patients are enrolled for a total of 6 to 8 patients treated at the same dose level; escalation occur if no additional DLTs are seen in that cohort.
If 2 or more treated patients at a dose level experience a DLT during the DLT period, enrollment at that dose and dose escalation stops. If at least 6 patients were already treated at the prior lower dose level, then this lower dose is considered the MTD. In case fewer than 6 patients have been treated at that prior lower dose level, then more patients are enrolled to evaluate at least 6 patients at that lower dose.

The dose levels for Compound 8a were 20, 40, and 80 mg once a day, and 80 and 160 mg, twice a day.

Although dose escalation was planned as described above, no DLTs were observed for the used dose levels.

Maximum Tolerated Dose

The MTD is defined as the highest dose at which ≤1 out of at least 6 evaluable patients experience a DLT.

Maximum Administered Dose

The maximum administered dose is defined as the highest dose at which at least 2 or more patients experience a DLT so that further dose escalation is not undertaken.

None of the dose levels used in this study for Compound 8a was found as maximum administered dose since no DLT was observed for the doses used.

Dose-Limiting Toxicities

A DLT is defined as a treatment-related toxicity, ie, considered possibly, probably, or definitely related to Compound 8a. Toxicities was graded and recorded according to the Common Terminology Criteria for Adverse Events (CTCAE) version 5.0.

Adverse events that are clearly not treatment related was not considered for the definition of DLTs—an example is an injury caused by a third person. When there was ambiguity about whether an AE is related to the underlying disease or another cause as opposed to the IP, the most conservative approach was taken, and the AE was considered as treatment related.

Compound 8a monotherapy was found to be well tolerated with no treatment-emergent adverse events higher than grade 2 and no dose-limiting toxicities observed (Table 3). The most common adverse events which were considered at least possibly related to treatment were fatigue, transaminase elevation, decreased appetite and diarrhea. Asymptomatic interstitial pneumonitis was observed in one patient (Table 4).

TABLE 3

| | | |
|---|---|---|
| | Drug- | Any |
| Treatment-Emergent Adverse | Related | Attribution |

| | |
|---|---|
| Events (n = 21) | Number of Patients (%) |

| | | | |
|---|---|---|---|
| Any Grade | 15 (71.4%) | 21 | (100.0%) |
| Grade 1-2 | 15 (71.4%) | 21 | (100.0%) |
| Grade 3-4 | 0 (0.0%) | 8 | (38.1%) |
| Grade 5 | 0 (0.0%) | 0 | (0.0%) |
| Led to discontinuation | 0 (0.0%) | 2 | (9.5%) |

TABLE 4

| Drug Related TEAEs | | |
|---|---|---|
| (Grade 1-2), n = 21 | Number of Patients (%) | |
| Fatigue | 6 | (28.6%) |
| Alanine aminotransferase increased | 4 | (19.0%) |
| Decreased appetite | 4 | (19.0%) |
| Aspartate aminotransferase increased | 3 | (14.3%) |
| Diarrhoea | 3 | (14.3%) |
| Gamma-glutamyltransferase increased | 2 | (9.5%) |
| Blood alkaline phosphatase increased | 1 | (4.8%) |
| Hyperbilirubinaemia | 1 | (4.8%) |
| Constipation | 1 | (4.8%) |
| Myalgia | 1 | (4.8%) |
| Dizziness | 1 | (4.8%) |
| Eosinophilia | 1 | (4.8%) |
| Interstitial Pneumonitis | 1 | (4.8%) |
| Flushing | 1 | (4.8%) |

2.6 Study Duration

The start of the study was defined as the first visit for the first patient providing informed consent. Similarly, the end of the study was defined as the last visit or scheduled procedure shown in the schedule of assessments for the last patient. Primary study completion was defined as the final date on which data for the primary endpoint are expected to be collected. The study duration was expected to be around 3 years, subject to change on an ongoing basis.

For the patient, the study started once written consent for study participation was provided. After the screening period of up to 28 days, the patients started their first cycle and receive Compound 8a once per day or twice per day according to their assigned dose, every day until one of the criteria to discontinue the treatment met (described below). 16 out of 21 patients had to discontinue the treatment due to disease progression.

2.7 Selection of Study Population

Adult patients with histologically or cytologically confirmed advanced solid tumors for which no standard treatment available was included in the first part of this study.

2.7.1 Inclusion Criteria

Patients meeting all of the following inclusion criteria was considered for admission to the dose-escalation or dose-expansion cohorts:
1. Patient provided a signed written informed consent and consent for biopsies before and during administration of IP
2. Patient is male or female, ≥18 years of age
3. Patient with histologically or cytologically confirmed advanced solid tumor for whom no standard treatment is further available
4. Patient with evaluable disease per RECIST v1.1 in dose escalation or measurable disease per RECIST v1.1 in expansion and at least 1 lesion accessible for sequential biopsy (with the exception of metastatic prostate cancer, which can have only bone metastasis, and be evaluable according to Prostate Cancer Working Group 3 criteria)
5. Patient with a predicted life expectancy of ≥16 weeks
6. Patient with ECOG performance status of grade 0-1
7. Patient with serum albumin ≥25 g/L (2.5 g/dL)
8. Patient with hemoglobin ≥9.0 g/dL, platelet count ≥75×109/L, and absolute neutrophil count ≥1.2×109/L; a patient with a lower level of at least one of these parameters can be considered as eligible provided that recovery is expected during the screening period and demonstrated on a control blood test prior to the start of therapy
9. Patient with adequate renal function (creatinine level within normal institutional limit, or calculated creatinine clearance ≥60 mL/min/1.73 m2 for patients with creatinine levels above institutional normal)
10. Patient with adequate liver function (aspartate transaminase/alanine transaminase <3× institutional ULN, ≤5× if liver metastases, total bilirubin ≤1.5× ULN)
11. Patient with adequate coagulation tests: international normalized ratio within 1.5× ULN
12. Female patient of childbearing potential (defined as <12 continuous months of amenorrhea with no identified cause other than menopause or not surgically sterile), must have a negative pregnancy test within 7 days before first administration of IP and agree to use adequate contraception during the treatment until 60 days after the last administration of the IP (documentation required for surgical sterilization to exclude childbearing potential).

Examples of contraceptive methods with a failure rate of <1% per year include bilateral tubal ligation, male sterilization, hormonal contraceptives that inhibit ovulation, hormone-releasing intrauterine devices, and copper intrauterine devices. Hormonal contraceptive methods must be supplemented by a barrier method.

The reliability of sexual abstinence was evaluated in relation to the duration of the clinical study and the preferred and usual lifestyle of the patient. Periodic abstinence (eg, calendar, ovulation, symptothermal, or postovulation methods) and withdrawal were not acceptable methods of contraception.
13. Male patient with female partner of childbearing potential must agree to remain sexually abstinent or use condoms during the treatment phase and 100 days after the last dose of the IP. In addition, male patients must be willing to stop sperm donation during this time.
14. Patient is able to swallow and retain orally administered capsules, according to investigator's judgment.

Additional inclusion criteria for expansion phase:

Inclusion criteria for patients with lung cancer:
1. Patient has advanced or metastatic non-small cell lung cancer with squamous or non-squamous histological or cytological confirmation
2. Patient has previously received the following, and progressed or relapsed thereafter, or was ineligible for all systemic standard therapies with proven clinical benefit:
   Prior platinum-based combination
   One (but no more) line of previous therapy with an anti-PD-1/Programmed Cell Death Ligand-1 (PD-L1) monoclonal antibody (mAb) therapy either alone or in combination
3. Patient with non-squamous histology must have known epidermal growth factor receptor (EGFR) and anaplastic lymphoma kinase (ALK) status;
   Patient with an activating EGFR mutation must have received previous therapy with an EGFR tyrosine kinase inhibitor
   Patient with an ALK translocation must have received previous therapy with an ALK inhibitor.

Inclusion criteria for patients with bladder cancer:
1. Patient has histologically or cytologically documented, locally advanced or metastatic transitional cell carcinoma of the urothelium (including renal pelvis, ureters, urinary bladder, urethra)
2. Patient has previously received the following, and progressed or relapsed thereafter, or was ineligible for all systemic standard therapies with proven clinical benefit:
   Patient has previously received a prior platinum-based regimen (eg, gemcitabine and cisplatin, methotrexate, vinblastine, doxorubicin, and cisplatin, CarboGem, etc.) for inoperable locally advanced or metastatic urothelial carcinoma or disease recurrence
   Patient has previously received 1 (but no more) line of previous therapy with an anti-PD-1/PD-L1 mAb therapy either alone or in combination.

2.7.2 Exclusion Criteria

Patients meeting any of the following exclusion criteria were not enrolled in the dose-escalation or dose-expansion cohorts.
1. Patient with any anti-cancer therapy, unless at least 4 weeks (or 5 half-lives, whichever is shorter) since the last dose of prior cytotoxic, biologic or any investigational agents (6 weeks for mitomycin C or nitrosoureas), have elapsed before the first administration of IP, or at least 2 weeks since receiving non-palliative radiotherapy. Chronic treatment with non-investigational gonadotropin-releasing hormone analogs or other hormonal or supportive care is permitted
2. Patient with known CNS metastases, unless previously treated and well controlled for at least 1 month (defined as clinically stable, no edema, no steroids and stable in 2 scans at least 4 weeks apart)
3. Patient with concomitant second malignancies unless a complete remission was achieved at least 2 years before study entry with no additional therapy required or anticipated to be required during the study period
4. Patient with major surgery (defined as any surgical procedure that requires more than 24 hours admission in a hospital) within 5 weeks before initiating treatment or with minor surgical procedure within 7 days before initiating treatment (with the exception of minor surgical procedure needed for tumor biopsy)
5. Patient with a history of grade ≥2 pneumonitis or active autoimmune disease or persistent immune-mediated toxicity caused by immune checkpoint inhibitor therapy of grade ≥2, with the exception of residual endocrinopathy adequately substituted, vitiligo, type 1 diabetes mellitus, or psoriasis not requiring systemic therapy
6. Patient with toxicity (except for alopecia) related to prior anti-cancer therapy and/or surgery, unless the toxicity is either resolved, returned to baseline or grade 1, or deemed irreversible
7. Patient with any active neuropathy >grade 2 (CTCAE v5.0)
8. Patient with history of life-threatening toxicity related to prior immune therapy or any toxicity that resulted in permanent discontinuation from prior immune therapy
9. Patient with any condition requiring concurrent use of systemic immunosuppressants or corticosteroids >10 mg daily prednisone equivalents or other immunosuppressive medications within 14 days of IP administration (permitted: premedication for IV contrast, treatment with a short course of steroids (<5 days) up to 7 days before initiating IP, and topical glucocorticoids, or steroid replacement doses for adrenal insufficiency)
10. Patient with evidence of active infection that requires systemic antibacterial, antiviral, or antifungal therapy ≤7 days before the first dose of IP (except for viral infections that are presumed to be associated with the underlying tumor type required for study entry)
11. Patient with uncontrolled or significant cardiovascular disease including, but not limited to, any of the following:
   Left ventricular ejection fraction ≤50% determined by echocardiogram or other approved method of evaluation
   Myocardial infarction or stroke/transient ischemic attack within the past 2 years
   Uncontrolled angina within the past 6 months
   History of clinically significant arrhythmias (such as atrial fibrillation and conduction disorders, ventricular tachycardia, ventricular fibrillation, or torsade de pointes)
   Treatment with drugs that are generally considered to have a high risk of causing torsade de pointes (it will be at the determination of the treating physician to discontinue or substitute as appropriate; if discontinued, the washout period needs to be at least 5 half-lives of the drug)
   QT interval corrected for heart rate using Fridericia's formula (QTcF) prolongation >480 msec (except right bundle branch block)
   History of other clinically significant heart disease (eg, cardiomyopathy, congestive heart failure with New York Heart Association functional classification III-IV (NYHA 2016), pericarditis, significant pericardial effusion, or myocarditis)
   Cardiovascular disease-related requirement for daily supplemental oxygen therapy
   Diagnosis of deep vein thrombosis or pulmonary embolism within the past 6 months
12. Patient with symptomatic or previously symptomatic gastro-duodenal ulcer or reflux esophagitis, who requires permanent use of proton pump inhibitor, with previous complication of gastro-duodenal ulcer (ie, perforation, pyloric stenosis, bleeding), or with previous gastrectomy
13. Patient with known active or chronic hepatitis B or C (unless treated with no detectable virus) (patients are NOT required to be tested for the presence of such viruses before therapy on this protocol)
14. Patient with known history human immunodeficiency virus (HIV) (patients are NOT required to be tested for the presence of HIV before therapy on this protocol)
15. Patient with any known or underlying medical, psychiatric condition, and/or social situations that, in the opinion of the investigator, would limit compliance with study requirements
16. Patient with history of allergy to IP(s) or any of its components
17. Patient received packed red blood cells or platelet transfusion within 2 weeks of the first dose of IP
18. Female patient: pregnant or breastfeeding
19. Patient taking any drug that is an inducer or inhibitor of CYP3A4, inhibitors of P-glycoprotein, or substrates of breast cancer resistance protein
20. Patient consuming grapefruit, pomelo, Seville orange, grapefruit juice, Seville orange juice, or pomelo juice within 7 days before the first dose of IP
21. Patient who has received vaccine containing live virus within 4 weeks prior to first dose of study treatment.

2.8 Details of Investigational Product

Information about the Compound 8a is provided in Table 5.

TABLE 5

| | Compound |
|---|---|
| Active ingredient | Compound 8a |
| Manufacturer | Quay Pharmaceuticals Limited and CSM |
| | Europe SA |
| Planned Dose(s) | 20, 40, or 80 mg once daily (QD), and 80 or |
| | 160 mg, twice daily (BID). |
| | Other doses could be explored during the dose |
| | escalation based on the evaluation of all |
| | available safety, PK, PD, and efficacy data |
| Route | Oral |
| Formulation | Capsule |
| Strength(s) | 20 mg, 40 mg, 80 mg |
| Storage requirements | Capsules should be stored in a secure |
| | location at room temperature (up to 25° C.) |
| Container | Compound 8a capsules will be supplied in |
| | white high-density polyethylene bottles |

2.8.1 Mode of Administration and Dosage Schedule

The patients took Compound 8a once or twice daily on Days 1 through 28 of every 28-day cycle until unacceptable toxicity or disease progression or any criteria described in this document.

Compound 8a was administered once daily every 24 hours (±4 hours) (dose levels 1-3) or twice daily every 12 hours (±2 hours) (dose levels 4 and 5) by mouth with 250 mL of permitted beverage on an empty stomach (no food or drink other than permitted beverage for 2 hours before dose and 1-hour post-dose). Permitted beverages had low pH value (pH lower than 7, 5, preferably lower than 3). Beverages can be a soda or juice. In the dose- escalation phase, patients will be assigned to one of the planned 5 Compound 8a dose levels:
Dose level 1: 20 mg QD (daily dose: 1 capsule containing 20 mg) on Days 1 through 28 of each treatment cycle
Dose level 2: 40 mg QD (daily dose: 1 capsule containing 40 mg) on Days 1 through 28 of each treatment cycle
Dose level 3: 80 mg QD (daily dose: 1 capsule containing 80 mg) on Days 1 through 28 of each treatment cycle
Dose level 4: 80 mg BID (daily dose: 2 capsules containing 80 mg each) on Days 1 through 28 of each treatment cycle
Dose level 5: 160 mg BID (daily dose: 4 capsules containing 80 mg each) on Days 1 through 28 of each treatment cycle
   The 80 mg BID dose, which provided the highest exposure, was selected as the recommended phase 2 dose. In the expansion phase, patients will receive Compound 8a at the RP2D level.

2.10 Screening Procedures (Day −28 to −1)

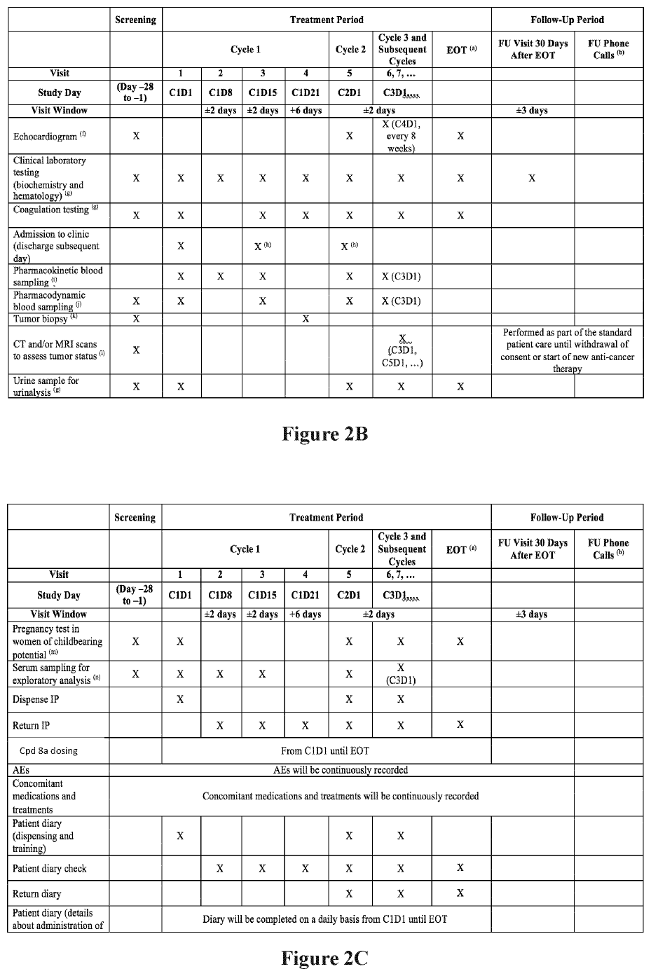

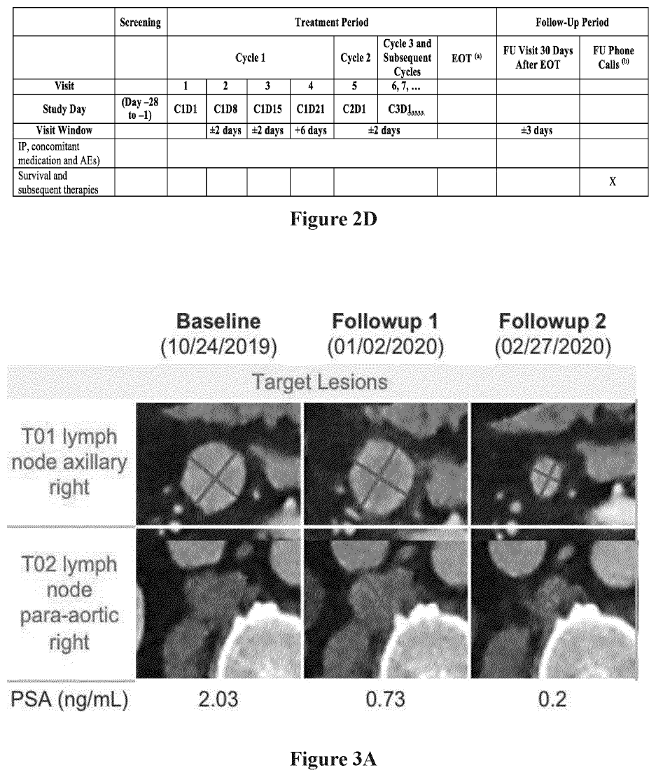

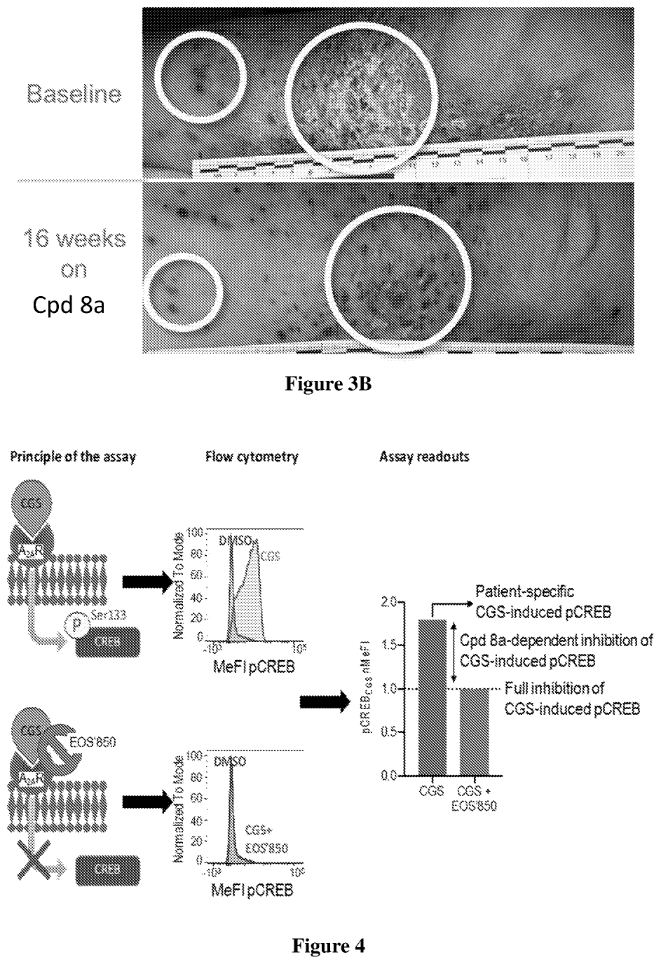

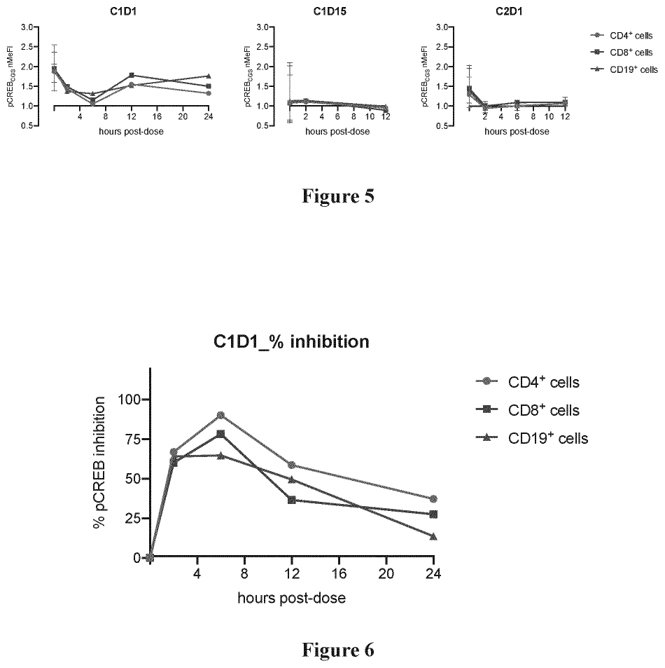

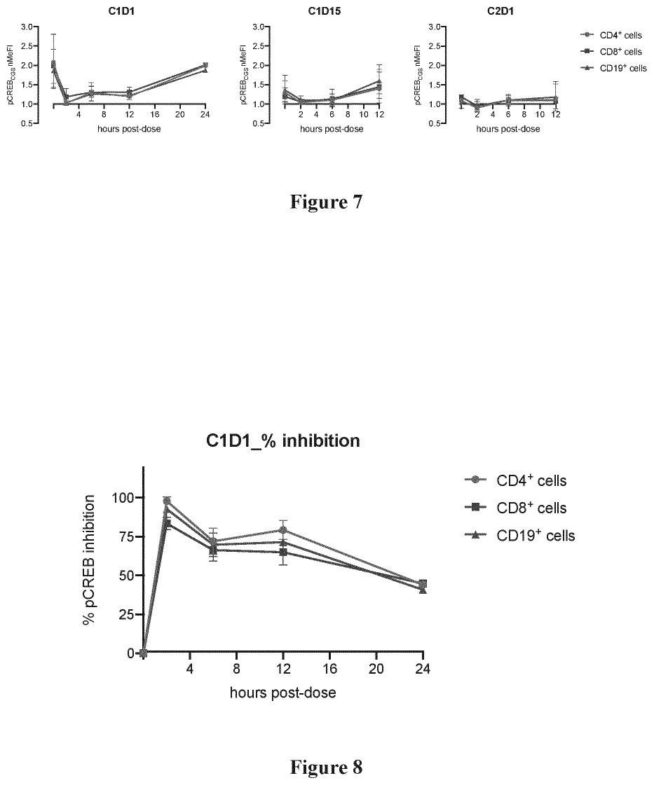

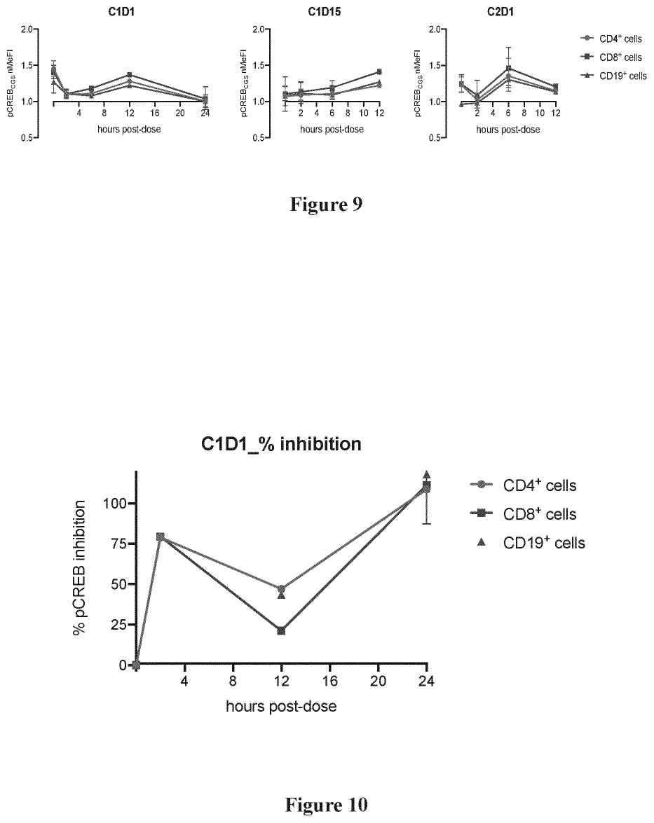

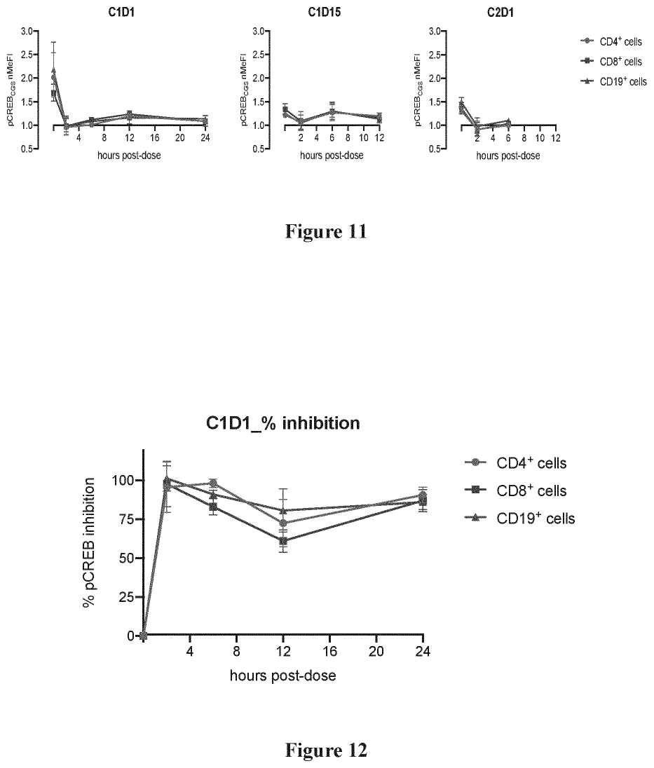

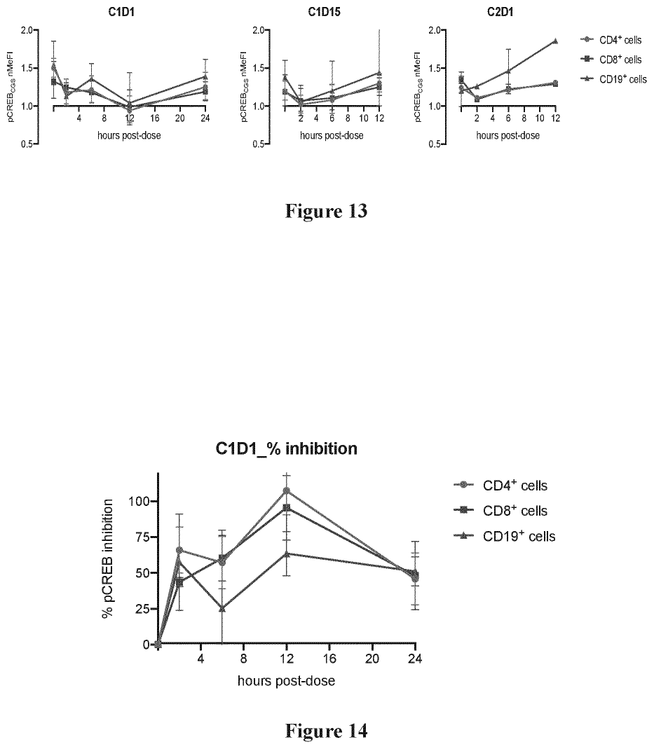

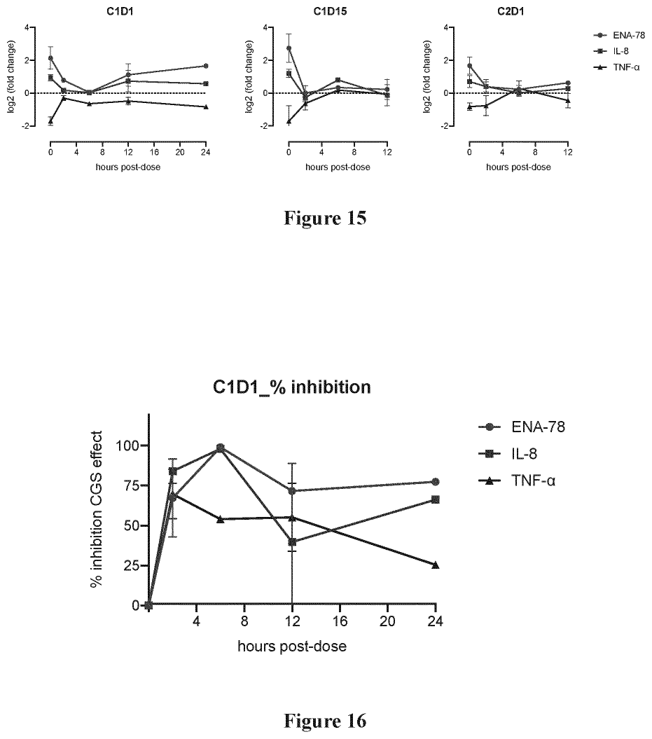

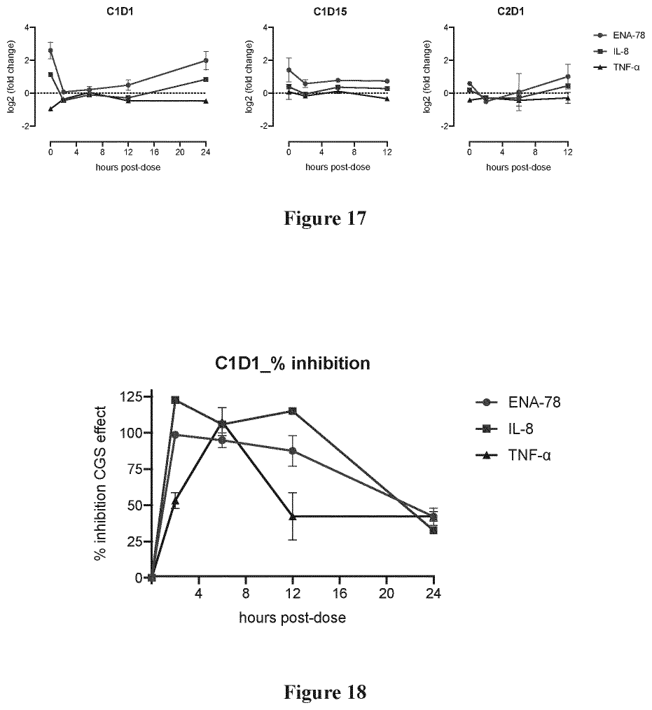

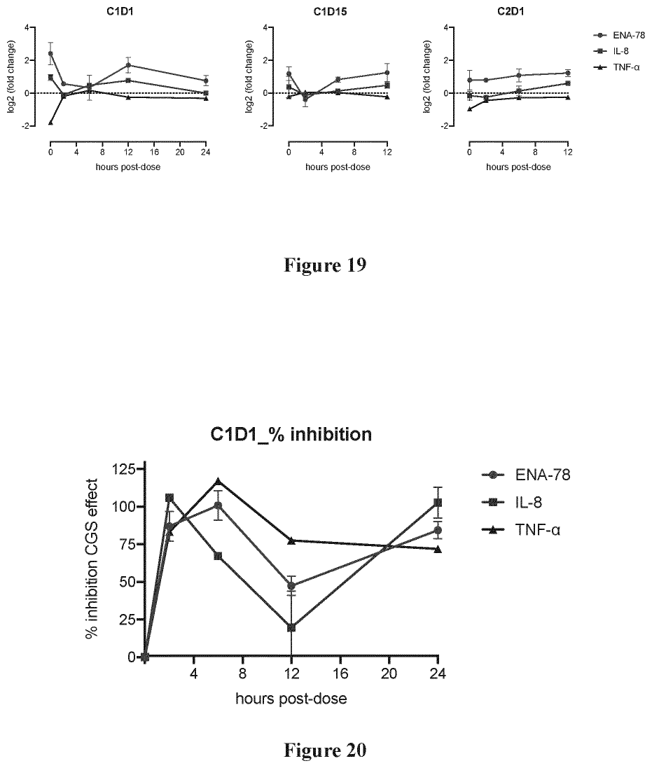

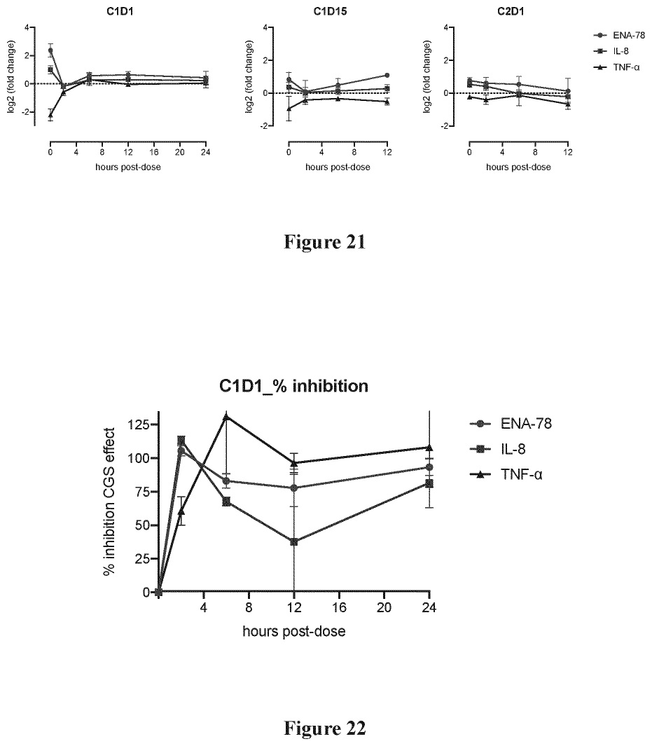

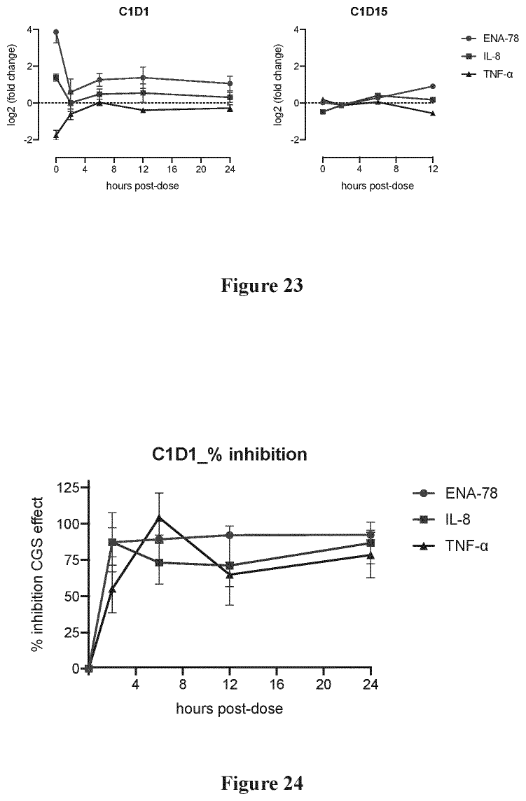

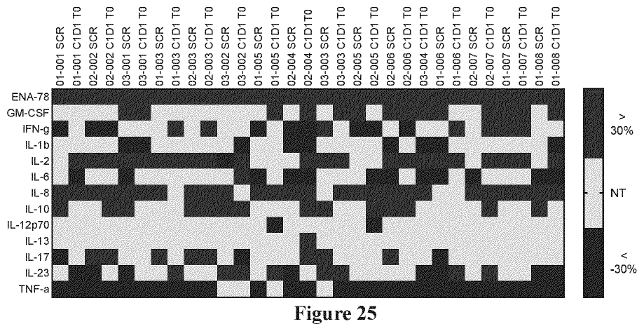

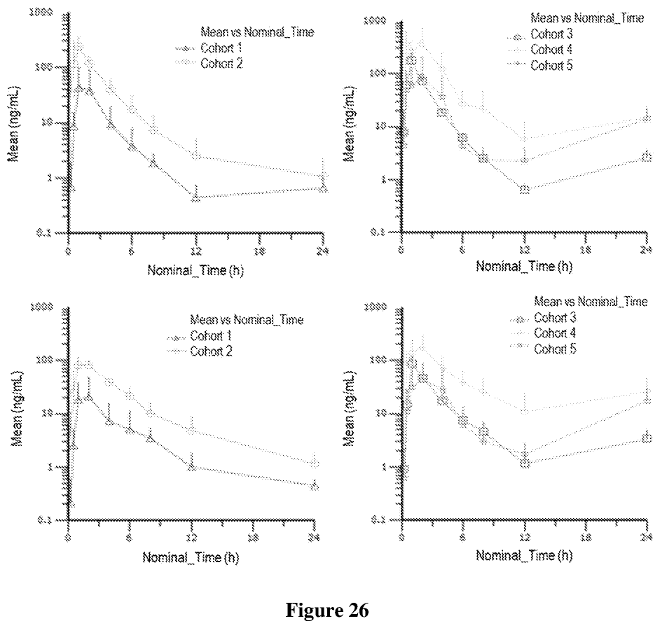

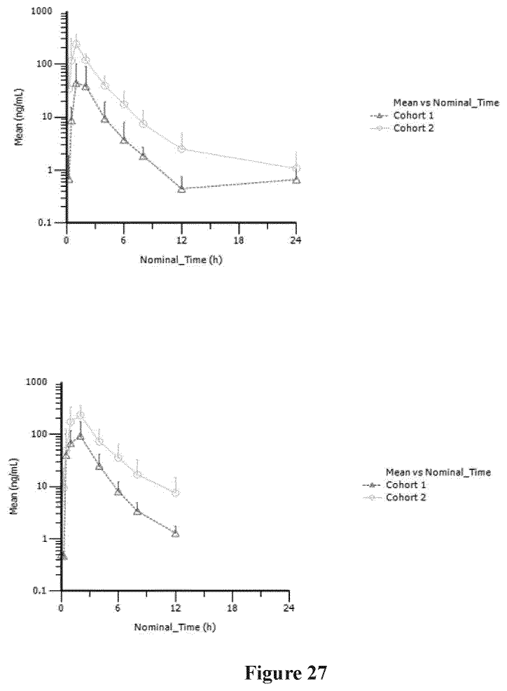

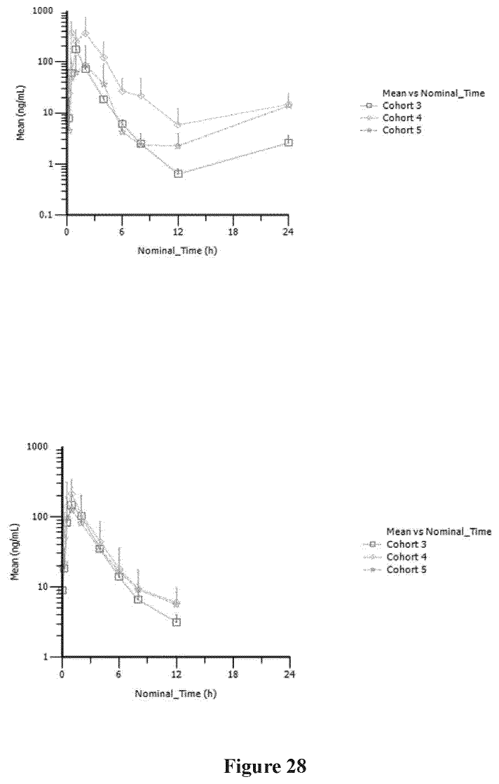

The assessments during the screening phase determined patient eligibility for the study and a patient's ability to comply with protocol requirements by completing all screening assessments. The procedures to be performed throughout the study are outlined in the schedule of assessments (FIG. 2A-D).

The following procedures were performed and recorded during the screening period:
Written informed consent
Inclusion and exclusion criteria
Cancer history
Current and prior cancer therapy/medications/related side effects (eg, immune-mediated, liver toxicity)
Other medical history
Demographic data (age, gender, race)
Other current and prior medications (taken in the past month)
Height and body weight
Vital signs: respiratory rate, heart rate, blood pressure, and body temperature
Physical examination: examination of general appearance, skin, neck (including thyroid), eyes, ears, nose, throat, heart, lungs, abdomen, lymph nodes, extremities, and nervous system
ECOG performance status
12-lead ECG
Echocardiogram or any validated left ventricular ejection fraction assessment method used by site (ie, multigated acquisition)
Urine sample for urinalysis
Blood sample for hematology, biochemistry, and coagulation tests
Blood samples for PD:
Blood sample for PD1: pCREB analysis and other exploratory analysis
Blood samples for PD2: cytokine/chemokine analysis
Blood sample (serum) for exploratory analysis
Blood sample for serum pregnancy test (for females of childbearing potential)
Tumor biopsy: the pre-treatment biopsy was performed at any time during the screening period once it had been checked that the patient meets the other inclusion criteria (Day −28 to Day −1). An existing biopsy taken within 1 month before enrollment into the study was acceptable if no intervening anti-cancer therapy had been administered.
CT and/or MRI scans for assessment of tumor status

Cycle 1

The visit windows during cycle 1 were not cumulative. Study staff tried to compensate the windows when scheduling the next visit so that Visit 5 occurs on Day 28. Each visit or event was recorded in the eCRF with the calendar day of the actual visit date and at the corresponding chronologic cycle and day. For example, if a visit occurs on Day 7 after the first treatment instead of Day 8, then it should be recorded as the C1D8 visit occurring on Day 7, the next visit (C1D15) is kept for Day 15, 8 days later, not Day 14, 7 days later.

Cycle 1—Day 1

The following procedures were performed and recorded during this visit:
Admission to clinic
Inclusion and exclusion criteria
Body weight
Vital signs: respiratory rate, heart rate, blood pressure, and body temperature (within 30 minutes before dosing and within 90 minutes after dosing)
Physical examination: examination of general appearance, skin, neck (including thyroid), eyes, ears, nose, throat, heart, lungs, abdomen, lymph nodes, extremities, and nervous system
ECOG performance status
12-lead ECG: ECGs will be obtained in triplicate within 30 minutes before dosing and within 90 minutes after dosing with Compound 8a. At least one of the triplicates must be reviewed by the investigator at the center before continue dosing. All triplicates may be reviewed by a central reader
Urine sample for urinalysis (may be performed up to 72 hours before the visit)
Urine or serum pregnancy test (for females of childbearing potential; dipstick test)
Blood sample for hematology, biochemistry, and coagulation tests (may be performed up 72 hours before the visit)
Blood sample for PK at pre-dose
Blood samples for PD at pre-dose
Blood sample for PD1: pCREB analysis and other exploratory analysis
Blood samples for PD2: cytokine/chemokine analysis
Blood sample (serum) for exploratory analysis at pre-dose
Dispensing and administration of IP with 250 mL of authorized beverage
PK blood sampling at 0.25, 0.5, 1, 2, 4, 6, 8, 12, and 24 hours after dosing
PD blood sampling at 2, 6, 12, and 24 hours after dosing
Blood sample for PD1: pCREB analysis and other exploratory analysis
Blood samples for PD2: cytokine/chemokine analysis
Concomitant medications and treatments
AEs
Patient diary dispensing and training
Discharge from clinic on subsequent day after the last foreseen PK and PD blood sampling, after a performance of a physical examination and assessment of vital signs and AEs

Cycle 1—Day 8

The following procedures were performed and recorded during this visit:
Vital signs: respiratory rate, heart rate, blood pressure, and body temperature (within 30 minutes before dosing and within 90 minutes after dosing)
12-lead ECG: ECGs were obtained in triplicate within 30 minutes before dosing and within 90 minutes after dosing with Compound 8a.
Concomitant medications and treatments
AEs
Blood sample for hematology and biochemistry tests (may be performed up to 72 hours before the visit)
Return IP
Blood sample for PK at pre-dose
Blood sample (serum) for exploratory analysis at pre-dose
Administration of IP with 250 mL of authorized beverage
Review of diary

Cycle 1—Day 15

The following procedures were performed and recorded during this visit:
Vital signs: respiratory rate, heart rate, blood pressure, and body temperature (within 30 minutes before dosing and within 90 minutes after dosing)
12-lead ECG: ECGs were obtained in triplicate within 30 minutes before dosing and within 90 minutes after dosing with Compound 8a.
Concomitant medications and treatments
AEs
Blood sample for hematology, biochemistry, and coagulation tests up to 72 hours before the visit
Return IP
Blood sample for PK at pre-dose
Blood samples for PD at pre-dose
Blood sample for PD1: pCREB analysis and other exploratory analysis
Blood samples for PD2: cytokine/chemokine analysis)
Blood sample (serum) for exploratory analysis at pre-dose
Administration of IP with 250 mL of authorized beverage
Review of diary
Blood sampling for PK at 0.25, 0.5, 1, 2, 4, 6, 8, and 12 hours after dosing
Blood sampling for PD at 2, 6, and 12 hours after dosing
Blood sample for PD1: pCREB analysis and other exploratory analysis
Blood samples for PD2: cytokine/chemokine analysis,
Optional overnight stay, discharge on subsequent day after a performance of a physical examination and assessment of vital signs and AEs

Cycle 1—Day 21 (+6 Days) with Biopsy

The following procedures was performed and recorded during this visit:
Vital signs: respiratory rate, heart rate, blood pressure, and body temperature (within 30 minutes before dosing and within 90 minutes after dosing)
12-lead ECG: ECGs were obtained in triplicate within 30 minutes before dosing and within 90 minutes after dosing with Compound 8a.
Concomitant medications and treatments
AEs
Blood sample for hematology, biochemistry, and coagulation tests (may be performed up to 72 hours before the visit)
Return IP
Tumor biopsy
Administration of IP with 250 mL of authorized beverage
Review of diary

Cycle 2 and Subsequent Cycles—Day 1

The following procedures was performed and recorded during these visits
Body weight
Vital signs: respiratory rate, heart rate, blood pressure, and body temperature (at any time before dosing)
Physical examination: examination of general appearance, skin, neck (including thyroid), eyes, ears, nose, throat, heart, lungs, abdomen, lymph nodes, extremities, and nervous system
ECOG performance status
12-lead ECG
For C2D1 and C3D1, triplicate ECGs were obtained before dosing.
For C4D1 and subsequent cycles, only 1 ECG will be obtained at the time of visit.
Echocardiogram or any validated left ventricular ejection fraction assessment method used by site (ie, multigated acquisition) (C2D1 ±7 days), then every subsequent 8 weeks (±7 days)
Concomitant medications and treatments.
AEs
Urine sample for urinalysis (may be performed up to 72 hours before the visit)
Urine or serum pregnancy test (for females of childbearing potential; dipstick test)
Blood sample for hematology, biochemistry, and coagulation tests
Return IP
Blood sample for PK at pre-dose (C2D1 and C3D1 only)
Blood samples for PD at pre-dose (C2D1 and C3D1 only)
Blood sample for PD1: pCREB analysis and other exploratory analysis
Blood samples for PD2: cytokine/chemokine analysis
Blood sample (serum) for exploratory analysis at pre-dose (C2D1 and C3D1 only)
Dispensing of Compound 8a
Administration of IP with 250 mL of authorized beverage
Return, review, and dispensing of diary
Blood sampling for PK at 2, 6, and 12 hours after dosing (C2D1 only)
Blood sampling for PD at 2, 6, and 12 hours after dosing (C2D1 only)
Blood sample for PD1: pCREB analysis and other exploratory analysis
Blood sample for PD2: cytokine/chemokine analysis
Optional overnight stay, discharge on subsequent day after a performance of a physical examination and assessment of vital signs and AEs (C2D1 only)
CT and/or MRI scans for assessment of tumor status every 8 weeks starting from C1D1 until EOT. These can be performed up to 7 days before starting the next treatment cycle or at any time during cycles, if clinically indicated (for example, symptoms of tumor progression).

2.9.1 End of Treatment

The patients received Compound 8a until unacceptable toxicity, disease progression, withdrawal of consent, or any of other reasonable criteria. Any patient who has received at least 1 dose of Compound 8a underwent an EOT and follow-up visit 30 days after EOT. The EOT visit occurred within 7 days after the EOT. The following procedures were performed and recorded during the EOT visit:
Body weight
Vital signs: respiratory rate, heart rate, blood pressure, and body temperature
Physical examination: examination of general appearance, skin, neck (including thyroid), eyes, ears, nose, throat, heart, lungs, abdomen, lymph nodes, extremities, and nervous system
ECOG performance status
12-lead ECG: triplicate ECGs were obtained at any time at the EOT visit. At least one of the triplicates were reviewed.
Echocardiogram or any validated left ventricular ejection fraction assessment method used by site (ie, multigated acquisition) (to be performed between EOT and the 30-day follow-up visit)
Concomitant medications and treatments
AEs
Urine sample for urinalysis
Urine or serum pregnancy test (for females of childbearing potential; dipstick test)
Blood sample for hematology, biochemistry, and coagulation tests
Return IP
Return and review of diary

2.9.2 Follow-Up Phase

Follow-Up Visit at 30 Days After End of Treatment (End of Study Visit)

The following procedures were performed and recorded during this visit:
Body weight
Vital signs: respiratory rate, heart rate, blood pressure, and body temperature
Physical examination: examination of general appearance, skin, neck (including thyroid), eyes, ears, nose, throat, heart, lungs, abdomen, lymph nodes, extremities, and nervous system
ECOG performance status
12-lead ECG (for local reading by investigator)
Concomitant medications and treatments.
AEs
Blood sample for hematology and biochemistry

Follow-Up Until Withdrawal or Start of New Anti-Cancer Therapy

After EOT, tumor assessment was followed at regular intervals based on imaging procedures (CT/MRI) until withdrawal or start of new anti-cancer therapy. The imaging procedures were performed as part of the standard patient care (schedule according to standard of practice). The purpose of following tumor assessment is to assess duration of response or confirm progression in patient for whom the decision to stop the treatment was not related to disease progression. The patients also were followed via regular phone calls until withdrawal of consent or start of new anti-cancer therapy in order to assess survival and subsequent cancer therapies.

2.11 Efficacy Assessments

Tumor Response: tumor response to Compound 8a was assessed by means of RECIST v1.1 (Eisenhauer et al. 2009). CT and/or MRI scans to assess tumor status were obtained at baseline and approximately every 8 weeks from C1D1 until EOT.

2.11 Safety Assessments

Physical Examination: Physical examinations included examination of general appearance, skin, neck (including thyroid), eyes, ears, nose, throat, heart, lungs, abdomen, lymph nodes, extremities, and nervous system.

Height without shoes recorded in meters at screening.

Vital Signs: Vital signs will include body temperature (° C.), respiratory rate, heart rate, and blood pressure.

Body Weight: Body weight without shoes in kilograms.

Electrocardiogram: Standard 12-lead ECGs will be measured and PR interval, QRS interval, QT interval, QT interval corrected for heart rate using Bazett's formula (QTcB), and QTcF were determined.

During the dose-escalation phase, ECGs were obtained in triplicate (within 5 minutes between each ECG), performed within 30 minutes before dosing and within 90 minutes after dosing with Compound 8a on C1D1, C1D8, C1D15, and C1D21. For C2D1 and C3D1, triplicate ECGs were obtained before dosing.

Echocardiogram: Left ventricular ejection fraction was determined by cardiac ultrasound at the center during screening period, before start of treatment with Compound 8a, at (C2D1 ±7 days), then every subsequent 8 weeks (±7 days) and between EOT and the 30-day follow-up visit. Echocardiogram or any validated left ventricular ejection fraction assessment method used by site was acceptable.

Laboratory Parameters: The following clinical laboratory tests were performed as indicated by the schedule of assessments:
Hematology: Red blood cell count, hemoglobin, hematocrit, white blood cell count (with differential), and platelet count.
Coagulation: Prothrombin time, activated partial thromboplastin time, and international normalized ratio.
Chemistry: Aspartate aminotransferase, alanine aminotransferase, total bilirubin, direct bilirubin, alkaline phosphatase, lactate dehydrogenase, creatinine, blood urea nitrogen, uric acid, glucose, lipase, amylase, gamma glutamyl transferase, thyroid stimulating hormone, free T3 and T4, total protein, albumin, C-reactive protein, sodium, potassium, chloride, calcium, phosphorus, magnesium, creatinine clearance (Cockcroft-Gault method) (screening only), BNP, and troponin (any troponin subtype consistent with the regular practice of the site laboratory may be used).
Urinalysis: pH of freshly voided specimen, specific gravity, protein, glucose, ketones, blood, and microscopic examination of the sediment.
Pregnancy test: A serum pregnancy test for females of childbearing potential was performed at screening (and reflex serum testing if urine pregnancy test is positive thereafter), and a urine or serum pregnancy test (dipstick test) for all subsequent tests.

Laboratory samples were analyzed by the local laboratories.

Eastern Cooperative Oncology Group (ECOG): ECOG Performance Status were recorded.

Adverse Events: All AEs occurring after the patient signs the ICF and up to 30 days after last study drug administration were recorded, thereafter, only drug-related AEs/SAEs were reported.

2.11 Pharmacokinetic Assessments

Blood samples (2 mL each) for PK analysis were obtained during the dose-escalation phase at the time points indicated in the schedules of assessment (FIG. 2B).

Plasma samples prepared from the collected blood samples were analyzed for both Compound 8a (parent molecule) as well as Compound 77 (active metabolite), using a validated liquid chromatography/tandem mass spectrometry method; ie, plasma concentrations for parent Compound 8a and metabolite Compound 77 were determined and reported separately.

The following PK parameters (but not limited to) were evaluated:

AUC0-inf AUC from zero up to infinity with extrapolation of the terminal phase

AUC0-tlast Area under the concentration-time curve from zero up to the last concentration□ lower limit of quantification

AUC0-τ Area under the concentration-time curve during a dosage interval

C max maximum plasma concentration

Lambda z Terminal rate constant

t½ Plasma concentration half-life

t max Time to reach C max

AR Accumulation ratio 2.12 Pharmacodynamic Assessments

Blood samples for PD analysis were obtained during the dose-escalation phase at the time points indicated in the schedules of assessment (FIG. 2A). Two whole blood cell-based immunoassays were used to assess the PD of target (A2AR) engagement by Compound 8a in dosed patients.

2.12.1 PD Assay 1: pCREB Analysis

To assess target engagement by Compound 8a, peripheral blood samples (4 mL) were collected in sodium heparin tubes before initiation of therapy and at the time points indicated in FIG. 2A.

Whole blood was stimulated ex vivo with the A2AR-selective agonist CGS-21680 to induce pCREB, and inhibition of CGS-21680-induced pCREB in peripheral blood lymphocytes of patients dosed with Compound 8a was analyzed. Levels of pCREB were determined with flow cytometry using a qualified method.

Summary measures of pCREB levels and derived PD parameters (including inhibition of pCREB) were presented at each IP dose and in relation to PK.

2.12.2 PD Assay 2: Cytokine/Chemokine Analysis

To further assess target engagement by Compound 8a at a downstream level, peripheral blood samples were drawn in 2 TruCulture tubes (2×1 mL of blood) before initiation of therapy and at the time points indicated in FIG. 2A.

Blood was stimulated with lipopolysaccharide in the presence or in the absence of the A2AR-selective agonist CGS-21680, and cytokine and chemokine secretion in the 2 conditions were analyzed using qualified multi-analyte profiling assays.

Summary measures of cytokine and chemokine levels and derived PD parameters will be presented at each IP dose and in relation to PK.

2.13 Other Exploratory Biomarkers

Three types of specimens (tumor tissue, blood and serum) were obtained from all patients for exploratory biomarker analysis at the certain time points indicated in FIG. 2. The purpose of specimen collection is to: 1) assess the immune response to therapy in the tumor tissue and in the peripheral blood; 2) gain important insights into Compound 8a mechanism of action; 3) assess the associations of potential biomarkers and Compound 8a antitumor activity.

2.13.1 Characterization of Tumor Microenvironment

To characterize the TME before and during exposure to Compound 8a, archival tumor tissue and paired biopsies were analyzed using immunohistochemistry and multiplex gene expression profiling platforms Immunohistochemical stainings may include, but are not necessarily limited to, the analysis of A2AR, CD39 and CD73 (markers of the adenosine cloud), CD3, CD8 and FoxP3 (markers of immune cell infiltration), granzyme B and Ki-67 (markers of T cell activation and proliferation). Gene expression profiling may cover, but is not necessarily limited to, genes involved in adaptive and innate immune responses, including immune checkpoints, cytokine and chemokine transcripts.

2.13.2 Characterization of Circulating Immune Cell Populations

Residual peripheral blood collected for PD1, pCREB analysis, were used to characterize circulating immune cell populations before and during exposure to Compound 8a using flow cytometry.

Flow cytometry panels designed for immunophenotyping of immune cell populations may be used including, but not necessarily limited to, T lymphocyte subpopulations and their activation and functional status.

2.13.3 Evaluation of Serum Biomarkers

Blood samples for exploratory analysis (2.5 mL each) were drawn at the times indicated in FIG. 2, and serum was isolated.

Samples may be assessed by enzyme-linked immunosorbent assays, multiplex analyte profiling panels, and/or other relevant techniques for quantification of serum markers, including but not limited to cytokines, chemokines, inflammatory factors, growth factors or other soluble mediators.

2.14 Response Criteria
Complete Response (CR): Disappearance of all target lesions. Any pathological lymph nodes (whether target or non-target) must have reduction in short axis to <10 mm
Partial Response (PR): At least a 30% decrease in the sum of diameters of target lesions, taking as reference the baseline sum diameters.
Progressive Disease (PD): At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm
Stable Disease (SD): Neither sufficient shrinkage to qualify for PR nor sufficient increase to qualify for PD, taking as reference the smallest sum diameters while on study.
Not Evaluable (NE): If 1 or more target lesions cannot be measured or adequately assessed as either fully resolved or too small to measure (due to missing or poor-quality images), and the sum of diameters of the remaining measured target lesions (if any) has not increased sufficiently to meet PD as defined above. Table 6 shows the response of the 21 patients enrolled in the study.

TABLE 6

| | QD doses | BID doses | Total | |
|---|---|---|---|---|
| Best Response | (n = 6), n (%) | (n = 15), n (%) | (n = 21), n (%) | |
| Complete Response | 0% | 0% | 0% | |
| Partial Response | 0% | 2 (13%) | 2 | (9.5%) |
| Stable Disease | 1 (16.5%) | 4 (27%) | 5 | (24%) |
| Progressive Disease | 4 (67%) | 8 (53%) | 12 | (57%) |
| Not Assessed | 1 (16.5%) | 1 (7%) | 2 | (9.5%) |

Out of the 21 patients were treated with Compound 8a monotherapy on this study, 5 patients had stable disease as their best response. Two of these patients have now been on therapy for over 9 months and are continuing treatment. Two out of the 12 patients treated at the highest dose levels had a partial response (FIG. 3A-B).

Preliminary evidence of clinical study showed that Compound 8a benefited 7 patients with 2 ongoing partial responses in a checkpoint inhibitor-refractory melanoma patient and a patient with metastatic prostate cancer.

2.15 Detailed Analysis of Pharmacodynamic Assays

Due to the long receptor residence time of Compound 8a, exposures at both doses (80 mg BID dose and the 160 mg BID) dose-maintained inhibition of A2AR activity in two pharmacodynamic assays which were conducted to assess target engagement, one assessing at the phosphorylation of CREB downstream of the A2AR, and the second assessing the induction of the inflammatory cytokine, TNF-alpha.

2.15.1 pCREB Pharmacodynamic Assay: Assessment of CREB Phosphorylation Induced by an A2AR Agonist in Peripheral Blood Samples

### LIST OF ABBREVIATIONS

A_{2A}R Adenosine A_{2A} receptor

BID Bis in die (twice daily)

C1D1 Cycle 1 Day 1

C1D15 Cycle 1 Day 15

C2D1 Cycle 2 Day 1

C3D1 Cycle 3 Day 1

CD Cluster of differentiation

CGS CGS-21680, a specific adenosine A_{2A} receptor agonist

CREB cAMP responsive element-binding protein

DMSO Dimethyl sulfoxide

CV Coefficient of variation

Geomean Geometric mean

h hour

HD Healthy donor

MeFI Median fluorescence intensity

min minute

nMeFI DMSO-normalized median fluorescence intensity

pCREB CREB protein phosphorylated at serine residue 133

PD Pharmacodynamic

PMA Phorbol-12-myristate-13 acetate+Ionomycin cocktail

PROT1 PROT1 buffer

QC Quality check

QD Quaque die (once a day)

SAP Statistical analysis plan

SD Standard deviation

T Time point

Pharmacodynamic (PD) activity using “PD1-pCREB assay” showed adenosine A2A receptor (A2AR) engagement in the peripheral blood of patients dosed with Compound 8a through the quantification of CREB phosphorylation at serine 133 (pCREB) in lymphocytes using flow cytometry. Principle of the assay and assay readouts is shown in FIG. 4.

Patients were enrolled in 5 dose levels (5 cohorts), receiving Compound 8a orally either once a day or twice a day, in a 3+3 design.

Each cohort was received the following dose of Compound 8a :

Cohort 1 (3 patients): 20 mg—once a day (QD)

Cohort 2 (3 patients): 40 mg—once a day (QD)

Cohort 3 (3 patients): 40 mg—twice a day (BiD)

Cohort 4 (5 patients): 80 mg—twice a day (BiD)

Cohort 5 (4 patients): 160 mg—twice a day (BiD)

General Assessments for Each Cohort:

Cohort 1: Although 54% of data (including 100% of data from one subject) had to be excluded from the analysis for technical reasons, PD1-pCREB assay showed A_{2A}R engagement by Compound 8a in the 2/2 evaluable subjects dosed with 20 mg QD Compound 8a:
Compound 8a inhibited CREB phosphorylation induced by the A_{2A}R agonist CGS-21680 in CD4⁺, CD8⁺ and CD19⁺ cells
PD activity of Compound 8a was observed after first drug exposure (i.e. C1D1) and at steady state (i.e. C1D15 and C2D1)
Compound 8a effect declined below 50% 24 hours after drug intake (C1D1 data)

Cohort 1 summary data (pCREB_{CGS} nMeFI) are tabulated by subject and summarized by dose group using the geometric mean (Geomean) in Table 7. Summary time profiles are shown in FIG. 5.

TABLE 7

| | | CD4⁺ cells | | | | CD8⁺ cells | | | | CD19⁺ cells | | | |
|---|---|---|---|---|---|---|---|---|---|---|---|---|---|
| | | 01- | 02- | 03- | Geo- | 01- | 02- | 03- | Geo- | 01- | 02- | 03- | Geo- |
| Visit | Time | 001 | 002 | 001 | mean | 001 | 002 | 001 | mean | 001 | 002 | 001 | mean |
| SCR | . | . | . | . | . | . | . | . | . | . | . | . | . |
| C1D1 | 0 | 1.51 | 2.33 | . | 1.88 | 1.69 | 2.23 | . | 1.94 | 1.88 | 2.03 | . | 1.95 |
| | 2 | . | 1.44 | . | 1.44 | . | 1.49 | . | 1.49 | . | 1.37 | . | 1.37 |
| | 6 | 1.05 | | . | 1.05 | 1.15 | . | . | 1.15 | 1.31 | . | . | 1.31 |
| | 12 | . | 1.55 | . | 1.55 | | 1.78 | . | 1.7S | . | 1.52 | . | 1.52 |
| | 24 | 1.32 | . | . | 1.32 | 1.50 | . | . | 1.50 | 1.76 | . | . | 1.76 |
| C1D15 | 0 | 1.54 | 0.75* | . | 1.07 | 1.68 | 0.69* | . | 1.08 | 1.75 | 0.73 | . | 1.13 |
| | 2 | 1.10 | . | . | 1.10 | 1.13 | . | . | 1.13 | 1.14 | . | . | 1.14 |
| | 6 | . | . | . | . | . | . | . | . | . | . | . | . |
| | 12 | 0.95 | . | . | 0.95 | 0.89 | . | . | 0.89 | 0.99 | . | . | 0.99 |
| C2D1 | 0 | 1.05 | 1.59 | . | 1.29 | 1.17 | 1.79 | . | 1.45 | 1.07 | 1.82 | . | 1.40 |
| | 2 | 1.03 | 0.87 | . | 0.95 | 1.08 | 0.93 | . | 1.00 | 1.03 | 0.86 | . | 0.94 |
| | 6 | 0.98 | 1.02 | . | 1.02 | 1.05 | 1.13 | . | 1.09 | 0.92 | 1.10 | . | 1.01 |
| | 12 | 1.04 | 1.09 | . | 1.06 | 1.02 | 1.18 | . | 1.10 | 1.03 | 1.11 | . | 1.07 |
| C3D1 | 0 | . | 1.27 | . | 1.27 | . | 1.26 | . | 1.26 | . | 1.59 | . | 1.59 |

Legend:
“.” sample not available for analysis.
“*” pCREB_{CGS} nMeFI <0.8

The percent inhibition of CGS-21680 induced pCREB at C1D1 is tabulated by subject, time point and cell population, and summarized by dose group using the arithmetic mean (Mean) in Table 8. Time profiles are shown in FIG. 6.

TABLE 8

| | CD4⁺ cells | | | | CD8⁺ cells | | | | CD19⁺ cells | | | |
|---|---|---|---|---|---|---|---|---|---|---|---|---|
| Time | 01- | 02- | 03- | | 01- | 02- | 03- | | 01- | 02- | 03- | |
| point | 001 | 002 | 001 | Mean | 001 | 002 | 001 | Mean | 001 | 002 | 001 | Mean |
| 0 | 0 | 0 | . | 0 | 0 | 0 | . | 0 | 0 | 0 | . | 0 |
| 2 | . | 67 | . | 67 | . | 60 | . | 60 | . | 64 | . | 64 |
| 6 | 90 | . | . | 90 | 78 | . | . | 78 | 65 | . | . | 65 |
| 12 | . | 59 | . | 59 | . | 37 | . | 37 | . | 50 | . | 50 |
| 24 | 37 | . | . | 37 | 28 | . | . | 28 | 14 | . | . | 14 |

Cohort 2: PD1-pCREB assay showed A2AR engagement by Compound 8a in the 3/3 subjects dosed with 40 mg QD Compound 8a:
Compound 8a inhibited CREB phosphorylation induced by the A2AR agonist CGS-21680 in CD4+, CD8+ and CD19+ cells
PD activity of Compound 8a was observed after first drug exposure (i.e. C1D1) and at steady state (i.e. C1D15 and C2D1)
Compound 8a effect declined 24 hours after drug intake (C1D1 data)

Inhibition of pCREB seemed to be maintained at steady state, with low activity at T0 (pre-dose samples) on C1D15 and C2D1.

Cohort 2 summary data (pCREB_{CGS} nMeFI) are tabulated by subject and summarized by dose group using the geometric mean (Geomean) in Table 9. Summary time profiles are shown in FIG. 7.

TABLE 9

| | | CD4⁺ cells | | | | CD8⁺ cells | | | | CD19⁺ cells | | | |
|---|---|---|---|---|---|---|---|---|---|---|---|---|---|
| | | 01- | 02- | 03- | Geo- | 01- | 02- | 03- | Geo- | 01- | 02- | 03- | Geo- |
| Visit | Time | 001 | 002 | 001 | mean | 001 | 002 | 001 | mean | 001 | 002 | 001 | mean |
| SCR | . | . | 1.48 | 1.72 | 1.60 | . | 1.37 | 1.22 | 1.29 | . | 1.39 | 1.88 | 1.62 |
| C1D1 | 0 | 2.14 | 2.77 | 1.51 | 2.08 | 1.94 | 2.83 | 1.42 | 1.98 | 1.76 | 2.47 | 1.48 | 1.86 |
| | 2 | 1.02 | 1.05 | 1.01 | 1.03 | 1.00 | 1.43 | 1.07 | 1.19 | 0.99 | 1.00 | 1.11 | 1.03 |
| | 6 | 1.13 | 1.59 | 1.20 | 129 | 1.26 | 1.47 | 1.20 | 1.31 | 1.12 | 1.49 | 1.20 | 1.26 |
| | 12 | 1.30 | 1.15 | 1.14 | 1.19 | 1.41 | 1.34 | 1.18 | 1.31 | 1.22 | 1.25 | 1.19 | 1.22 |
| | 24 | . | 1.99 | . | 1.99 | . | 2.01 | . | 2.01 | . | 1.87 | . | 1.87 |
| C1D15 | 0 | 1.28 | 1.05 | 1.60 | 1.29 | 1.27 | 0.98 | 1.36 | 1.19 | 1.25 | 1.12 | 1.80 | 1.36 |
| | 2 | 1.06 | 1.00 | 1.06 | 1.04 | 1.08 | 1.04 | 1.04 | 1.05 | 1.02 | 1.22 | 1.00 | 1.11 |
| | 6 | 1.24 | 1.12 | 0.96 | 1.10 | 1.34 | 1.20 | 0.91 | 1.14 | 1.20 | 1.17 | 0.92 | 1.09 |
| | 12 | 0.98 | 1.66 | 1.68 | 1.40 | 1.14 | 1.82 | 1.45 | 1.44 | 1.24 | 1.96 | 1.68 | 1.60 |
| C2D1 | 0 | 1.10 | 1.08 | . | 1.09 | 1.19 | 1.18 | . | 1.18 | 1.18 | 0.94 | . | 1.05 |
| | 2 | 0.83 | 0.85 | 1.03 | 0.90 | 0.85 | 0.96 | 1.06 | 0.95 | 0.80 | 0.98 | 1.10 | 0.95 |
| | 6 | 1.10 | 1.00 | 1.22 | 1.10 | 1.23 | 0.95 | 1.13 | 1.10 | 1.13 | 1.03 | 1.16 | 1.11 |
| | 12 | 0.90 | . | 1.39 | 1.12 | 1.04 | . | 1.15 | 1.09 | 0.96 | . | 1.45 | 1.18 |
| C3D1 | 0 | . | . | 1.11 | 1.11 | . | . | 1.05 | 1.05 | . | . | 1.11 | 1.11 |

Legend:
“.” sample not available for analysis.

The percent inhibition of CGS-21680 induced pCREB at C1D1 is tabulated by subject, time point and cell population, and summarized by dose group using the arithmetic mean (Mean) in Table 10. Time profiles are shown in FIG. 8.

TABLE 10

| Time | CD4⁺ cells | | | | CD8⁺ cells | | | | CD19⁺ cells | | | |
|---|---|---|---|---|---|---|---|---|---|---|---|---|
| point | 01-003 | 02-003 | 03-002 | Mean | 01-003 | 02-003 | 03-002 | Mean | 01-003 | 02-003 | 03-002 | Mean |
| 0 | 0 | 0 | 0 | 0 | 0 | 0 | 0 | 0 | 0 | 0 | 0 | 0 |
| 2 | 98 | 97 | 98 | 98 | 90 | 77 | 83 | 83 | 101 | 100 | 77 | 93 |
| 6 | 89 | 67 | 61 | 72 | 72 | 74 | 52 | 66 | 84 | 67 | 58 | 70 |
| 12 | 74 | 92 | 73 | 79 | 56 | 81 | 57 | 65 | 71 | 83 | 60 | 71 |
| 24 | • | 44 | • | 44 | • | 45 | • | 45 | • | 41 | • | 41 |

Cohort 3: PD1-pCREB assay showed A_{2A}R engagement by Compound 8a in the at least 2 of the 3 subjects dosed with 40 mg BID Compound 8a:
Compound 8a inhibited CREB phosphorylation induced by the A_{2A}R agonist CGS-21680 in CD4⁺, CD8⁺ and CD19⁺ cells
PD activity of Compound 8a was observed after first drug exposure (i.e. C1D1) and at steady state (i.e. C1D15 and C2D1)
With BID dosing, almost full inhibition of CGS-21680 effect was observed at 24 hours post-dose (C1D1 data)

Cohort 3 summary data (pCREB_{CGS} nMeFI) are tabulated by subject and summarized by dose group using the geometric mean (Geomean) in Table 11. Summary time profiles are shown in FIG. 9.

TABLE 11

| | | CD4⁺ cells | | | | CD8⁺ cells | | | | CD19⁺ cells | | | |
|---|---|---|---|---|---|---|---|---|---|---|---|---|---|
| Visit | Time | 01-005 | 02-004 | 03-003 | Geomean | 01-005 | 02-004 | 03-003 | Geomean | 01-005 | 02-004 | 03-003 | Geomean |
| SCR | • | 1.48 | 1.49 | 1.58 | 1.52 | 1.44 | 1.42 | 1.61 | 1.49 | 1.23 | 1.46 | 1.11 | 1.26 |
| C1D1 | 0 | 1.38 | 1.53 | • | 1.45 | 1.34 | 1.47 | • | 1.40 | 1.16 | 1.39 | • | 1.27 |
| | 2 | 1.08 | • | 1.12 | 1.10 | 1.07 | • | 1.14 | 1.10 | 1.06 | • | 1.15 | 1.10 |
| | 6 | • | • | 1.11 | 1.11 | • | • | 1.18 | 1.18 | • | • | 1.08 | 1.08 |
| | 12 | • | 1.28 | • | 1.28 | • | 1.37 | • | 1.37 | • | 1.22 | • | 1.22 |
| | 24 | 0.97 | 0.95 | 1.10 | 1.00 | 0.88 | 1.06 | 1.19 | 1.04 | 0.98 | 0.93 | 1.10 | 1.00 |
| C1D15 | 0 | 1.18 | 1.12 | 0.93 | 1.07 | 1.14 | 1.18 | 1.00 | 1.10 | 1.30 | 1.13 | 0.85 | 1.08 |
| | 2 | 1.03 | 1.06 | 1.18 | 1.09 | 1.04 | 1.09 | 1.28 | 1.13 | 1.20 | 0.95 | 1.20 | 1.11 |
| | 6 | 1.02 | 1.12 | 1.19 | 1.11 | 1.09 | 1.22 | 1.27 | 1.19 | 1.04 | 1.15 | 1.08 | 1.09 |
| | 12 | • | 1.22 | • | 1.22 | • | 1.41 | • | 1.41 | • | 1.27 | • | 1.27 |
| C2D1 | 0 | 1.16 | • | 1.31 | 1.24 | 1.16 | • | 1.33 | 1.24 | 0.97 | • | 0.96 | 0.97 |
| | 2 | 0.97 | • | 1.08 | 1.02 | 0.96 | • | 1.23 | 1.09 | 0.98 | • | 0.98 | 0.98 |
| | 6 | 1.18 | 1.29 | 1.63 | 1.35 | 1.19 | 1.61 | 1.63 | 1.46 | 1.18 | 1.33 | 1.41 | 1.30 |
| | 12 | 1.17 | 1.13 | • | 1.15 | 1.23 | 1.18 | • | 1.20 | 1.14 | 1.13 | • | 1.13 |
| C3D1 | 0 | • | • | 1.18 | 1.18 | • | • | 1.23 | 1.23 | • | • | 1.17 | 1.17 |

Legend: “•”: sample not available for analysis.

The percent inhibition of CGS-21680 induced pCREB at C1D1 is tabulated by subject, time point and cell population, and summarized by dose group using the arithmetic mean (Mean) in Table 12. Time profiles are shown in FIG. 10.

TABLE 12

| Time | CD4⁺ cells | | | | CD8⁺ cells | | | | CD19⁺ cells | | | |
|---|---|---|---|---|---|---|---|---|---|---|---|---|
| point | 01-005 | 02-004 | 03-003 | Mean | 01-005 | 02-004 | 03-003 | Mean | 01-005 | 02-004 | 03-003 | Mean |
| 0 | 0 | 0 | • | 0 | 0 | 0 | • | 0 | • | 0 | • | 0 |
| 2 | 79 | • | • | 79 | 79 | • | • | 79 | • | • | • | • |
| 6 | • | • | • | • | • | • | • | • | • | • | • | • |
| 12 | • | 47 | • | 47 | • | 21 | • | 21 | • | 44 | • | 44 |
| 24 | 108 | 109 | • | 109 | 135 | 87 | • | 111 | • | 118 | • | 118 |

Legend: “•”: sample not available for analysis.

Cohort 4: PD1-pCREB assay showed A2AR engagement by Compound 8a in the all evaluable subjects dosed with 80 mg BID Compound 8a:
Compound 8a inhibited CREB phosphorylation induced by the A2AR agonist CGS-21680 in CD4+, CD8+ and CD19+ cells
PD activity of Compound 8a was observed after first drug exposure (i.e. C1D1) and at steady state (i.e. C1D15 and C2D1)
Almost full inhibition of CGS-21680 effect was observed at 24 hours post-dose (C1D1 data)

Cohort 4 summary data (pCREB_{CGS} nMeFI) are tabulated by subject and summarized by dose group using the geometric mean (Geomean) in Table 13. Summary time profiles are shown in FIG. 11. One subject was excluded from the summary analysis due to high unexplicable variability of the results from that patient.

TABLE 13

| | | CD4⁺ cells | | | | | | CD8⁺ cells | | | |
|---|---|---|---|---|---|---|---|---|---|---|---|
| Visit | Time | 02-005 | 02-006 | 03-004 | 01-006 | 02-008 | Geomean | 02-005 | 02-006 | 03-004 | 01-006 |
| SCR | • | • | 1.51 | • | 1.70 | | 1.60 | • | 1.47 | • | 1.46 |
| C1D1 | 0 | 1.45 | 2.09 | • | 2.48 | 2.20 | 2.02 | 1.52 | 1.87 | • | 1.55 |
| | 2 | 1.16 | 0.79* | • | • | 0.96 | 0.96 | 1.13 | 0.78* | • | • |
| | 6 | 1.02 | 1.05 | • | • | 0.96 | 1.01 | 1.13 | 1.16 | • | • |
| | 12 | 1.26 | • | • | 1.14 | 1.18 | 1.19 | 1.27 | • | • | 1.21 |
| | 24 | 1.08 | 0.94 | • | 1.16 | 1.17 | 1.08 | 1.09 | 0.95 | • | 1.07 |
| C1D15 | 0 | 1.23 | 1.20 | • | • | • | 1.21 | 1.26 | 1.42 | • | • |
| | 2 | 1.33 | 0.97 | • | 0.95 | • | 1.07 | 1.32 | 0.99 | • | 0.98 |
| | 6 | 1.31 | 1.08 | • | 1.44 | • | 1.27 | 1.33 | 1.11 | • | 1.47 |
| | 12 | • | 1.15 | • | 1.24 | • | 1.19 | • | 1.08 | • | 1.23 |
| C2D1 | 0 | 1.26 | 1.36 | • | • | • | 1.31 | 1.28 | 1.45 | • | • |
| | 2 | 0.75* | 0.98 | • | 1.04 | • | 0.92 | 0.76* | 1.02 | • | 0.96 |
| | 6 | 1.00 | 1.00 | • | • | • | 1.00 | 1.06 | 1.01 | • | • |
| | 12 | • | • | • | • | • | • | • | • | • | • |
| C3D1 | 0 | • | 1.40 | • | • | • | 1.40 | • | 1.57 | • | • |

| | | CD8⁺ cells | | CD19⁺ cells | | | | | |
|---|---|---|---|---|---|---|---|---|---|
| Visit | Time | 02-008 | Geomean | 02-005 | 02-006 | 03-004 | 01-006 | 02-008 | Geomean |
| SCR | • | | 1.46 | • | 1.68 | • | 1.28 | | 1.47 |
| C1D1 | 0 | 1.82 | 1.68 | 1.68 | 1.92 | • | 2.86 | 2.44 | 2.18 |
| | 2 | 1.06 | 0.98 | 1.05 | 0.84 | • | • | 1.09 | 0.99 |
| | 6 | 1.06 | 1.12 | 1.08 | 1.11 | • | • | 1.05 | 1.08 |
| | 12 | 1.22 | 1.23 | 1.32 | • | • | 1.05 | 1.12 | 1.16 |
| | 24 | 1.23 | 1.08 | 1.18 | 1.11 | • | 1.05 | 1.21 | 1.14 |
| C1D15 | 0 | • | 1.34 | 1.27 | 1.22 | • | • | • | 1.24 |
| | 2 | • | 1.09 | 1.24 | 0.93 | • | 0.99 | • | 1.05 |
| | 6 | • | 1.29 | 1.46 | 1.19 | • | 1.26 | • | 1.30 |
| | 12 | • | 1.15 | • | 1.10 | • | 1.16 | • | 1.13 |
| C2D1 | 0 | • | 1.36 | 1.43 | 1.56 | • | • | • | 1.49 |
| | 2 | • | 0.91 | 0.83 | 0.97 | • | 1.16 | • | 0.98 |
| | 6 | • | 1.03 | 1.10 | 1.10 | • | • | • | 1.10 |
| | 12 | • | • | • | • | • | • | • | • |
| C3D1 | 0 | • | 1.57 | • | 1.59 | • | • | • | 1.59 |

Legend: “•”: sample not available for analysis.
*pCREB_{CGS} nMeFI < 0.8

The percent inhibition of CGS-21680 induced pCREB at C1D1 is tabulated by subject, time point and cell population, and summarized by dose group using the arithmetic mean (Mean) in Table 14. Time profiles are shown in FIG. 12.

TABLE 14

| Time | CD4⁺ cells | | | | | | CD8⁺ cells | | |
|---|---|---|---|---|---|---|---|---|---|
| point | 02-005 | 02-006 | 03-004 | 01-006 | 02-008 | Mean | 02-005 | 02-006 | 03-004 |
| 0 | 0 | 0 | • | • | 0 | 0 | 0 | 0 | • |
| 2 | 64 | 119 | • | • | 103 | 96 | 75 | 125 | • |
| 6 | 96 | 95 | • | • | 103 | 98 | 75 | 82 | • |
| 12 | 42 | • | • | 91 | 85 | 73 | 48 | • | • |
| 24 | 82 | 106 | • | 89 | 86 | 91 | 83 | 106 | • |

| Time | CD8⁺ cells | | | CD19⁺ cells | | | | | |
|---|---|---|---|---|---|---|---|---|---|
| point | 01-006 | 02-008 | Mean | 02-005 | 02-006 | 03-004 | 01-006 | 02-008 | Mean |
| 0 | 0 | 0 | 0 | 0 | 0 | • | 0 | 0 | 0 |
| 2 | • | 93 | 98 | 93 | 117 | • | • | 94 | 101 |
| 6 | • | 93 | 83 | 88 | 88 | • | • | 97 | 91 |
| 12 | 62 | 73 | 61 | 53 | • | • | 97 | 97 | 81 |
| 24 | 87 | 72 | 87 | 74 | 88 | • | 97 | 85 | 86 |

Legend: “•”: sample not available for analysis.

Cohort 5: PD1-pCREB assay showed A2AR engagement by Compound 8a in the all evaluable subjects dosed with 160 mg BID Compound 8a:
Compound 8a inhibited CREB phosphorylation induced by the A2AR agonist CGS-21680 in CD4+, CD8+ and CD19+ cells
PD activity of Compound 8a was observed after first drug exposure (i.e. C1D1) and at steady state (i.e. C1D15 and C2D1), although variability among subjects was high.
Differently from previous cohorts, the peak of PD activity and inhibition of CGS effect at C1D1 was at 12 hours
Compound 8a effect declined 24 hours after drug intake (C1D1 data)

Cohort 5 summary data (pCREB_{CGS} nMeFI) are tabulated by subject and summarized by dose group using the geometric mean (Geomean) in Table 15. Summary time profiles are shown in FIG. 13. One subject was excluded from the summary analysis due to high unexplicable variability of the results from that patient.

TABLE 15

| | | CD4⁺ cells | | | | | CD8⁺ cells | | |
|---|---|---|---|---|---|---|---|---|---|
| Visit | Time | 01-007 | 01-008 | 02-007 | 01-009 | Geomean | 01-007 | 01-008 | 02-007 |
| SCR | • | 1.46 | 1.56 | 1.83 | • | 1.61 | 1.37 | 1.59 | 1.75 |
| C1D1 | 0 | 1.53 | 1.61 | 1.33 | 1.54 | 1.50 | 1.49 | 1.54 | 1.30 |
| | 2 | 1.30 | 1.26 | 1.01 | • | 1.18 | 1.29 | 1.27 | 1.18 |
| | 6 | 1.42 | 1.17 | 1.07 | • | 1.21 | 1.35 | 1.13 | 1.07 |
| | 12 | 1.09 | 0.76* | 1.00 | • | 0.94 | 1.15 | 0.78* | 1.07 |
| | 24 | 1.06 | 1.49 | 1.29 | 1.20 | 1.25 | 1.04 | 1.31 | 1.27 |
| C1D15 | 0 | 1.12 | 1.35 | 1.13 | • | 1.20 | 1.01 | 1.42 | 1.17 |
| | 2 | 1.09 | 0.92 | 0.93 | 1.16 | 1.02 | 1.18 | 0.96 | 0.94 |
| | 6 | 1.23 | 1.14 | 1.16 | 0.83 | 1.08 | 1.22 | 1.09 | 1.25 |
| | 12 | • | 1.17 | 1.36 | 1.38 | 1.30 | • | 1.17 | 1.39 |
| C2D1 | 0 | 1.29 | 1.20 | • | • | 1.24 | 1.38 | 1.31 | • |
| | 2 | 1.11 | • | • | • | 1.11 | 1.09 | • | • |
| | 6 | 1.20 | 1.22 | • | • | 1.21 | 1.18 | 1.27 | • |
| | 12 | 1.31 | • | • | • | 1.31 | 1.29 | • | • |
| C3D1 | 0 | • | • | • | • | • | • | • | • |

| | | CD8⁺ cells | | CD19⁺ cells | | | | |
|---|---|---|---|---|---|---|---|---|
| Visit | Time | 01-009 | Geomean | 01-007 | 01-008 | 02-007 | 01-009 | Geomean |
| SCR | • | • | 1.56 | 1.19 | 1.11 | 2.52 | • | 1.49 |
| C1D1 | 0 | 1.03 | 1.32 | 1.38 | 1.26 | 1.79 | 1.82 | 1.54 |
| | 2 | • | 1.25 | 1.29 | 1.04 | 1.07 | • | 1.13 |
| | 6 | • | 1.18 | 1.49 | 1.45 | 1.16 | • | 1.36 |
| | 12 | • | 0.99 | 1.08 | 0.74* | 1.41 | • | 1.04 |
| | 24 | 1.15 | 1.19 | 1.13 | 1.57 | 1.54 | 1.36 | 1.39 |
| C1D15 | 0 | • | 1.19 | 1.17 | 1.57 | 1.42 | • | 1.38 |
| | 2 | 1.23 | 1.07 | 0.96 | 0.86 | 1.12 | 1.33 | 1.05 |
| | 6 | 0.90 | 1.11 | 1.47 | 1.05 | 1.56 | 0.86 | 1.20 |
| | 12 | 1.20 | 1.25 | • | 1.12 | 2.18 | 1.22 | 1.44 |
| C2D1 | 0 | • | 1.34 | 1.37 | 1.05 | • | • | 1.20 |
| | 2 | • | 1.00 | 1.26 | • | • | • | 1.26 |
| | 6 | • | 1.22 | 1.66 | 1.29 | • | • | 1.40 |
| | 12 | • | 1.29 | 1.86 | • | • | • | 1.86 |
| C3D1 | 0 | • | • | • | • | • | • | • |

Legend: “•”: sample not available for analysis.
*pCREB_{CGS} nMeFI < 0.8

The percent inhibition of CGS-21680 induced pCREB at C1D1 is tabulated by subject, time point and cell population, and summarized by dose group using the arithmetic mean (Mean) in Table 16. Time profiles are shown in FIG. 14.

TABLE 16

| Time | CD4⁺ cells | | | | | CD8⁺ cells | | | | | CD19⁺ cells | | | | |
|---|---|---|---|---|---|---|---|---|---|---|---|---|---|---|---|
| point | 01-007 | 01-008 | 02-007 | 01-009 | Mean | 01-007 | 01-008 | 02-007 | 01-009 | Mean | 01-007 | 01-008 | 02-007 | 01-009 | Mean |
| 0 | 0 | 0 | 0 | 0 | 0 | 0 | 0 | 0 | • | 0 | 0 | • | 0 | 0 | 0 |
| 2 | 43 | 57 | 97 | • | 66 | 41 | 50 | 40 | • | 44 | 24 | • | 91 | • | 57 |
| 6 | 21 | 72 | 79 | • | 57 | 29 | 76 | 77 | • | 60 | −29 | • | 80 | • | 25 |
| 12 | 83 | 139 | 100 | • | 107 | 69 | 141 | 77 | • | 96 | 79 | • | 48 | • | 64 |
| 24 | 89 | 20 | 12 | 63 | 46 | 92 | 43 | 10 | • | 48 | 66 | • | 32 | 56 | 51 |

Legend: “•”: sample not available for analysis.

2.15.2 Cytokine Pharmacodynamic Assay: Assessment of Cytokine Levels in the Presence of an A2AR Agonist in Peripheral Blood Samples Stimulated with LPS

### LIST OF ABBREVIATIONS

C1D1 Cycle 1 Day 1

C1D15 Cycle 1 Day 15

C2D1 Cycle 2 Day 1

C3D1 Cycle 3 Day 1

CGS CGS-21680, a specific adenosine A_{2A} receptor agonist

CXCL Chemokine (C-X-C motif) ligand

CV Coefficient of variation

ENA-78 Epithelial-derived neutrophil-activating protein 78

FC Fold change

GM-CSF Granulocyte-macrophage colony stimulating factor

IFN Interferon

IL Interleukin

LPS Lipopolysaccharide

mL milliliter

NT Not targeted

PD Pharmacodynamic

QC Quality check

QD Quaque die (once a day)

SAP Statistical analysis plan

SD Standard deviation

T Time point

TNF Tumor necrosis factor

PD2-cytokine assay estimates adenosine A2A receptor (A2AR) engagement in the peripheral blood of patients dosed with Compound 8a through the quantification of secreted cytokines and chemokines in peripheral blood of cancer patients dosed with Compound 8a following ex vivo stimulation with LPS in the absence or in the presence of CGS.

Patients were divided into 5 cohort for this study. Each cohort was received the following dose of Compound 8a :

Cohort 1 (3 patients): 20 mg—once a day (QD)

Cohort 2 (3 patients): 40 mg—once a day (QD)

Cohort 3 (3 patients): 40 mg—twice a day (BiD)

Cohort 4 (5 patients): 80 mg—twice a day (BiD)

Cohort 5 (4 patients): 160 mg—twice a day (BiD)

General Assessment for Each Cohort:

Cohort 1: Although 25% samples had to be excluded from the analysis for technical reasons, PD2-cytokine assay showed A_{2A}R engagement by Compound 8a in 3/3 subjects dosed with 20 mg QD Compound 8a:
Compound 8a rescued ENA-78, IL-8 and TNF-alpha secretion in LPS-induced peripheral blood in the presence of the A_{2A}R agonist CGS-21680
PD activity of Compound 8a was observed after first drug exposure (i.e. C1D1) and at steady state (i.e. C1D15 and C2D1)

Cohort 1 summary data (pCREB_{CGS} nMeFI) for each cohort are tabulated by subject and summarized by dose group using the geometric mean (Geomean) in Table 17. Summary time profiles are shown in FIG. 15.

TABLE 17

| | | ENA-78 | | | | IL-8 | | | | TNF-alpha | | | |
|---|---|---|---|---|---|---|---|---|---|---|---|---|---|
| Visit | Time | 01-001 | 02-002 | 03-001 | Geomean | 01-001 | 02-002 | 03-001 | Geomean | 01-001 | 02-002 | 03-001 | Geomean |
| SCR | • | 6.25 | 4.32 | 12.06 | 6.88 | 1.72 | 2.02 | 2.48 | 2.05 | 0.39 | 0.44 | 0.11 | 0.27 |
| C1D1 | 0 | 2.14 | 10.48 | 3.73 | 4.37 | 1.55 | 2.45 | 1.81 | 1.90 | 0.32 | 0.41 | 0.22 | 0.31 |
| | 2 | 1.65 | 1.80 | • | 1.72 | 1.13 | 1.12 | • | 1.12 | 0.90 | 0.73 | • | 0.81 |
| | 6 | • | • | 1.03 | 1.03 | • | • | 1.02 | 1.02 | • | • | 0.64 | 0.64 |
| | 12 | 1.35 | 3.47 | • | 2.16 | 1.06 | 2.59 | • | 1.66 | 0.84 | 0.61 | • | 0.72 |
| | 24 | • | 3.14 | • | 3.14 | • | 1.49 | • | 1.49 | • | 0.56 | • | 0.56 |
| C1D15 | 0 | 12.16 | 3.67 | • | 6.68 | 1.92 | 2.72 | • | 2.29 | 0.16 | 0.58 | • | 0.31 |
| | 2 | 0.72 | 0.75 | 1.87 | 1.00 | 0.68 | 0.71 | 1.19 | 0.83 | 0.77 | 0.92 | 0.38 | 0.65 |
| | 6 | • | 1.26 | • | 1.26 | • | 1.74 | • | 1.74 | • | 1.13 | • | 1.13 |
| | 12 | • | 1.78 | 0.76 | 1.16 | • | 1.43 | 0.58 | 0.91 | • | 0.88 | 1.03 | 0.95 |
| C2D1 | 0 | 2.75 | 6.25 | 1.85 | 3.17 | 1.29 | 2.67 | 1.24 | 1.62 | 0.42 | 0.67 | 0.64 | 0.57 |
| | 2 | 1.94 | 1.21 | 0.95 | 1.31 | 1.30 | 2.20 | 0.80 | 1.20 | 0.60 | 1.21 | 0.28 | 0.59 |
| | 6 | 1.25 | 1.53 | 0.85 | 1.18 | 0.90 | 1.33 | 0.85 | 1.14 | 0.86 | 0.92 | 2.28 | 1.22 |
| | 12 | 1.56 | 1.52 | • | 1.54 | 0.98 | 1.49 | • | 1.16 | 0.54 | 0.99 | • | 0.73 |
| C3D1 | 0 | • | 2.88 | • | 2.88 | • | 1.85 | • | 1.85 | • | 0.72 | • | 0.72 |

Legend: “•”: sample not available for analysis.

The percent inhibition of CGS-21680 effect on LPS-induced analytes at C1D1 is tabulated by subject, time point and analyte, and summarized by dose group using the arithmetic mean (Mean) in Table 18. Time profiles are shown in FIG. 16.

TABLE 18

| Time | ENA-78 | | | | IL-8 | | | | TNF-alpha | | | |
|---|---|---|---|---|---|---|---|---|---|---|---|---|
| point | 01-001 | 02-002 | 03-003 | Mean | 01-001 | 02-002 | 03-003 | Mean | 01-001 | 02-002 | 03-003 | Mean |
| 0 | 0 | 0 | 0 | 0 | 0 | 0 | 0 | 0 | 0 | 0 | • | 0 |
| 2 | 43 | 92 | • | 67 | 76 | 92 | • | 84 | 85 | 54 | • | 70 |
| 6 | • | • | 99 | 99 | • | • | 98 | 98 | • | • | 54 | 54 |
| 12 | 69 | 74 | • | 72 | 89 | −10 | • | 40 | 76 | 34 | • | 55 |
| 24 | • | 77 | • | 77 | • | 66 | • | 66 | • | 25 | • | 25 |

Legend: “•”: sample not available for analysis.

Cohort 2: PD2-cytokine assay showed A_{2A}R engagement by Compound 8a in 2/2 evaluable subjects dosed with 40 mg QD Compound 8a:
Compound 8a rescued ENA-78, IL-8 and TNF-alpha secretion in LPS-induced peripheral blood in the presence of the A_{2A}R agonist CGS-21680
PD activity of Compound 8a was observed after first drug exposure (i.e. C1D1) and at steady state (i.e. C1D15 and C2D1)
Compound 8a effect declined 24 hours after drug intake (C1D1 data)

Cohort 2 summary data (pCREB_{CGS} nMeFI) for each cohort are tabulated by subject and summarized by dose group using the geometric mean (Geomean) in Table 19. Summary time profiles are shown in FIG. 17.

TABLE 19

| | | ENA-78 | | | | IL-8 | | | | TNF-alpha | | | |
|---|---|---|---|---|---|---|---|---|---|---|---|---|---|
| Visit | Time | 01-003 | 02-003 | 03-002 | Geomean | 01-003 | 02-003 | 03-002 | Geomean | 01-003 | 02-003 | 03-002 | Geomean |
| SCR | • | 6.00 | 2.17 | • | 3.61 | • | 2.07 | • | 2.07 | 0.31 | 0.61 | • | 0.44 |
| C1D1 | 0 | 4.25 | 8.60 | • | 6.05 | • | 2.19 | • | 2.19 | 0.54 | 0.49 | • | 0.51 |
| | 2 | 1.07 | 1.03 | • | 1.05 | • | 0.73 | • | 0.73 | 0.76 | 0.79 | • | 0.78 |
| | 6 | 1.33 | 1.00 | • | 1.15 | • | 0.93 | • | 0.93 | 1.08 | 0.99 | • | 1.03 |
| | 12 | 1.75 | 1.13 | • | 1.41 | • | 0.82 | • | 0.82 | 0.66 | 0.79 | • | 0.72 |
| | 24 | 2.68 | 5.85 | • | 3.96 | • | 1.80 | • | 1.80 | 0.75 | 0.69 | • | 0.72 |
| C1D15 | 0 | 1.61 | 4.39 | • | 2.66 | • | 1.31 | • | 1.31 | 1.44 | 0.77 | • | 1.05 |
| | 2 | 1.76 | 1.26 | • | 1.49 | • | 0.96 | • | 0.96 | 0.87 | 0.90 | • | 0.89 |
| | 6 | • | 1.72 | • | 1.72 | • | 1.28 | • | 1.28 | • | 1.08 | • | 1.08 |
| | 12 | • | 1.66 | • | 1.66 | • | 1.21 | • | 1.21 | • | 0.79 | • | 0.79 |
| C2D1 | 0 | 1.58 | 1.42 | • | 1.50 | • | 1.14 | • | 1.14 | 0.70 | 0.79 | • | 0.74 |
| | 2 | • | 0.70 | • | 0.70 | • | 0.82 | • | 0.82 | • | 0.82 | • | 0.82 |
| | 6 | 0.48 | 2.29 | • | 1.05 | • | 0.82 | • | 0.82 | 0.58 | 0.94 | • | 0.74 |
| | 12 | 1.20 | 3.37 | • | 2.01 | • | 1.37 | • | 1.37 | 0.65 | 1.03 | • | 0.82 |
| C3D1 | 0 | • | • | • | • | • | • | • | • | • | • | • | • |

Legend: “•”: sample not available for analysis.

The percent inhibition of CGS-21680 effect on LPS-induced analytes at C1D1 is tabulated by subject, time point and analyte, and summarized by dose group using the arithmetic mean (Mean) in Table 20. Time profiles are shown in FIG. 18.

TABLE 20

| Time | ENA-78 | | | | IL-8 | | | | TNF-alpha | | | |
|---|---|---|---|---|---|---|---|---|---|---|---|---|
| point | 01-003 | 02-003 | 03-002 | Mean | 01-003 | 02-003 | 03-002 | Mean | 01-003 | 02-003 | 03-002 | Mean |
| 0 | 0 | 0 | • | 0 | • | 0 | • | 0 | 0 | 0 | • | 0 |
| 2 | 98 | 100 | • | 99 | • | 123 | • | 123 | 48 | 59 | • | 53 |
| 6 | 90 | 100 | • | 95 | • | 106 | • | 106 | 117 | 98 | • | 108 |
| 12 | 77 | 98 | • | 88 | • | 115 | • | 115 | 26 | 59 | • | 42 |
| 24 | 48 | 36 | • | 42 | • | 33 | • | 33 | 46 | 39 | • | 42 |

Legend: “•”: sample not available for analysis.

Cohort 3: PD2-cytokine assay showed A_{2A}R engagement by Compound 8a in 3/3 evaluable subjects dosed with 40 mg BID Compound 8a:
Compound 8a rescued ENA-78, IL-8 and TNF-alpha secretion in LPS-induced peripheral blood in the presence of the A_{2A}R agonist CGS-21680
PD activity of Compound 8a was observed after first drug exposure (i.e. C1D1) and at steady state (i.e. C1D15 and C2D1)
With BID dosing, almost full inhibition of CGS-21680 effect was observed at 24 hours post-dose (C1D1 data)

Cohort 3 summary data (pCREB_{CGS} nMeFI) for each cohort are tabulated by subject and summarized by dose group using the geometric mean (Geomean) in Table 21. Summary time profiles are shown in FIG. 19.

TABLE 21

| | | ENA-78 | | | | IL-8 | | | | TNF-alpha | | | |
|---|---|---|---|---|---|---|---|---|---|---|---|---|---|
| Visit | Time | 01-005 | 02-004 | 03-003 | Geomean | 01-005 | 02-004 | 03-003 | Geomean | 01-005 | 02-004 | 03-003 | Geomean |
| SCR | • | 8.35 | 8.15 | 2.58 | 5.60 | 1.68 | 1.50 | • | 1.59 | • | 0.22 | • | 0.22 |
| C1D1 | 0 | 3.17 | 13.48 | 3.47 | 5.29 | 1.70 | 2.19 | • | 1.93 | • | 0.29 | • | 0.29 |
| | 2 | • | 1.39 | 1.57 | 1.48 | • | 0.93 | • | 0.93 | • | 0.88 | • | 0.88 |
| | 6 | • | 2.11 | 0.74 | 1.25 | • | 1.39 | • | 1.39 | • | 1.12 | • | 1.12 |
| | 12 | 2.40 | 6.29 | 2.26 | 3.24 | 1.73 | 1.67 | • | 1.70 | • | 0.84 | • | 0.84 |
| | 24 | 1.58 | 2.50 | 1.20 | 1.68 | 0.91 | 1.09 | • | 1.00 | • | 0.80 | • | 0.80 |
| C1D15 | 0 | 3.33 | 1.29 | 2.70 | 2.26 | 2.03 | 0.82 | • | 1.29 | • | 0.86 | • | 0.86 |
| | 2 | 0.80 | 1.25 | 0.44 | 0.76 | 0.97 | 0.86 | • | 0.91 | • | 1.03 | • | 1.03 |
| | 6 | 1.70 | 1.49 | 2.19 | 1.77 | 1.11 | 1.08 | • | 1.09 | • | 1.02 | • | 1.02 |
| | 12 | 2.00 | 1.34 | 4.91 | 2.36 | 1.56 | 1.23 | • | 1.39 | • | 0.85 | • | 0.85 |
| C2D1 | 0 | 3.82 | 0.95 | 1.42 | 1.73 | 1.08 | 0.74 | • | 0.89 | • | 0.51 | • | 0.51 |
| | 2 | 1.70 | 1.57 | 1.92 | 1.72 | 0.80 | 0.88 | • | 0.84 | • | 0.73 | • | 0.73 |
| | 6 | 1.31 | 2.12 | 3.41 | 2.12 | 0.89 | 1.34 | • | 1.09 | • | 0.82 | • | 0.82 |
| | 12 | 3.14 | 2.07 | 1.94 | 2.33 | 1.42 | 1.60 | • | 1.51 | • | 0.84 | • | 0.84 |
| C3D1 | 0 | • | 3.57 | 1.95 | 2.64 | • | 1.00 | • | 1.00 | • | 0.79 | • | 0.79 |

Legend: “•”: sample not available for analysis.

The percent inhibition of CGS-21680 effect on LPS-induced analytes at C1D1 is tabulated by subject, time point and analyte, and summarized by dose group using the arithmetic mean (Mean) in Table 22. Time profiles are shown in FIG. 20.

TABLE 22

| Time | ENA-78 | | | | IL-8 | | | | TNF-alpha | | | |
|---|---|---|---|---|---|---|---|---|---|---|---|---|
| point | 01-005 | 02-004 | 03-003 | Mean | 01-005 | 02-004 | 03-003 | Mean | 01-005 | 02-004 | 03-003 | Mean |
| 0 | 0 | 0 | 0 | 0 | 0 | 0 | • | 0 | • | 0 | • | 0 |
| 2 | • | 97 | 77 | 87 | • | 106 | • | 106 | • | 83 | • | 83 |
| 6 | • | 91 | 111 | 101 | • | 67 | • | 67 | • | 117 | • | 117 |
| 12 | 35 | 58 | 49 | 47 | −4 | 44 | • | 20 | • | 77 | • | 77 |
| 24 | 73 | 88 | 92 | 84 | 113 | 92 | • | 103 | • | 72 | • | 72 |

Legend: “•”: sample not available for analysis.

Cohort 4: Although with some variability among individuals, PD2-cytokine assay showed A_{2A}R engagement by Compound 8a in all evaluable subjects dosed with 80 mg BID Compound 8a:
Compound 8a rescued ENA-78, IL-8 and TNF-alpha secretion in LPS-induced peripheral blood in the presence of the A_{2A}R agonist CGS-21680
PD activity of Compound 8a was observed after first drug exposure (i.e. C1D1) and was sustained at steady state (i.e. C1D15 and C2D1)
Almost full inhibition of CGS-21680 effect was observed at 24 hours post-dose (C1D1 data)

Cohort 4 summary data (pCREB_{CGS} nMeFI) for each cohort are tabulated by subject and summarized by dose group using the geometric mean (Geomean) in Table 23. Summary time profiles are shown in FIG. 21.

TABLE 23

| | | ENA-78 | | | | | IL-8 | | |
|---|---|---|---|---|---|---|---|---|---|
| Visit | Time | 02-005 | 02-006 | 03-004 | 01-006 | Geomean | 02-005 | 02-006 | 03-004 |
| SCR | • | 2.50 | 10.85 | • | • | 5.21 | 1.47 | 1.82 | • |
| C1D1 | 0 | 3.24 | 9.79 | 4.39 | • | 5.18 | 1.45 | 2.82 | 1.93 |
| | 2 | 0.72 | 1.03 | 0.85 | • | 0.86 | 0.92 | 0.86 | 0.86 |
| | 6 | 1.26 | • | 1.75 | • | 1.48 | 1.16 | • | 1.27 |
| | 12 | 2.10 | 1.19 | 1.52 | • | 1.56 | 1.71 | 0.77 | 1.39 |
| | 24 | 1.20 | 2.41 | 0.83 | • | 1.34 | 1.16 | 1.69 | 0.83 |
| C1D15 | 0 | 1.80 | 1.04 | 2.96 | • | 1.77 | 1.25 | 0.89 | 1.87 |
| | 2 | 0.92 | 0.51 | 2.54 | • | 1.06 | 0.95 | 0.77 | 1.51 |
| | 6 | 1.91 | 0.79 | 1.82 | • | 1.40 | 1.46 | 0.88 | 1.01 |
| | 12 | 2.36 | 1.95 | 2.07 | • | 2.12 | 1.65 | 0.94 | 1.14 |
| C2D1 | 0 | 1.33 | 1.86 | 1.96 | • | 1.69 | 1.13 | 1.73 | 1.53 |
| | 2 | 0.97 | 1.59 | 2.27 | • | 1.52 | 0.99 | 1.35 | 1.74 |
| | 6 | 1.17 | 2.76 | 0.94 | • | 1.45 | 1.09 | 1.20 | 0.73 |
| | 12 | 2.31 | 0.39 | 1.43 | • | 1.09 | 1.14 | 0.53 | 1.06 |
| C3D1 | 0 | • | • | • | • | • | • | • | • |

| | | IL-8 | | TNF-alpha | | | | |
|---|---|---|---|---|---|---|---|---|
| Visit | Time | 01-006 | Geomean | 02-005 | 02-006 | 03-004 | 01-006 | Geomean |
| SCR | • | • | 1.64 | 0.30 | 0.34 | • | • | 0.32 |
| C1D1 | 0 | • | 1.99 | 0.31 | 0.27 | 0.12 | • | 0.22 |
| | 2 | • | 0.88 | 0.82 | 0.77 | 0.47 | • | 0.67 |
| | 6 | • | 1.21 | 0.92 | • | 1.65 | • | 1.2 |
| | 12 | • | 1.22 | 0.88 | 1.05 | 1.00 | • | 0.97 |
| | 24 | • | 1.18 | 0.65 | 1.21 | 1.41 | • | 1 |
| C1D15 | 0 | • | 1.28 | 0.67 | 1.08 | 0.19 | • | 0.52 |
| | 2 | • | 1.03 | 0.96 | 0.85 | 0.51 | • | 0.75 |
| | 6 | • | 1.09 | 0.86 | 0.80 | 0.72 | • | 0.79 |
| | 12 | • | 1.21 | 0.53 | 0.75 | 0.86 | • | 0.7 |
| C2D1 | 0 | • | 1.44 | 0.79 | 0.89 | 0.89 | • | 0.86 |
| | 2 | • | 1.32 | 0.78 | 1.05 | 0.53 | • | 0.76 |
| | 6 | • | 0.99 | 2.17 | 0.65 | 0.53 | • | 0.91 |
| | 12 | • | 0.86 | 0.80 | 0.78 | 0.40 | • | 0.63 |
| C3D1 | 0 | • | • | • | • | • | • | • |

Legend: “•”: sample not available for analysis.

The percent inhibition of CGS-21680 effect on LPS-induced analytes at C1D1 is tabulated by subject, time point and analyte, and summarized by dose group using the arithmetic mean (Mean) in Table 24. Time profiles are shown in FIG. 22.

Cohort 5: Although data for cohort 5 are preliminary, PD2-cytokine assay showed A_{2A}R engagement by Compound 8a in 3/3 subjects dosed with 160 mg BID Compound 8a:
Compound 8a rescued ENA-78, IL-8 and TNF-alpha secretion in LPS-induced peripheral blood in the presence of the A_{2A}R agonist CGS-21680 at C1D1
PD activity of Compound 8a was observed after first drug exposure (i.e. C1D1) and seemed to be sustained at steady state (i.e. C1D15, n=1 for the moment)
Almost full inhibition of CGS-21680 effect was observed at 24 hours post-dose (C1D1 data)

Cohort 5 summary data (pCREB_{CGS} nMeFI) for each cohort are tabulated by subject and summarized by dose group using the geometric mean (Geomean) in Table 25. Summary time profiles are shown in FIG. 23.

TABLE 25

| | | ENA-78 | | | | IL-8 | | | | TNF-alpha | | | |
|---|---|---|---|---|---|---|---|---|---|---|---|---|---|
| Visit | Time | 02-007 | 01-007 | 01-008 | Geomean | 02-007 | 01-007 | 01-008 | Geomean | 02-007 | 01-007 | 01-008 | Geomean |
| SCR | • | 6.41 | 6.04 | 7.76 | 6.70 | 1.82 | 2.50 | 2.80 | 2.34 | 0.31 | 0.38 | 0.22 | 0.30 |
| C1D1 | 0 | 13.33 | 31.15 | 7.50 | 14.60 | 3.10 | 2.94 | 1.99 | 2.63 | 0.31 | 0.40 | 0.21 | 0.30 |
| | 2 | 1.94 | 0.58 | 3.08 | 1.51 | 1.37 | 0.52 | 1.45 | 1.01 | 0.91 | 0.68 | 0.46 | 0.66 |
| | 6 | 1.77 | 3.87 | 2.05 | 2.41 | 1.11 | 2.07 | 1.20 | 1.40 | 0.83 | 1.02 | 1.27 | 1.02 |
| | 12 | 1.75 | 3.91 | • | 2.62 | 1.03 | 2.09 | • | 1.47 | 0.70 | 0.84 | • | 0.77 |
| | 24 | 2.73 | 2.82 | 1.19 | 2.09 | 1.18 | 1.78 | 0.91 | 1.24 | 0.69 | 1.05 | 0.78 | 0.83 |
| C1D15 | 0 | 1.02 | • | • | • | 0.72 | • | • | • | 1.13 | • | • | • |
| | 2 | 0.93 | • | • | • | 0.92 | • | • | • | 0.91 | • | • | • |
| | 6 | 1.21 | • | • | • | 1.33 | • | • | • | 1.04 | • | • | • |
| | 12 | 1.88 | • | • | • | 1.13 | • | • | • | 0.68 | • | • | • |
| C2D1 | 0 | • | • | • | • | • | • | • | • | • | • | • | • |
| | 2 | • | • | • | • | • | • | • | • | • | • | • | • |
| | 6 | • | • | • | • | • | • | • | • | • | • | • | • |
| | 12 | • | • | • | • | • | • | • | • | • | • | • | • |
| C3D1 | 0 | • | • | • | • | • | • | • | • | • | • | • | • |

Legend: “•”: sample not available for analysis.

The percent inhibition of CGS-21680 effect on LPS-induced analytes at C1D1 is tabulated by subject, time point and analyte, and summarized by dose group using the arithmetic mean (Mean) in Table 26. Time profiles are shown in FIG. 24.

TABLE 26

| Time | ENA-78 | | | | IL-8 | | | | TNF-alpha | | | |
|---|---|---|---|---|---|---|---|---|---|---|---|---|
| point | 02-007 | 01-007 | 01-008 | Mean | 02-007 | 01-007 | 01-008 | Mean | 02-007 | 01-007 | 01-008 | Mean |
| 0 | 0 | 0 | 0 | 0 | 0 | 0 | 0 | 0 | 0 | 0 | 0 | 0 |
| 2 | 92 | 101 | 68 | 87 | 82 | 125 | 55 | 87 | 87 | 47 | 32 | 55 |
| 6 | 94 | 90 | 84 | 89 | 95 | 45 | 80 | 73 | 75 | 103 | 134 | 104 |
| 12 | 94 | 90 | • | 92 | 99 | 44 | • | 71 | 57 | 73 | • | 65 |
| 24 | 86 | 94 | 97 | 92 | 91 | 60 | 109 | 87 | 55 | 108 | 72 | 79 |

Legend: “•”: sample not available for analysis.

CGS-induced changes of analyte secretion are shown in FIG. 25.

2.16 Detailed Analysis of Bioanalysis and Pharmacokinetics for Compound 8a and Compound 77

### LIST OF ABBREVIATIONS

AUC Area under the concentration versus time curve

BID Bis in die (twice daily)

C1D1 Cycle 1 Day 1

C1D15 Cycle 1 Day 15

C2D1 Cycle 2 Day 1

C3D1 Cycle 3 Day 1

CV Coefficient of variation

Geomean Geometric mean

h hour

LLOQ Lower limit of quantification

min minute

PD Pharmacodynamic

PK Pharmacokinetic

QD Quaque die (once a day)

The plasma bioanalytical and subsequent pharmacokinetic assessment of Compound 8a and its circulating metabolite Compound 77 were performed.

2.16.1 Bioanalytical Quantitation

The bionalysis was performed using the fully validated method at Eurofins Bioanalytical Services (Vergeze, France) with the title “Validation of a LC-MS/MS analytical method for Compound 8a and its metabolite Compound 77 in human plasma” (validation report number Eurofins 18-107). In brief, plasma samples prepared from the collected blood samples were analyzed for both Compound 8a (parent molecule) as well as Compound 77 (active metabolite) using solid phase extraction for sample cleanup followed by LC-MS/MS analysis. The method included the use of stable label internal standards Compound 8a-D (=deuterated Compound 8a) and Compound 77-D (=deuterated Compound 77).

Dose proportional increases in exposure were observed through the 80 mg BID dose. At the highest dose (160 mg BID), the exposure did not further increase. Study showed good dose-proportionality through 80 mg BID.

2.16.2 Determination of Pharmacokinetic Parameters

The determination of all PK parameters was performed by Eurofins Bioanalytical Services (Vergeze, France) using a noncompartmental approach with Phoenix WinNon Software after internal quality control together with the bioanalytical data.

The following PK parameters are deemed essential for this report:

| | |
|---|---|
| AUCt | AUC from zero up to the last concentration (above LLOQ) |
| AUC12 | AUC over the 12-hour dosing interval for BID dosing |
| AUC24 | AUC over the 24-hour dosing interval for QD dosing |
| Cₘₐₓ | Maximum plasma concentration |
| Tₘₐₓ | Time to reach Cₘₐₓ |
| Cₘᵢₙ | Plasma concentration at trough for BID dosing (before the next |
| (12 h) | dose) |
| Cₘᵢₙ | Plasma concentration at trough for QD dosing (before the next |
| (24 h) | dose) |
| T_{1/2} | Plasma concentration half-life |
| M/P- | Metabolite-to-Parent ratio of AUCs (Compound 77/Compound |
| Ratio | 8a) |

Data Presentation

The pharmacokinetic summary tables list the metabolite-to-parent (M/P) ratios calculated using respective AUCs Importantly, the plasma concentrations versus time curves for metabolite Compound 77 and parent Compound 8a were quasi-parallel which indicates “formation rate-limited PK” for metabolite Compound 77 (i.e. metabolite is not accumulating but minors the plasma-concentration versus time profile of the parent). Therefore, it is sufficient for this report to provide the M/P-ratios because the PK data for the metabolite Compound 77 are similar as the data shown for parent with the only difference being reflected in the M/P-ratio which was M/P ˜0.7, i.e. plasma concentrations of Compound 77 at any given time point were always approximately 70% compared to those of Compound 8a (see FIG. 26 for a representative example of Cohort 4).

COHORT 1—20 mg QD

Cohort 1: Individual and Mean Plasma Concentrations for Compound 8a are shown in table 27 and 28.

Compound 8a CYCLE 1—Cohort 1—Plasma Concentrations (ng/mL)

TABLE 27

| | | Subject | Subject | Subject | |
|---|---|---|---|---|---|
| Day | Hour | 01001 | 02002 | 03001 | Mean |
| 1 | 0 | BLQ | BLQ | BLQ | BLQ |
| 1 | 0.25 | BLQ | 2.0 | BLQ | 0.7 |
| 1 | 0.5 | 8.6 | 2.3 | 15.2 | 8.7 |
| 1 | 1 | 107.7 | 1.7 | 22.1 | 43.9 |
| 1 | 2 | 97.8 | 1.0 | 16.1 | 38.3 |
| 1 | 4 | 20.1 | 0.3 | 7.6 | 9.4 |
| 1 | 6 | 8.1 | 0.2 | 2.9 | 3.7 |
| 1 | 8 | 2.5 | BLQ | 1.2 | 1.2 |
| 1 | 12 | 0.8 | 0.3 | 0.3 | 0.4 |
| 1 | 24 | 0.4 | 1.1 | 0.5 | 0.7 |
| 8 | 0 | 0.3 | 0.9 | 0.5 | 0.6 |
| 15 | 0 | 0.3 | 0.6 | 0.7 | 0.5 |
| 15 | 0.25 | 0.4 | 0.5 | 0.6 | 0.5 |
| 15 | 0.5 | 0.7 | 107.7 | 11.9 | 40.1 |
| 15 | 1 | 33.9 | 121.9 | 48.0 | 67.9 |
| 15 | 2 | 182.9 | 59.7 | 38.1 | 93.6 |
| 15 | 4 | 41.5 | 24.6 | 8.6 | 24.9 |
| 15 | 6 | 11.7 | 9.1 | 3.5 | 8.1 |
| 15 | 8 | 3.2 | 4.9 | 2.1 | 3.4 |
| 15 | 12 | BLQ | 1.6 | 1.0 | 0.9 |

TABLE 28
Compound 8a CYCLE 2 - Cohort 1 -
plasma concentrations (ng/mL)

| | | Subject | Subject | Subject | |
|---|---|---|---|---|---|
| Day | Hour | 01001 | 02002 | 03001 | Mean |
| 1 | 0 | 1.2 | 0.4 | 0.6 | 0.7 |
| 1 | 2 | 112.5 | 46.8 | 18.7 | 59.3 |
| 1 | 6 | 26.8 | 8.2 | 4.4 | 13.1 |
| 1 | 12 | 3.4 | 1.6 | 1.2 | 2.1 |

Mean trough concentrations at steady state (C_{min,ss}) are in bold (note: 24-h dosing interval).

Cohort 1: Individual and Mean Plasma Concentrations for Compound 77 are shown in table 29 and 30.

TABLE 29
Compound 77 CYCLE 1 - Cohort 1 -
plasma concentrations (ng/mL)

| | | Subject | Subject | Subject | |
|---|---|---|---|---|---|
| Day | Hour | 01001 | 02002 | 03001 | Mean |
| 1 | 0 | BLQ | BLQ | BLQ | BLQ |
| 1 | 0.25 | BLQ | 0.6 | BLQ | 0.2 |
| 1 | 0.5 | 1.3 | 1.0 | 5.3 | 2.5 |
| 1 | 1 | 39.0 | 0.8 | 15.2 | 18.3 |
| 1 | 2 | 52.4 | 0.7 | 10.0 | 21.0 |
| 1 | 4 | 15.8 | 0.4 | 5.5 | 7.2 |
| 1 | 6 | 11.5 | 0.3 | 3.5 | 5.1 |
| 1 | 8 | 4.7 | BLQ | 2.2 | 2.3 |
| 1 | 12 | 1.6 | BLQ | 0.4 | 0.7 |
| 1 | 24 | 0.5 | 0.6 | 0.3 | 0.4 |
| 8 | 0 | 0.4 | 1.0 | 0.3 | 0.6 |
| 15 | 0 | 0.3 | 0.8 | 0.4 | 0.5 |
| 15 | 0.25 | 0.3 | 0.6 | 0.4 | 0.4 |
| 15 | 0.5 | 0.3 | 14.2 | 3.1 | 5.9 |
| 15 | 1 | 8.1 | 69.0 | 28.1 | 35.1 |
| 15 | 2 | 84.1 | 42.3 | 29.7 | 52.1 |
| 15 | 4 | 28.8 | 25.8 | 7.6 | 20.7 |
| 15 | 6 | 11.4 | 11.0 | 3.7 | 8.7 |
| 15 | 8 | 4.5 | 7.9 | 2.5 | 5.0 |
| 15 | 12 | 0.4 | 3.7 | 0.9 | 1.7 |

TABLE 30
Compound 77 CYCLE 2 - Cohort 1 -
plasma concentrations (ng/mL)

| | | Subject | Subject | Subject | |
|---|---|---|---|---|---|
| Day | Hour | 01001 | 02002 | 03001 | Mean |
| 1 | 0 | 1.1 | 0.6 | 0.3 | 0.7 |
| 1 | 2 | 60.6 | 35.2 | 10.6 | 35.4 |
| 1 | 6 | 21.8 | 13.1 | 3.5 | 12.8 |
| 1 | 12 | 6.1 | 3.2 | 0.8 | 3.3 |

Mean trough concentrations at steady state (C_{min,ss}) are in bold (note: 24-h dosing interval).

Cohort 1: Summary of Major Pharmacokinetic Data

Table 31 summarizes relevant PK data obtained for Compound 8a. Besides standard PK parameters, this table also includes the minimal concentrations on each day of dosing, i.e. 24-hour trough levels (for Day 1 this was 24 hours after dosing and for Day 15 this was the pre-dose concentration).

TABLE 31
Cohort 1 (20 mg QD): Summary of major Compound 8a PK Parameters

| | | | | | | | M/P- |
|---|---|---|---|---|---|---|---|
| | | | | Cmin | | | Ratio |
| | | Cmax | Tmax | (24 h) | AUC24 | T_{1/2} | (via |
| | Subject | (ng/mL) | (h) | (ng/mL) | (h*ng/mL) | (h) | AUCt) |
| C1D1 | 01001 | 108 | 1.0 | 0.4 | 303 | 3.9 | 0.63 |
| | 02002* | 2.34 | 0.5 | 1.1 | 14.2 | NC | 0.49 |
| | 03001 | 22.1 | 1.0 | 0.5 | 76.1 | 4.4 | 0.77 |
| | Geo | 48.8 | 1.0 | 0.4 | 152 | 4.1 | 0.70 |
| | Mean | | | | | | |
| C1D15 | 01001 | 183 | 2.0 | 0.3 | 410 | 1.1 | 0.55 |
| | 02002 | 122 | 1.0 | 0.6 | 307 | 2.4 | 0.74 |
| | 03001 | 48.0 | 1.0 | 0.7 | 130 | 3.3 | 0.76 |
| | Geo | 102 | 1.3 | 0.5 | 254 | 2.1 | 0.68 |
| | Mean | | | | | | |
| C2D1 | 01001 | 113 | 2.0 | 1.2 | 483 | NC | 0.64 |
| | 02002 | 46.8 | 2.0 | 0.4 | 187 | NC | 0.97 |
| | 03001 | 18.7 | 2.0 | 0.6 | 82.6 | NC | 0.63 |
| | Geo | 46.2 | 2.0 | 0.7 | 195 | | 0.73 |
| | Mean | | | | | | |

*outlier since D 15 data show exposure (not used);
NC: not calculated;
M/P: Metabolite-to-Parent

Compound 8a showed measurable concentrations in all subjects at the lowest dose (starting dose) of 20 mg. The inter-subject variability was acceptable with all subjects showing exposures deviating no more than approximately factor of 2 from the geometric mean (geomean). There was one notable exception, i.e. subject 02002 on C1D1 had very low exposure, however, the data for this subject on the following days of measurement suggested that this was incidental. The metabolite to parent ratio on all days was very stable at geomean M/P ˜0.7 (i.e. metabolite Compound 77 plasma exposure was high with approximately 70% of parent Compound 8a exposure).

COHORT 2—40 mg QD

Cohort 2: Individual and Mean Plasma Concentrations for Compound 8a are showed in Table 32 and 33.

Compound 8a CYCLE 1—Cohort 2—Plasma Concentrations (ng/mL)

TABLE 32

| | | Subject | Subject | Subject | |
|---|---|---|---|---|---|
| Day | Hour | 01003 | 02003 | 03002 | Mean |
| 1 | 0 | BLQ | BLQ | BLQ | BLQ |
| 1 | 0.25 | 101.7 | BLQ | BLQ | 33.9 |
| 1 | 0.5 | 335.8 | 6.9 | 6.8 | 116.5 |
| 1 | 1 | 273.4 | 345.5 | 89.1 | 236.0 |
| 1 | 2 | 149.8 | 125.4 | 79.1 | 118.1 |
| 1 | 4 | 62.2 | 26.6 | 28.8 | 39.2 |
| 1 | 6 | 32.1 | 7.8 | 12.1 | 17.3 |
| 1 | 8 | 14.8 | 2.6 | 4.9 | 7.4 |
| 1 | 12 | 5.5 | 0.7 | 1.3 | 2.5 |
| 1 | 24 | 2.4 | 0.2 | 0.6 | 1.1 |
| 8 | 0 | 13.6 | 0.3 | 2.6 | 5.5 |
| 15 | 0 | 9.9 | 2.8 | 1.4 | 4.7 |
| 15 | 0.25 | 24.1 | 2.6 | 1.4 | 9.4 |
| 15 | 0.5 | 139.2 | 2.7 | 4.9 | 48.9 |
| 15 | 1 | 343.8 | 112.0 | 63.3 | 173.0 |
| 15 | 2 | 364.6 | 135.7 | 195.0 | 231.8 |
| 15 | 4 | 120.0 | 26.8 | 69.2 | 72.0 |
| 15 | 6 | 68.3 | 9.5 | 28.0 | 35.3 |
| 15 | 8 | 33.6 | 4.6 | 12.4 | 16.9 |
| 15 | 12 | 15.6 | 3.3 | 3.6 | 7.5 |

TABLE 33
Compound 8a CYCLE 2 - Cohort 2 -
plasma concentrations (ng/mL)

| | | Subject | Subject | Subject | |
|---|---|---|---|---|---|
| Day | Hour | 01003 | 02003 | 03002 | Mean |
| 1 | 0 | 5.4 | 7.2 | 2.1 | 4.9 |
| 1 | 2 | 299.4 | 164.4 | 128.4 | 197.4 |
| 1 | 6 | 71.5 | 14.6 | 18.1 | 34.7 |
| 1 | 12 | 16.0 | 9.5 | 3.2 | 9.6 |

Mean trough concentrations at steady state (C_{min,ss}) are in bold (note: 24-h dosing interval).

Cohort 2: Individual and Mean Plasma Concentrations for Compound 77 are shown in table 34 and 35.

TABLE 34
Compound 77 CYCLE 1 - Cohort 2 -
plasma concentrations (ng/mL)

| | | Subject | Subject | Subject | |
|---|---|---|---|---|---|
| Day | Hour | 01003 | 02003 | 03002 | Mean |
| 1 | 0 | BLQ | BLQ | BLQ | BLQ |
| 1 | 0.25 | 11.4 | BLQ | BLQ | 3.8 |
| 1 | 0.5 | 72.5 | 1.1 | 0.5 | 24.7 |
| 1 | 1 | 108.3 | 94.2 | 40.0 | 80.8 |
| 1 | 2 | 73.7 | 104.9 | 67.5 | 82.0 |
| 1 | 4 | 38.2 | 41.5 | 38.3 | 39.3 |
| 1 | 6 | 30.8 | 13.6 | 20.4 | 21.6 |
| 1 | 8 | 15.8 | 4.7 | 10.2 | 10.2 |
| 1 | 12 | 9.3 | 1.5 | 3.8 | 4.9 |
| 1 | 24 | 1.6 | BLQ | 0.7 | 0.8 |
| 8 | 0 | 6.9 | 0.2 | 1.6 | 2.9 |
| 15 | 0 | 4.3 | 1.4 | 0.6 | 2.1 |
| 15 | 0.25 | 5.1 | 1.2 | 0.5 | 2.3 |
| 15 | 0.5 | 26.9 | 1.2 | 1.1 | 9.7 |
| 15 | 1 | 108.3 | 21.6 | 14.5 | 48.1 |
| 15 | 2 | 154.3 | 67.9 | 76.7 | 99.7 |
| 15 | 4 | 79.1 | 22.0 | 42.3 | 47.8 |
| 15 | 6 | 53.4 | 9.4 | 23.6 | 28.8 |
| 15 | 8 | 30.4 | 4.2 | 12.3 | 15.6 |
| 15 | 12 | 15.3 | 2.0 | 3.5 | 6.9 |

TABLE 35
Compound 77 CYCLE 2 - Cohort 2 -
plasma concentrations (ng/mL)

| | | Subject | Subject | Subject | |
|---|---|---|---|---|---|
| Day | Hour | 01003 | 02003 | 03002 | Mean |
| 1 | 0 | 3.0 | 3.6 | 1.2 | 2.6 |
| 1 | 2 | 120.3 | 83.3 | 59.2 | 87.6 |
| 1 | 6 | 56.3 | 13.4 | 13.1 | 27.6 |
| 1 | 12 | 19.7 | 5.4 | 2.8 | 9.3 |

Mean trough concentrations at steady state (C_{min, ss}) are in bold (note: 24-h dosing interval).

Cohort 2: Summary of Major Pharmacokinetic Data

Table 36 summarizes relevant PK data obtained for Compound 8a. Besides standard PK parameters, this table also includes the minimal concentrations on each day of dosing, i.e. 24-hour trough levels (for Day 1 this was 24 hours after dosing and for Day 15 this was the pre-dose concentration).

TABLE 36
Cohort 2 (40 mg QD): Summary of
major Compound 8a PK Parameters

| | | | | | | | M/P- |
|---|---|---|---|---|---|---|---|
| | | | | Cmin | | | Ratio |
| | | Cmax | Tmax | (24 h) | AUC24 | T_{1/2} | (via |
| | Subject | (ng/mL) | (h) | (ng/mL) | (h*ng/mL) | (h) | AUCt) |
| C1D1 | 01003 | 336 | 0.5 | 2.4 | 872 | 3.5 | 0.56 |
| | 02003 | 346 | 1.0 | 0.2 | 533 | 4.0 | 0.67 |
| | 03002 | 89.1 | 1.0 | 0.6 | 299 | 3.3 | 1.05 |
| | Geo | 218 | 0.8 | 0.7 | 518 | 3.6 | 0.73 |
| | Mean | | | | | | |
| C1D15 | 01003 | 365 | 2.0 | 9.9 | 1430 | 2.7 | 0.54 |
| | 02003 | 136 | 2.0 | 2.8 | 394 | 2.8 | 0.52 |
| | 03002 | 195 | 2.0 | 1.4 | 592 | 2.1 | 0.52 |
| | Geo | 213 | 2.0 | 3.4 | 694 | 2.5 | 0.53 |
| | Mean | | | | | | |
| C2D1 | 01003 | 299 | 2.0 | 5.4 | 1309 | NC | 0.54 |
| | 02003 | 164 | 2.0 | 7.2 | 602 | NC | 0.56 |
| | 03002 | 128 | 2.0 | 2.1 | 488 | NC | 0.52 |
| | Geo | 184 | 2.0 | 4.4 | 727 | | 0.54 |
| | Mean | | | | | | |

NC: not calculated;
M/P: Metabolite-to-Parent

Compound 8a showed measurable concentrations in all subjects at the dose of 40 mg QD. The inter-subject variability was acceptable with all subjects showing exposures deviating no more than approximately factor of 2 from the geometric mean (geomean). The metabolite to parent ratio on all days was stable at geomean M/P ˜0.5-0.7 (i.e. metabolite Compound 77 plasma exposure was high with approximately 50-70% of parent Compound 8a exposure).

COHORT 3—40 mg BID

Cohort 3: Individual and Mean Plasma Concentrations for Compound 8a are shown table 37 and 38.

TABLE 37
Compound 8a CYCLE 1 - Cohort 3 -
plasma concentrations (ng/mL)

| | | Subject | Subject | Subject | |
|---|---|---|---|---|---|
| Day | Hour | 01005 | 02004 | 03003 | Mean |
| 1 | 0 | BLQ | BLQ | BLQ | BLQ |
| 1 | 0.25 | 21.4 | 2.4 | BLQ | 7.9 |
| 1 | 0.5 | 87.1 | 69.2 | 23.1 | 59.8 |
| 1 | 1 | 112.5 | 90.5 | 315.7 | 172.9 |
| 1 | 2 | 78.1 | 46.4 | 93.6 | 72.7 |
| 1 | 4 | 20.6 | 15.8 | 18.5 | 18.3 |
| 1 | 6 | 6.9 | 4.7 | 6.4 | 6.0 |
| 1 | 8 | 3.0 | 1.8 | 2.7 | 2.5 |
| 1 | 12 | 0.7 | 0.4 | 0.8 | 0.6 |
| 1 | 24 | 2.2 | 1.9 | 3.7 | 2.6 |
| 8 | 0 | 16.0 | 13.9 | 2.3 | 10.7 |
| 15 | 0 | 6.5 | 18.6 | 1.9 | 9.0 |
| 15 | 0.25 | 34.8 | 19.2 | 1.5 | 18.5 |
| 15 | 0.5 | 196.9 | 17.4 | 33.9 | 82.7 |
| 15 | 1 | 241.2 | 20.2 | 178.9 | 146.8 |
| 15 | 2 | 196.2 | 10.2 | 104.6 | 103.6 |
| 15 | 4 | 62.2 | 6.4 | 34.8 | 34.5 |
| 15 | 6 | 25.0 | 5.0 | 11.4 | 13.8 |
| 15 | 8 | 11.3 | 3.2 | 5.3 | 6.6 |
| 15 | 12 | 4.0 | 3.0 | 2.4 | 3.2 |

TABLE 38
Compound 8a CYCLE 2 - Cohort 3 -
plasma concentrations (ng/mL)

| | | Subject | Subject | Subject | |
|---|---|---|---|---|---|
| Day | Hour | 01005 | 02004 | 03003 | Mean |
| 1 | 0 | 12.3 | 6.2 | 4.0 | 7.5 |
| 1 | 2 | 258.2 | 6.7 | 148.7 | 137.9 |
| 1 | 6 | 39.2 | 2.5 | 5.7 | 15.8 |
| 1 | 12 | 6.1 | 1.1 | 3.0 | 3.4 |

Mean trough concentrations at steady state (C_{min,ss}) are in bold (note: 12-h dosing interval).

Cohort 3: Individual and Mean Plasma Concentrations for Compound 77 are shown in table 39 and 40.

TABLE 39
Compound 77 CYCLE 1 - Cohort 3 -
plasma concentrations (ng/mL)

| | | Subject | Subject | Subject | |
|---|---|---|---|---|---|
| Day | Hour | 01005 | 02004 | 03003 | Mean |
| 1 | 0 | BLQ | BLQ | BLQ | BLQ |
| 1 | 0.25 | 2.5 | 0.2 | BLQ | 0.9 |
| 1 | 0.5 | 27.5 | 14.5 | 2.5 | 14.8 |
| 1 | 1 | 63.1 | 64.5 | 128.6 | 85.4 |
| 1 | 2 | 53.9 | 31.7 | 54.7 | 46.8 |
| 1 | 4 | 24.4 | 13.0 | 13.2 | 16.9 |
| 1 | 6 | 10.1 | 5.8 | 6.9 | 7.6 |
| 1 | 8 | 6.9 | 2.6 | 4.1 | 4.5 |
| 1 | 12 | 2.0 | 0.8 | 0.7 | 1.2 |
| 1 | 24 | 5.1 | 2.2 | 2.7 | 3.3 |
| 8 | 0 | 15.0 | 11.0 | 2.2 | 9.4 |
| 15 | 0 | 11.7 | 17.6 | 2.9 | 10.7 |
| 15 | 0.25 | 12.8 | 15.4 | 2.7 | 10.3 |
| 15 | 0.5 | 45.7 | 16.2 | 9.7 | 23.9 |
| 15 | 1 | 135.0 | 15.6 | 128.5 | 93.0 |
| 15 | 2 | 136.7 | 8.9 | 100.9 | 82.1 |
| 15 | 4 | 58.8 | 7.0 | 35.2 | 33.7 |
| 15 | 6 | 36.7 | 5.9 | 18.1 | 20.3 |
| 15 | 8 | 20.5 | 3.0 | 8.7 | 10.7 |
| 15 | 12 | 6.9 | 2.5 | 3.5 | 4.3 |

TABLE 40
Compound 77 CYCLE 2 - Cohort 3 -
plasma concentrations (ng/mL)

| | | Subject | Subject | Subject | |
|---|---|---|---|---|---|
| Day | Hour | 01005 | 02004 | 03003 | Mean |
| 1 | 0 | 23.7 | 5.5 | 2.4 | 10.5 |
| 1 | 2 | 183.9 | 5.1 | 62.3 | 83.8 |
| 1 | 6 | 53.7 | 1.7 | 4.9 | 20.1 |
| 1 | 12 | 10.3 | 0.8 | 1.7 | 4.3 |

Mean trough concentrations at steady state (C_{min,ss}) are in bold (note: 12-h dosing interval).

Cohort 3: Summary of Major Pharmacokinetic Data

Table 41 summarizes relevant PK data obtained for Compound 8a. Besides standard PK parameters, this table includes minimal concentrations on each day of dosing, i.e. the 12-hour trough levels.

TABLE 41
Cohort 3 (40 mg BID): Summary of major Compound 8a PK Parameters

| | | | | | | | M/P- |
|---|---|---|---|---|---|---|---|
| | | | | Cmin | | | Ratio |
| | | Cmax | Tmax | (12 h) | AUC12 | T_{1/2} | (via |
| | Subject | (ng/mL) | (h) | (ng/mL) | (h*ng/mL) | (h) | AUCt) |
| C1D1 | 01005 | 113 | 1.0 | 0.7 | 305 | 1.7 | 0.76 |
| | 02004 | 90.5 | 1.0 | 0.4 | 211 | 1.6 | 0.70 |
| | 03003 | 316 | 1.0 | 0.8 | 445 | 1.8 | 0.52 |
| | Geo | 148 | 1.0 | 0.6 | 306 | 1.7 | 0.66 |
| | Mean | | | | | | |
| C1D15 | 01005 | 241 | 1.0 | 4.0 | 775 | 2.1 | 0.77 |
| | 02004 | 20.2 | 1.0 | 3.0 | 82.3 | 5.6 | 0.93 |
| | 03003 | 179 | 1.0 | 2.4 | 418 | 2.8 | 0.94 |
| | Geo | 95.6 | 1.0 | 3.1 | 299 | 3.2 | 0.88 |
| | Mean | | | | | | |
| C2D1 | 01005 | 258 | 2.0 | 6.1 | 1001 | NC | 0.87 |
| | 02004 | 6.68 | 2.0 | 1.1 | 41.9 | NC | 0.76 |
| | 03003 | 149 | 2.0 | 3.0 | 488 | NC | 0.45 |
| | Geo | 63.5 | 2.0 | 2.7 | 274 | | 0.67 |
| | Mean | | | | | | |

NC: not calculated;
M/P: Metabolite-to-Parent

Compound 8a showed measurable concentrations in all subjects at the dose of 40 mg BID. The inter-subject variability was acceptable on C1D1 with all subjects showing exposures deviating no more than approximately factor of 2 from the geomean. The inter-subject variability was higher on C1D15 and C2D1 (within factor of ˜3 from geomean), however, this was due to one subject showing comparatively low exposures (subject 02004). The metabolite to parent ratio on all days was stable at geomean M/P ˜0.7-0.9 (i.e. metabolite Compound 77 plasma exposure was high with approximately 70-90% of parent Compound 8a exposure).

Cohort 4: Individual and Mean Plasma Concentrations for Compound 8a are shown in table 42 and 43.

TABLE 42
Compound 8a CYCLE 1 - Cohort 4 -
plasma concentrations (ng/mL)

| | | Subject | Subject | Subject | Subject | |
|---|---|---|---|---|---|---|
| Day | Hour | 01006 | 02005 | 02006 | 03004 | Mean |
| 1 | 0 | BLQ | BLQ | BLQ | BLQ | BLQ |
| 1 | 0.25 | 52.8 | 1.5 | 59.1 | 6.3 | 29.9 |
| 1 | 0.5 | 425.2 | 36.3 | 436.2 | 221.4 | 279.8 |
| 1 | 1 | 456.6 | 46.5 | 280.8 | 216.7 | 250.2 |
| 1 | 2 | 137.9 | 516.1 | 113.0 | 95.3 | 215.6 |
| 1 | 4 | 34.1 | 199.1 | 53.9 | 25.2 | 78.1 |
| 1 | 6 | 12.4 | 58.1 | 25.1 | 11.1 | 26.7 |
| 1 | 8 | 4.6 | 21.2 | 11.5 | 5.6 | 10.7 |
| 1 | 12 | 0.9 | 4.6 | 3.7 | 3.4 | 3.2 |
| 1 | 24 | 4.5 | 6.5 | 21.9 | 14.8 | 11.9 |
| 8 | 0 | 7.6 | 4.0 | 15.2 | 8.7 | 8.9 |
| 15 | 0 | 4.7 | 4.7 | 19.9 | 6.4 | 8.9 |
| 15 | 0.25 | 148.3 | 10.6 | 22.0 | 18.3 | 49.8 |
| 15 | 0.5 | 391.7 | 66.6 | 69.6 | 27.2 | 138.8 |
| 15 | 1 | AQL | 289.1 | 266.9 | 23.1 | 193.1 |
| 15 | 2 | 207.7 | 109.4 | 105.9 | 16.9 | 110.0 |
| 15 | 4 | 45.3 | 23.4 | 99.0 | 8.7 | 44.1 |
| 15 | 6 | 17.1 | 7.4 | 40.4 | 7.5 | 18.1 |
| 15 | 8 | 8.3 | 3.5 | 18.9 | 6.5 | 9.3 |
| 15 | 12 | 6.4 | 1.6 | 10.4 | 5.8 | 6.1 |

TABLE 43
Compound 8a CYCLE 2 - Cohort 4 -
plasma concentrations (ng/mL)

| | | Subject | Subject | Subject | Subject | |
|---|---|---|---|---|---|---|
| Day | Hour | 01006 | 02005 | 02006 | 03004 | Mean |
| 1 | 0 | 14.1 | 7.8 | 36.3 | 3.3 | 15.4 |
| 1 | 2 | 565.0 | 212.0 | no sample | 289.2 | 355.4 |
| 1 | 6 | 53.2 | 18.6 | 58.0 | 32.9 | 40.7 |
| 1 | 12 | 12.9 | 4.0 | 15.8 | 27.3 | 15.0 |

Mean trough concentrations at steady state (C_{min,ss}) are in bold (note: 12-h dosing interval).

Cohort 4: Individual and Mean Plasma Concentrations for Compound 77 are shown in table 44 and 45.

TABLE 44
Compound 77 CYCLE 1 - Cohort 4 -
plasma concentrations (ng/mL)

| | | Subject | Subject | Subject | Subject | |
|---|---|---|---|---|---|---|
| Day | Hour | 01006 | 02005 | 02006 | 03004 | Mean |
| 1 | 0 | BLQ | BLQ | BLQ | BLQ | BLQ |
| 1 | 0.25 | 8.0 | BLQ | 6.6 | 0.9 | 3.9 |
| 1 | 0.5 | 127.2 | 9.1 | 92.4 | 82.6 | 77.8 |
| 1 | 1 | 276.8 | 22.6 | 140.0 | 114.8 | 138.6 |
| 1 | 2 | 135.7 | 230.1 | 76.8 | 71.0 | 128.4 |
| 1 | 4 | 41.5 | 159.4 | 57.3 | 25.6 | 71.0 |
| 1 | 6 | 15.2 | 62.2 | 40.3 | 14.9 | 33.2 |
| 1 | 8 | 7.0 | 35.6 | 22.8 | 8.6 | 18.5 |
| 1 | 12 | 2.1 | 8.8 | 9.1 | 3.0 | 5.8 |
| 1 | 24 | 6.4 | 10.6 | 50.8 | 15.3 | 20.8 |
| 8 | 0 | 5.7 | 3.8 | 25.4 | 6.3 | 10.3 |
| 15 | 0 | 3.7 | 5.7 | 31.1 | 5.5 | 11.5 |
| 15 | 0.25 | 31.2 | 6.0 | 34.9 | 8.4 | 20.1 |
| 15 | 0.5 | 130.9 | 24.0 | 37.7 | 18.4 | 52.8 |
| 15 | 1 | ALQ | 130.0 | 84.8 | 16.4 | 77.1 |
| 15 | 2 | 108.5 | 102.3 | 62.6 | 14.2 | 71.9 |
| 15 | 4 | 46.2 | 27.1 | 99.9 | 7.5 | 45.2 |
| 15 | 6 | 17.0 | 13.9 | 49.5 | 5.2 | 21.4 |
| 15 | 8 | 8.4 | 7.9 | 29.2 | 4.7 | 12.5 |
| 15 | 12 | 6.2 | 2.8 | 14.6 | 3.2 | 6.7 |

TABLE 45
Compound 77 CYCLE 2 - Cohort 4 -
plasma concentrations (ng/mL)

| | | Subject | Subject | Subject | Subject | |
|---|---|---|---|---|---|---|
| Day | Hour | 01006 | 02005 | 02006 | 03004 | Mean |
| 1 | 0 | 14.4 | 7.7 | 27.5 | 4.6 | 13.6 |
| 1 | 2 | 502.9 | 162.7 | no sample | 273.6 | 313.1 |
| 1 | 6 | 75.6 | 19.3 | 53.4 | 47.3 | 48.9 |
| 1 | 12 | 14.1 | 3.8 | 17.6 | 10.4 | 11.4 |

Mean trough concentrations at steady state (C_{min,ss}) are in bold (note: 12-h dosing interval).

Cohort 4: Summary of Major Pharmacokinetic Data

Table 46 summarizes relevant PK data obtained for Compound 8a. Besides standard PK parameters, this table includes minimal concentrations on each day of dosing, i.e. the 12-hour trough levels.

TABLE 46
Cohort 4 (80 mg BID): Summary of major Compound 8a PK Parameters

| | | | | | | | M/P- |
|---|---|---|---|---|---|---|---|
| | | | | Cmin | | | Ratio |
| | | Cmax | Tmax | (12 h) | AUC12 | T_{1/2} | (via |
| | Subject | (ng/mL) | (h) | (ng/mL) | (h*ng/mL) | (h) | AUCt) |
| C1D1 | 01006 | 457 | 1.0 | 0.9 | 831 | 1.4 | 0.72 |
| | 02005 | 516 | 2.0 | 4.6 | 1410 | 1.5 | 0.66 |
| | 02006 | 436 | 0.5 | 3.7 | 759 | 2.0 | 0.71 |
| | 03004 | 221 | 0.5 | 3.4 | 486 | 1.8 | 0.69 |
| | Geo Mean | 388 | 0.8 | 2.7 | 811 | 1.7 | 0.70 |
| C1D15* | 01006 | 392 | 0.5 | 6.4 | 906 | 2.1 | 0.53 |
| | 02005 | 289 | 1.0 | 1.6 | 484 | 2.9 | 0.77 |
| | 02006 | 267 | 1.0 | 10 | 749 | 2.7 | 0.80 |
| | 03004 | 27.2 | 0.5 | 5.8 | 122 | 14 | 0.73 |
| | Geo Mean | 169 | 0.7 | 5.0 | 447 | 3.9 | 0.70 |
| C2D1 | 01006 | 565 | 2.0 | 12.9 | 2014 | NC | 0.96 |
| | 02005 | 212 | 2.0 | 4.0 | 749 | NC | 0.81 |
| | 02006 | 58.0 | 6.0 | 15.8 | 504.6 | NC | 0.90 |
| | 03004 | 289 | 2.0 | 27.3 | 1117 | NC | 0.98 |
| | Geo Mean | 212 | 2.6 | 12.2 | 960 | | 0.91 |

*If subject 03004 on D 15 is excluded Cmax and AUC are similar to D 1;
NC: not calculated;
M/P: Metabolite-to-Parent

Compound 8a showed measurable concentrations in all subjects at the dose of 80 mg BID. The inter-subject variability was acceptable on all days with all subjects showing exposures deviating no more than approximately factor of 2 from the geomean with one notable exception, i.e. Subject 03004 on C1D15. If the data for 53004 on C1D15 are excluded, the overall exposures would be similar to C1D1 (12-h AUC would be ˜700 h*ng/mL). Overall the Cohort 4 data indicate that a 24-hour AUC at steady state of ˜1500 to 2000 h*ng/mL is achieved with this dosing regimen. The metabolite to parent ratio on all days was stable indicating that metabolite Compound 77 plasma exposure was high with approximately 70% of parent Compound 8a exposure on Day 1 as well as at steady state on Day 15.

COHORT 5—160 mg BID

Cohort 5: Individual and Mean Plasma Concentrations for Compound 8a are shown in table 47 and 48.

TABLE 47
Compound 8a CYCLE 1 - Cohort 5 -
plasma concentrations (ng/mL)

| | | Subject | Subject | Subject | Subject | |
|---|---|---|---|---|---|---|
| Day | Hour | 01007 | 01008 | 01009 | 02007 | Mean |
| 1 | 0 | BLQ | BLQ | BLQ | BLQ | BLQ |
| 1 | 0.25 | 17.1 | BLQ | BLQ | 0.8 | 4.5 |
| 1 | 0.5 | 38.4 | 0.7 | 23.6 | 132.6 | 48.8 |
| 1 | 1 | 65.6 | 1.6 | 20.6 | 158.6 | 61.6 |
| 1 | 2 | 25.8 | 2.1 | 257.0 | 57.5 | 85.6 |
| 1 | 4 | 4.7 | 6.1 | 116.9 | 17.8 | 36.4 |
| 1 | 6 | 1.6 | 4.1 | no sample | 6.9 | 4.2 |
| 1 | 8 | 0.7 | 3.7 | no sample | 2.7 | 2.4 |
| 1 | 12 | 2.2 | 3.9 | no sample | 0.6 | 2.3 |
| 1 | 24 | 20.9 | 10.5 | 19.1 | 5.2 | 13.9 |
| 8 | 0 | 23.9 | 7.5 | 17.4 | 9.6 | 14.6 |
| 15 | 0 | 12.1 | 10.7 | 41.2 | 11.7 | 18.9 |
| 15 | 0.25 | 14.1 | 8.1 | 33.0 | 32.6 | 21.9 |
| 15 | 0.5 | 52.9 | 8.0 | 92.5 | 232.9 | 96.6 |
| 15 | 1 | 85.0 | 7.8 | 242.3 | 159.9 | 123.7 |
| 15 | 2 | 36.2 | 10.6 | 224.1 | 53.7 | 81.1 |
| 15 | 4 | 21.2 | 8.4 | 90.2 | 15.8 | 33.9 |
| 15 | 6 | 7.2 | 8.1 | 45.3 | 6.2 | 16.7 |
| 15 | 8 | 2.7 | 8.0 | 21.4 | 4.9 | 9.3 |
| 15 | 12 | 4.2 | 8.1 | 8.0 | 2.3 | 5.7 |

TABLE 48
Compound 8a CYCLE 2 - Cohort 5 -
plasma concentrations (ng/mL)

| | | Subject | Subject | Subject | |
|---|---|---|---|---|---|
| Day | Hour | 01007 | 01008 | 02007 | Mean |
| 1 | 0 | 1.1 | 23.8 | 15.6 | 13.5 |
| 1 | 2 | 22.2 | 78.6 | 64.2 | 55.0 |
| 1 | 6 | 2.6 | 22.3 | 7.7 | 10.8 |
| 1 | 12 | 0.6 | 7.7 | 3.1 | 3.8 |

Mean trough concentrations at steady state (C_{min,ss}) are in bold (note: 12-h dosing interval).

Cohort 5: Individual and Mean Plasma Concentrations for Compound 77 are shown in table 49 and 50.

TABLE 49
Compound 77 CYCLE 1 - Cohort 5 -
plasma concentrations (ng/mL)

| | | Subject | Subject | Subject | Subject | |
|---|---|---|---|---|---|---|
| Day | Hour | 01007 | 01008 | 01009 | 02007 | Mean |
| 1 | 0 | BLQ | BLQ | BLQ | BLQ | BLQ |
| 1 | 0.25 | 2.5 | BLQ | BLQ | BLQ | 0.6 |
| 1 | 0.5 | 11.2 | BLQ | 2.6 | 30.4 | 11.0 |
| 1 | 1 | 30.3 | 0.6 | 9.0 | 88.4 | 32.1 |
| 1 | 2 | 13.7 | 1.2 | 97.2 | 62.9 | 43.8 |
| 1 | 4 | 3.7 | 3.2 | 82.0 | 25.6 | 28.6 |
| 1 | 6 | 1.7 | 2.9 | no sample | 14.1 | 6.2 |
| 1 | 8 | 0.7 | 3.2 | no sample | 5.2 | 3.0 |
| 1 | 12 | 0.9 | 2.9 | no sample | 1.5 | 1.7 |
| 1 | 24 | 16.0 | 8.9 | 33.9 | 11.0 | 17.5 |
| 8 | 0 | 18.7 | 5.7 | 23.0 | 12.7 | 15.0 |
| 15 | 0 | 15.1 | 9.2 | 45.6 | 15.9 | 21.4 |
| 15 | 0.25 | 12.8 | 7.5 | 37.4 | 15.1 | 18.2 |
| 15 | 0.5 | 16.0 | 7.5 | 45.4 | 53.6 | 30.6 |
| 15 | 1 | 37.7 | 5.9 | 87.4 | 77.6 | 52.2 |
| 15 | 2 | 26.5 | 8.9 | 110.3 | 49.4 | 48.8 |
| 15 | 4 | 16.3 | 8.1 | 62.0 | 15.1 | 25.4 |
| 15 | 6 | 6.9 | 5.7 | 40.4 | 6.5 | 14.9 |
| 15 | 8 | 3.4 | 6.6 | 26.9 | 4.9 | 10.5 |
| 15 | 12 | 1.9 | 5.5 | 9.8 | 2.3 | 4.9 |

TABLE 50
Compound 77 CYCLE 2 - Cohort 5 -
plasma concentrations (ng/mL)

| | | Subject | Subject | Subject | |
|---|---|---|---|---|---|
| Day | Hour | 01007 | 01008 | 02007 | Mean |
| 1 | 0 | 0.6 | 15.5 | 8.3 | 8.2 |
| 1 | 2 | 23.7 | 46.7 | 25.9 | 32.1 |
| 1 | 6 | 3.7 | 21.1 | 4.8 | 9.9 |
| 1 | 12 | 0.7 | 6.9 | 1.5 | 3.0 |

Mean trough concentrations at steady state (C_{min, ss}) are in bold (note: 12-h dosing interval).

Cohort 5: Summary of Major Pharmacokinetic Data

Table 51 summarizes relevant PK data obtained for Compound 8a. Besides standard PK parameters, this table includes minimal concentrations on each day of dosing, i.e. the 12-hour trough levels.

TABLE 51
Cohort 5 (160 mg BID): Summary of major Compound 8a PK Parameters

| | | | | | | | M/P- |
|---|---|---|---|---|---|---|---|
| | | | | Cmin | | | Ratio |
| | | Cmax | Tmax | (12 h) | AUC12 | T_{1/2} | (via |
| | Subject | (ng/mL) | (h) | (ng/mL) | (h*ng/mL) | (h) | AUCt) |
| C1D1 | 01007 | 66 | 1.0 | 2.2 | 126 | 2.9 | 0.50 |
| | 01008 | 10.5 | 24.0 | 3.9 | 44.2 | NC | 0.67 |
| | 01009 | 257 | 2.0 | NS | 1306 | NC | 0.62 |
| | 02007 | 159 | 1.0 | 0.6 | 314 | 1.5 | 0.86 |
| | Geo Mean | 72.9 | 2.6 | 1.7 | 218 | 2.1 | 0.65 |
| C1D15 | 01007 | 85 | 1.0 | 4.2 | 216 | 2.9 | 0.65 |
| | 01008 | 10.7 | 2.0 | 8.1 | 101 | NC | 0.81 |
| | 01009 | 242 | 1.0 | 8.0 | 917 | 2.3 | 0.62 |
| | 02007 | 233 | 0.5 | 2.3 | 361 | 4.1 | 0.61 |
| | Geo Mean | 84.6 | 1.0 | 5.0 | 292 | 3.0 | 0.67 |
| C2D1 | 01007 | 22.2 | 2.0 | 0.6 | 83 | NC | 1.12 |
| | 01008 | 78.6 | 2.0 | 7.7 | 394 | NC | 0.71 |
| | 01009 | NS | NS | NS | NS | NS | NS |
| | 02007 | 64.2 | 2.0 | 3.1 | 256 | NC | 0.45 |
| | Geo Mean | 48.2 | 2.0 | 2.5 | 203 | | 0.71 |

NC: not calculated;
NS: No Sample;
M/P: Metabolite-to-Parent

Compound 8a showed measurable concentrations in all subjects at the dose of 160 mg BID. The inter-subject variability was high on all days with subjects showing exposures deviations from the geomean of up to ˜5-fold. The overall plasma exposures achieved were lower than those of Cohort 4. For example, the 24-hour AUC at steady state of ˜600 h*ng/mL for Cohort 5 is approximately 3 times lower compared to the same of Cohort 4. The decrease in exposure in Cohort 5 compared to lower doses had no negative influence on the metabolite to parent ratio on all days. M/P-ratios were stable indicating that metabolite Compound 77 plasma exposure was high with approximately 70% of parent Compound 8a exposure (same range as for all other cohorts).

The data further indicate that there may be considerable re-precipitation of dissolved Compound 8a in the small intestine at this dose level since lower doses (Cohort 3 and Cohort 4) resulted in higher exposures on Day 1 and at steady state.

### Summary of PK Parameters

QD Administration: Cohorts 1-2

Table 52 summarizes all major PK parameters obtained for the once daily (QD) administration Cohorts 1 and 2 in a way that allows direct comparison of mean exposures, accumulation following multiple dosing as well as dose-proportionality.

TABLE 52
QD Cohorts: Summary of mean EOS100850 PK Parameters

| | | | | | | | M/P- |
|---|---|---|---|---|---|---|---|
| | | | | Cmin | | | Ratio |
| | | Cmax | Tmax | (24 h) | AUC24 | T_{1/2} | (via |
| | Subject | (ng/mL) | (h) | (ng/mL) | (h*ng/mL) | (h) | AUCt) |
| C1D1 | Cohort 1 | 49 | 1.0 | 0.4 | 152 | 4.1 | 0.70 |
| | Cohort 2 | 218 | 0.8 | 0.7 | 518 | 3.6 | 0.73 |
| C1D15 | Cohort 1 | 102 | 1.3 | 0.5 | 254 | 2.1 | 0.68 |
| | Cohort 2 | 213 | 2.0 | 3.4 | 694 | 2.5 | 0.53 |

| | | | | |
|---|---|---|---|---|
| | | | | Comment |
| D 15/D 1 AR | Cohort 1 | 2.1 | 1.7 | overall little or |
| D 15/D 1 AR | Cohort 2 | 1.0 | 1.3 | no accumulation |

| | | | |
|---|---|---|---|
| DP on D 1 | 4.5 | 3.4 | good dose-proportionality |
| DP at Steady State (D 15) | 2.1 | 2.7 | in particular at steady state |
| | | | (theoretical = 2.0) |

AR: Accumulation Ratio;
DP: Dose-Proportionality

FIG. 27 provides mean plasma concentration versus time profiles for the QD cohorts on Day 1 and Day 15.

BID Administration: Cohorts 3-5

Table 53 summarizes all major PK parameters obtained for the twice daily (BID) administration Cohorts 3, 4 and 5 in a way that allows direct comparison of mean exposures, accumulation following multiple dosing as well as dose-proportionality. It is of note that the somewhat lower than expected 12-h AUC of Cohort 4 at steady state is a result of low exposure of one subject (S3004). If data for S3004 on Day 15 would be excluded (the subject had normal exposure on Day 1), the dose-proportionality between Cohort 3 and Cohort 4 on Day 15 would be close to theoretical.

TABLE 53
BID Cohorts: Summary of mean Compound 8a PK parameters

| | | | | | | | M/P- |
|---|---|---|---|---|---|---|---|
| | | | | Cmin | | | Ratio |
| | | Cmax | Tmax | (12 h) | AUC12 | T_{1/2} | (via |
| | Subject | (ng/mL) | (h) | (ng/mL) | (h* ng/mL) | (h) | AUCt) |
| C1D1 | Cohort 3 | 148 | 1.0 | 0.6 | 306 | 1.7 | 0.66 |
| | Cohort 4 | 388 | 0.8 | 2.7 | 811 | 1.7 | 0.70 |
| | Cohort 5 | 73 | 2.6 | 1.7 | 218 | 2.1 | 0.65 |
| C1D15 | Cohort 3 | 96 | 1.0 | 3.1 | 299 | 3.2 | 0.88 |
| | Cohort 4 | 169 | 0.7 | 5.0 | 447 | 3.9 | 0.70 |
| | Cohort 5 | 85 | 1.0 | 5.0 | 292 | 3.0 | 0.67 |
| | | | | | | Comment | |
| D 15/D 1 AR | Cohort 3 | 0.6 | | | 1.0 | overall little or | |
| D 15/D 1 AR | Cohort 4 | 0.4 | | | 0.6 | no accumulation | |
| D 15/D 1 AR | Cohort 5 | 1.2 | | | 1.3 | | |

| | | | |
|---|---|---|---|
| DP* or D 1 | 2.6 | 2.6 | good dose-proportionality |
| DP* at Steady State(D 15) | 1.8 | 1.5 | (theoretical = 2.0) |

AR: Accumulation Ratio;
DP: Dose-Proportionality;
*DP only calculated for Cohort 3 to Cohort 4 due to reduced exposure in Cohort 5

FIG. 28 provides mean plasma concentration versus time profiles for the BID cohorts on Day 1 and Day 15.

To sum up, Compound 8a demonstrated safe profile with good target coverage and initial clinical benefit in multiple indications. Compound 8a monotherapy was well tolerated with no treatment-emergent adverse events higher than grade 2 and no dose-limiting toxicities observed. The most common adverse events which were considered at least possibly related to treatment were fatigue, transaminase elevation, decreased appetite and diarrhea. Asymptomatic interstitial pneumonitis was observed in one patient. The 80 mg BID dose, which provided the highest exposure, was selected as the recommended phase 2 dose.

### INCORPORATION BY REFERENCE

The entire disclosures of all patent and non-patent publications cited herein are each incorporated by reference in their entireties for all purposes.

Other Embodiments

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

What is claimed is:

1. A method of treating a subject having cancer comprising administering to the subject a pharmaceutical composition comprising (a) therapeutically effective amount of compound of Formula (I) or a pharmaceutically acceptable salt or solvate thereof, 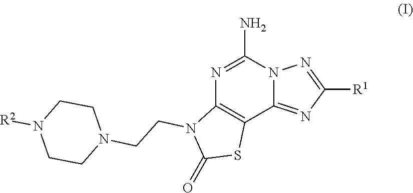
wherein:
the therapeutically effective amount is from about 20 mg per day to about 320 mg per day;
R¹ represents 5- or 6-membered heteroaryl or 5- or 6-membered aryl, wherein heteroaryl or aryl groups are optionally substituted by one or more substituent selected from C1-C6 alkyl (preferably methyl) and halo (preferably fluoro or chloro); preferably R¹ represents 5-membered heteroaryl; more preferably R¹ represents furyl;
R² represents 6-membered aryl or 6-membered heteroaryl,
wherein heteroaryl or aryl groups are optionally substituted by one or more substituent selected from halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino and alkylsulfonealkyl;
said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
or the heteroaryl or aryl groups are optionally substituted with two substituents that form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
(b) one or more pharmaceutically acceptable carrier, diluent, excipient and/or adjuvant, and
(c) optionally a lipid carrier.

2. The method according to claim 1, wherein the compound is of Formula (Ia) 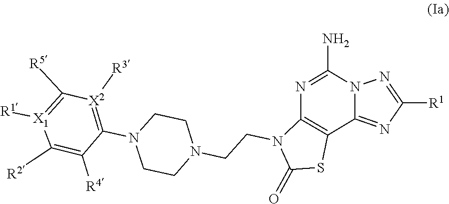
or a pharmaceutically acceptable salt or solvate thereof, wherein:
R¹ is as defined in claim 1;
X¹ and X² represent each independently C or N;
R^{1′} is absent when X¹ is N; or when X¹ is C, R^{1′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino or alkylsulfonealkyl;
said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
R^{2′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino, or alkylsulfonealkyl;
said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
or R^{1′} and R^{2′} form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
R^{3′} is absent when X² is N; or when X² is C, R^{3′} represents H or halo, preferably H or F;
R^{4′} represents H or halo, preferably H or F; and
R^{5′} represents H or halo, preferably H or F.

3. The method according to claim 1 or claim 2, wherein the compound is of Formula (Ia-1) 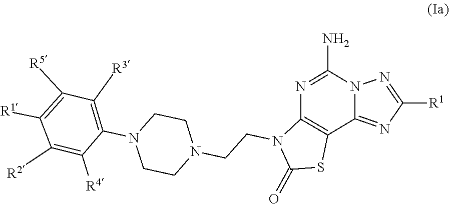
or a pharmaceutically acceptable salt or solvate thereof, wherein R¹, R^{1′}, R^{2′}, R^{3′}, R^{4′} and R^{5′} are as defined in claim 2.

4. The method to any one of claims 1 to 3, wherein the compound is of Formula (Ia-1a) 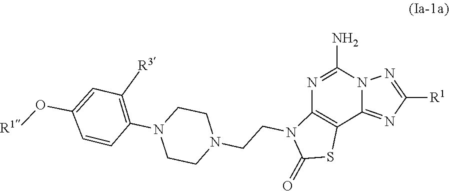
or a pharmaceutically acceptable salt or solvate thereof, wherein:
R¹ and R^{3′} are as defined in claim 2; and
R^{1″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

5. The method according to any one of claims 1 to 3, wherein the compound is of Formula (Ia-1b) 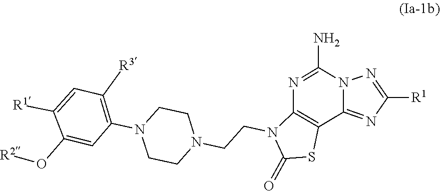
or a pharmaceutically acceptable salt or solvate thereof, wherein:
R¹ and R^{3′} are as defined in claim 2;
R^{1′} represents H or halo, preferably H or F; and
R^{2″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

6. The method according to any one of claims 1 to 3, wherein the compound is of Formula (Ia-1c) or (Ia-1d) 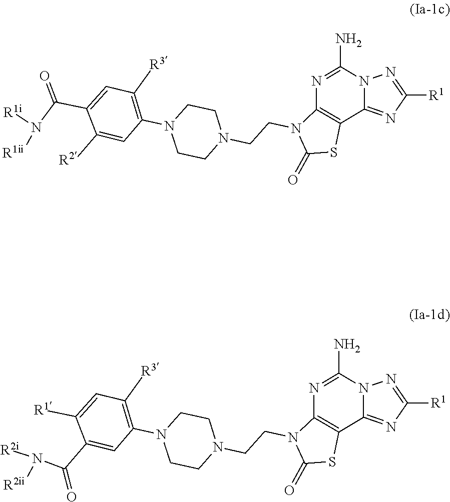
or a pharmaceutically acceptable salt or solvate thereof, wherein:
R¹ and R^{3′} are as defined in claim 2;
R^{1′} represents H or halo, preferably H or F;
R^{2′} represents H or halo, preferably H or F;
R¹ⁱ and R¹ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl; and
R²ⁱ and R²ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl.

7. The method according to any one of claims 1 to 6, wherein the compound of Formula (I) is selected from the group consisting of:
3-(2-(4-(4-((1H-1,2,3-triazolo-4yl)methoxy-2fluorophenyl)piperazine-1-yl)ethyl)-5-amino-(8-(furan-2-yl)thiazolo[5,4-e][1,2,4]triazolo[1,5-c]pyrimidine-2(3H)-one;
5-((4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)methyl)-1,3,4-oxadiazol-2(3H)-one;
5-amino-3-(2-(4-(3-fluoropyridin-4-yl)piperazin-1-yl)ethyl)-8-(furan-2-yl)thiazolo[5,4-e][1,2,4]triazolo[1,5-c]pyrimidin-2(3H)-one;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)acetamide;
(S)-5-amino-3-(2-(4-(2-fluoro-4-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2-fluoro-4-(2-(methylsulfinyl)ethoxy)phenyl)-piperazin-1-yl)ethyl)-8-(furan-2-yl)thiazolo[5,4-e][1,2,4]triazolo[1,5-c]pyrimidin-2(3H)-one;
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
5-amino-8-(furan-2-yl)-3-(2-(4-(4-(2-hydroxyethoxy)phenyl)piperazin-1-yl)ethyl)thiazolo[5,4-e][1,2,4]triazolo[1,5-c]pyrimidin-2(3H)-one;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)phenoxy)acetic acid;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)phenoxy)acetamide;
5-amino-3-(2-(4-(4-(2,3-dihydroxypropoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(4-(2-aminoethoxy)phenyl)piperazin-1-yl)ethyl)-8-(furan-2-yl)thiazolo[5,4-e][1,2,4]triazolo[1,5-c]pyrimidin-2(3H)-one;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-methylbenzamide;
5-amino-8-(furan-2-yl)-3-(2-(4-(4-(2-morpholinoethoxy)phenyl)piperazin-1-yl)ethyl)thiazolo[5,4-e][1,2,4]triazolo[1,5-c]pyrimidin-2(3H)-one;
5-amino-3-(2-(4-(4-(2-(dimethylamino)ethoxy)phenyl)piperazin-1-yl)ethyl)-8-(furan-2-yl)thiazolo[5,4-e][1,2,4]triazolo[1,5-c]pyrimidin-2(3H)-one;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)benzenesulfonamide;
4-(4-(2-(5-amino-8-(furan-2-yl)-2-oxothiazolo[5,4-e][1,2,4]triazolo[1,5-c]pyrimidin-3(2H)-yl)ethyl) piperazin-1-yl)-N-methylbenzenesulfonamide;
5-amino-8-(furan-2-yl)-3-(2-(4-(4-(methylsulfonyl) phenyl)piperazin-1-yl)ethyl)thiazolo[5,4-e][1,2,4]triazolo[1,5-c]pyrimidin-2(3H)-one;
5-amino-8-(furan-2-yl)-3-(2-(4-(4-(methylsulfinyl)phenyl)piperazin-1-yl)ethyl)thiazolo[5,4-e][1,2,4]triazolo[1,5-c]pyrimidin-2(3H)-one;
3-(4-(2-(5-amino-8-(furan-2-yl)-2-oxothiazolo[5,4-e][1,2,4]triazolo[1,5-c]pyrimidin-3(2H)-yl)ethyl)piperazin-1-yl)benzamide;
5-amino-8-(furan-2-yl)-3-(2-(4-(3-(2-hydroxyethoxy) phenyl)piperazin-1-yl)ethyl)thiazolo[5,4-e][1,2,4]triazolo[1,5-c]pyrimidin-2(3H)-one;
5-amino-3-(2-(4-(2-fluoro-4-(2-oxo-2-(piperazin-1-yl)ethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(piperidin-4-ylmethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(piperazine-1-carbonyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(2-(piperazin-1-yl)ethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(piperazin-1-ylsulfonyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(methylsulfonyl)phenyl) piperazin-1-yl)ethyl)-8-(furan-2-yl)thiazolo[5,4-e][1,2,4]triazolo[1,5-c]pyrimidin-2(3H)-one;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-aminoethyl)-3-fluorobenzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylamino)ethyl)benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-3-fluorobenzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-hydroxyethyl)benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-3-fluorobenzamide;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)acetic acid;
2-(4-(4-(2-(5-amino-8-(furan-2-yl)-2-oxothiazolo[5,4-e][1,2,4]triazolo[1,5-c]pyrimidin-3(2H)-yl)ethyl) piperazin-1-yl)-3,5-difluorophenoxy)acetic acid;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)propanoic acid;
(S)-2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)propanoic acid;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-2-methylpropanoic acid;
3-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenyl)propanoic acid;
4-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)butanoic acid;
2-(3-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,6-difluorophenoxy)acetic acid;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)acetic acid;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorobenzoic acid;
2-((2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)ethyl)amino)acetamide;
2-((2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)ethyl)(methyl)amino)acetamide;
5-amino-3-(2-(4-(2-fluoro-4-(piperidin-4-yloxy)phenyl)piperazin-1-yl)ethyl)-8-(furan-2-yl) thiazolo[5,4-e][1,2,4]triazolo[1,5-c]pyrimidin-2(3H)-one;
5-amino-3-(2-(4-(2-fluoro-4-(pyrrolidin-3-yloxy)phenyl)piperazin-1-yl)ethyl)-8-(furan-2-yl)thiazolo[5,4-e][1,2,4]triazolo[1,5-c]pyrimidin-2(3H)-one;
3-(2-(4-(4-((1H-1,2,4-triazol-3-yl)methoxy)-2-fluorophenyl)piperazin-1-yl)ethyl)-5-amino-8-(furan-2-yl)thiazolo[5,4-e][1,2,4]triazolo[1,5-c]pyrimidin-2(3H)-one;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-(2-(methylamino)ethyl)acetamide;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-(2-(dimethylamino)ethyl)acetamide;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-(2-aminoethyl)acetamide;
(R)-2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)propanoic acid;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)acetamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-methyl-N-(2-(methylamino)ethyl)benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-3-fluoro-N-methylbenzamide;
(R)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(1-(dimethylamino)propan-2-yl)-3-fluorobenzamide;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-methyl-N-(2-(methylamino)ethyl)acetamide;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-2-methylpropanoic acid;
(S)-2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)propanoic acid;
(R)-2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)propanoic acid;
2-(5-4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-N-(2-(methylamino)ethyl)acetamide;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-N-(2-(dimethylamino)ethyl)acetamide;
5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-2,4-difluoro-N-methylbenzamide;
4-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)butanoic acid;
3-(2-(4-(5-((1H-tetrazol-5-yl)methoxy)-2,4-difluorophenyl)piperazin-1-yl)ethyl)-5-amino-8-(furan-2-yl)thiazolo[5,4-e][1,2,4]triazolo[1,5-c]pyrimidin-2(3H)-one;
5-amino-3-(2-(4-(2-fluoro-4-((1-methyl-1H-1,2,4-triazol-3-yl)methoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-((1-methyl-1H-1,2,4-triazol-3-yl)methoxy)phenyl)piperazin-1-yl)ethyl)-8-(furan-2-yl)thiazolo[5,4-e][1,2,4]triazolo[1,5-c]pyrimidin-2(3H)-one;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methyl (oxetan-3-yl)amino)ethyl)benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-((2-hydroxyethyl)amino)ethyl)benzamide;
2-amino-N-(2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)ethyl)acetamide;
(S)-2-amino-N-(2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)ethyl)-3-methylbutanamide;
ethyl 2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)acetate;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)acetonitrile;
5-amino-8-(furan-2-yl)-3-(2-(4-(pyridin-4-yl)piperazin-1-yl)ethyl)thiazolo[5,4-e][1,2,4]triazolo[1,5-c]pyrimidin-2(3H)-one;
5-amino-8-(furan-2-yl)-3-(2-(4-(pyrimidin-4-yl)piperazin-1-yl)ethyl)thiazolo[5,4-e][1,2,4]triazolo[1,5-c]pyrimidin-2(3H)-one;
5-amino-3-(2-(4-(2,4-difluoro-5-(2-(methylsulfonyl)ethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(2-(methylsulfonyl)ethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(6-fluoro-2-oxoindolin-5-yl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(S-methylsulfonimidoyl)phenyl)piperazin-1-yl)ethyl)-8-(furan-2-yl)thiazolo[5,4-e][1,2,4]triazolo[1,5-c]pyrimidin-2(3H)-one;
5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-2,4-difluorobenzamide;
5-amino-3-(2-(4-(5-fluoro-2-methylpyridin-4-yl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3R,4R)-4-hydroxytetrahydrofuran-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3S,4S)-4-hydroxytetrahydrofuran-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(2-hydroxy-2-methylpropoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(2-hydroxypropan-2-yl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(3,3,3-trifluoro-2-hydroxypropoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-5-(2-hydroxyethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(morpholin-2-ylmethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(morpholin-3-ylmethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((3R,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((3S,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(2,4-difluoro-5-((2-oxopyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-((2-oxopyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-N-(2-morpholinoethyl)acetamide;
5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(morpholin-3-ylmethyl)benzamide;
5-amino-3-(2-(4-(2-fluoro-4-(morpholin-3-ylmethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(morpholin-2-ylmethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3R,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3R,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3S,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-(2-morpholinoethyl)acetamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-morpholinoethyl)benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(morpholin-3-ylmethyl)benzamide;
5-amino-3-(2-(4-(4-(azetidin-3-yloxy)phenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((1s,4s)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((1r,4r)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one;
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(2-(methylsulfinyl)ethyl)benzamide;
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(2-(methylsulfinyl)ethyl)benzamide;
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-methyl-N-(2-(methylsulfinyl)ethyl)benzamide;
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-methyl-N-(2-(methylsulfinyl)ethyl)benzamide;
5-amino-3-(2-(4-(2,4-difluoro-5-(1-oxidothiomorpholine-4-carbonyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(1-oxidothiomorpholino)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((1s,4s)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((1r,4r)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one;
(S)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide;
(R)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide;
5-amino-3-(2-(4-(2-fluoro-4-(1-oxidothiomorpholine-4-carbonyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(1-oxidothiomorpholino)phenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(5-(2,3-dihydroxypropoxy)-2,4-difluorophenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(5-(2,3-dihydroxypropoxy)-2,4-difluorophenyl)piperazin-1-yl)ethyl)-8-(furan-2-yl)thiazolo[5,4-e][1,2,4]triazolo[1,5-c]pyrimidin-2(3H)-one;
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-2,4-difluorobenzamide;
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-2,4-difluorobenzamide;
5-amino-3-(2-(4-(4-(azetidin-3-yloxy)-2-fluorophenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(5-(azetidin-3-yloxy)-2,4-difluorophenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(2,4-difluoro-5-(3-(methylsulfinyl)propoxy)phenyl)piperazin-1-yl)ethyl)-8-(furan-2-yl)thiazolo[5,4-e][1,2,4]triazolo[1,5-c]pyrimidin-2(3H)-one;
and pharmaceutically acceptable salts or solvates thereof.

8. The method according to any one of claims 1 to 7, wherein the compound of Formula (I) is selected from:
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one; and
(S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one.
5-amino-3-(2-(4-(2,4-difluoro-5-(2-(methylsulfonyl)ethoxy)phenyl)piperazin-1-yl)ethyl)-8-(furan-2-yl)thiazolo[5,4-e][1,2,4]triazolo[1,5-c]pyrimidin-2(3H)-one

9. The method according to any one of claims 1 to 8, wherein the lipid carrier comprises lauroyl polyoxyl-32 glycerides.

10. The method according to any one of claims 1 to 8, wherein the pharmaceutical composition is administered in the form of a tablet, a suspension, granules or a capsule.

11. The method according to any one of claims 1 to 8, wherein the pharmaceutical composition is administered orally.

12. The method according to any one of claims 1 to 8, wherein the therapeutically effective amount is administered in separate administrations of 2, 3, 4, or 6 equal doses.

13. The method according to any one of claims 1 to 8, wherein the therapeutically effective amount is administered as a single dose.

14. The method according to any one of claims 1 to 8, wherein the therapeutically effective amount is about 160 mg per day, wherein the therapeutically effective amount is administered in separate administrations of 2, 3, 4, or 6 equal doses.

15. The method according to any one of claims 1 to 8, wherein the therapeutically effective amount is about 160 mg per day, wherein the therapeutically effective amount is administered in separate administrations of 2 equal doses, whereby each dose is about 80 mg.

16. The method according to any one of claims 1 to 8, wherein the subject is a human

17. The method according to claim 16, wherein the human subject is older than 18 years of age.

18. The method according to any one of claim 16 or 17, wherein the human subject has a confirmed metastatic solid tumor.

19. The method according to claim 18, wherein the metastatic solid tumor is confirmed by international RECIST v1.1 criteria.

20. The method according to any one of claims 1 to 8, wherein the pharmaceutical composition is administered to the subject for at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the subject.

21. The method according to any one of claims 1 to 8, further comprising orally administering a beverage with a pH lower than or equal to about 3, about 5, or about 7.

22. The method according to the claim 21, wherein the beverage and the pharmaceutical composition are to be administered to a subject in need thereof sequentially and/or concurrently.

23. The method according to any one of claims 1 to 8, wherein the level of pCREB in T-cells of the subject is decreased after administering to the subject the pharmaceutical composition.

24. The method according to any one of claims 1 to 8, wherein the level of TNFα in the subject is increased after administering to the subject the pharmaceutical composition.

25. The method according to any one of claims 1 to 8, wherein the salt of the compound is the hydrochloride or esylate salt.

26. The method according to any one of claims 1 to 8, the pharmaceutical composition further comprises PEG 400, PEG 2000 and/or caprylic acid.

27. The method according to any one of claims 1 to 8, the pharmaceutical composition further comprises an antioxidant; preferably the antioxidant is selected from butylated hydroxytoluene (BHT), butylated hydroxyanisole (BHA), citric acid, sodium metabisulfite, ascorbic acid, methionine and vitamin E; more preferably the antioxidant is BHT.

28. The method according to any one of claims 1 to 8, the pharmaceutical composition further comprises a wetting agent; preferably the wetting agent is selected from sodium lauryl sulphate, vitamin E TPGS, sodium docusate, polysorbate 80 and poloxamer 407; more preferably the wetting agent is sodium lauryl sulphate.

29. The method according to any one of claims 1 to 8, the pharmaceutical composition further comprises a precipitation inhibitor, preferably the precipitation inhibitor is selected from hydroxypropylmethylcellulose, methylcellulose, polyvinylpyrrolidone, polyvinylpyrrolidone polyvinylacetate copolymer; more preferably hydroxypropylmethylcellulose.

30. The method according to any one of claims 1 to 8, wherein the compound of Formula (I) is present in an amount ranging from 1% to 20% w/w, preferably from 5% to 15% w/w, more preferably about 10% w/w.

31. The method according to any one of claims 1 to 8, wherein lauroyl polyoxyl-32 glycerides is present in an amount ranging from 55% to 99% w/w, preferably from 60% to 95% w/w, more preferably from 70% to 85% w/w.

32. The method according to any one of claims 1 to 8, wherein the pharmaceutical composition is formulated as capsules, wherein the capsule shells are constructed from gelatin and wherein additional components are optionally included in the capsule shells, such as for example polyethylene glycol and sodium lauryl sulphate.

33. The method according to any one of claims 1 to 8, wherein the cancer is selected from breast, bladder, carcinoid, cervical, colorectal, endometrial, glioma, head and neck, liver, lung, melanoma, ovarian, pancreatic, prostate, metastatic castrate resistant prostate, renal, gastric, sarcoma, thyroid and urothelial cancers.

34. The method according to any one of claims 1 to 8, further comprising administering at least one pharmaceutically active ingredient.

35. The method according to claim 34, the pharmaceutically active ingredient is selected from anticancer agents.

36. The method according to claim 35, wherein the anticancer agent comprises pembrolizumab.

37. The method according to claim 34, wherein the anticancer agent is selected from immunotherapeutic agents, chemotherapeutic agents, antiangiogenic agents, multidrug resistance-associated proteins inhibitors, radiotherapeutic agents, and any combination thereof.

38. The method according to claim 37, wherein the immunotherapeutic agent is selected from checkpoint inhibitors, checkpoint agonists, IDO inhibitors, PI3K inhibitors, adenosine receptor inhibitors, adenosine-producing enzymes inhibitors, immune cells, therapeutic vaccines, or any combination thereof.

39. The method according to claim 38, wherein the checkpoint inhibitor is an inhibitor of a checkpoint protein selected from PD-1, PD-L1, CTLA-4 and TIGIT.

40. The method according to claim 39, wherein the inhibitor of PD-1 is an anti-PD-1 antibody; the inhibitor of PD-L1 is an anti-PD-L1 antibody; the inhibitor of CTLA-4 is an anti-CTLA-4 antibody and the inhibitor of TIGIT is an anti-TIGIT antibody.

41. The method according to claim 37, wherein the chemotherapeutic agent is selected from anticancer alkylating agents, anticancer antimetabolites, anticancer antibiotics, plant-derived anticancer agents, anticancer platinum coordination compounds and any combination thereof.

42. The method according to claim 41, wherein the anticancer antibiotic is doxorubicin; and the anticancer platinum coordination compound is oxaliplatin.

43. The method according to any one of claims 35 to 42, wherein the anticancer agent and the pharmaceutical composition are to be administered to a patient in need thereof sequentially and/or concurrently.

44. The method according to any one of claims 35 to 42, wherein the anticancer agent and the pharmaceutical composition are to be administered to a subject in need thereof via different administration routes.
